SEQUENCE LISTING (618 sequences)

SEQ ID NO: 1 - protein, 252 aa, Phakopsora pachyrhizi
MLLSLGFKQFLIISFFIFRTLNATSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQSSSIIVTATLVNDGRINGIPLSIPTGTSNSSSTSKGSNNDTAPNTLPTTAPTPFQNTNTSPINNDNSNRQNSGSNGLTIWFDRKRMKFDYRMFLDSNLLFFICFTLLLV

SEQ ID NO: 2 - protein, 233 aa, Phakopsora pachyrhizi
MLNATSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQSSSIIVTATLVNDGRINGIPLSIPTGTSNSSSTSKGSNNDTAPNTLPTTAPTPFQNTNTSPINNDNSNRQNSGSNGLTIWFDRKRMKFDYRMFLDSNLLFFICFTLLLV

SEQ ID NO: 3 - protein, 252 aa, Phakopsora pachyrhizi
MLLSLGFKQFLIISFFIFRTLNATSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQLSSIIVTATLVNDGRINGIPLSIPTGTSNSSSTSKGSNNDTAPNTLPTTAPTPFQNTNTSPINNDNSNRQNSGSNGLTIWFDRKRMKFDYRMFLDSNLLFFICFTLLLV

SEQ ID NO: 4 - protein, 230 aa, Phakopsora pachyrhizi
MTSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQLSSIIVTATLVNDGRINGIPLSIPTGTSNSSSTSKGSNNDTAPNTLPTTAPTPFQNTNTSPINNDNSNRQNSGSNGLTIWFDRKRMKFDYRMFLDSNLLFFICFTLLLV

SEQ ID NO: 5 - protein, 252 aa, Phakopsora pachyrhizi
MLMSLGFKQFLIISFFIFRTLNATSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQSSSIIVTATLVNDGRINGIPLSIPTGSSNSSSTSKGSNNDTAPNTLPTTAPTPFQNTNTSPINNDNSNRQNSGSNRLTIWFDRKRMKFDYRMFLDSNLLFFICFTLLLV

SEQ ID NO: 6 - protein, 230 aa, Phakopsora pachyrhizi
MTSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQSSSIIVTATLVNDGRINGIPLSIPTGSSNSSSTSKGSNNDTAPNTLPTTAPTPFQNTNTSPINNDNSNRQNSGSNRLTIWFDRKRMKFDYRMFLDSNLLFFICFTLLLV

SEQ ID NO: 7 - protein, 252 aa, Phakopsora pachyrhizi
MLMSLGFKQFLIISFFIFRTLNATSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQSSSIIVTATLVNDGRINGIPLSIPTGSSNSSSTSKGSNNDTTPNTLPTTAPTPFQNTNTSPINNDNSNRQNSGSNRLTIWFDRKRMKFDYRMFLDSNLLFFICFTLLLV

SEQ ID NO: 8 - protein, 230 aa, Phakopsora pachyrhizi
MTSDIKTTDENKKLLKEVGGEDVEPISYENQGDENHLNKRQLSLADLPPCGQTCYVGSLTVSGGCSITDFPCLCQSKSFMTQAQECFKQKCSQSELSTVLSLSLQTCQVLNIPFYRIPEFQSSSIIVTATLVNDGRINGIPLSIPTGSSNSSSTSKGSNNDTTPNTLPTTAPTPFQNTNTSPINNDNSNRQNSGSNRLTIWFDRKRMKFDYRMFLDSNLLFFICFTLLLV

SEQ ID NO: 9 - protein, 925 aa, Cajanus cajan
MADSVVSFVLDHLSQLVEHEARLLSGVEDKVKSLERELQMINVILRTTNSNNDIQKTVVSQIRDVAHEAEDVIDTYVAKVALHNRRTMLGRLLHGVDQTKLLHDVSEKIDEIITTLNQIRENKIKYSEFQERNHQSIAEEEEEEKERERLLHKLRRNVEEEHVVGFIRGSQAIIKLLKEGGSRRNVVSIIGMGGLGKTTLARKVYNDSKVKQGFSCCVWVYVSNECRAKELLLSLLKHLRPNLETELQEENNKGKKFTEEQDIFNLSVEELKKLVRQYLERKTRYLVVLDDLWKTQDWDEVQDAFPDNNRGNRILITSRLKEVALHTSLHPPYYLQFLSQEESWELFRRKVFRGEECPFELEPLGKQIVASCRGLPLSIVVLAGLLANKEKSHREWSKVVGHVNWYLTQDETQVKDIVLKLSYDNLPRRLKPCFLYFGIFPEDFEIPVRPLLQQWVAEGFIQETRNRDPDDVAEDYLYELIDRSLVQVAAIKTSGGVKTCHIHDLLRDLCVSQSKGDKIFEVCSDNDIQILTKPRRLSFHCDMGHYISSSNKDHSCIRSLFFFGIYSNFTGNEWEWLFKGFKLVRVLELGKNHCAGKIPSNLGDFIHLRYLRIDSNFGIIIPTSILTLQNLQTVDLGNWFREIPICFPAQMWKLKHLRHLYGQGPVKLQGHYSGSNEVMWNLRTIFPIDIDTQTLSLMKKGSFPNLVKLGLSINSDRQGKWPKLLQSLQELSHLNILKICLRGDFDASIGTVSSIWRFGCEPQELLQSLGLLTHITTLKITNICSLMITVPPNVTKLTLRGISSITREGLNALRNHTKLQILSLYGDYGSNINLNCVVGGFPQLQVLQLKKFTSVNWKLGNGAMPRLHTLVIINCQSLDDLPNELWSLTAFRKLHVKQPSQPMLRMLRDLKIKDRVQVIVDDHDN

SEQ ID NO: 10 - protein, 218 aa, Phakopsora pachyrhizi
MSLSFKALLVFLPLIFFYLVLAVKQPSDQTTHSTQPQKHNTHQTSINSKRELQQQLSQFNPHHQSLASSTRMSSVNDRTASPITDIDSNTNVPTAAKGEEESTKQKLEDQPAGDGTKASLSANPGESQQSDEKNLKSQNVSPSQEHMSRYIVTFSRETTSAQLKEYADKIIQRGGKINHTYDSAILKGFAVSISDSLVTTLDDDPNVKSVEPDGEVHI

SEQ ID NO: 11 - protein, 218 aa, Phakopsora pachyrhizi
MSLSFKALLVFLPVIFFYLVLAVKQPSDQTTHSTQPQKHNTHQTSINSKRELQQQLSQFNPHHQSLASSTRMSSVNDRTASPITDIDSNTNVPTAAKGEEESTKQKLEDQPAGDGTKASLSANPGESQQSDEKNLKSQNVSPSQEHMSRYIVTLSRETTSAQLKEYADKIIQRGGKINHTYDSAILKGFAVSISDSLVTTLDDDPNVKSVEPDGEVHI

SEQ ID NO: 12 - protein, 97 aa, Phakopsora pachyrhizi
MRFSASIPLFLALVFGMLASVSLGLSQQDEVRMLLSLPTCMKNCYLQNLNAAPSCKGRANCLCSSQIYTYKVKACATGCDLSEQLQGLSYSRTVCNQ

SEQ ID NO: 13 - protein, 127 aa, Phakopsora pachyrhizi
MFGLKAIIIATGLIYQVTAAANTSATTAPGQDQGQGQGPESGGCAAKCMAAKLDDAAKWFGPGNLASYCQHAEFITAYDTCLGDNCANHEELDAGKKSGREACAAAGVTSPGSIGFCCCQQYSFNTH

SEQ ID NO: 14 - protein, 115 aa, Phakopsora pachyrhizi
MFGLKAIIIATGLIYQVTAAANTSATTAPGQDQGQGQGPESGGCAAKCMAAKLDDAAKWFGPGNLASYCQHAEFITAYDTCLGDNCANHEELDAGKKSGREACAAAGVTSPGSIG

SEQ ID NO: 15 - protein, 145 aa, Phakopsora pachyrhizi
MVFSSLMLIVNILNVFLTLTNQLVQGASVEIGRGLGRRQANLKGMGQATFYTPGLGACGETHTESEMVVAISKDLFQSLGGASSNPNQNPVCGKTVTASYQGKSCSVKIIDLCMSCAQGDLDLSPAAFKQLSDLSAGRISGVTWG

SEQ ID NO: 16 - protein, 215 aa, Phakopsora pachyrhizi
MFGLKAIIIATGLIYQVTAAANTSATTAPGQDQGQGQGPESGGCAAKCMAAKLDDAAKWFGPGNLASYCQHAEFITAYDTCLGDNCANHEELDAGKKSGREACAAAGVTSPGSIAPPTGHTPPTSGNNSLADLPAGATNHSMSTNSTPSATTTNSTLGGTLNNTLHTPNTTSTATNSRFSSAVNSASAAANSTASTLTSSSILLGISSVLVIANL

SEQ ID NO: 17 - protein, 216 aa, Phakopsora pachyrhizi
MFGLKAIIIATGLIYQVTAAANTSATTAPGQDQGQGQGPESGGCAAKCMAAKLDDAAKWFGPGNLASYCQHAEFITAYDTCLGDNCANHEELDAGKKSGREACAAAGVTSPGSIAPPTGHTPPTSGNNSLADLPAGATNHSMSTNSTPSATTTNSTLGGTLNNTLHTPNTTSTATNSRFSSAVNSAASAAANSTASTLTSSSILLGISSVLVIANL

SEQ ID NO: 18 - protein, 97 aa, Phakopsora pachyrhizi
MRFSASIPLFLALVFGMLANVSLGLSQQDEVRMLLSLPTCMKNCYLQNLNAAPSCKGRANCLCSSQIYTYKVKACATGCDLSEQLQGLSYSRTVCNQ

SEQ ID NO: 19 - protein, 161 aa, Phakopsora pachyrhizi
MLFNSNVILFALTFALGSLYQGSEAQDLAGLPNCGQNCLLTAFTTSSDGCTQTDFACLCKSQKFTGTSNSCYSSSCTSTEAATAKSWGAKTCASVGVNVTATLGNTTSVGTVIGNGTNPNTNSTLHNGTTASTHAQNSGGLINVQPIFFMSLVSVLAAVFM

SEQ ID NO: 20 - protein, 188 aa, Phakopsora pachyrhizi
MFGLKTIIVAIAAILQVAEAIDDSGCASKCMAVKLTEAAVWFGQGNLSTYCQHPEFITAYDSCLGDNCTSRDELEVAKRNGRAACAAASINSVANNVSSIGRNNSPADMPAGAGNRSTGSPSPTVPTNTSVNINSTLLTPNSTVNATNSRFSNAPFPAISAAANSTASTITCSSMIIGLTSALVLASL

SEQ ID NO: 21 - protein, 188 aa, Phakopsora pachyrhizi
MFGLKTIIVAIAAILQVAEAIDDSGCASKCMAVKLTEAAVWFGQGNLSTYCQHPEFITAYDSCLGDNCTSRDELEVAKRNGRAACAAASINSVANNVSSIGRNNSPADMPAGAGNRSTGSPSPTVPTNTSVNINSTLLTPNSTVNATNSRFSNAPFPAISAAANSTASTKTCSSMIIGLTSALVLASL

SEQ ID NO: 22 - protein, 252 aa, Phakopsora pachyrhizi
MIVIAQLLITSFLFTLSLIRIPASTNESFSWVDSFPRLASSPQCHLSSASAKVAKGTQPLHKRKSYQNSLLISTTSGNFLGFTNRSVTNNVDLWRGIPYAEPPIGTRRFRAPILAKPPLDDPSAIQAAYEFGNACPQQPTQFVPVPISEDCLSLNIYRPENTEKASAQLPVLLWVHGGGFTMGVSASADGSILVSSSVDLEKPIVFVSINYRLNTFGFLNTDELPLEDLQVGLKDQILSLEWLKKNIANFGG

SEQ ID NO: 23 - protein, 159 aa, Phakopsora pachyrhizi
MGYTRILIILTIFQAFYLTLGDSLTKSIHSRQNSLSTCARECYASALANSSASGSCSQTDNLCLCRRDEFSDNVKDCWDKCTDIEEAAAKTWFKTECAARGVAVTFKNVTSAVSGAASSVANTITNARNSQPKTVPIPVTLALCSVVLYALLGLSEVLS

SEQ ID NO: 24 - protein, 133 aa, Phakopsora pachyrhizi
MGYTRILIILTIFQAFYLTLGDSLTKSIHSRQNSLSTCARECYASALANSSASGSCSQTDNLCLCRRDEFSDNVKDCWDKCTDIEEAAAKTWFKTVEQPTFCPDFLPLLLKLMFWLAHFINKGVRCSRGCCDF

SEQ ID NO: 25 - protein, 159 aa, Phakopsora pachyrhizi; identical to SEQ ID NO: 23

SEQ ID NO: 26 - protein, 249 aa, Phakopsora pachyrhizi
MAFRSFSTSIFALKSLFLIVNPIQTWAAVGLGNIPSCSHLCFDQSSSQSSCSAMTDLKCICHDQGFLTALHSCFQLRCDPDSLPGALRLMNTCKESFGITVESAKPQLGGQSSPLVLYLGAQPPLPNNSSFSTLTSTGFVSSSSGGVNVISPPAPVPARVPAQSTGYVPTTPPELAPSGSALAHSINESGVSAASPSTNAPSRVSNQTPGLAPPPAQATAQNSASLRYSSSLNKPVIMVAASSFAMLFV

SEQ ID NO: 27 - protein, 137 aa, Phakopsora pachyrhizi
MGYTRILIILTIFQAFYLTLGDSLTKSIHSRQNSLSTCARECYASALANSSASGSCSQTDNLCLCRRDEFSDNVKDCWDKCTDIEEAAAKTWFKTECAARGVAVTFKNVTSAVSGHLLWRTRSLTREIRNPRQYLYQ

SEQ ID NO: 28 - protein, 427 aa, Phakopsora pachyrhizi
MQSASKLLINIVLIYNLIVHIACHGFISYCSADGGKFEKAQKTPLSDTSFRGASSNTGWIGSKFIDSESFTCGSSDTPKGTVAPPGGTIFSRPEQSAKKTLKVKAGGKVDIIISGEKGKGFPHPKGHIETFLGNCGPTESDCQNFDASKAKYFKIQSEMDGISNSLRTKYNSELDGNPWGIPIPSNVQPGSYIIRVQMIAFGQSGEVEGNQDQYYVYCGQIFIDDGQKDSTLKSTTQFIQQEPQDLLSFPGAYKTGNIDPKTLPDPINVASSAASSSADDVNSGVPPKAADKDKLQIKSVQPPSISDDGYGGNDQGAETVSKSEPSKIACADKCYKATLPKIKDFAKSCTAEDFNCLCNSPRFIKAYENCCKDNCDEGNVKAAIKEIHSRCDSAGPSRKRSKPSNLHSSTPLSTDRLMHKRRSIRKR

SEQ ID NO: 29 - protein, 541 aa, Phakopsora pachyrhizi
MHSSSVGFLIAALAGAAAASPYQEQDDLTLAPLVTPPPPPMGGPLSQFAIQSGRSPSDYLIKDHYIVVFKKDIHEKHARDHKHKVHKLWKQDRNERIQVQGFGIGDIFAGIKHHFDIGTSVFKGYSIHVPESLISIIRTLPEVAYVERDSKVWINDPYFATNTVPANEPKASSASDVDVEYGAPWGLARISHRHKLKFSTFNVYPFEDPAGKGVDVYVIDTGINIEHEELEGRAVWGKTIPQDADKDLNGHGSHVAGTIASRSYGVAKNATVIAVKVLGAGGSGTMSDVVAGVVWAAESALNKAKTESTKSDSKHKGSVANMSLGGGKSPSLDDAVNAAVETGLHFAVAAGNDNRDACTFSPAAAANPITVGASTIQDERAYFSNHGKCVDIFAPGLNIKSIWNSGPQSTNTISGTSMASPHIAGLAAYFLSIYPESLDLAALGLQEDDDVEDEVVASNGEAYDLRQYFTWNSANSPGQIAFGKFKEAVGFGQAQKTRASRKPLSTTLLKKAMLKLSTPGALADLPAQTVNYLAYNNVTAT

SEQ ID NO: 30 - protein, 215 aa, Phakopsora pachyrhizi
MSLSFKALLVFLPVIFFYLVLAVKQPSDQTTHSTQPQKHNTHQTSINSKRELQQQLSQFNPNHQSLASSTRMSSVNASPITDIDSNTNVPTAAKGEEESTKQKLEDQPAGDGTKASLSANPGESQQSDEKNLKSQNVSPSQEHMSRYIVTLSRETTSAQLKEYADKIIQRGGKINHTYDSAILKGFAVSISDSLVTTLDDDPNVKSVEPDGEVHI

SEQ ID NO: 31 - protein, 218 aa, Phakopsora pachyrhizi
MSLSFKALLVFLPVIFFYLVLAVKQPSDQTTHSTQPQKHNTHQTSINSKRELQQQLSQFNPNHQSLASSTRMSSVNDRTASPITDIDSNTNVPTAAKGEEESTKQKLEDQPAGDGTKASLSANPGESQQSDEKNLKSQNVSPSQEHMSRYIVTLSRETTSAQLKEYADKIIQRGGKINHTYDSAILKGFAVSISDSLVTTLDDDPNVKSVEPDGEVHI

SEQ ID NO: 32 - protein, 529 aa, Phakopsora pachyrhizi
MHSSSVGFLIAALAGAAAASPYQEQDDLTLAPLVTPPPPPMGGPLSQFAIQSGRSPSDYLIKDHYIVVFKKDIHEKHARDHKHKVHKLWKQDRNERIQVQGFGIGDIFAGIKHHFDIGTSVFKGYSIHVPESLISIIRTLPEVAYVERDSKVWINDPYFATNTVPANEPKASSASDVDVEYGAPWGLARISHRHKLKFSTFNVYPFEDPAGKGVDVYVIDTGINIEHEELEGRAVWGKTIPQDADKDLNGHGSHVAGTIASRSYGVAKNATVIAVKVLGAGGSGTMSDVVAGVVWAAESALNKAKTESTKSDSKHKGSVANMSLGGGKSPSLDDAVNAAVETGLHFAVAAGNDNRDACTFSPAAAANPITVGASTIQDERAYFSNHGKCVDIFAPGLNIKSIWNSGPQSTNTISGTSMASPHIAGLAAYFLSIYPESLDLAELGLQEDDDVEDEAVASNGEAYDLRQYFTWNSANSPGQIAFGKFKEAVGFGQAQKTRASRKPLSTTLLKKAMLKLSTPGALAVSHCLS

SEQ ID NO: 33 - protein, 529 aa, Phakopsora pachyrhizi
MHSSSVGFLIAALAGAAAASPYQEQDDLTLAPLVTPPPPPMGGPLSQFAIQSGRSPSDYLIKDHYIVVFKKDIHEKHARDHKHKVHKLWKQDRNERIQVQGFGIGDIFAGIKHHFDIGTSVFKGYSIHVPESLISIIRTLPEVAYVERDSKVWINDPYFATNTVPANEPKASSASDVDVEYGAPWGLARISHRHKLKFSTFNVYPFEDPAGKGVDVYVIDTGINIEHEELEGRAVWGKTIPQDADKDLNGHGSHVAGTIASRSYGVAKNATVIAVKVLGAGGSGTMSDVVAGVVWAAESALNKAKTESTKSDSKHKGSVANMSLGGGKSPSLDDAVNAAVETGLHFAVAAGNDNRDACTFSPAAAANPITVGASTIQDERAYFSNHGKCVDIFAPGLNIKSIWNSGPQSTNTISGTSMASPHIAGLAAYFLSIYPESLDLAALGLQEDDDVEDEVVASNGEAYDLRQYFTWNSANSPGQIAFGKFKEAVGFGQAQKTRASRKPLSTTLLKKAMLKLSTPGALAVSHCLS

SEQ ID NO: 34 - protein, 541 aa, Phakopsora pachyrhizi
MHSSSVGFLIAALAGAAAASPYQEQDDLTLAPLVTPPPPPMGGPLSQFAIQSGRSPSDYLIKDHYIVVFKKDIHEKHARDHKHKVHKLWKQDRNERIQVQGFGIGDIFAGIKHHFDIGTSVFKGYSIHVPESLISIIRTLPEVAYVERDSKVWINDPYFATNTVPANEPKASSASDVDVEYGAPWGLARISHRHKLKFSTFNVYPFEDPAGKGVDVYVIDTGINIEHEELEGRAVWGKTIPQDADKDLNGHGSHVAGTIASRSYGVAKNATVIAVKVLGAGGSGTMSDVVAGVVWAAESALNKAKTESTKSDSKHKGSVANMSLGGGKSPSLDDAVNAAVETGLHFAVAAGNDNRDACTFSPAAAANPITVGASTIQDERAYFSNHGKCVDIFAPGLNIKSIWNSGPQSTNTISGTSMASPHIAGLAAYFLSIYPESLDLAELGLQEDDDVEDEAVASNGEAYDLRQYFTWNSANSPGQIAFGKFKEAVGFGQAQKTRASRKPLSTTLLKKAMLKLSTPGALADLPAQTVNYLAYNNVTAT

SEQ ID NO: 35 - protein, 541 aa, Phakopsora pachyrhizi; identical to SEQ ID NO: 29

SEQ ID NO: 36 - protein, 147 aa, Phakopsora pachyrhizi
MVFSSLMLIVNILNVFLTLTNQLVQGASVEIGRGLGRRQANLKGMGQATFYTPGLGACGETHTESEMVVAISKDLFQSLGGASSNPNQNPVCGKTVTASYQGKSCSVKIIDLCMSCAQGDLDLSPAAFKQLSDLSAGRISGVTKQWG

SEQ ID NO: 37 - protein, 218 aa, Phakopsora pachyrhizi; identical to SEQ ID NO: 31

SEQ ID NO: 38 - protein, 339 aa, Phakopsora pachyrhizi
MVEAKVLVVVASLLFFSFLSASPAGSHVRHLTSSKRPPPGALVVRNQRTRVGEFSTINDAVKELKNKKGPQFIFIHSGTYNEAVHIKYKSALTIQGYSENPLSYEDNQVTVIKPTVASEAGSDSKSSVIWAQVDDFKMYNINAINSYGAGKDKQAVAVTADGDRQMYKSCRFDSYQDTLYIRSTASFFDKCLISGGVDVIFGAGSAWFEKCTIGVKPSPDNGISTITAQKRERGSNSRFVFSRCVVVGLRNSKTGSVYLGRPWSEYASVAFQFCFLPDLINPEGWMSWQPNDPKTRHVKFLEFGNSGDGSRGQRKYGTQARMPFTVDEVLGSFPATWDQ

SEQ ID NO: 39 - protein, 338 aa, Phakopsora pachyrhizi
MVEAKILVVISSLLFFSFLSANPASLHGRDLANSRSPPPGALVVRNQGTRVGEFPTINDAVRELENKKGPQYIFIHSGTYNEAVHIKYKSALTIQGYSENPSSYEDNQVTVIKPTVASEAGSDSKSSVIWAKVDDFKMYNINAINSYGAGKDKQAVAVTADGDRQIYKGCKFDSYQDTLYVRSTASYFDRCSISGGVDFIFGAGSAWFEKCTIGIKPSPNNGISTITAQKRERGGNSWFVFNGCKVVGLGNLKIGSVFLGRPWSEYASVAFQFCFLPDLINPEGWMEWQPSDPKTQHVEFLEYRNSGAGSLGQRKYGTQARRAFTIDEVLGSSPATWG

SEQ ID NO: 40 - protein, 339 aa, Phakopsora pachyrhizi; identical to SEQ ID NO: 38

SEQ ID NO: 41 - protein, 109 aa, Phakopsora pachyrhizi
MLLSLGFKQFLIISFFIFRTLNATSDIKTTDENKKLLKEVGGEDVEPISYENQGDENHLNKRQLSLADLPPCGQTCYVGSLTVSGGCSIVSTCLAKLLIFHAYVKVNRL

SEQ ID NO: 42 - protein, 110 aa, Phakopsora pachyrhizi
MLLSLGFKQFLIISFFIFRTLNATSDIKTTDENKKLLKEVGGEDVEPISYENQGDENHLNKRQLSLADLPPCGQTCYVGSLTVSGGCSIVSTCLAKLYDRNLQINFFNRN

SEQ ID NO: 43 - protein, 427 aa, Phakopsora pachyrhizi; identical to SEQ ID NO: 28

SEQ ID NO: 44 - protein, 155 aa, Phakopsora pachyrhizi
MSLINRSILIVVVVLSLTSLGSVLSADGQKQPNGKMIGLLREIQSLPPCAQQCLRIAPPSGCGQTDIACLCKSEARKNQIKECGDKCKPADNTALSNFGQKTCDTVVKGASTNKDAVAPDEEDSTKKGGAETSNKGPASASSSTGRKKVTLPGRR

SEQ ID NO: 45 - protein, 229 aa, Phakopsora pachyrhizi
SKLFFIWIGLFTISKLSTTLSVSFGKGINPLSLADEGESVLKQFQIFGKNNEVVNVKIVSKFNSDSNSLRFTKRDLLGIEDEKSNEKGNDKVKNQAQNRNYTRVDLTPARPTSETCYSGSFQGPNQSDCDVIFYAQKYNSYGSLTAFPGTFVYVYYSSCVVAFQNPNHSNYSLDYNWAMLGAKAEQIKNRCLRDEEQSIGGSFLFENYLGYTFQNVLISLQRYAGNITA

SEQ ID NO: 46 - protein, 144 aa, Phakopsora pachyrhizi
MVFSSLMLIVNILNVFLTLTNQLVQGASVESRGLGRRQANLKGMGQATFYTPGLGACGETHTESEMVVAISKDLFQSLGGASSNPNQNPVCGKTVTASYQGKSCSVKIIDLCMSCAQGDLDLSPAAFKQLSDLSAGRISGVTWG

SEQ ID NO: 47 - protein, 128 aa, Phakopsora pachyrhizi
MPTFSFTWTDAALFGLTAGLIATIALIGRSSSSQSKRSVLDSSRTNQSQSSKKSKKKNKPKKSPPQPSSQTTIGSNQKSEPRHTDERKASPPKSSNPSPKPISEPKKKKIQQPQIPKLDDNEFPTLAN

SEQ ID NO: 48 - protein, 102 aa, Phakopsora pachyrhizi
MSLASSLSVAFVIALGIGQGTAQVPESAPACVRNCARVKIAEGYCSTPGVLTAAFNQCLSENCNSADAASGAAFANVVCEGANPNSNFLDLSQYSVVDTDII

SEQ ID NO: 49 - protein, 162 aa, Phakopsora pachyrhizi
MLFNSNVILFALTFALGSLYQGSEAQDLAGQPNCGQNCLLTAFTTLSNGCTQTDFACLCKSQKFTGTSNLRYSLSCTSTEAATTKSWGAKTCASVGVNVTATLGNTTSVSSDKQQQQQQSSLSSLLAPLSLLPAEVGAGGLVPPLLEQADSDVVNTSKLSTS

SEQ ID NO: 50 - protein, 170 aa, Phakopsora pachyrhizi
MLFNSNVILFALTFALGSLYQGSEAQDLAGQPNCGQNCLLTAFTTLSNGCTQTDFACLCKSQKFTGTSNLRYSLSCTSTEAATTKSWGAKTCASVGVNVTATLGNTTSVSSDKQQQQQQSSLSSLLAPLSLLPAEVGAGGLVPPLLEQADSDVVNVGSGTEEQTDINLSQ

SEQ ID NO: 51 - protein, 223 aa, Phakopsora pachyrhizi
MVEAKVLVVVASLLFFSFLSASPAGSHVRHLTSSKRPPPGALVVRNQRTRVGEFSTINDAVKELKNKKGPQFIFIHSGTYNEAVHIKYKSALTIQGYSENPLSYEDNQVTVIKPTVASEAGSDSKSSVIWAQVDDFKMYNINAINSYGAGKDKQAVAVTADGDRQMYKSCRFDSYQDTLYIRSTASFFDKCLISGGVDVIFGAGSAWFEKCTIGVKPSPDNDV

SEQ ID NO: 52 - protein, 48 aa, Phakopsora pachyrhizi
MRSQSPLLLILTLISWRKIHWLVLNFCSYFRNFQKEKKKGFSAQRLLR

SEQ ID NO: 53 - protein, 90 aa, Phakopsora pachyrhizi
MFLLNERFLGLILLLISTLVTTIVSIAWFRFKTLNPGRIAQLIELEMDQLSNNRQRSLIDDKELWNDLDPIPQVDSERPMVQIVYSTKCN

SEQ ID NO: 54 - protein, 145 aa, Phakopsora pachyrhizi
MSLSFKALLVFLPLIFFYLVLAVKQPSDQTTHSTQPQKHNTHQTSINSKRELQQQLSQFNPHHQSLASSTRMSSVNEQLHQSQILTQTQTFQQLPRVRRSPQNRSLKINQQAMVQRHHYQLTLVNRNRVTRRILNLRMFPRVKNT

SEQ ID NO: 55 - protein, 189 aa, Phakopsora pachyrhizi
MFILSWFWDVLAQFGLVNKNAKILFLGLDNAGKTTLLHMLKNDRLATLQPTLHPTSEELAIGNIKFTTYDLGGHQQARRLWKEYFPEVDGIVFLVDSQDLERFPESKAELDALLSIEELFKVPFLILGNKIDAPGAVSEEELRHALGLYQTTGKGKVPLTDIRPIEIFMCSIVMRQGYGDGFRWLAQYI

SEQ ID NO: 56 - protein, 202 aa, Phakopsora pachyrhizi
MNMKLRVFISLTAALSLIAQSPSLAVTLNRDRPSNAHALQARQEQAAELAAVTVPEEQAAASAPEQKAAAAASAPEHAAASSAPEHAASAAAPQSDLDKAANRLYDLMVNLGNGLEIVTNLEASPQDIKAQTQKVKQYFGEMNELRNKLLQMSPIHSGNLPKSVQDANEAQETLQKALIAISDSAEDANVLKKELPSPFEDF

SEQ ID NO: 57 - protein, 482 aa, Phakopsora pachyrhizi
MKNIFRGSLYIFLTLLNLIVSLEAALVAFDEEVSLLRNTDRISQTQPKTLELQKYSKAYNGPLIYAPSSSVEIFKSAAQLHTSPKSDKSFTRDWRSIHMNDLLNPRITQPIKSKKGSLLLYLFRKKPKTQYEVKKTLVLSDKGSTDTSSLFNKESLALSYPRKEKNSGNYLEGLEIEVPTLSDEALVDLRDWKRSTSVLYFIEQIELRTNKVVDLLKRPDLNIEDVEGLSNFLKDLNVDENGDLKNLREGETIYLMIQDVWKASKSNFPAEVKTQIFYRYEYRLKKIFDKLSEQRFFIKKGKKETLIKESMEIVLYRLKKFKEKLEDEIIMLDNWRDESMKIIEKFNSLWWMISDDSNLRVKQEKNLDKILTVSAVRSGNEKLFKEFYKISVKVRGDSEENEGLLQSLISEISPLKKKDPKKRAIFYYQTHSISEELYHATIAQAKNVGTKILLNENDFDRLYDQKLLSKKANLLKPKDVIF

SEQ ID NO: 58 - protein, 46 aa, Phakopsora pachyrhizi
MNCQKKKKHYKLILLYKFDWREDYLVLFFCFILFIMRRLRIVEMNT

SEQ ID NO: 59 - protein, 142 aa, Phakopsora pachyrhizi
MLFNSNVILFALTFALGSLYQGSEAQDLAGLPNCGQNCLLTAFTTSSDGCTQTDFACLCKSQKFTGTSNSCYSSSCTSTEAATAKSWGAKTCASVGVNVTATLGNTTSVGTVIGNGTNPNTNSTLHNGTTVSSILIKYTIAV

SEQ ID NO: 60 - protein, 145 aa, Phakopsora pachyrhizi
MSLSFKALLVFLPVIFFYLVLAVKQPSDQTTHSTQPQKHNTHQTSINSKRELQQQLSQFNPHHQSLASSTRMSSVNEQLHQSQILTQTQTFQQLPRVRRSPQNRSLKINQQAMVQRHHYQLTLVNRNRVTRRILNLRMFPRVKNT

SEQ ID NO: 61 - protein, 261 aa, Phakopsora pachyrhizi
MGVSKLFFIWIGLFTISKLSTTLSVSFGKGINPLSLADEGESVLKQFQIFGKNNEVVNVKIVSKFNSDSNSLRFTKRDLLGIEDEKSNEKGNDKVKNQAQNRNYTRVDLTPARPTSETCYSGSFQGPNQSDCDVIFYAQKYNSYGSLTAFPGKSTRLFLFIFIFWRVGEVQYLYNCEPYTGTFVYVYYSSCVVAFQNPNHSNYSLDYNWAMLGAKAEQIKNRCLRDEEQSIGGSFLFENYLGYTFQNVLISLQRYAGNITA

SEQ ID NO: 62 - protein, 232 aa, Phakopsora pachyrhizi
MGVSKLFFIWIGLFTISKLSTTLSVSFGKGINPLSLADEGESVLKQFQIFGKNNEVVNVKIVSKFNSDSNSLRFTKRDLLGIEDEKSNEKGNDKVKNQAQNRNYTRVDLTPARPTSETCYSGSFQGPNQSDCDVIFYAQKYNSYGSLTAFPGTFVYVYYSSCVVAFQNPNHSNYSLDYNWAMLGAKAEQIKNRCLRDEEQSIGGSFLFENYLGYTFQNVLISLQRYAGNITA

SEQ ID NO: 63 - protein, 139 aa, Phakopsora pachyrhizi
MIGKFLLMVLFSSIFLAFGTIGQRIPEGLYVSCANAVVRQSCSAALDVAFTEGRIRRFTTARQYIQTQGCRLSWWVDTGILRIAQKDVLQAALQQIDDSCTANGVSVINPSNLPGRGVYVGQMVGNQYAVLRAEIGAFA

SEQ ID NO: 64 - protein, 126 aa, Phakopsora pachyrhizi
MTIGHLNKGFIFYWVALIFLPKIVIGSFTPKSKGFETIIGDENKIKNVPLKDLSKESSSQLKESRDDVFGPGSYAMSYNAKSQATKHAPGDDVSKSEAGVSYNIARDVQDSKRSKSEGRYDELHIK

SEQ ID NO: 65 - protein, 498 aa, Phakopsora pachyrhizi
MFSLTASILILSLIARTSAHLSLLAIYGSNNVIGRGFGVNTDGKYPRKLGRSGDAGGDSTVFETGTDNPSPACGRTPELGELDIPAWLSQAEGEGLPAAYSNGSIVAEAFQVNRDGGGPMSCEYNQDATATSWKSMFLTLNQAGNSGIYNVNRFNSTVVINFPQGAKCTGGWTGTACIVRCRTGVNKRFGGCFAAKLSSSSAPTLVHSANAVSGSQSLSDAQLDVIAQKIVSQMKLEGLLVKDDSQSTNSGGLTSRSSSNSQNVIAYVSHSKKKSATSSLKSNAKPKTKKHSGMLPNLGEAAPTPVNEEQLFRFPKEDEDIREGEDINQPLTNSDIIINDIPVPPLATPINSSTSRKTSNSCNLNPNSHQKTSKSTLPPTTYAELYEAKTDRRSDYESGRISWEREKWSKESELRMKQEEVKKELEYKRLEYAKEEKDKDLIYAREEKEKDHELKLFLAEKEREALKRKEDRDMLLAVVGSSRSLAEISEIAKILNIA

SEQ ID NO: 66 - protein, 218 aa, Phakopsora pachyrhizi
MLFATLIAVCLLALGGKAESDVQSDTASKLQRRGHDDSLPPVTFIMRDSNEHVGGKLLIYNSDGTLAFTFRRAVLNSDGLSNVEVRDVRNNFSINLESDDDTCFKKSHYVEREKNLGQFKIDPRGAKADRWHFTRKTPHGDFKYDFHRKYFSKDGNIYINDTHVRVASLTSEIRHEAWLQPGKHGVPTFSLHLQYNTEPIFFVALMGLDLTRVDTCGL

SEQ ID NO: 67 - protein, 285 aa, Phakopsora pachyrhizi
MLSSTQLSIILALSISYAFVSAQSVANSTATAPTPTTGAGGLTAGLSPGCQAAAGGLLSTEFSSCANIIGLVSLISAQGGLIDPLSNWISGVCSASPCSSATLSTTSTSLNAGCGADIQKGVPSAIALNTVITNYNPIREMICTQYSTNSTFCATSILSNIQTATGKSITLNEVQAVVSGGLNALPLSSVPASAYCNDCGHALFTKSKAINVNTGSSNLTSASSGASDAAAQTCGASFNDGKVPSTVRVAGSTKAGAPNAGGKNKASVVNAVAMGLMALVAICIV

SEQ ID NO: 68 - protein, 125 aa, Phakopsora pachyrhizi
MAFVSLSFAQLLPSAYSCSRVDLYKPPLRREDCRDSLNLFPVPNGDNVIYLGSGNWRGCGSCKVTIYNRGSRESRVTAPKGWAATAVHQAFDHCEGKPGSATIGDDGKIIAKIDYGNSGQGDCPP

SEQ ID NO: 69 - protein, 232 aa, Phakopsora pachyrhizi; identical to SEQ ID NO: 62

SEQ ID NO: 70 - protein, 380 aa, Phakopsora pachyrhizi
MSVKGFDKRALLLIIFGYNSFISCHGFIKSWSPDDGKTWILGQKQHLEKSSIRSFSENTGWIGSNFLTSPAIVCGSSKTPSQRVAPPSGEMFSQADQSAGKTLPVESGATVRLLINDEEGKVYPHNEGHIQAYLGRCGDAENSCQNFDAAAIGYFKILSEKDGLASTKIFPYNKAKDGNVLKVHMPRNLPKGGYILRVELIATGESSEKEGKQDQYYVTCGQISLSEPKSTPAILLQAIPVVKFPGAYKNGNLELESKLPGPRIFEGFSTDKKGAANQRNSNGSDSSQQLTPRCAVRCLQKISTDQEQGLFDVCHQKEGQAPCQCQNKQFVKAFFDCSQENCKGECELENGIAAFDIMCKVRTGDNFCHPKSKKDDKALA

SEQ ID NO: 71 - protein, 116 aa, Phakopsora pachyrhizi
MATFLAKNVALVPLFVAVGLGLGGGIGFGIHYLKNNQDVVLRKKSNPDPWNKVPQDNNTKFFSFNPDFWRARAQLTDPRLSFMESKPENERTLHEQAMVERAKQIRLNDKERTIHS

SEQ ID NO: 72 - protein, 96 aa, Phakopsora pachyrhizi
MATFLAKNVALVPLFVAVGLGLGGGIGFGIHYLKNNQDVVLRKKSNPDPWNKVPQDNNTKFFSFNPDFWRARAQLTDPRLSFMESKPENERTLHEQ

SEQ ID NO: 73 - protein, 142 aa, Phakopsora pachyrhizi
MHAANMKVSTLMIVVLSFIQLYSFTLSQITNGGITQDPNDKYRVRCLADSKSGDIISDDCYDSLDDFSQDDGIIKFNAKGEDNDEDSCRLELVGYPNQITNSDEIKVPKTSVISAINFGLDGCKNSSAIVEATGFILNIYKN

SEQ ID NO: 74 - protein, 142 aa, Phakopsora pachyrhizi
MHAANMKVSTLMIVVLSFIQLYSFTLSQITNGGITQDPNDKYRVRCLADSKSGDIISDDCYDSLDDFSQDDGIIKFNAKGEDNDEDSCRLELVGYPNQITNNDEIKVPKTSVISAINFGLDGCKNSSAIVEATGFILNIYKN

SEQ ID NO: 75 - protein, 274 aa, Phakopsora pachyrhizi
MNSHIGSKTIPHILLLPYFLILLTSIYHSVIGTSIESLVTAVDQKDVHEGINFMLDGSTHGARHELKVGEEDKNVTLKDHGQSHLLEIVRKLEKRYSSHTTTIEKTENFPKSSARSHHFKRSTPSSGEIRRWLRSHNKFRAQYSASPLVWDQNLADKANSETNTCVWRHSYNDIYGENIAAGQESIEEVVDEWVTGSEERRVYSPNNPTYSHFTQVVWGDTRRLGCAMTSCRNIRGSGLPQSPVKFWACEYYPPGNVDGQYRQNVKARYGGSPL

SEQ ID NO: 76 - protein, 256 aa, Phakopsora pachyrhizi
MKVLNFSLLTILLLALLVSISQGKEDQEEISPLEEYFGRQDLESRGLEDDWIDISVGVMMKREEDNQFNTSSSLTKRSTGDWETSEILVARDPKSNSLIFPSSWSEASQMIKRDIPQFVGRHYKRAKSAMSVKITWYTGHDLLNPACVQGGSGWAPVDKSMAAAVTIEWPGKPKCGKFVRIQKKEDPSVSVVVRVVDSCGGCRPYSQHIDLTIGAFKKLYAQEVGQVEGLRARIVKAPVGHKWTKKDTKIYGPKNL

SEQ ID NO: 77 - protein, 226 aa, Phakopsora pachyrhizi
MKVLNFSLLTILLLALLVSISQGKEDQEEISPLEEYFGRQDLESRGLEDDWIDISVGVMMKREEDNQFNTSSSLTKRSTGDWETSEILVARDPKSNSLIFPSSWSEASQMIKRDIPQFVGRHYKRAKSAMSVKITWYTGHDLLNPACVQGGSGWAPVDKSMAAAVTIEWPGKPKCGKFVRIQKKEDPSLYAQEVGQVEGLRARIVKAPVGHKWTKKDTKIYGPKNL

SEQ ID NO: 78 - protein, 529 aa, Phakopsora pachyrhizi
MLSILRWALLMGCLTYSAMAEAEQDDVVKLDGSNFATIVNPAPLILVKFMAPWCGHCKALKPEYHHAATELKEHSIPLGVVDCTADADLCSTHEIQGYPTLKVFSHGKASEYGGTRKADGIVTFMKKRSLPVISIVTSGNHTDFTQSDNVVIVAYLDESDKKSYETFQRYAETKRDEFIFGAVHEHSKIDSVSSLPKPSIVLWKKFDEGRNDFHGQEFTDENVSKFVSENSIPLLNEVSPKNFAMYEAAGIPLAYLFIEPNNPEREALIKALEPVAKEFKGKISFVWIDANQFSDHGKTLGLSATNWPAFTIQDLGKQTKYPFDEKKAIDQNSIMNFVKSFVDGKLSPSLKSQPIPQKQGPGSHVLVSDSFDDVVFGDDSRRDVFIKFYAPWCGHCKRLKPIWDNLAASFNQSSTDVVITKFDATENDIPPTAEISVSGYPTLKFKAAGQKGFIDYEGDRSLDSLIAFVEEHSNNKVKATKVELSEEEFEYGTGGEQVVFDKEEADASGDLDDEGVGGDEDKDAEHDEL

SEQ ID NO: 79 - protein, 456 aa, Phakopsora pachyrhizi
MKYFVLLAVAFHNFALNNAQGVGTTTPETQPKFVYHSCTGKDSCKPVDGTVTVDSNWRWAHDASGKNCYTGNTWDTTICPDGATCAKNCVLDGADYKSTYGVTSDKDGASLTLKFVTNGPYSKNYGSRLYALASDGNYQMFMLKNKRFSFTVDVSQLPCGLNGALYFSQMNADGDKSATNLAGAKYGTGYCDAQCPTDIKWIKGKANSEGWKPAENDPGKNTGNGNLGNCCPEVDIWEANSISQAFTPHPCKSAKPTVCEKEACGHFEGTRYKSICDKDGCDFASYRWGAKDFYGKGKKVDTSLPITVHTDFVTKDGTDNGKLVEIRRSYVQNGKTIANEPVKFQGLFKPADSITQEFCDATKKFTGDVNDFKAKGGMEGVDAAMTAGMVLVFSIWDDQEAQMAWLDGTYPPNKSGAGVERGTCKAGEGKPETVRQEHPNASVTFSDVRIGAITDK

SEQ ID NO: 80 - protein, 254 aa, Phakopsora pachyrhizi
MISLRFLSILLVLISTSQLTGSSPITGASEENVGKSSKQNNLVPRNIPGLDQVPGLDQVPGGVPIIGKNVAADQLAGIVRRNIPGLDQVPGLDQVPGGVPIIGKNVATDQLAGIVRRNIPGLDQVPGLDQVPGGVPIIGKNVATDQLAGIVRRNIPGLDQVPGLDQVPGGVPIIGKNVATDQLAGIVRRNIPGLDQVPGLDQVPGGVPIIGKNVATDQLAGFVRRNIPGLDQVPGLDQVPGGVPIIGKNAVTDQ

SEQ ID NO: 81 - protein, 163 aa, Phakopsora pachyrhizi
MAKRLLSFYYFLTCFANILSTPKLQPLVCPNDWSLDPISSSQNTRRNVYCYDHQFLCEESDCYEYPVCSSCTSDKGKLAKTVACKNEYHASDPTTDQQAICVSSDETFWQCSGPCEGYLKCINFSRKGPFIKSQLRKRGNPFAVAMNGSPGGTSSNFDSVLSF

SEQ ID NO: 82 - protein, 175 aa, Phakopsora pachyrhizi
MAQRFLPSILLYLVVLSLVSAQDGTRNLVFPNQNSNNQSNTNTNTNTNTNNNANTNTNTNAVTNTHQSNTTATNNNSTSSGNISSNSTNRANTSTTTTTNLTSIKDSTGANQVPAPGATGGKANGRYGPDDQYISSAPRRTLAARSTDLIIIFSSLESQAWLLPAHCSFKIHRSY

SEQ ID NO: 83 - protein, 481 aa, Phakopsora pachyrhizi
MEKLFTLQNLLFLLIFWSQLTDKTSYWLIGIPFGCEAADVVDDASRAEAMIMRYQNTLFDTEEEEIEQMHLILNPQISGPLNPTQIGKETAEGGSQKTNTYTMVNSINDQKEEKTRAESSSLKNQFPGKFESVQKQTLDDKKKRAEQDPLNQLKVLNFIIQVENDEIAVGIVKSSLVNGWPSLKYLQSLIISYFEHDAEILWGYPTLKDFLLSVNKGRGFERVLRVLDDENWKNIFPQLCSTLFAHLKKTSNLEDDEGTITGLEIKEFNKIYSADNFMAMMSPEDKNKQTIKLFNSLVSLMNIKISKVASMKHPGASNLWFLTYYVFEFLVNRKELKFSKQIRNGIRMNSIALKVEVYLKLFTVFSKARVLVELPRRLENKKQITVDIPNGLQNTDLIEFFRERWLVMEALENDITQLIPLISNLYKINKSSTAIEIFDDLYDWAYKTQVNLKILRMNEAGTLSTVDAKSPKISPNAIKEE

SEQ ID NO: 84 - protein, 255 aa, Phakopsora pachyrhizi
MFSKSFQGLSLLVVLMASIIFNCDESNAQNNFNSFQQPGTNCYEQSKSYGINFEHCEKALRKISYDSNGNLDNSSKTVFVWHKSCVVKVQKATYAQPSRQQVEYGVRNLLQTCPTRGGIYIPSNDFRTYVYSSNRENVYNLNSPVCIKHQCHINPNDCLMAFNNIPLSTKGFFLPSGSTSSNVIKTSSGNCTVKLVTTDGAGFRINHPEINSGIKTLLSKCGSRPGYNYFGGGSLGMNGDIKIITQNSYNNGQCN

SEQ ID NO: 85 - protein, 412 aa, Phakopsora pachyrhizi
MIKSNSVYALVGLIPIVWVLLPMTCKSAMVFHTLREPPIGKMPQELSGCKMEILSNKNFKESEYKAGESLASRKSTDVQNVLDEHLNSAMESTKKKTPVNRNSIIFDDGNARANDFISASRAEHITSKELGDLFENYMREGKDKIIKPEIMLREIAFGFSRFSKRVIQLPSGPLSKVLLSEENLKLIPDSIAFYVKNLKEEKFESFIGYVIGNELNRLNDLYLNDFFGQLSIEFSKPGFYSSLKELKTDDSKSLAQGLFSYFSSAPVGKGENLEVAKEKYLLVYRIIDSLSHDGPIFATSFQEQARDRAAFEKFNIISKIQKLTDKINNSGKTAEDKLLQEALELSDKLFDIESNVSEQTHAELLSTWTDSTVVNSIIRDFDEKLYKQIDFDDDADFYATHSQNIKHAIKAV

SEQ ID NO: 86 - protein, 58 aa, Phakopsora pachyrhizi
MKFFLGLTSLFLIASIVGVVFSSPVNTNGEDTRLQKRKTVAPATCTRGGRVVKNKCKV

SEQ ID NO: 87 - protein, 67 aa, Phakopsora pachyrhizi
MPSKLSGLTADVVLTTLVTAYQLELDNLSFGSISATGQVARKTLVEWVLSGQISPGGDRQDLVINKK

SEQ ID NO: 88 - protein, 39 aa, Phakopsora pachyrhizi
MRGAVVELNEAHEYQKKAGKKMLCLLILFIVIFAIILIA

SEQ ID NO: 89 - protein, 48 aa, Phakopsora pachyrhizi
MYSVYTCYLSIFFFLRTRAKRNSFSFRGKSLSTHPLDGIALDRQFNKK

SEQ ID NO: 90 - protein, 47 aa, Phakopsora pachyrhizi
MGAIFAKMMMELSAVNLTSVALLLVKAVLMHESFLKQVTISLFFSKK

SEQ ID NO: 91 - protein, 116 aa, Phakopsora pachyrhizi
MWIRRLMISTRILPVIVITIYILKNRRVLIKVEQNNSTSFRTQRSVRFRGMTDLKNISSKEVSTHTTVDSAWTIIDGKVYDVTEFLEEHPGGKKILLKNCGKDSTELFNQYHNEKV

SEQ ID NO: 92 - protein, 71 aa, Phakopsora pachyrhizi
MIRVSISNKVLKYIALYFLISCIGHSTQESQHPYDLEGHDQAPNQKLKILNRKLEGETEATIPIRDHSLYA

SEQ ID NO: 93 - protein, 42 aa, Phakopsora pachyrhizi
MLSTVIVKLLFWVHRSHELQFFYVHYQPFGLRLVCFHKCLCL

SEQ ID NO: 94 - protein, 42 aa, Phakopsora pachyrhizi
MKDIFVRAVFLFWFCLDFIEPIEIHTDKTPKISKKVHIIIDT

SEQ ID NO: 95 - protein, 70 aa, Phakopsora pachyrhizi
MTNLRVAPFFFLLFCFLLLSFWYDLTLPSTCDNLKSVLKDDLLLNCFCLPLISQNHFRLMTLNIFFYKNN

SEQ ID NO: 96 - protein, 190 aa, Phakopsora pachyrhizi
MKSFNLILFITVALALITQIDCLPKPRSLVSFNHLPAKEIVKRQEDRFNDADALSRETEGDGEGEGENDIGGEGENGSDNDDEGGGESEDGSVGDGEEEAEGEEVNEDVVEVDGDGDKDKEVEGEEGGGVETVEKVEGVDIGSKEVNGNVEEGDNNDNENAEDFSRGVNDLQENQFVDDDSEDEALADER

SEQ ID NO: 97 - protein, 52 aa, Phakopsora pachyrhizi
MRRWMQTMMMRRTATRKRTKRKRMVMLRLKKHEVFFFFLSMSLSFYIYIKIR

SEQ ID NO: 98 - protein, 140 aa, Phakopsora pachyrhizi
MKSYIPFIVTILLLAELSARNLKLSSRSDQGVLSCTTYSDTNTDAASCNNLTNIYCAQCEPGKTVGNNCVKKLESVANQTVVTNLTVINNNNNSSRSTQGCTISFKMDEDGSYICQNDDGTFGCESYRRSPITCEGCVDA

SEQ ID NO: 99 - protein, 71 aa, Phakopsora pachyrhizi
MASEYLKIKEIYEASKAFEPNLSPQLHSPIGFALLASAFGIGFLFTTLPKRGIPLAELFPAILSSVLTGFG

SEQ ID NO: 100 - protein, 70 aa, Phakopsora pachyrhizi
MSCRLLLFFFFFFFFFRLTVLYFILPKTLSDSLKNSQQLRSWNLVSKVHPIKNKKHCSNSKCITYSNIYM

SEQ ID NO: 101 - protein, 140 aa, Phakopsora pachyrhizi
MKSYIPFIVTILLLAELSARNIKLSSRIDQGVLSCTTYSDTNTDAASCNNLTNIYCAQCEPGKTVGNNCVKKLESVANQTVVTNLTVINNNNNSSRSTQGCTISFKKDEDGSYICQNDDGTFGCESYKRSPITCEGCVDA

SEQ ID NO: 102 - protein, 28 aa, Phakopsora pachyrhizi
MLTVGLSFFFFFDQITCTKNKIKCVKKR

SEQ ID NO: 103 - protein, 184 aa, Phakopsora pachyrhizi
MSGQLLNLAIPLIGMQIARKIPFDDPQVLTGMRMAYILSQLSCLSIYYYTASKIKSKNDTTVLKYVEPKAPMSPDQAKLVTTTVRDYDLNQISTGLRSVMMGVLMMLFLHLYMKYDAPLFLQSLMPLKTTYENKVIQIHLLGRSATGDLKRPFKTGGFMGAATDAKTDAKSIKEAEKSSVKKDQ

SEQ ID NO: 104 - protein, 40 aa, Phakopsora pachyrhizi
MNKLFFLSSATSFRGFYYCYYCFFLFPPLFFFFFFFFFFF

SEQ ID NO: 105 - protein, 340 aa, Phakopsora pachyrhizi
MPSINFLIFAAIFLLGTTKHVAWASKNELDLITGLETSTDLINVNVGINNDPTIINNKNIPYRPGSDVNFNPNDFDNNESENGNTIENHTVLKKPRVFLPMKTYFSKENITSEDSIVKTDSVTSAANEPTNSKFPGVHQTAPVKCGNGKPPPCNEDTKPNNTRFSGTHQTAPVTCGNGKPPPCSESSQYTSPITVHGTQRTAPVTCGDGKTPPCSGSKPHTTRIPGTHQTAPVTCGSGKPPPCSDSSHHTSPRNIPGAHQTAPVTCGNGKAPPCSGSNSNNQSGHFPGTHQTAPVKCGNGKPPPCTGSVNSSVEPKNHPATQRSASASSKNPKRSRRTVF

SEQ ID NO: 106 - protein, 355 aa, Phakopsora pachyrhizi
MPSINFLIFAAIFLLGTTKHVAWASKNQIYDCSNSILCIYLSELDLITGLETSTDLINVNVGINNDPTIINNKNIPYRPGSDVNFNPNDFDNNESENGNTIENHTVLKKPRVFLPMKTYFSKENITSEDSIVKTDSVTSAANEPTNSKFPGVHQTAPVKCGNGKPPPCNEDTKPNNTRFSGTHQTAPVTCGNGKPPPCSESSQYTSPITVHGTQRTAPVTCGDGKTPPCSGSKPHTTRIPGTHQTAPVTCGSGKPPPCSDSSHHTSPRNIPGAHQTAPVTCGNGKAPPCSGSNSNNQSGHFPGTHQTAPVKCGNGKPPPCTGSVNSSVEPKNHPATQRSASASSKNPKRSRRTVF

SEQ ID NO: 107 - protein, 337 aa, Phakopsora pachyrhizi
MPSINFLIFAAIFLLGTTKHVAWASKNLITGLETSTDLINVNVGINNDPTIINNKNIPYRPGSDVNFNPNDFDNNESENGNTIENHTVLKKPRVFLPMKTYFSKENITSEDSIVKTDSVTSAANEPTNSKFPGVHQTAPVKCGNGKPPPCNEDTKPNNTRFSGTHQTAPVTCGNGKPPPCSESSQYTSPITVHGTQRTAPVTCGDGKTPPCSGSKPHTTRIPGTHQTAPVTCGSGKPPPCSDSSHHTSPRNIPGAHQTAPVTCGNGKAPPCSGSNSNNQSGHFPGTHQTAPVKCGNGKPPPCTGSVNSSVEPKNHPATQRSASASSKNPKRSRRTVF

SEQ ID NO: 108 - protein, 147 aa, Phakopsora pachyrhizi
MHSKVLISWVMSAFVAIVLHAANSIQDNEEPPIVAKVYFDMKQGSKNLGRIEIGLFKNTPKTSENFRALAVGDKKSKDGIPLAYKGSKFHRVINQFMIQGGDFTKGDGTGGLSIYGPKFQDENFINKHTGPGTVSMANSGPDTNGSQ

SEQ ID NO: 109 - protein, 100 aa, Phakopsora pachyrhizi
MWIRRLMISTRILPVIVITIYILKNRRVLIKVEQNNSTSFRTQRSVRFRGMTDLKNISSKEVSTHTTVDSAWTIIDGKVYDVTEFLEEHPGGKKVQSKFC

SEQ ID NO: 110 - protein, 294 aa, Phakopsora pachyrhizi
MQTKILLALCLVAISQVNAHGAITAVQGSNGMTGEAFGVDQSTPRDGTKRNPFQTDSSIIRDREIASGKSSACGRTLAGGNNEIGAAMSKAESAGIPSVSSDGKVQMTLHQVNGDGGGPYTCDVNASGDGKTFTPMTISTNIPGKNSRSNAKAEDIPLIAEMPKGMTCTGGSDGQTCIVRCRNAANAGPFGGCVAVTQATDATKQKRELSAEQRIDEMIKQAEAAEAFGDETLAKREMSADERLDEMIRQAEAAEAFGDETLAKRELSADQRLDEMIKQAEDAELAEENLSKRD

SEQ ID NO: 111 - protein, 208 aa, Phakopsora pachyrhizi
MRSLIIPALLCVLFIHKSCATVHTQCYNYFLQKDGCVFSAADDRNRCSADPKPSTAVGVVQASNTNVKRHTLARRYDTTLPSPSVRGEGICGNYNTAEAEGASLWVGPNPDSTNPEEAGWLNKGKTSNCNKQLYIINPRTRKTVYVNVIDGHDFQTTQPDVGCFQIALTQNTFLQLDPTDEEKEKGSIGSLTWDFNNLNGASPQDGPV

SEQ ID NO: 112 - protein, 373 aa, Phakopsora pachyrhizi
MQTKILLALCLVAISQVNAHGAITAVQGSNGMTGEAFGVDQSTPRDGTKRNPFQTDSSIIRDREIASGKSSACGRTLAGGNNEIGAAMSKAESAGIPSVSSDGKVQMTLHQVNGDGGGPYTCDVNASGDGKTFTPMTISTNIPGKNSRSNAKAEDIPLIAEMPKGMTCTGGSDGQTCIVRCRNAANAGPFGGCVAVTQATDATKQKRELSAEQRIDEMIKQAEAAEAFGDETLAKREMSADERLDEMIRQAEDAELAEENLSKRELTADQRLDEMIKQAEAAEIAEENLVKRELTADQRLDEMIKQAEAAELAEENLAKRELTADQRLDQMIKQAEAAEIAEENLAKRELTAGQRLDEMIKQAEAAELAEELI

SEQ ID NO: 113 - protein, 160 aa, Phakopsora pachyrhizi
MRSLIIPALLCVLFIHKSCATVHTQCYNYFLQKDGCVFSAADDRNRCSADPKPSTAVGVVQASNTNVKRHTLARRYDTTLPSPSVRGEGICGNYNTAEAEGASLWTTQPDVGCFQIALTQNTFLQLDPTDEEKEKGSIGSLTWDLLVLCLQLKEEEEGMI

SEQ ID NO: 114 - protein, 82 aa, Phakopsora pachyrhizi
MAKPGVGRLPPGKALGLLTFGTGLGWIVMKIVTPSEEKLYSRLSPDLKLKVDQRRAESKEWDKTYGNTTVIEQYEKSVKRDK

SEQ ID NO: 115 - protein, 402 aa, Phakopsora pachyrhizi
MQTKILLALCLVAISQVNAHGAITAVQGSNGMTGEAFGVDQSTPRDGTKRNPFQTDSSIIRDREIASGKSSACGRTLAGGNNEIGAAMSKAESAGIPSVSSDGKVQMTLHQVNGDGGGPYTCDVNASGDGKTFTPMTISTNIPGKNSRSNAKAEDIPLIAEMPKGMTCTGGSDGQTCIVRCRNAANAGPFGGCVAVTQATDATKQKRELSAEQRIDEMIKQAEAAEAFGDETLAKREMSADERLDEMIRQAEAAEAFGDETLAKRELSADQRLDEMIKQAEDAELAEENLSKRELTADQRLDEMIKQAEAAEIAEENLVKRELTADQRLDEMIKQAEAAELAEESLSKRELTADQRLDEMIKQAEAAEIAEENLAKRELTAGQRLDEMIKQAEAAELAEELI

SEQ ID NO: 116 - protein, 493 aa, Phakopsora pachyrhizi
MNYQRINYGLISYVAFGMLLLCTVAQCSFARTENRLRSLRDLPVLSDLKLEERVDTEKYDTITQKSGRFLVVNSFPAPKDRYPEENFFSKNLLKTLRSDVEDGREFNEDQKLQIYQGLKIYFEHQSFQFLPGIGITKVPGLSEILARLSQLAGTSKSNGLDINQIFSDIDALKGVVWYIGSKITENLGFSGIFKTIDIRQFILEHPDLISIKTLLKNFEDKQWKAIELGFLRCHLEIGYNPHLLSDVGYRYLIKKFEKVIPRVENHESFNPEVLNSAKSVINYEYEERYDQFLIKNVFVYHIINYFERYFNTDLDDSIKYQTYMNRYMIQMIKTSLEFRESVIDPLYFKKVIFRHNHPTFSIIEELQPNRNLPLVNARGTIRIDIRNRSFFLRKDDIKTLEPWLKKVKLQAQAVKEQESIFTSVLNDLYKISETDLGFKQWLMSDKMQILLDDIRKSLEDATHYSSVDIGLAPKLKEQFKLSNVEGKTIVNQS

SEQ ID NO: 117 - protein, 88 aa, Phakopsora pachyrhizi
MVPTNQKTLILPRMVKLVPALLVSLLRLLRHRVIFVTGVAKRVIGSRRVRQMITLNLKEGLESSERLEFLKVFYRRLKASQAMGRMSW

SEQ ID NO: 118 - protein, 36 aa, Phakopsora pachyrhizi
MASKVIVVGGGLSGLSAAHTLYDRGANLLVLDKNPL

SEQ ID NO: 119 - protein, 65 aa, Phakopsora pachyrhizi
MKPVGPVNCRRGKGFLLFFLFLFFILNLSFDVPGTQLVVCPDGECYLFLSLYLSLRRDNLVVGCG

SEQ ID NO: 120 - protein, 51 aa, Phakopsora pachyrhizi
MMMMMMIKSQKIYYIYIFICLELMITDRNYELKNRQSSKACPVSIQSRGTH

SEQ ID NO: 121 - protein, 256 aa, Phakopsora pachyrhizi
MTWKTRLVSSLLSQLRISYKSVGSISNLSVKKPESSLKFRDFFSTKSIASATSSTSLPIRPTLKSSSGTFKESLLNDNLSSISTSMALTKLRSQPNHYVISFLVGKRYRLTTNDLVTVPHIKDLRVGDLIKLTRLTEVGSRNFTIRAIGNGTVGHAALETKPPPSNQKFTNTFKNSDDAEESPGLKRVICFKPEIPHYLDDGLVDATAIVVEHTRGKMSTVIKKKRRKGYRKTIKSKPYYTQIRLCEIKFPTTAET

SEQ ID NO: 122 - protein, 71 aa, Phakopsora pachyrhizi
MMMMMMMKFIRNCHQQQSIVPTAATTLSHLKSSSILSINNHNHNSHHLNRRYRSNLTLDDILPPNNQQQIQ

SEQ ID NO: 123 - protein, 238 aa, Phakopsora pachyrhizi
MANNLTVSTFLLLALSPFVSYAAVHPQLVAFKTFSFPKGTSPSSENGKNLQFFKAADSPAATEVPSQGNNLVNSGLTSTVCKSSEGKEFTFTNQDCLNAARVIANESTSSAQCGNCVIGLFDVNKKKAFTPPGPVSPSVLENQVNKVLSECSNRASSTNPPVNNKRSLLPSQSGVESVSGSDSIANHPAVANAGGHNNLATQPGTTSPGGSQPGSGSGNSDSATVQMVMGYNPTSKAC

SEQ ID NO: 124 - protein, 91 aa, Phakopsora pachyrhizi
MKVFTIFSAVMLLFAAGAMAAPEEISANSADNVDEKQFRPYPTWRNGFNYYRYGNYWLPPNAYTRLPGGRIRVTQGYFDQGRRWVPFVGIL

SEQ ID NO: 125 - protein, 441 aa, Phakopsora pachyrhizi
MRMFEDFIKSSKSTHLTLAVLGTLIFHEVKGSFAQSGEMIKTSMLTHPDGFLNKYPDSKIISSPLFENDIRSSQGVRALAGSLKNEQLSYVGKDSESFAKSIKGLPISQEDELLEQTTKYLTSKSHPDLILQNIDNLCQKILGSTSKNLDGAMSLNYNVKRTWLTKLINVFSKISDDETRSKLFGNNNVISLLASEYHFIINKPDHKLALQTSVAGMIFIEKEIDHLQPILSFFNEEKQKLFRHKYFVYEVESLSAFGSQKLNKKFYELFSLLPSLKKEKGLEVKKDLIEKLLMKKDLPKLDKFLTYNEAREQYETLAQILDYLIHYNDEIKAEDFENIPENKLYELLKLISDEHENFQSQVYNDKYLTTYKNKIDEVTKTLNEQQKDSSIFKISKELLVHLEYLKYSNNEMKQLENLLDARVSGLARGQSKNSRMSRKLL

SEQ ID NO: 126 - protein, 116 aa, Phakopsora pachyrhizi
MATFLAKNVALVPLFVAVGLGLGGGIGFGIHYLKNNQDVVLRKKSNPDPWNKVPQDNNTKLFSFNPDFWRARAQLTDPRLSFMESKPENERTLHEQAMVERAKQIRLNDKERTIHS

SEQ ID NO: 127 - protein, 98 aa, Phakopsora pachyrhizi
MAKQGVKGRLVLTSSILGFFGLPGYSAYTTSKHALRGLAESLRLEMILYDIKVHCYFPATILSPGFDEEQKCKPELTKEIEGVDEGQSPEKCAKKLIR

SEQ ID NO: 128 - protein, 70 aa, Phakopsora pachyrhizi
MRCFIFALALLSSAQFVLGAGDSSQKVNRRSLVQVNALTSGRRPFNSGNSLVEVNALKNYQKDNSKISGY

SEQ ID NO: 129 - protein, 74 aa, Phakopsora pachyrhizi
MRCFIFALALLSSAQFVLGAGDSSQKVNRRSLVQVNALTSGRRPFNSGNSLVEVNALKNYQKDNSKISGNAQQY

SEQ ID NO: 130 - protein, 456 aa, Phakopsora pachyrhizi
MALKAHFLRLLSSLSLNLLIAELAACEYNGATAITSSSSTYMMQSSSQASVDLTVIPPISNLTGDHGKKRNKKKYSAYYPSYNADQRPVSDIKYEHYDELVFFVATTNQNFTIGLGDIEQEVWDSRAHEFVHRCKKAGVTPFYCLGGWTGSIYFSSLVSTASNRTEYATSAVNFAKKYGFEGIDIDWEYPSIQGIGCNIVSDADVENYQLFLKEIKRIWPEGKLSTAVSIGGIRASDYSPLPASNLTTLIDVVDKLNIMAYDVYGSWSTTTGPLSPLQSTCADDENQISVESAVEVYLQQGFKPSQMALGIPTYGRSWTLASPILTPRTVQNYTTYYYQNFTDIPRGGQFDDEPGVDVCGQNSTSWGGTILFTELLSRGYLDEDEMASAGFVRYYDECSRQPFIVNGTLMISYDDTRSTLRKVQYAKSKNLAGVYFFDTLGTTERSLRAVRQVLNS

SEQ ID NO: 131 - protein, 306 aa, Phakopsora pachyrhizi
MISLRFLSILLVLISTSQLTGSSPITGASEENVGKSSKQNNLVPRNIPGLDQVPGLDQVPGGVPIIGKNVAADQLAGIVRRNIPGLDQVPGLDQVPGGVPIIGKNVVTDQLAGIVRRNIPGLDQVPGLDQVPGGVPIIGKNVATDQLAGIVRRNIPGLDQVPGLDQVPGGVPIIGKNVATDQLAGIVRRNIPGLDQVPGLDQVPGGVPIIGKNVATDQLAGIVRRNIPGLDQVPGLDQVPGGVPIIGKNVATDQLAGFVRRNIPGLDQVPGLDQVPGGVPIIGKNAVTDQLAGLVRRNSVASRVGL

SEQ ID NO: 132 - protein, 178 aa, Phakopsora pachyrhizi
MKYNLVTILLSIYVFNLYSCSPIPNHHQSGAQGISGQESNNNSLNQRRAESQQTKSDSNNQIQNTNNEINQNQLNRREIINDSKSQDHSSTTNVAQNDQHTNNQSRGDSIENKSIWEIGNKADHSNNIQQNENIISKNVQDFFFFFFYSNLITTVATRQIRLIRLFKMTPNIKTSNQI

SEQ ID NO: 133 - protein, 259 aa, Phakopsora pachyrhizi
MKYNLVTILLSIYVFNLYSCSPIPNHHQSGAQGISGQESNNNSLNQRRAESQQTKSDSNNQIQNTNNEINQNQLNRREIINDSKSQDHSSTTNVAQNDQHTNNQSRGDSIENKSIWEIGNKADQSNNIQQNENNSQQTKSSNNQNTNNQSNQNQLNRRESINDSKSQDHSSTTNIDQNEQHTNNQRRGDSIENKSIWEIGNKADHSNNIQQNENIISKNVQDFFFFFFYSNLITTVATRQIRLIRLFKMTPNIKTSNQI

SEQ ID NO: 134 - protein, 247 aa, Phakopsora pachyrhizi
MKYNLVTILLSIYVFNLYSCSPIPNHHQSGAQGISGQESNNNSLNQRRAESQQTKSDSNNQIQNTNNEINQNQLNRREIINDSKSQDHSSTTNIDQNEQHTNNQRRGDSIENKSIWEIGNKADHSNNIQQNENIISKNVQENNDASNKQQSNNNGSNKADSIDKIIQNDSQHQNEQPNIVRRSNVGTENVQGQSQEKALNQGSEQNWAPIQGNNWNVAVRRTVNENVQSQNNPTSQLQNPQNNWSNQ

SEQ ID NO: 135 - protein, 88 aa, Phakopsora pachyrhizi
MVLPLQIIVISIFISLNNLSAARLFGSDGIRRIISGRSTHPSVGDRHGLSARNLLNQESYDDYKSDENLYGSTTKTTGLTTEPVNLVP

SEQ ID NO: 136 - protein, 182 aa, Phakopsora pachyrhizi
MSSILKSLLSWIRSIFFSKELEITIIGLQNSGKTSLVNVLMSGQFSETMVPTVGFNMRKYTKGSVTIKLWDIGGQPRFRSMWERYCRGVSAILFVVDSADKQSIDQSKVELQSLISQPQLSGIPLLVLGNKNDLAESLKVGELIEKLELSKVTGREVSIYSISCKESINVDVTLNWLLKRAT

SEQ ID NO: 137 - protein, 137 aa, Phakopsora pachyrhizi
MSNKAKASTKFRMTLALPVGAVVNCADNSGAKNLYIISVVGFGARLNRLPAASCGDMVMATVKKGKPELRKKVHPAIIVRQRKPWRRRDGIFLYFEDNAGVIVNPKGEMKGSAIAGPVAKECADLWPRIASNAGTVV

SEQ ID NO: 138 - protein, 127 aa, Phakopsora pachyrhizi
MYSIRFNPVTLLSFLFVFSIVHSTPMDSLVPSNYGLRPTGPYTAYPKGGDHPDWKYLNQREWYKIDATQQDHARFHSYLSMSIYGDYNKTCPQTFTQGYKIIQSFSNFYVAYIPEMDKIVVVFKGPG

SEQ ID NO: 139 - protein, 184 aa, Phakopsora pachyrhizi
MGVSKLFFIWIGLFTISKLSTTLSVSFGKGINPLSLADEGESVLKQFQIFGKNNEVVNVKIVSKFNSDSNSLRFTKRDLLGIEDEKSNEKGNDKVKNQAQNRNYTRVDLTPARPTSETCYSGSFQGPNQSDCDVIFYAQKYNSYGSLTAFPGTFVYVYYSSCVVAFQNPNHSNYSLDVEFQISI

SEQ ID NO: 140 - protein, 100 aa, Phakopsora pachyrhizi
MNFGSSSKKNLVARPSVGIRRLVLVCTAMVWSITLILSQPSGVNSSPFLRVAKSIGPEVAKGAAGGVGLVAVEQIANKVIGRSTDGKNVEDENSGSKNFP

SEQ ID NO: 141 - protein, 137 aa, Phakopsora pachyrhizi; identical to SEQ ID NO: 137

SEQ ID NO: 142 - protein, 100 aa, Phakopsora pachyrhizi
MNFGSSSKKNLVARPSVGIRRLVLVCTAMVWSITLILSQPSGVNSSPFLRVAKSIGPEVAKGAAGGVGLVAVEQIANKVIGRSTDGKNVEEENSGSKNFS

SEQ ID NO: 143 - protein, 285 aa, Phakopsora pachyrhizi
MFKGLSLAVLSIALRETIGQATTVSTDSLNAISNQFDSSYLSSQYPDGFGIPLKAQAILNVNYPFGNVQLGKAYNASDVANQPTISISPLESSQASFKAPSAFTLMLADANALGNPDPKGNYRHFLENGVTFGDPTSNGTMSMNPGSGTVVTSYAGPGPLPNEGPHRYAWLLFNQPGDFQAPSNLSSAGVSPGHWYVNGYISGSKLGDLVAASFFTVENGHANFTPKPLVSPVHNGNKTNNTSNLSGNSTNSSGNAKISGAAVSARVLDGTLMGLVAVGLGLAFV

SEQ ID NO: 144 - protein, 233 aa, Phakopsora pachyrhizi
MKYNLVTILLSIYVFNLYSCSPIPNHHQSGAQGISGQESNNNSLNQRRAESQQTKSDSNNQIQNTNNEINQNQLNRREIINDSKSQDHSSTTNVAQNDQHTNNQSRGDSIENKSIWEIGNKADQSNNIQQNENNSQQTKSSNNQNTNNQSNQNQLNRRESINDSKSQDHSSTTNIDQNEQHTNNQRRGDSIENKSIWEIGNKADHSNNIQQNENIISKNVQENNDASNKQVIF

SEQ ID NO: 145 - protein, 328 aa, Phakopsora pachyrhizi
MKYNLVTILLSIYVFNLYSCSPIPNHHQSGAQGISGQESNNNSLNQRRAESQQTKSDSNNQIQNTNNEINQNQLNRREIINDSKSQDHSSTTNVAQNDQHTNNQSRGDSIENKSIWEIGNKADQSNNIQQNENNSQQTKSSNNQNTNNQSNQNQLNRRESINDSKSQDHSSTTNIDQNEQHTNNQRRGDSIENKSIWEIGNKADHSNNIQQNENIISKNVQENNDASNKQQSNNNGSNKADSIDKIIQNDSQHQNEQPNIVRRSNVGTENVQGQSQEKALNQGSEQNWAPIQGNNWNVAVRRTVNENVQSQNNPTSQLQNPQNNWSNQ

SEQ ID NO: 146 - protein, 163 aa, Phakopsora pachyrhizi
MAPQILLVLPFPGSPTMVLTHPCHSSDQDSILEAVCRQNQLPLSFASSLRLSRCGRPWNGILAEDEFSDVNFVVADVAMRLRGGGPKKRCQHAKNSVNESQCGQPALRLVGDCPHCTLQFCARHRLPEDHACLNMTSCREEAFAKNKAKLESERTVGSKMVGA

SEQ ID NO: 147 - protein, 255 aa, Phakopsora pachyrhizi
MVEAKVLVVVASLLFFSFLSASPAGSHVRHLTSSKRPPPGALVVRNQRTRVGEFSTINDAVKELKNKKGPQFIFIHSGTYNEAVHIKYKSALTIQGYSENPLSYEDNQVTVIKPTVASEAGSDSKSSVIWAQVDDFKMYNINAINSYGAGKDKQAVAVTADGDRQMYKSCRFDSYQDTLYIRSTASFFDKCLISGGVDVIFGAGSAWFEKCTIGVKPSPDNGISTITAQKRERGSNSRKALVRICQRRIPVLLFT

SEQ ID NO: 148 - protein, 41 aa, Phakopsora pachyrhizi
MQKLYILLLFSIIKLCSSGLIGFEAEIINNPNTWKTVGEIH

SEQ ID NO: 149 - protein, 94 aa, Phakopsora pachyrhizi
MFGLGKIFYVSLLLINSVAILNEDRFLARIGWSSNSGNPNFGTTNQFHHDPSISPNGQSVKEKIINLIGAVRTLMRIPLIGLNIVVIIYELILG

SEQ ID NO: 150 - protein, 61 aa, Phakopsora pachyrhizi
MIALLGFLSSPRLLTVNNSLIKLCQITPNLSLTCLLASTPSQRKKIKNGTVKSRILIRPGS

SEQ ID NO: 151 - protein, 61 aa, Phakopsora pachyrhizi
MTCCTPTLPLIFILTGYKLPNHHIYFYFIFISPISHNLLLPGRLVNCQLSQFFYITVYRSC

SEQ ID NO: 152 - protein, 86 aa, Phakopsora pachyrhizi
MKFFCSSICFSTFYFLMILGFSTFNFTLAFRCGVTSVGFCANTNGGGKLQAQFLRQRLNSNEYVNCPANRPLNLCCSAGGRGCSPA

SEQ ID NO: 153 - protein, 63 aa, Phakopsora pachyrhizi
MVLDIKKLDGFSVESLYKFQTRTVILIFLIAVTLFESARAAVMTAKDTECFRFKYCACRCSIV

SEQ ID NO: 154 - protein, 91 aa, Phakopsora pachyrhizi
MRFNNFHRFSSFEASKVKLLFIVCALSISLTSSLQLNILKGTEPGCGSLPENNMINMQLSQYSDARDPGKGSSPKTNKEWPYSDDNQTDLK

SEQ ID NO: 155 - protein, 143 aa, Phakopsora pachyrhizi
MNFLLMNFMSMVVLIILSTSSLSRSSPSADPSTHNHHSMLIRTNDELLSKPSIKKRKIVDFDVDKKVMKKKRNDVVYQEGDEGSEERMKELDGDVEISEEDFDQLAVIPDGSGSERQYIWSRWSVARNIFGAGDPKKEVKSNS

SEQ ID NO: 156 - protein, 236 aa, Phakopsora pachyrhizi
MKFSYSQLIVLLSVISLSFVASEETKSQRAESSDEKWFGLGYNSLRWNYWNGLAGWGGIGSLYPWLGAYSGAYGIGLCSGNYLNSWFKSAQGEKQRRSFEFNSGAQLQTRGEPDLLETVTCKNTKGESQQFLTSSCLKAAEQLVEKQTSSATCGSCTLQLHGPSGDLSAKSIPASELTTAASNILKACSKAENKILSTSELQRRGNEAENSSSDNNAKDSKNSFAVVLLKGNGPEC

SEQ ID NO: 157 - protein, 280 aa, Phakopsora pachyrhizi
MFKGLSLAVLSIALRETIGQATTVSTDSLNAISNQFDSSYPDGFGIPLKAQAILNVNYPFGNVQLGKAYNASDVANQPTISISPLESSQASFKAPSAFTLMLADANALGNPDPKGNYRHFLENGVTFGDPTSNGTMSMNPGSGTVVTSYAGPGPLPNEGPHRYAWLLFNQPGDFQAPSNLSSAGVSPGHWYVNGYVSGSKLGDLVAASFFTVENGHANFTPKPLVSPVHNGNKTNNTSNLSGNSTNSSGNAKISGAAVSARVLDGTLMGLVAVGLGLAFV

SEQ ID NO: 158 - protein, 285 aa, Phakopsora pachyrhizi
MFKGLSLAVLSIALRETIGQATTVSTDSLNAISNQFDSSYLSSQYPDGFGIPLKAQAILNVNYPFGNVQLGKAYNASDVANQPTISISPLESSQASFKAPSAFTLMLADANALGNPDPKGNYRHFLENGVTFGDPTSNGTMSMNPGSGTVVTSYAGPGPLPNEGPHRYAWLLFNQPGDFQAPSNLSSAGVSPGHWYVNGYVSGSKLGDLVAASFFTVENGHANFTPKPLVSPVHNGNKTNNTSNLSGNSTNSSGNAKISGAAVSARVLDGTLMGLVAVGLGLAFV

SEQ ID NO: 159 - protein, 236 aa, Phakopsora pachyrhizi
MFNMLIALAITLVHAGSVFCQQAASAQQDSSTTIVANTNLNGAYGVSGQISFTLIPTTEEVAVKISVKGLMAINSTAQFGYHIHTNPISSDGNCSSALGHLDPLQVTDSVVCNGTARLCQIGDLSGRNGKLPGNQSVANVSYTDKFLRFWPQPFTLLGRSIVIHAPNTTRIACGNITSVVDGTADNQGKPTNQSSNYVTVYAFLSCMLFTRPLLTSFKKTATLHEHLLLLITLPSE

SEQ ID NO: 160 - protein, 85 aa, Phakopsora pachyrhizi
MLNLSFSGLLIFACVLFQFTSISAKNLTATAVKNALYSSCEKSALAKPCGGALDKLYDTGRFKSFTIEKQVHFDKTSNCKFTWWT

SEQ ID NO: 161 - protein, 85 aa, Phakopsora pachyrhizi
MMGVLMMLFLHLYMKYDAPLFLQSLMPLKTTYENKVIQIHLLGRSATGDLKRPFKTGGFMGAATDAKTDAKSIKEAEKSSVKKDQ

SEQ ID NO: 162 - protein, 47 aa, Phakopsora pachyrhizi
MKGNLFSLSYFFAFKILNLTRFLQICFDCIIQKRTIIKVLIDIGYFG

SEQ ID NO: 163 - protein, 126 aa, Phakopsora pachyrhizi
MSFAKQVLLSICFFAVFSKINALTVAQPSVRCLGDTNPAAIKAEDCDNAMSQFSVDSNGYIQYDPQGEKRDFGSCRIQIQSTSAASNLATINPEVLKIYISTGLTSCNGGTSAIEPQGLVVNVIKL

SEQ ID NO: 164 - protein, 126 aa, Phakopsora pachyrhizi; identical to SEQ ID NO: 163

SEQ ID NO: 165 - protein, 354 aa, Phakopsora pachyrhizi
MRFSLIVAVYLAFSAYSLALPVALNSLTPLQATPNYLGRETSSNLTSSVYPQNDYEDMQVGSSNLGNQLGSYGGEESYEEEGNENQEGYGEDGNGYEDESYGNQNYEYQDYDNQYEAGKEQGGYQTQGNNQGYRSGSNKNYSPGFNAGSYGFGSVNNRDYGIVGGYGSIQGSNQNYNFRSNIGYSSGIGAGIFGGYGSANSRDYGIPYYAPRDYSEAGYGAGTGDEGQVYRYTVGEGMPNGAGLGAGVGAGVGAGVEYGEGSSLGKGIEYGVGAGYGAGYGAGVGAGEGAGYGAGVGAGVGAWLWCRCWCRSRRRLWCRRQLLDMVSTLTLALMISLRVENIIQNIISRSITLV

SEQ ID NO: 166 - protein, 352 aa, Phakopsora pachyrhizi
MRFSLIVAVYLAFSAYSLALPVALNSLTPLQATPNYLGRETSSNLTSSVYPQNDYEDMQVGSSNLGNQLGSYGGEESYEEEGNENQEGYGEDGNGYEDESYGNQNYEYQDYDNQYEAGKEQGGYQTQGNNQGYRSGSNKNYSPGFNAGSYGFGSNIGYSSGIGAGIFGGYGSANSRDYGIPYYAPRDYSEAGYGAGTGDEGQVYRYTVGEGMPNGAGLGAGVGAGVGAGVEYGEGSSLGKGIEYGVGAGYGAGYGAGVGAGEGAGYGAGVGAGVGAWLWCRCWCRSRRRIWCRCWCWARCWCWRRCRGWCWRRLWCRRQLLDMVSTLTLALMISLRVENIIQNIISRSITLV

SEQ ID NO: 167 - protein, 364 aa, Phakopsora pachyrhizi
MRFSLIVAVYLAFSAYSLALPVALNSLTPLQATPNYLGRETSSNLTSSVYPQNDYEDMQVGSSNLGNQLGSYGGEESYEEEGNENQEGYGEDGNGYEDESYGNQNYEYQDYDNQYEAGKEQGGYQTQGNNQGYRSGSNKNYSPGFNAGSYGFGSNIGYSSGIGAGIFGGYGSANSRDYGIPYYAPRDYSEAGYGAGTGDEGQVYRYTVGEGMPNGAGLGAGVGAGVGAGVEYGEGSSLGKGIEYGVGAGYGAGYGAGVGAGEGAGYGAGVGAGVGAWLWCRCWCRSRRRLWCRCWCGSRYRIWCRCWCWARCWCWRRCRGWCWRRLWCRRQLLDMVSTLTLALMISLRVENIIQNIISRSITLV

SEQ ID NO: 168 - protein, 116 aa, Phakopsora pachyrhizi
MSFAKQVLLSICFFAVFSKINALTVAQPSVRCLGDTNPAAIKAEDCDNAMSQFSVDSNGYIQYDPQGEKRDFGSCRIQIQSTSAASNLATINPEVLKIYISTGLTSCNGGPLSHKA

SEQ ID NO: 169 - protein, 100 aa, Phakopsora pachyrhizi
MNFGSSSKKNLVARPSVGIRRLVLVCTAMVWSITLILSQPSGVNSSPIFKIAKTIAPTVVKAAAAGGGLIAVEQIASKVIGRSTDGQNIEEETSGLINFS

SEQ ID NO: 170 - protein, 248 aa, Phakopsora pachyrhizi
MFSKSFQGLSLLVVLMASIIFNCDESNAQSNFNSFQQPGTNCYEQSKSYGINFEHCEKALRKISYDSNGNLDNSSKTVFVWHKSCVVKVQKATYAQPSRQQVEYGVRNLLQTCPTRGGIYIPSNDFRTYVYSSNRENVYNLNSPVCIKHQCHINPNDCLMAFNNIPLSTKGFFLPSGSTSSNVIKTSSGNCTVKLVTTDGAGFRINHPEINSGIKTLLSKCGSRPGYNYFGGGSLGMNGDIKIITQNS

SEQ ID NO: 171 - protein, 519 aa, Phakopsora pachyrhizi
MALLNRIVFVLLALTFARVIESRTGVKLDSRLNTRNTQEQLNHPRVKCGRSTATQATGSTYRCRNSSNDGKKHKKVPKGYGKKEKGKYTPKGPKHESYAPKAPSKLPTGGYGGHEQEQYAPKGHSSEPYVPKGVVSSPVTLAALVTVITYVSKVNWLLCIIKQRFQHDLVRQSATAKVIQSSGPSGRHSNSGLVATVFGATGFLGRYLVHKLAKKGTQVIVPYREEDSKRHLRVMGDLGQIVPLEFDLQNETSINECVRHSHLVYNLIGRDYETKNYSFDSVFVDGAAKITQITKDNGVNRLIHISHLNASHDSPSKFYRAKASGEDKVKSIFPDVTIVRPSTFFGQEDRFLNKMVTKPFTFNVNRLETITRPVHVLDVAEAISLMGTAQSTVGKTFELAGPRTYSYEDLFETLENLTLKSLTPSLNLPLPIFKLFNKIVSRAVWWPTLSEDEIVRRCIDDKEELSEGALGFDYFGIEPDLVEDAAITYLRFYRSAANFDKPIQRGGFKVKPKTYNVVN

SEQ ID NO: 172 - protein, 40 aa, Phakopsora pachyrhizi
MQTLQLMVLAKVEVAHLVTSVEHQLVEGQRTQGKIGPKLL

SEQ ID NO: 173 - protein, 33 aa, Phakopsora pachyrhizi
MNCNDACSWVILAVFLGISALNIACVEGASVVG

SEQ ID NO: 174 - protein, 42 aa, Phakopsora pachyrhizi
MNYIFRCTIICIWLLRKKVVNLAEVYLIKPVVSPTNHLIPID

SEQ ID NO: 175 - protein, 80 aa, Phakopsora pachyrhizi
MVLPRNTYRDPWAKNEAWRNHPIFSRRQQLRNFFPGFGWASVAFTIYVLYDNNYRAKNKNIEDVKGDFNKIVDDVKNLKF

SEQ ID NO: 176 - protein, 176 aa, Phakopsora pachyrhizi
MLRLISLGLLVCSAARLTVSSPSDLSLRPRQLGLGAGGAAAGSFSSSTSVSTFSSFVQGWSVIPTAFGTCAATFQQRVTVQVAVQSVQQLYSTVNGVLGHYGGCGGCGGASAAGAYASKYQSIITQSFTSWQTILQVGQSSYANAWDSQFIPLFRQFNPFLTAVQQNSGLFGINLD

SEQ ID NO: 177 - protein, 368 aa, Phakopsora pachyrhizi
MVLLNLRAQILISLNARLFKVFNLRDNKVFTISLLKNILGSSFHVPEHLIQFTLVSGRYTKDSDALVDLRASDGNFASLDVGVLPPHYCSSLSEAIVSFEFRANPTPTYLDGVSVAGIQEIPLMYQTLRPVSDRLNNELNNLNISNKKLTKDQRIALRARYEDPFSISSKSCFEEDSDDDSLYEDEDSESELGDSDDDLYSDDEVESTGFTESRSAFSNYEACHKADSGQYSSFWSSDSSKLNPYKLRQLLSVVPNSDSHKEIPLRESVLRLQFQTRVYNHLIKTYNHQSRSSTSSDSSSSSISSQTSSLSSSSMVSNLSDHESDESQLYSENYQNKFTNATSSIAQKPTQSKRKQEFLKILIPNKFF

SEQ ID NO: 178 - protein, 132 aa, Phakopsora pachyrhizi
MFHRATVFITLFSALSILNDNRVSALMASVPDTQTCSFYTGAYTNSATCNEQPNVVCTQGCYGPYVVATGCVLENDSRAGASSSSQVCTMGFGRNTAAAKACINELGTFRCTGQTSGSATCYGCRNSAQNSK

SEQ ID NO: 179 - protein, 188 aa, Phakopsora pachyrhizi
MFMTSLCTSAVRSTAIGGGIKVLSSLTSLVSVPSSTSLLPQPGNQSRRGKVILRGMSSQTKSTIVSTEDAPKAIGPYSQAIKTANGLVFSSGCIPLDPKTMEIVGENDVQAQTEQCLKNLESVLKASGSCLNSVVKTTVYIKDMNQFSKINEVYSKFFKDFKPARSCVEVARLPKDVLVEIEAIATVI

SEQ ID NO: 180 - protein, 58 aa, Phakopsora pachyrhizi
MKFFLGLTNLFLIASIVGVVFSSPVNTNGEDTRLQKRKTVAPATCTQGGRVIKNKCKI

SEQ ID NO: 181 - protein, 183 aa, Phakopsora pachyrhizi
MLFCKFLLIFSLMCYGALAAPIMRVFWAAFPLLNLPVFKFLIHPQESLKLLLGLERGFQEIKDLELSKSAFRLQKLILGDQKVFLKEIDGKIENEATTFANVMRLDNKKQFENIPFRLPLNTKDLHESIYNSLVEVVNDPKIIGLQRIPNPKLTQKRLSELKPISEPIADINILRTAVAHSKL

SEQ ID NO: 182 - protein, 75 aa, Phakopsora pachyrhizi
MNCNDACSWVILAVFLGISALNIACVEGASVVGPEKSIKARQNEVSSYCRVPGGCRHTVEFREGVVDEMAFSLPC

SEQ ID NO: 183 - protein, 76 aa, Phakopsora pachyrhizi
MNCNDACSWVILAVFLGISALNIACVEGASVVGPEKSIKARQNEVSSYCRVPGGCGRRDGLLASLRSQRSWVRKIN

SEQ ID NO: 184 - protein, 67 aa, Phakopsora pachyrhizi
MNCNDACSWVILAVFLGISALNIACVEGASVVGPEKSIKARQNEVSSYCRVPGGCGRRDGLLASLLG

SEQ ID NO: 185 - protein, 52 aa, Phakopsora pachyrhizi
IPLILAWAMSIHKSQGQTLNRVKVDLGKVLKKLFSRATSLEGLQVLRFDHRK

SEQ ID NO: 186 - protein, 296 aa, Phakopsora pachyrhizi
MTLRLFKWVGFLQLLLRLTSAQDTSGTTPLYQKKFPWPNVPYRADTGDGPRGTQQGYNKCNSTTQNQDSLCQTTFLNSIQEFCLWGAIKPNSIVGNIEGEMVAYCTTKKFGTRLIPSGAITGVQLTRTPGYIQVVGFIDQTALNLQANDTGGEEDPHGADQRGNPLGALMYSSAFNSNSGASSGQSSNSTSSSSYTQVSEWSFFIGSGVFCYKACDPAGPNAPQLCQHIYDRIGCAYNAPANYPSINGTYQSCQGENQLPAGTYIENGVTKTYQQPPESLGPITLVDLITSSDYLG

SEQ ID NO: 187 - protein, 148 aa, Phakopsora pachyrhizi
MFVSLKAIISTTIATLAVIGVVSGQNITIVSPQSGQVIRAGDVLTVELAQQSTTSSVQNIAFSTGFSSRPTDTISLGRPLGGTQFLTTRGNITFQINVPKASEFIDGISNVPYEFKVAHYYFLGPAFQLAYNNNLKIKSKRVLSILNF

SEQ ID NO: 188 - protein, 85 aa, Phakopsora pachyrhizi
MVGCESGATKKSMIIKMIGFLCIWFQLLEIVLTQSTAATTSATETKNYNSYFKRLEKRSPSTSSGRIEKRHRRCGPPYGGYYGPG

SEQ ID NO: 189 - protein, 103 aa, Phakopsora pachyrhizi
MVGCESGATKKSMIIKMIGFLCIWFQLLEIVLTQSTAATTSATETKNYNSYFKRLEKRSPSTSSGRIEKRHRRCGPPYGGYYGPGYSSGYYNYGYYSGYKKKQ

SEQ ID NO: 190 - protein, 304 aa, Phakopsora pachyrhizi
MFFSKSLLITIFMGCLIDRLQSLPIPSFYGTSSVDQNPDSTQVGLNNCNSQLSGTELGSSGGRSTITSPSIASGVSGNGEISSADRNKYPVTQNAGKINQMAGSEKEVGSGLAKEQSERDAKIQKDTQTRQAQEQAITNAHQQKDDDSRKAQDQEEKRRVKEEENRKEADEEKRKEADEEKKKEVGEEEENKKREEARQEEENKKKEEARQEEENKKKEEARQKEENKKKEEIRQEEERKAKEAEDRQAAEQANEQESRKHQDKIQAPPPGTAGDDSGNKPSKPSQSSSQLKSGDATCKISSDC

SEQ ID NO: 191 - protein, 121 aa, Phakopsora pachyrhizi
MFHDLNYFAPALLAFTCLLELSAAQEQRSCSFYTGANTTSATCNEQPNVVCTKGCTGPFVTATECTPVNESEEAIASTQVCSFGFGRNTAAAKACINELGTFRCTGQTTGSATCDGCQARS

SEQ ID NO: 192 - protein, 202 aa, Phakopsora pachyrhizi
MMRASKSIVFISACFYLFSGETLAAPALKGVETEANSIPDAAKFAESLSIKTDSLSELPLKSSPTIPSTPSGLDGNPQSSLLDKSAMKEIDASESAEKIKSHSFTSGISAKRPTKSLDASATGSDGLVQQVHVGDSQIAVDAKTEVPPAAAYEHNKVTRKCNHDHDHDHHHHHHHHHGPGDNHDHDHHHHHHHGHDHDHDHA

SEQ ID NO: 193 - protein, 400 aa, Phakopsora pachyrhizi
MFFSKSLLITIFMGCLIDRLQSLPIPSFYGTSSVDQNPDSTQVGLNNCNSQLSGTELGSSGGRSTITSPSIASGVSGNGEISSADRNKYPVTQNAGKINQMAGSEKEVGSGLAKEQSERDAKIQKDTQTRQAQEQAITNAHQQKDDDSRKAQDQEEKRRVKEEEEKRRVKEEENRKEAGEEKRKGADEEKKKEVGEEEENKKREEARQEEENKKKEEARQEEENKKKEEARQKEENKKKEEIRQEEERKAKEAEDRQAAEQANEQESRKHQDKIQAPPPGTAGDDSGNKPSKPSQSSSQLKSGDATFYGTGLGSCGKTDTDTDYICAVSKILYDSFNDMGNPNNNPVCGKKIRAHYNGNSVVVTVHDRCEACAVHDLDFSPAAFQQLGSKDEGRLHGMQV

SEQ ID NO: 194 - protein, 256 aa, Phakopsora pachyrhizi
MFKSNFLLLSILIALSGVYSLPNGNNRPSDSSDHPQSEPQEGRGVGRERVRVRYSYRGDFRDGDGGHRSSHHRHGRKRPHHRHRGDQDDDSKGDKSFGDEGKDQYANDLSSQSSNGYVHGSEGGYKRDPSGPQGYTSNPSAEEASKELYSSSAPATPTNDHPDSSTSTSGHDNSGGDDKPSKDPSGYSSSGQDPSKDYSKPSKDSYTADQSSGNKQGGSGSPTKEESGSGDKPNSGGSGSEPYFSAGKSSKNQYRG

SEQ ID NO: 195 - protein, 335 aa, Phakopsora pachyrhizi
MRIKILLLITLWPALLSFVAATPALFQAPNSYSGDQPPLGALVVRQIQAVPGEFSNLSQAIGSLATKEGPQVIFIYPGHYRESVKVNYKFPLIIQGYTKNSSDYRHNQVKVENAVSAASVGGNAQSSAIWARTDFFTMLNINVTNSYGNGTDTQAVAVTADGDYQVYKGCVIDSFQDTLYIKSTSSYFFRCLIAGSVDFIFGAGTAWFEKSKIAVKQSHSINIITAQRKTVLKTQFVFNDSRVVALGNVANQSTYLGRPWSENASVTYQFCKLSNIIKPEGWRFWNPNDQRTQNIKFFEFQNTGPGSLGERKYGRRAEAALNITEVLGSDPESWI

SEQ ID NO: 196 - protein, 310 aa, Phakopsora pachyrhizi
MVLPLQIIVISIFISLNNLSAARLFGSDGIRRIISGRSTHPSVGDRHGLSPRNLLNQESYDDYKSDENLYGSTTKTTGLTTEPVNLVPDQSKFKVAGTSTKQDYVQAVPASGLSTVSSESPKVRQMSQAAVTDLNTCDKGHPKAEINRYLLDCNAGIGSYSAVIIANCKSINKSNAQQTGTLILDGLRGMNNVLKLTSSSITSCGMDSKPLAVDKGVDLSILANSAVGIVGTLYPVFESISKLYEPYPLIEKSCSETLTNVSTSLSNVIASCGEQVNLFDTKFFKEIKNYMSQFSDVTNNFNAFIGSFDS

SEQ ID NO: 197 - protein, 310 aa, Phakopsora pachyrhizi
MVLPLQIIVISIFISLNNLSAARLFGSDGIRRIISGRSTHPSVGDRHGLSARNLLNQESYDDYKSDENLYGSITKTTGLTTEPVNLVPDQSKFKVAGTSTKQDYVQAVPASGLSTVSSESPKVRQMSQAAVTDLNTCDKGHPKAEINRYLLDCNAGIGSYSAVIIANCKSINKSNAQQTGTLILDGLRGMNNVLKLTSSSITSCGMDSKPLAVDKGVDLSILANSAVGIVGTLYPVFESISKLYEPYPLIEKSCSETLTNVSTSLSNVIASCGEQVNLFDTKFFKEIKNYMSQFSDVTNNFNAFIGSFDS

SEQ ID NO: 198 - protein, 248 aa, Phakopsora pachyrhizi
MHSKVLISWVMSAFVAIVLHAANSIQDNEEPPIVAKVYFDMKQGSKNLGRIEIGLFKNTPKTSENFRALAVGDKKSKDGIPLAYKGSKFHRVINQFMIQGGDFTKGDGTGGLSIYGPKFQDENFINKHTGPGTVSMANSGPDTNGSQFFICTALTSWLDGRHVVFGKVLRGMDVVFRIETLPTDNGDRPKEDIVIADSGELKIDDELDADGNKFEKVKLVTGSNKQLDQSINSKSDSVQFSREADGLK

SEQ ID NO: 199 - protein, 225 aa, Phakopsora pachyrhizi
MFRSLFLGILACSAVKLTICSPSDEVVVKPRQIGYGAGLGAGGASSSSFSSSTSVSSFSSFVQGWSVIPTAFGTCAAVFQRQVTVEVAIQSVQELHSTVSGVLGNYGSCSSCGGASAASSYSSHYQSIIVKTFTSWQSIMSVGHSLYDNVWEPQFAPLFRQFNPFLTAVQQNSGFFGINLGNILGGLHLNLNLFSSCGLNIGGLLGGVLSTVGGLLGGLKDHEEA

SEQ ID NO: 200 - protein, 116 aa, Phakopsora pachyrhizi
MFTVLKVVPSMFLILQTLLALLATSLNIGMAKDIPPASVCYSYSNSDNDRATCHGATCPDGCSWPFITAQNCVPTGPTSSKSTTQTCHIGYWKDGTDGICTNRLREMYRCSGKTTG

SEQ ID NO: 201 - protein, 187 aa, Phakopsora pachyrhizi
FFSLSLLLLSASSSSLSIICPLPRRSQTHHPYQRIRPRSNLIRIKHSSNDSSSESDSDSDSSLKSNQIGSGNDGQTFGQDDEVLSEFLSLFNPSSKGSFLILSENLSNNMTHGFENAECTSVGSNLNDFNQSIHQVSGSEQDWIASSPISRCLNPFSRQEGNKNLRSDDKIQDKEGGLIENFKELLK

SEQ ID NO: 202 - protein, 291 aa, Phakopsora pachyrhizi
MYHLTFVLFSLFFLSANGASTSEPSGQTLDCSHYTGANTKEATCNEFPGRICHGGCTGAVVASNCTLNPGEEPKDQTCTIAFGKSSATISICRNEKGSYSCTGPIKGSAKCSVCVDPPTGSNQSPTTPAPAPGNNGSTSKEGQTLQCTHFTGANTQSATCNEVPGRVCNKGCTSSVVATKCTLNPGDQESQQNCTQAFGKSSAAISICINDKGSFSCTGSVSGNATCSGCTDQSSSGSSEYPGSSGTTKPPTDPTGGENKDQDKKAEATSLQFAMSSFCLALSLMIGVAVL

SEQ ID NO: 203 - protein, 310 aa, Phakopsora pachyrhizi
MVLPLQIIVISIFISLNNLSAARLFGSDGIRRIISGRSTHPSVGDRHGLSPRNLLNQESYDDYKSDENLYGSITKTTGLTTEPVNLVPDQSKFKVAGTSTKQDYVQAVPASGLSTVSSESPKVRQMSQAAVTDLNTCDKGHPKAEINRYLLDCNAGIGSYSAVIIANCKSINKSNAQQTGTLILDGLRGMNNVLKLTSSSITSCGMDSKPLAVDKGVDLSILANSAVGIVGTLYPVFESISKLYEPYPLIEKSCSETLTNVSTSLSNVIASCGEQVNLFDTKFFKEIKNYMSQFSDVTNNFNAFIGSFDS

SEQ ID NO: 204 - protein, 335 aa, Phakopsora pachyrhizi; identical to SEQ ID NO: 195

SEQ ID NO: 205 - protein, 68 aa, Phakopsora pachyrhizi
MKKILDLYMICALSFLYSSLASMAMCQLGFYSLRTILLLLSTCPKALLKKELTFNSKTRATSISLMVE

SEQ ID NO: 206 - protein, 54 aa, Phakopsora pachyrhizi
MVLGRVTHYAFDLALVSTVLAGVKRNTGYSLKTDSLPEGVPQSLISTYLVVGEN

SEQ ID NO: 207 - protein, 141 aa, Phakopsora pachyrhizi
MALLNRIVFVLLALTFAGAIESRTGVKLDSRLNTRNTQEQLTHPRVKCGRSTATQATGSTYRCRNSSNDGKKHKKVPKGYGKKEKGKYTPKGPKHESYAPKAPSKLPTGGYGGHEQEQYAPKGHSSEPYVPKGVVSSPGYS

SEQ ID NO: 208 - protein, 46 aa, Phakopsora pachyrhizi
MVFPQQISRSRKIPLRLVLLSLEMVVLLTRDSPLVSTTDQRYFDLN

SEQ ID NO: 209 - protein, 74 aa, Phakopsora pachyrhizi
MRGRQIDTGFLVFWVKLYFLLKTVTGGLVTKPDSIEKLSGSGVKIEDYTSKGYSPLFFLPFYLPSQDVIKNFIS

SEQ ID NO: 210 - protein, 72 aa, Phakopsora pachyrhizi
MARSVLKFKDYLQLAIVLLTIYQSILCVGSNVRNHIHRRHQPSASDPKASPTRPLEWGDLNIIHTTDSHVCL

SEQ ID NO: 211 - protein, 57 aa, Phakopsora pachyrhizi
MNMILKKINSLILAFLLKLKSSLQGQKYHHLIIDIDLQFLKAHFAFNGMVSRHSKNT

SEQ ID NO: 212 - protein, 38 aa, Phakopsora pachyrhizi
MTIMIMKVMMMMTMKGRIKKMSRIQMTITITCRDGYNF

SEQ ID NO: 213 - protein, 82 aa, Phakopsora pachyrhizi
MKVWIGSLAVLSVLSVYMLVHGREFINLPKLNTREVIIKYDGPGYQSSKNGTGLRGHQSYTSLTSSNKRSGGGDKDFIKKTF

SEQ ID NO: 214 - protein, 86 aa, Phakopsora pachyrhizi
MFIKSYCFLLGLTLFLSCLSTTITGELNNMDDQILAAKFRARRSIETIKENSPIKQERKKLRNEPVNTLVAYPVRDPNCVDDDAPR

SEQ ID NO: 215 - protein, 84 aa, Phakopsora pachyrhizi
MRCFILSIVILAVAQAVTAIPLVERAEVNSSKGDTLKKRFGCIDPLVSVDVLNRGNGARGSLLDVNILNGFLKDAQKKSQAKNE

SEQ ID NO: 216 - protein, 141 aa, Phakopsora pachyrhizi
MALLNRIVFVLLALTFARVIESRTGVKLDSRLNTRNTQEQLTHPRVKCGRSTATQATGSTYRCRNSSNDGKKHKKVPKGYGKKEKGKYTPKGPKHESYAPKAPSKLPTGGYGGHEQEQYAPKGHSSEPYVPKGVVSSPGYS

SEQ ID NO: 217 - protein, 258 aa, Phakopsora pachyrhizi
MFKKSTMILTFLFLALHGTLSSPVPDSLTSNDNNDINFSSEIFARAVQDEYPQEQENSRSGIPGNEFENSNVVNNARDPAELNEQNEQIRGDNQPTTNSNIQLNLKSSTEDKTAKDSPRKRSPESFDTTSSVQPSPRESNGEITNPQVNDVSHSDQINSGNSPTEGFHPGDLNTSGDQGLSKIWPNKDEPQVNYDPHKDQTTSESFPTQGSTQGNFQPQPSRDTTQINENPSIDQTNSGTTTPNPVNSQDGNKPHGDK

SEQ ID NO: 218 - protein, 50 aa, Phakopsora pachyrhizi
MVMILTVYWYSILQSFVEDDDEKNQMFSFYSAYSPLYSSVSVSKDLSPGA

SEQ ID NO: 219 - protein, 321 aa, Phakopsora pachyrhizi
MMMMMMMMMRQATKKSLGSVNLSSTRAIHHIAPFSKIASVAPLLSALTSSSHSQRFSLNNQLTHTSSSVRSFFSLPSFPPSLNVSKDIHGYEDLNVRDRYSQRGDVLTYKETKRLSFSKHQLYYIAADIESYSSFLPYCAQSNLRSVRALDKTSDDSLESNLITNDERRQWLRGGKVGETFLIELEQVNGFKGLDELNRTSVECRKFDLVKLTMCDSKLFKSLSITWKFKSPREIFNIDSILNNQNGLTSQSSSLREEDNEETGDAFVSIETSLTLLSGDINQQSGQVKGPKFSVNDLSGELYWKAMSEKVLASYESRLNQ

SEQ ID NO: 220 - protein, 299 aa, Phakopsora pachyrhizi
MMFQSKLLGLLILSLYLCKTSFAIPLGDDQKADPVDTSGFTIISDLYNKSYNNHNVKTDHPEPPKYTDPKKPDPPNWKEPKHPEPPKYTDPKKPDPPNWKEPKHPEPPKYTDPKKPEPPNWKEPQHPEPPNWKEPNHPDPPEWKGPEHPEPPKLKEPKHPEPPKWKVPKHPEPPKLKEPKHPEPPKWKEPKHPEPPKWKVPKHPEPPNWKEPKHPEPPKWKEPKHPEPPKWKEPEKPEPPKWKEPKHPEPPKWKEPEKPEPPKWKEPEKPEPPKWKEPEKPEPPKSKEPGYTDPPKERN

SEQ ID NO: 221 - protein, 57 aa, Phakopsora pachyrhizi
MTNGSWVIESGVMRLKMFIREKQPKTDNILSIISYRLSISLSLSLSLSLSHAQRLQK

SEQ ID NO: 222 - protein, 124 aa, Phakopsora pachyrhizi
MRIIAFFALLAAICLPFVENRCHRSGATFGSSPNRAYATQLLPGVCKEFQGTYGPNQDKSVCRNGRDRDTSFRYYIKHVSDGYRSIGPTECTDGLNKEIVNCDRGGKTAYGNWEYNVDPNKGRC

SEQ ID NO: 223 - protein, 141 aa, Phakopsora pachyrhizi
MISKNLKLDLKSILIIGCFLTSCLLHMVVTIDITCDTKVELNDDDKDNPKVICSSFGLPPAPFECPRKSCNDFPIGLQCVQFMNQKNLPNFRPSKIHKSELTCQTFEFDLGKNVCITEDGEKYRCAGNREPMKCTDCKRIR

SEQ ID NO: 224 - protein, 32 aa, Phakopsora pachyrhizi
MRNKWRVIYFCFIKLIISFGFRKFNFFYKVKT

SEQ ID NO: 225 - protein, 81 aa, Phakopsora pachyrhizi
MRCFILSIVILAVAQAVTAIPLVERAEVNSSKGDTLKKRFGCIDPLVSVDVLNRGNGARGSLIDLNILNGFLKDASKKSQA

SEQ ID NO: 226 - protein, 138 aa, Phakopsora pachyrhizi
MMMKSLYLSTFLFGIFLRSSAGLPHHSDPSTILARGLENRYNNWGKGTYYYVSDGNQVACGGYYQDSDMVCAASATDFNNKNSCGELITVNHSGKSVQCTIVDQCEGCSAGSLDLSPAAFQALAELSVGRIDISWNFN

SEQ ID NO: 227 - protein, 57 aa, Phakopsora pachyrhizi
MVRSTIKLLFFFFNLSSLSVFLNSTKDASFLSSEILLQINQTFRFVSIKKKKKKKKR

SEQ ID NO: 228 - protein, 61 aa, Phakopsora pachyrhizi
MVRSTIKLLFFFFNLSSLSVFLNSTKDASFLTNFLSSEILLQINQTFRFVSIKKKKKKKKR

SEQ ID NO: 229 - protein, 51 aa, Phakopsora pachyrhizi
MFFFTRSPLKLCFRTLCYQRTIKCCLGFFGNKFSFISRSMSTPVVIRFYCI

SEQ ID NO: 230 - protein, 139 aa, Phakopsora pachyrhizi; identical to SEQ ID NO: 63

SEQ ID NO: 231 - protein, 66 aa, Phakopsora pachyrhizi
MNTISSICLAIAAFLIAQNNFAGATDLNPRDEVANSAHTQLEPRAVGRYNHLEARKKKPKTRKGGR

SEQ ID NO: 232 - protein, 220 aa, Phakopsora pachyrhizi
MVYSKATFTLVSLLPMILAQQSINNSDTTTTAATPASPTIGSTGTTNSSSSTTTSTTTSNTASGAAQSFPAPTVSGTSCGTAQQNFNQCVTKVSKDISSCPSTDNTCLCQTYANLAYCYNACPDLASSGAGYLQQSTVNCDAAGIKPNATSNVTTTPVTSTTNRNTTNTSPISNTNKNNSTSGNTAATFAAGNSKASGIEAPLLSVAVVGLCGIIASLFA

SEQ ID NO: 233 - protein, 289 aa, Phakopsora pachyrhizi
MMSKLFLIAAITAFVPSSSYGSSNTPASPQGSLGNISPFKENVLSLSCGLNLLVGGRVAPLAQSGYANLCLQCKIDLFGVRVSFAYTSALSESINTHGLSVSDTSRLQGFVQGRLLDSAAQCSAKNECSRKCSDDQFCQSSDFSNGRCTFTAKNLASNNKNGALLAQAFADVFNQGGSQYCGFCSGDSSCVNPSARARSYKRHAEIARRSQLQTKCPTGLTACPIVQMNQTAAEVWSPSPAVSYECLKVDEELSSCGGCATLGKGTDCSAVEGVLSAGCSDVQLGRIRS

SEQ ID NO: 234 - protein, 327 aa, Phakopsora pachyrhizi
MMSKLFLIAAITAFVPSSSYGSSNTPASPQGSLGNISPFKENVLSLSCGLNLLVGGRVAPLAQSGYANLCLQCKIDLFGVRVSFAYTSALSESINTHGLSVSDTSRLQGFVQGRLLDSAAQCSAKNECSRKCSDDQFCQSSDFSNGRCTFTAKNLASNNKNGALLAQAFADVFNQGGSQYCGFCSGDSSCVNPSARARSYKRHAEIARRSQLQTKCPTGLTACPIVQMNQTAAEVWSPSPAVSYECLKVDEELSSCGGCATLGKGTDCSAVEGVLSAGCSDGKCYTFSCQDGYKLSNITNKCTMIKSKSNLYQKYPPSVVQLGRIRS

SEQ ID NO: 235 - protein, 198 aa, Phakopsora pachyrhizi
MQSNFLLAFCLIAAVSVPVSRAHGVITSVEGANGQTGSAFGMVESTPRDGTRTNPFQTDSSIIRDREVSSGKASACGRTLAGGNNDIASDMSSAESAGLASIGPDGKVRMTLHQVNGDGGGPYSCEVDTTATGDNFKKMNIDTNVPGKNSRSRAKATEFPLVATMPAGATCTGGADGQTCLVRCRNVRFIFKYQMFIS

SEQ ID NO: 236 - protein, 60 aa, Phakopsora pachyrhizi
MIMDLLLVVSLVGLSYGSEHQEYRSYYPDFEERNLFQQTGENLLQSLGLEYGCLKFWLGA

SEQ ID NO: 237 - protein, 127 aa, Phakopsora pachyrhizi
MFISPLILAFTYIIASIGQSCCLGSLDQNQPQKSHSSLQKRAVCLVPNARKGCKIWNIDACCQSCCSQKYATRRWLEDCHCVNQGSSVVYQAPGTVSYVPVQQPPTTYVVSSPPPPPPPTVVTVYHG

SEQ ID NO: 238 - protein, 145 aa, Phakopsora pachyrhizi
MNSKIFLMLVVCTFFYTPSGASKSLKVRAEKGYHSSGHKGHGHKFKIRAETGDNIVTQPTINRSYGHHKYHHKGYSKHYRKRALKDVGKEALEANAASGKAAPTADSGAKASVVSRKHGHGHHKMHNAYHKGYRKHYRKREKAVY

SEQ ID NO: 239 - protein, 233 aa, Phakopsora pachyrhizi
MNSKIFLMLVVCTFFYTPSGASKSLKVRAEKGYHSSGHKGHGHKFKIRAETGDNIVTQPTINRSYGHHKYHHKGYSKHYRKRALKDVGKEALEANAASGKAAPTADSGAKASVVSRKHGHGHHKMHNAYHKGYRKHYRKRAFGSEKNKANKTQASTKKNFGEKKLSIRATPELSGKTASIDQTPVAKAGLTEKAEKSIANGTVPSQGTAVLRKTESYYRPKHYGYNGYRNSYY

SEQ ID NO: 240 - protein, 206 aa, Phakopsora pachyrhizi
MNSKIFLMLVVCTFFYTPSGASKSLKVRAEKGYHSSGHKGHGHKFKIRAETGDNIVTQPTINRSYGHHKYHHKGYSKHYRKRALKDVGKEALEANAASGKAAPTADSGAKASVVSRKHGHGHHKMHNAYHKGYRKHYRKRAFGSEKNKANKTQASTKKNFGEKKLSIRATPELSGKTASIDQTPVAKAGLTEKAEKSIANGTVPSQ

SEQ ID NO: 241 - protein, 401 aa, Phakopsora pachyrhizi
MQTKILLALCLVAISQVNAHGAITAVQGSNGMTGEAFGVDQSTPRDGTKRNPFQTDSSIIRDREIASGKSSACGRTLAGGNNEIGAAMSKAESAGIPSVSSDGKVQMTLHQVNGDGGGPYTCDVNASGDGKTFTPMTISTNIPGKNSRSNAKAEDIPLIAEMPKGMTCTGGSDGQTCIVRCRNAANAGPFGGCVAVTQATDATKQKRELSAEQRIDEMIKQAEAAEAFGDETLAKREMSADERLDEMIRQAEDAELAEENLSKRELTADQRLDEMIKQAEAAEIAEENLVKRELTADQRLDEMIKQAEAAELAEENLAKRELTADQRLDQMIKQAEDAELAEESLSKRELTADQRLDEMIKQAEAAEIAEENLAKRELTAGQRLDEMIKQAEAAELAEELI

SEQ ID NO: 242 - protein, 401 aa, Phakopsora pachyrhizi
MQTKILLALCLVAISQVNAHGAITAVQGSNGMTGEAFGVDQSTPRDGTKRNPFQTDSSIIRDREIASGKSSACGRTLAGGNNEIGAAMSKAESAGIPSVSSDGKVQMTLHQVNGDGGGPYTCDVNASGDGKTFTPMTISTNIPGKNSRSNAKAEDIPLIAEMPKGMTCTGGSDGQTCIVRCRNAANAGPFGGCVAVTQATDATKQKRELSAEQRIDEMIKQAEAAEAFGDETLAKRELSADQRLDEMIKQAEDAELAEENLSKRELTADQRLDEMIKQAEAAEIAEENLVKRELTADQRLDEMIKQAEAAELAEENLAKRELTADQRLDQMIKQAEDAELAEESLSKRELTADQRLDEMIKQAEAAEIAEENLAKRELTAGQRLDEMIKQAEAAELAEELI

SEQ ID NO: 243 - protein, 211 aa, Phakopsora pachyrhizi
MNHALIICLLGLASFGLISGHFTMDYPPTRGFMEDDEDKFCGGFPTNATGRHPFPISGPAHVALMAHHGSPKISISISFKHNFTSEAEFNGTELISNAPLPHPGNACFEIDLSNVTSKMTPKPSNGTLATLEVKYDAGGEALYQCSDLVLVQDAAVTNQSLSSCSAGRGSNGGSGSDAKQNAQNSSANSLWFQPTPTMICLSLILLSLSIL

SEQ ID NO: 244 - protein, 65 aa, Phakopsora pachyrhizi
MFLLLIIVGSLFIPTLTFIRSVEVSRPVIRFFDAHATCLVVPSRNIAAKIKKLILGIVKVVKAWY

SEQ ID NO: 245 - protein, 30 aa, Phakopsora pachyrhizi
MRILVRLGTRLFQSLGFSRILKDAPDFTEV

SEQ ID NO: 246 - protein, 48 aa, Phakopsora pachyrhizi
MRSTEIKKKKREREKDKEREKQRRYSKSQVFTRLFFSLAGFQILGVER

SEQ ID NO: 247 - protein, 430 aa, Phakopsora pachyrhizi
MQTKILLALCLVAISQVNAHGAITAVQGSNGMTGEAFGVDQSTPRDGTKRNPFQTDSSIIRDREIASGKSSACGRTLAGGNNEIGAAMSKAESAGIPSVSSDGKVQMTLHQVNGDGGGPYTCDVNASGDGKTFTPMTISTNIPGKNSRSNAKAEDIPLIAEMPKGMTCTGGSDGQTCIVRCRNAANAGPFGGCVAVTQATDATKQKRELSAEQRIDEMIKQAEAAEAFGDETLAKREMSADERLDEMIRQAEAAEAFGDETLAKRELSADQRLDEMIKQAEDAELAEENLSKRELTADQRLDEMIKQAEAAEIAEENLVKRELTADQRLDEMIKQAEAAELAEENLAKRELTADQRLDQMIKQAEDAELAEESLSKRELTADQRLDEMIKQAEAAEIAEENLAKRELTAGQRLDEMIKQAEAAELAEELI

SEQ ID NO: 248 - protein, 76 aa, Phakopsora pachyrhizi
MNIISSIYLAVAAILISQNNFTSAANLNTRDEVPKPVHFRYEARSAERYNHLEARRKRRKPQNFNGNSVNTVISSK

SEQ ID NO: 249 - protein, 35 aa, Phakopsora pachyrhizi
MNTHLGRDSRLLQLSLLLANTLPTVKYEMIQRVGC

SEQ ID NO: 250 - protein, 73 aa, Phakopsora pachyrhizi
MSKPSLFSLLFFLKKNRTNGIKKPPCPFLKFKHPGRNYKNFILKKCNGVNVGGILEICRDSSFLFEKVLQGAV

SEQ ID NO: 251 - protein, 39 aa, Phakopsora pachyrhizi
MVFLVSSVVEFRILFCSIAKKWVYSVLAYIVQFCYVLIY

SEQ ID NO: 252 - protein, 69 aa, Phakopsora pachyrhizi
MTLIIGPYGKIKLIAFVLHGFMAFQSVSCMYEDAIKSTGSKPFGELQKELKDLSDGENDRHTHSTFFST

SEQ ID NO: 253 - protein, 59 aa, Phakopsora pachyrhizi
MISAGHRESLLLSAGLAEDNCYRIRRKRTLLIYHIPILKSFVVLCFFVAAADCYFFKAL

SEQ ID NO: 254 - protein, 41 aa, Phakopsora pachyrhizi
MIDFDPVLGSCSCSCSCSFFLSFFFLIFNTAFLFINTAFLY

SEQ ID NO: 255 - protein, 135 aa, Phakopsora pachyrhizi
MMAFNRFAYLLLVSIMVASFTTGQLLPSAYKCNSVDSSRFPINREDCRDSLNLFLSPRNVLTFANGNTQSCGTCRVTIIKSGTSAPPRTAQKSWAVTALHEGYDNCNGNPVTATIGDSAMIDVSLSYGNGTPCPP

SEQ ID NO: 256 - protein, 48 aa, Phakopsora pachyrhizi
MYRFIFLFFCIFFLFTSNVFNFILNFHNSDKKRGERYLPIYFSCHPIV

SEQ ID NO: 257 - protein, 218 aa, Phakopsora pachyrhizi
MLFATLIAVCLLALGGKAESDVQSDTASKLQRRGHDDSLPPVTFIMRDSNEHVGGKLLIYNSDGTLAFTFRRAVLNSDGLSNVEVRDVRNNFSINLESNDDTCFKKSHYVEREKNLGQFKIDPRGAKADRWHFTRKTPHGDFKYDFHRKYFSKDGNIYINDTHVRVASLTSEIRHEAWLQPGKHGVPTFSLHLQYNTEPIFFVALMGLDLTRVDTCGL

SEQ ID NO: 258 - protein, 45 aa, Phakopsora pachyrhizi
MNTPSSMALGWGVLLLAAGGGLYFAKKDINQKRRDLHKSGAQILT

SEQ ID NO: 259 - protein, 144 aa, Phakopsora pachyrhizi
MLSTRVIMKLILLGVFSNLLIVACDEGFNCKDPSKGHPHRLVNATQCVNAIKSFPVINGDFSVSLNPAKSYVRHCGTCKLELSGSEKAKQAGLPLAKDETLIKIKDAVNHCNNDSYGDFSMYNERSKVTVRIDWEYSTKGAYCP

SEQ ID NO: 260 - protein, 327 aa, Phakopsora pachyrhizi
MMMMMMMRQATKKILGSVNLSSTRAIHHIAPFSKIASVAPLLSGLTSSSHSQRFSLNNQLTHTSSSVRSFFSLPSFPPSLNVSKDIHGYEDLNVRDRYSQRGDVLTYKETKRLPFSKHQLYYIAADIESYSSFLPYCAQSNLRSVRALDKTSDDSLESNLITNDERRQWLRGGKVGETFLIELEQVNGFKGLDELNRTSVECRKFDLVKLTMCDSKLFKSLSITWKFKSPREIFNIDSILNNQNGFTSQSSSLREEDNEGTGDAFVSIETSLTLLSGYINQQLGQVKGSKFSANDLSGELYWKAMSEKVLASYESRLNQVYLNNRQN

SEQ ID NO: 261 - protein, 321 aa, Phakopsora pachyrhizi
MMFQSKLLGLLILSLYLCKTSFAIPLGDDQKADPVDTSGFTIISDLYNKSYNNHNVKTDHPEPPKYTDPKKPDPPNWKEPKHPEPPKYTDPKKPEPPNWKEPQHPEPPNWKEPNHPDPPEWKGPEHPEPPKLKEPKHPEPPKWKVPKHPEPPKLKEPKHPEPPKWKVPKHPEPPKLKEPKHPEPPKWKEPKHPEPPKWKVPKHPEPPNWKEPKHPEPPKWKVPKHPEPPNWKEPKHPEPPKWKEPKHPEPPKWKEPEKPEPPKWKEPKHPEPPKWKEPEKPEPPKWKEPEKPEPPKWKEPEKPEPPKSKEPGYTDPPKERN

SEQ ID NO: 262 - protein, 288 aa, Phakopsora pachyrhizi
MMFQSKLLGLLILSLYLCKTSFAIPLGDDQKADPVDTSGFTIISDLYNKSYNNHNVKTDHPEPPKYTDPKKPDPPNWKEPKHPEPPKYTDPKKPEPPNWKEPQHPEPPNWKEPNHPDPPEWKGPEHPEPPKLKEPKHPEPPKWKVPKHPEPPKLKEPKHPEPPKWKVPKHPEPPNWKEPKHPEPPKWKVPKHPEPPNWKEPKHPEPPKWKEPKHPEPPKWKEPEKPEPPKWKEPKHPEPPKWKEPEKPEPPKWKEPEKPEPPKWKEPEKPEPPKSKEPGYTDPPKERN

SEQ ID NO: 263 - protein, 82 aa, Phakopsora pachyrhizi
MVRSLSFSLICLALSTMACSCKVLSFSGLLSIKLNLGSVNWALARQKSGLKENNFVLLSCGTLFHGSGFDFFLKTTSWLFFK

SEQ ID NO: 264 - protein, 31 aa, Phakopsora pachyrhizi
MKLQISALVISIVLGSLIGSVISNPTFTKRQ

SEQ ID NO: 265 - protein, 62 aa, Phakopsora pachyrhizi
MAIAPITGKLKKRLFLDLSIGLGLGTAGAYSWWYGYHVPKMRHQESIYAKMNADRAKHEAAI

SEQ ID NO: 266 - protein, 100 aa, Phakopsora pachyrhizi
MNFGSSSKKNLVARPSVGIRRLVLVCTAMVWSITLIINQPSGVNSSPFLRVAKSIGPEVAKGAAGGVGLVAVEQIANKVIGRSTDGQNVEEENSGSNKFS

SEQ ID NO: 267 - protein, 25 aa, Phakopsora pachyrhizi
MNYVEMLLSPLLLLKLKQSDFIWNK

SEQ ID NO: 268 - protein, 104 aa, Phakopsora pachyrhizi
MMISFKYRIKSSVVILFTILLSCLLIEIISSTQSPSTIQARSAVKKIEKDHQQRRLKIRKRQVTDAPNTPNLDLNITDAIAIQADTIPWYMLDQYQQDHIRFHW

SEQ ID NO: 269 - protein, 163 aa, Phakopsora pachyrhizi
MRSFVTCALLILFFQLIVSSPIEQSSEKQIRRPPPPPPPPVRRPPPPPHSGRPHPRDTGNKDNHQNEKRYGVEASQEQLSEKLIRRIASPQRPPVRRPPPPPSPGRPRPRDTGNKDNHQNEKRYGVEASQEQLSEKLIRRIASPQRPPVRRPTPPPPSGRPRQ

SEQ ID NO: 270 - protein, 61 aa, Phakopsora pachyrhizi
MWRGFWYGWVTAVCLFFMIRNKTCRIDRQSELPTVPGNQTHHHHYYCYHPYCYSFHQSICR

SEQ ID NO: 271 - protein, 76 aa, Phakopsora pachyrhizi
MAGKEIYEPRKSMMIKMIGFLCIWFQLLGVVFTQSTAATTSATKAKTYDTYFKRLAKRSPLTNSGRIERRHKCGPP

SEQ ID NO: 272 - protein, 201 aa, Phakopsora pachyrhizi
MQSNFLLAFCLIAAVSVPVSRAHGVITSVEGANGQTGSAFGMVESTPRDGTRTNPFQTDSSIIRDREVSSGKASACGRTLAGGNNDIASDMSSAESAGLASIGPDGKVRMTLHQVNGGGPYSCEVDTTATGDNFKKMNIDTNVPGKNSRSRAKATEFPLVATMPAGATCTGGADGQTCLVRCRNAANAGPFGGCVAVTQAN

SEQ ID NO: 273 - protein, 203 aa, Phakopsora pachyrhizi
MQSNFLLAFCLIAAVSVPVSRAHGVITSVEGANGQTGSAFGMVESTPRDGTRTNPFQTDSSIIRDREVSSGKASACGRTLAGGNNDIASDMSSAESAGLASIGPDGKVRMTLHQVNGDGGGPYSCEVDTTATGDNFKKMNIDTNVPGKNSRSRAKATEFPLVATMPAGATCTGGADGQTCLVRCRNAANAGPFGGCVAVTQAN

SEQ ID NO: 274 - protein, 52 aa, Phakopsora pachyrhizi
MFHRTSALKAVAFFFIFSWSVFILEVLSFPTKYSPSENKIVLEKGQGSSRGD

SEQ ID NO: 275 - protein, 69 aa, Phakopsora pachyrhizi
MRSFVYCALLVLICQLATSSPINERSEVEASKEQSSEKFFPLFPPPPPVPFIRPPLPPFGFKDKHQNEK

SEQ ID NO: 276 - protein, 190 aa, Phakopsora pachyrhizi
MKSFNLILFITVALALITQIDCLPKPRSLVSFNHLPAKEIVKRQEDRFNDADALSRETEGDGEGEGENDVGGEGENGSDNDDEGGGESEDGSVGDGEEEAEGEEVNEDVVEVDGDGDKDKEVEGEEGGGVETVEKVEGVDIGSKEVNGNVEEGDNNDNENAEDFSRGVNDLQENQFVDDDSEDEALADER

SEQ ID NO: 277 - protein, 81 aa, Phakopsora pachyrhizi
MRSFVYCALLVLICQLATSSPINERSEVEASKEQSSEKFFPLFPPPPPVPFIRPPLPPFGFKDKHQNEKVRFINQNIKGIR

SEQ ID NO: 278 - protein, 214 aa, Phakopsora pachyrhizi
MRSFVYCALLVLICQLATSSPINERSEVEASKEQSSEKFFPLFPPPPPVPFIRPPLPPFGFKDNHQNAKRSEVETAKEESSEKFFPILPPPPPPPFFRPHLPPFGFKDSHQNEKRSEVAAHQESQSTEKFFPGYGFPILPPPHPFFYHRRPFGYGFFPPPPFGFKDKHQNEKRSEVESSKEKSTEKFFPGLLPILPPFPPFGFLKDAKSNDESA

SEQ ID NO: 279 - protein, 206 aa, Phakopsora pachyrhizi
MRSFVYCALLVLICQLATSSPINERSEVEASKEQSSEKFFPLFPPPPPVPFIRPPLPPFGFKDNHQNAKRSEVETAKEESSEKFFPILPPPPPPPFFRPHLPPFGFKDSHQNEKSQSTEKFFPGYGFPILPPPHPFFYHRRPFGYGFFPPPPFGFKDKHQNEKRSEVESSKEKSTEKFFPGLLPILPPFPPFGFLKDAKSNDEKSA

SEQ ID NO: 280 - protein, 206 aa, Phakopsora pachyrhizi
MMKFFSIAFFSAIIALAVASGDQKDEVSAKTFVGSNGGIGFGNGGRYHGGYGGGASWGVGGSLPNFDRFYNEYYSLIPSFQRVHVTLESSQIDAVAATRLIGSLQNDFYSKVNSLGARCAGAQQLSLERKSSLRVCYRRWPNQIGNFKYSLARLGEAFKISFSLIQNLGFQPNEILGRLNLDIFSQFDLNLNSFAHSSGFVSGIGY

SEQ ID NO: 281 - protein, 192 aa, Phakopsora pachyrhizi
ARQATNFKEKMHTISQRLNSVSALTTTSIIILTIAITIISFIQRPSIQPARIEISDIETLWGHDKRDHYDRKDREWTNIRFDIEADLRPLFNYNTKQIFVFLSAKFGTDNFPNNEVILWDRIIKSSKDAKINLTGAKNKYAFKHIGGSFKNSNATYILHYNLMPKVGALIWGEAGKTKNQVPFPEVNYRKPI

SEQ ID NO: 282 - protein, 66 aa, Phakopsora pachyrhizi
YSAIAVVVALLIAIAVTSRILYIRRSRRLAAAPRSIDPEQSNNRENEERLAMERRGEEAGLPTYRQ

SEQ ID NO: 283 - protein, 168 aa, Phakopsora pachyrhizi
MYRFPLISLAIYASVVYGQGTTVTPTTNVNPNTSGNPPNQPTNGTPTNPATPTASPTTTPTPKILAVNCTQSYFEITDDSMNGTSIGACTKIGDGEKGGSFCQTKECFGFATCQDCNLATPGPNNTTVISNTTIKAVPCNKGYYIPGPGDNVTVTCKSVLMHIYHLKP

SEQ ID NO: 284 - protein, 184 aa, Phakopsora pachyrhizi
MYRFPLISLAIYASVVYGQGTTVTPTTNVNPNTSGNPPNQPTNGTPTNPATPTASPTTTPTPKILAVNCTQSYFEITDDSMNGTSIGACTKIGDGEKGGSFCQTKECFGFATCQDCNLATPGPNNTTVISNTTIKAVPCNKGYYIPGPGDNVTVTYCSAQNNANPDDYICKGPCTSFTGKLPKL

SEQ ID NO: 285 - protein, 198 aa, Phakopsora pachyrhizi
MYRFPLISLAIYASVVYGQGTTVTPTTNVNPNTSGNPPNQPTNGTPTNPATPTASPTTTPTPKILAVNCTQSYFEITDDSMNGTSIGACTKIGDGEKGGSFCQTKECFGFATCQDCNLATPGPNNTTVISNTTIKAVPCNKGYYIPGPGDNVTVTYCSAQNNANPDDYICKGPCTSFTACGDCININDPAIAGLKDTP

SEQ ID NO: 286 - protein, 104 aa, Phakopsora pachyrhizi
MFHRATVFITLFSALSILNDNRVSALMASVPDTQTCSFYTGAYTNSATCNEQPNVVCTQGCYGPYWLLAVSWRTAQRVVLQAPLKCALWDLDATRQLQKKKYGP

SEQ ID NO: 287 - protein, 156 aa, Phakopsora pachyrhizi
MLFATLIAVCLLALGGKAESDVQSDTASKLQRRGHDDSLPPVTFIMRDSNEHVGGKLLIYNSDGTLAFTFRRAVLNSDGLSNVEVRDVRNNFSINLESNDDTCFKKSHYVEREKNLGQFKIDPRGAKADRWHFTRKTPHGDFKYDFHRKYFSKDGE

SEQ ID NO: 288 - protein, 218 aa, Phakopsora pachyrhizi
MLFATLIAVCLLALGGKAESDVQSDTASKLQRRGHDDSLPPVTFIMRDSNEHVGGKLLIYNSDGTLAFTFRRAVLNSDGLSNVEVRDVRNNFSINLESNDDTCFKKSHYVEREKNLGQFKIDPRGAKADRWHFTRKTPHGDFKYDFHRKYFSKDGNIYIKDTHVRVASLTSEIRHEAWLQPGKHGVPTFSLHLQYNTEPIFFVALMGLDLTRVDTCGL

SEQ ID NO: 289 - protein, 93 aa, Phakopsora pachyrhizi
MMISFKYRIKSSVVILFTILLSCLLIEIISSTQSPSTIQARSAVKKIEKDHQQRQLKIRKRQVTDAPNTPNLDLNITDAIAIQADTIPWYMLD

SEQ ID NO: 290 - protein, 317 aa, Phakopsora pachyrhizi
MFNMLIALAITLVHAGSVFCQQAASAQQDSSTTIVANTNLNGAYGVSGQISFTLIPTTEEVAVKISVKGLMAINSTAQFGYHIHTNPISSDGNCSSALGHLDPLQVTDSVVCNGTARLCQIGDLSGRNGKLPGNQSVANVSYTDKFLRFWPQPFTLLGRSIVIHAPNTTRIACGNITSVVDGTADNQGKPTNQSSNYVTVYSTRAPPPSNNTANVAEPLPDVTETPNVILVKKQVTQMMNNSSETVEVNAAAPAATNFTYTPGAQLPSQEDFNILNGTNLNSTTSAKPQTGSSSGIEKIEYPLFFGFCAIMMISVVI

SEQ ID NO: 291 - protein, 203 aa, Phakopsora pachyrhizi; identical to SEQ ID NO: 273

SEQ ID NO: 292 - protein, 198 aa, Phakopsora pachyrhizi
MQSNFLLAFCLIAAVSVPVSRAHGVITSVEGANGQTGSAFGMVESTPRDGTRTNPFQTDSSIIRDREVSSGKASACGRTLAGGNNDIASDMSSAESAGLASIGPDGKVRMTLHQVNGDGGGPYSCEVDTTATGDNFKKMNIDTNVPGKNSRSRAKATEFPLVATMPAGATCTGGADGQTCLVRCRNVRFNFKYQIFIS

SEQ ID NO: 293 - protein, 201 aa, Phakopsora pachyrhizi
MQSNFLLAFCLIAAVSVPVSRAHGVITSVEGANGQTGSAFGMVESTPRDGTRTNPFQTDSSIIRDREVSSGKASACGRTLAGGNNDIASDMSSAESAGLASIGPDGKVRMTLHQVNGDGPYSCEVDTTATGDNFKKMNIDTNVPGKNSRSRAKATEFPLVATMPAGATCTGGADGQTCLVRCRNAANAGPFGGCVAVTQAN

SEQ ID NO: 294 - protein, 201 aa, Phakopsora pachyrhizi; identical to SEQ ID NO: 272

SEQ ID NO: 295 - protein, 105 aa, Phakopsora pachyrhizi
MFHRATVFITLFSALSILNDNRVSALMASVPDTQTCSFYTGAYTNSATCNEQPNVVCTQGCYGPYVVATGCVLENGSTGGASSSSQVCTMGFGRNTAAAKVLKML

SEQ ID NO: 296 - protein, 317 aa, Phakopsora pachyrhizi
MFNMLIALAITLVHAGSVFCQQAASAQQDSSTTIVANTNLNGAYGVSGQISFTLIPTTEEVAVKISVKGLMAINSTAQFGYHIHTNPISSDGNCSSALGHLDPLQVTDAVVCNGTARLCQIGDLSGRNGKLPGNQSVANVSYTDKFLRFWPQPFTLLGRSIVIHAPNTTRIACGNITSVVDGTADNQGKSTNQSSNYVTVYSTRAPPPSNNTANVAEPLPDVTETPNVILVKKQVTQIMNNSSETVEVNAAAPAATNFTYTTGAQLPSQEDFNILNGTNLNSTTSAKPQTGSSSGIEKIEYPLFFGICAIMMISVVI

SEQ ID NO: 297 - protein, 176 aa, Phakopsora pachyrhizi
MQSNFLLAFCLIAAVSVPVSRAHGVITSVEGANGQTGSAFGMVESTPRDGTRTNPFQTDSSIIRDREVSSGKASACGRTLAGGNNDIASDMSSAESAGLASIGPDGKVRMTLHQVNGDGGGPYSCEVDTTATGDNFKKMNIDTNVPGKNSRSRAKATEFPLVATMPAGGNYLHWWC

SEQ ID NO: 298 - protein, 457 aa, Phakopsora pachyrhizi
MKYLRFIQLFLILKAVDLYTLVAKKEEGINSHKAYDASAVLVISEKNIAADCTIRPSTVINGTLPGPLLRFKEGQKIRITVINLLQHENTTIHYHGLSQRGSPFSDGTQKTSQWAIPPRGGRFDYEFFLEKGSAGTYMYHAHNLFQVSTAYGPLIVDEATKPPFNYFEDRMFMIGDYYHSTDQELISKLLGTPFAWIGDPQSVLVNGQALNVCNTTSPHGCENSCAHHITYVKPDKVYRIRVAGITSLAYLYMAIESHTFFEIIEVDSAFVKPLKVNHMQLNSGQRYSFLLKTKSEAEIKKLGKTSFWIRFESKYRTTKVHGQFILQYQSVDKDDHLSKSKTAVVGDLPKKTSNSLLEPSNKEINNKSPPPPLLYFQKLVPIPSESSKDEWSTSDFSPLLEERPARRSEVNRRIIVRGQQIKSADGSIKWYANGQTVSYFDNLILGFSLLSTYKIKR

SEQ ID NO: 299 - protein, 254 aa, Phakopsora pachyrhizi
MNMKLRVFISLTVALSLIAQSPSLAVTLNRDRPSNAHALQARQEQAAELAAVTVPEEQAAASAPEQKAAAAASAPEHAAASSAPEHAASAAAPQSDLDKAANRLYDLMVNLGNGLEIVTNLEASPQDIKAQTQKVKQYFGEMNELRNKLLQMSPIHSGNLPKSVQDANEAQETLQKALIAISDSAEDANVLKKNYPVLSKTFKSVTTAGEDLFSTLYPGYGEKTQEGQDAQQHSQPQATQEGAAPVAAAEAPQA

SEQ ID NO: 300 - protein, 169 aa, Phakopsora pachyrhizi
MSYQRMFLMFQLVILAVMMNDQSNSARATLLIQDEPLHLIKTQFEIKPQKLITVIRQPTQQEALDLSRIGQSTGTTTTTGVDGSIIRVRPSSRGMMFRFEDGVGGGVESDSGIGGMLSKFLSRISRPSVSSGSSVESNLGGFDLGSMLKSVKSSISDGMTTVRRLVVVY

SEQ ID NO: 301 - protein, 231 aa, Phakopsora pachyrhizi
MNISLKNFVQVSILLILVSGVISLDASTPNSHHLSRRQNKDQVKDQTKKTQQQVQEIKDGKGGAAQGGTPTEKAANQMMQMLMQIGQSLDAVTSLGSTPETVKSTAEAIIKMVPDVGKATKSLIDTMPNKAQLEGVADKATQSAEQLGKVLKTIEQSPEDANFIKKHYKALGDAFTGIFGAVDPVFSSAFPDDKNGGQQQAGKTDGRQLNTQTEGAQKDQKDTKEKNKKKT

SEQ ID NO: 302 - protein, 72 aa, Phakopsora pachyrhizi
MDFFLKFTKWLLVASIIGAVMSSPPTITRKDASRVERRKTKPLSTQDPQKKVTCPPNEVVKGGKCVPFKPHF

SEQ ID NO: 303 - protein, 373 aa, Phakopsora pachyrhizi
MTLRLFKWVGFLQLLLRLTSAQDTSGTTPLYQKKFPWPNVPYRADTGDGPRGTQQGYNKCNSTTQNQDSLCQTTFLNSIQEFCLWGAIKPNSIVGNIEGEMVAYCTTKKFGTRLIPSGAITGVQLTRTPGYIQVVGFIDQTALNLQANDTGGEEDPHGADQRGNPLGALMYSSAFNSNSGASSGQSSNSTSSSSYTQVSEWSFFIGSGVFCYKACDPAGPNAPQLCQHIYDRIGCAYNAPANYPSINGTYQSCQGENQLPAGTYIENGVTKTYQQPPESLGPITSIPYTAPIPAVSNCVTYKDPVALWPDLPKTQTVANTSSTVNRKGVSSSTSDGSKQSAQGGAQSAASSIELPVISLLHPIVLGMVFVMLA

SEQ ID NO: 304 - protein, 240 aa, Phakopsora pachyrhizi
MKMNMLHHSLLLFTFFCIKLLICLPMNGKTPAVVADAVSAIQETGKVASTSIEVPSANLGAEVKELSAIDASVFTNVPAINPKDSAQVENLGQSTLQEKESPVILKGDVNHEKKNLIKTESKSADSEIKKEVDEKPLKEKSQGEDGPDGPEKKNIEGEGGDLKNDKNLNQSEQKTRLSRIKNWFIRQYLMLKNNVYPKIKEKSQQFWAWTKKKFSRGNNKVATNEIELKPCCRGKKTHFS

SEQ ID NO: 305 - protein, 117 aa, Phakopsora pachyrhizi
MSAITQTAKGAAVAGGLYFLLHSYIANRSQLISKSLSDLTNQFSYLTIQEANYGNNSKSDLFFYHQRLKETMAERIKFNWNQNIRKIYYSIYDFDPINRLNRFYIETKNTSNIASKN

SEQ ID NO: 306 - protein, 158 aa, Phakopsora pachyrhizi
MMKFLNVVLFSPLFLMLSSNSSPLLSSRQSIPIYNQCRVENSFSLTIDDGFSALSKSLTQVFDDNQVRATFFINGNNAACIYYNADDLKARHRSGHLLANHGWSHAHMTQLSRANMVREVERVEQAFIKILGLKPLYFRPPYGKLISFGNLRPTQKEL

SEQ ID NO: 307 - protein, 52 aa, Phakopsora pachyrhizi
MISITVAALVGPSTRLPINQSLSSVSGIIIILTTVVISICHFIVQCPRLFQR

SEQ ID NO: 308 - protein, 58 aa, Phakopsora pachyrhizi
MRSLIVPTLLCVLFIHKSCATVHTQCYNYFLQKDGCVFSAADDRNRCSADPKPSTAVG

SEQ ID NO: 309 - protein, 91 aa, Phakopsora pachyrhizi
MYFLTFIKKNAIYIYSVLILFFVNKKSINILCSESINNKIKCCFLVYFENFVLTNYKFSLFIIKIVSRNFQIQRSWTSSNSTTDIIVRSMT

SEQ ID NO: 310 - DNA, 759 bp, Phakopsora pachyrhizi
  source 1..759: mol_type = genomic DNA
atgttactgtctttaggattcaaacagttcttgattatttctttttttatcttcagaaccttgaatgctacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagtcatcatctataatagttacagcaactttggttaacgatggaagaatcaatggaattccactctcaattccaactggaacctcaaactcttcatcaacttctaaaggttcaaacaacgatacagcaccgaacactttaccaaccacagcaccaaccccattccaaaacactaatacctcaccaatcaataacgataactcaaatagacaaaactcgggatcgaacggattgacgatttggttcgatcgaaaaagaatgaaatttgattataggatgtttcttgattcaaatcttctattttttatctgtttcactctcctacttgtttaa

SEQ ID NO: 311 - DNA, 702 bp, Phakopsora pachyrhizi
  source 1..702: mol_type = genomic DNA
atgttgaatgctacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagtcatcatctataatagttacagcaactttggttaacgatggaagaatcaatggaattccactctcaattccaactggaacctcaaactcttcatcaacttctaaaggttcaaacaacgatacagcaccgaacactttaccaaccacagcaccaaccccattccaaaacactaatacctcaccaatcaataacgataactcaaatagacaaaactcgggatcgaacggattgacgatttggttcgatcgaaaaagaatgaaatttgattataggatgtttcttgattcaaatcttctattttttatctgtttcactctcctacttgtttaa

SEQ ID NO: 312 - DNA, 759 bp, Phakopsora pachyrhizi
  source 1..759: mol_type = genomic DNA
atgttactgtctttaggattcaaacagttcttgattatttctttttttatcttcagaaccttgaatgctacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagttatcatctataatagttacagcaactttggttaacgatggaagaatcaatggaattccactctcaattccaactggaacctcaaactcttcatcaacttctaaaggttcaaacaacgatacagcaccgaacactttaccaaccacagcaccaaccccattccaaaacactaatacctcaccaatcaataacgataactcaaatagacaaaactcgggatcgaacggattgacgatttggttcgatcgaaaaagaatgaaatttgattataggatgtttcttgattcaaatcttctattttttatctgtttcactctcctacttgtttaa

SEQ ID NO: 313 - DNA, 693 bp, Phakopsora pachyrhizi
  source 1..693: mol_type = genomic DNA
atgacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagttatcatctataatagttacagcaactttggttaacgatggaagaatcaatggaattccactctcaattccaactggaacctcaaactcttcatcaacttctaaaggttcaaacaacgatacagcaccgaacactttaccaaccacagcaccaaccccattccaaaacactaatacctcaccaatcaataacgataactcaaatagacaaaactcgggatcgaacggattgacgatttggttcgatcgaaaaagaatgaaatttgattataggatgtttcttgattcaaatcttctattttttatctgtttcactctcctacttgtttaa

SEQ ID NO: 314 - DNA, 759 bp, Phakopsora pachyrhizi
  source 1..759: mol_type = genomic DNA
atgttaatgtctttaggattcaaacagttcttgattatttctttttttatcttcagaaccttgaatgctacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagtcatcatctataatagttacagcaactttggttaacgatggaagaatcaatggaattccactctcaattccaactggaagctcaaactcttcatcaacttctaaaggttcaaacaacgatacagcaccgaacactttaccaaccacagcaccaaccccattccaaaacactaatacctcaccaatcaataacgataactcaaatagacaaaactcgggatcgaacagattgacgatttggttcgatcgaaaaagaatgaaatttgattataggatgtttcttgattcaaatcttctattttttatctgtttcactctcctacttgtttaa

SEQ ID NO: 315 - DNA, 693 bp, Phakopsora pachyrhizi
  source 1..693: mol_type = genomic DNA
atgacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagtcatcatctataatagttacagcaactttggttaacgatggaagaatcaatggaattccactctcaattccaactggaagctcaaactcttcatcaacttctaaaggttcaaacaacgatacagcaccgaacactttaccaaccacagcaccaaccccattccaaaacactaatacctcaccaatcaataacgataactcaaatagacaaaactcgggatcgaacagattgacgatttggttcgatcgaaaaagaatgaaatttgattataggatgtttcttgattcaaatcttctattttttatctgtttcactctcctacttgtttaa

SEQ ID NO: 316 - DNA, 759 bp, Phakopsora pachyrhizi
  source 1..759: mol_type = genomic DNA
atgttaatgtctttaggattcaaacagttcttgattatttctttttttatcttcagaaccttgaatgctacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagtcatcatctataatagttacagcaactttggttaacgatggaagaatcaatggaattccactctcaattccaactggaagctcaaactcttcatcaacttctaaaggttcaaacaacgatacaacaccgaacactttaccaaccacagcaccaaccccattccaaaacactaatacctcaccaatcaataacgataactcaaatagacaaaactcgggatcgaacagattgacgatttggttcgatcgaaaaagaatgaaatttgattataggatgtttcttgattcaaatcttctattttttatctgtttcactctcctacttgtttaa

SEQ ID NO: 317 - DNA, 693 bp, Phakopsora pachyrhizi
  source 1..693: mol_type = genomic DNA
atgacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcactgattttccatgcctatgtcaaagtaaatcgtttatgactcaggcccaagaatgttttaaacaaaaatgcagtcaatctgagttatcaacagttttaagtctaagcttgcaaacatgccaagttctgaacataccgttttacagaattcctgaatttcagtcatcatctataatagttacagcaactttggttaacgatggaagaatcaatggaattccactctcaattccaactggaagctcaaactcttcatcaacttctaaaggttcaaacaacgatacaacaccgaacactttaccaaccacagcaccaaccccattccaaaacactaatacctcaccaatcaataacgataactcaaatagacaaaactcgggatcgaacagattgacgatttggttcgatcgaaaaagaatgaaatttgattataggatgtttcttgattcaaatcttctattttttatctgtttcactctcctacttgtttaa

SEQ ID NO: 318 - DNA, 2778 bp, Cajanus cajan
  source 1..2778: mol_type = genomic DNA
atggcagacagtgtggtttcctttgttttagatcacttgtcccaactggtggaacatgaagcaaggttgctaagtggcgtggaagacaaggtgaagtccctcgagagggagcttcagatgatcaacgtcatcctaagaaccacaaacagcaacaacgacattcagaaaacagtggtgagccaaatcagagatgtggcccatgaagctgaggatgtcattgacacgtacgttgccaaagtggccctgcacaacaggagaaccatgctggggaggctactccatggagttgaccaaacaaagttgctccatgacgtatccgagaaaatagacgagatcataacaactctcaaccagatacgtgaaaacaagatcaaatacagcgagttccaagaaagaaatcatcaatccatagcagaagaggaggaggaggagaaggagagggagaggttacttcacaagctaagaagaaatgtagaggaggaacatgtagtgggttttatccgtggatctcaagcaatcatcaagctactcaaggaaggtggctcacggcgtaatgtggtctccatcatcggcatgggggggttgggcaagaccacccttgcccgaaaagtttataatgatagcaaggtgaaacaaggctttagttgttgtgtttgggtgtacgtgtcaaacgagtgtagagctaaggagcttttgcttagtcttcttaagcatttgaggccaaacctcgaaactgaacttcaagaagaaaacaacaaaggaaaaaaattcactgaagaacaagacatttttaacttgagtgtggaggagctgaagaaactggtgcggcaatacttggagaggaaaacaaggtatctggtggtcctcgatgacttgtggaaaacacaagattgggacgaggtgcaagatgcttttcccgacaacaacagaggcaacagaatattgatcactagtcgtttgaaagaggtggccttgcatactagtcttcatcctccatactaccttcaatttctcagccaagaagaaagctgggagctctttcgtaggaaagtgtttagaggggaagaatgcccttttgaactagagcctctaggcaaacaaatagtggcaagttgtcgcggtttgccactctctattgttgtattagcaggattgctagccaacaaggaaaagtcacacagggaatggtccaaagtggtgggtcacgtcaactggtatcttactcaagacgagactcaagtgaaggatatagttctgaagctcagttatgataacttgccaagaagattgaaaccatgctttctatattttgggatattccctgaagactttgaaatccctgttaggccattactacaacaatgggttgcagaagggtttatacaagaaacaagaaatagagacccagatgatgtggcagaagactacttgtacgagctcattgatcgtagtttggtccaagtagcagcaataaagactagtggaggtgtgaaaacttgtcacatccatgatcttctccgagatctttgtgtatcgcagagcaaaggggacaagatttttgaagtctgctcagataatgacattcaaattctaacaaaacctcgcaggttgtccttccattgtgacatgggccactacatttcttcaagcaacaaagaccattcatgtatccgttctttgttcttctttggaatatattccaattttactgggaacgagtgggaatggcttttcaaaggcttcaaattggttcgagtgttagagcttggaaaaaaccattgcgcaggaaagatcccatctaatttgggggactttatccacttaaggtatttgagaattgactcgaattttggtataattattccaacttccatacttacccttcagaatttacaaacagtagatttaggtaattggtttagggaaatcccaatttgtttccctgctcaaatgtggaagctcaaacatttaaggcacctgtatgggcaaggacctgtgaagcttcaaggccactattcaggatcaaatgaggttatgtggaatctccgaaccatcttccccattgatattgatacacaaacattgtctctgatgaagaaaggaagcttccccaatcttgtgaaattggggttgtcaatcaattcggaccgccaaggtaagtggccaaagttgttgcagagcttacaagaattaagtcatttgaatatcttaaagatttgcctccgaggggattttgatgcttcaataggcacagtgtcaagcatatggcggtttggttgtgagccacaggagctattacaaagcctagggttgttgactcatataactacgttgaaaatcaccaatatctgcagccttatgataacggttcctccaaatgtcaccaagttaacattgcgtggtattagtagcatcactagggaggggctgaatgcgttgagaaatcacaccaaactccaaattttgagtctatatggagactatggctctaacattaacctcaattgtgttgtaggcggctttccacaactgcaagtattgcaattgaaaaagttcacctctgtaaattggaaattaggcaatggtgcaatgccacgtcttcacactctagtcatcatcaactgtcaaagtttagatgatcttccaaatgaattgtggtctctcactgccttcagaaaactgcatgtaaaacaaccctcacaaccaatgcttcgtatgctacgggatttgaaaataaaggatagggttcaagtcatagtcgatgatcatgacaactag

SEQ ID NO: 319 - DNA, 657 bp, Phakopsora pachyrhizi
  source 1..657: mol_type = genomic DNA
atgagtctaagttttaaggctttactagtctttctaccactgatcttcttctacctggtgttggctgtaaaacaaccatcagatcaaacaactcattctacccaaccacagaagcataacactcatcaaacatctatcaactcaaagcgagaactacaacagcaactcagtcagttcaatccacatcatcaatctttagcttcatcaaccaggatgtcatcagtcaatgatagaacagcttcaccaatcacagatattgactcaaacacaaacgttccaacagctgccaagggtgaggaggagtccacaaaacagaagcttgaagatcaaccagcaggcgatggtacaaaggcatcattatcagctaaccctggtgaatcgcaacagagtgacgagaagaatcttaaatctcagaatgtttccccgagtcaagaacacatgagtagatacattgtcactttctcgcgtgagactacttctgcccagttgaaggaatacgctgataaaatcattcagagaggtggaaagattaaccatacgtatgattcagcgattcttaaaggatttgctgtttctataagtgatagtctggttacaaccctggatgatgatccaaatgtaaagagtgttgaacctgatggtgaagtacacatctga

SEQ ID NO: 320 - DNA, 657 bp, Phakopsora pachyrhizi
  source 1..657: mol_type = genomic DNA
atgagtctaagttttaaggctttactagtctttctaccagtgatcttcttctacctggtgttggctgtaaaacaaccatcagatcaaacaactcattctacccaaccacagaagcataacactcatcaaacatctatcaactcaaagcgagaactacaacagcaactcagtcagttcaatccacatcatcaatctttagcttcatcaaccaggatgtcatcagtcaatgatagaacagcttcaccaatcacagatattgactcaaacacaaacgttccaacagctgccaagggtgaggaggagtccacaaaacagaagcttgaagatcaaccagcaggcgatggtacaaaggcatcattatcagctaaccctggtgaatcgcaacagagtgacgagaagaatcttaaatctcagaatgtttccccgagtcaagaacacatgagtagatacattgtcactctctcgcgtgagactacttctgcccagttgaaggaatacgctgataaaatcattcagagaggtggaaagattaaccatacgtatgattcagcgattcttaaaggatttgctgtttctataagtgatagtctggttacaaccctggatgatgatccaaatgtaaagagtgttgaacctgatggtgaagtacacatctga

SEQ ID NO: 321 - DNA, 294 bp, Phakopsora pachyrhizi
  source 1..294: mol_type = genomic DNA
atgaggttctcagcttcaatcccactcttcctagcccttgttttcggtatgcttgcgagtgtgtccttaggtctgagtcagcaggacgaagtaaggatgctattgtcacttccaacgtgtatgaagaattgttatcttcagaacttgaatgctgccccatcgtgcaagggcagagcaaattgtttgtgtagttcccaaatttacacttataaagttaaggcctgtgcaacgggttgtgacttgagcgaacaattgcaaggtctgagttattcaagaactgtttgtaaccagtga

SEQ ID NO: 322 - DNA, 384 bp, Phakopsora pachyrhizi
  source 1..384: mol_type = genomic DNA
atgtttggtttaaaggctatcataatcgccactggcttgatctatcaggtcactgcggctgccaatacctctgccaccactgctcccggacaggatcagggccaaggtcaaggtcctgaatctggtggttgtgcagccaagtgtatggccgcaaagctggatgacgctgccaagtggtttggtccaggtaaccttgcatcctactgtcaacatgccgagttcatcactgcatacgacacatgtttgggcgataactgtgccaaccatgaagagctagatgccggtaagaaaagcggtcgtgaggcttgcgctgctgctggagtcacctcaccgggatcgattggcttctgctgctgccaacagtacagcttcaacactcactag

SEQ ID NO: 323 - DNA, 348 bp, Phakopsora pachyrhizi
  source 1..348: mol_type = genomic DNA
atgtttggtttaaaggctatcataatcgccactggcttgatctatcaggtcactgcggctgccaatacctctgccaccactgctcccggacaggatcagggccaaggtcaaggtcctgaatctggtggttgtgcagccaagtgtatggccgcaaagctggatgacgctgccaagtggtttggtccaggtaaccttgcatcctactgtcaacatgccgagttcatcactgcatacgacacatgtttgggcgataactgtgccaaccatgaagagctagatgccggtaagaaaagcggtcgtgaggcttgcgctgctgctggagtcacctcaccgggatcgattggttag

SEQ ID NO: 324 - DNA, 438 bp, Phakopsora pachyrhizi
  source 1..438: mol_type = genomic DNA
atggttttttcatccttgatgctaatagttaacattttaaatgtttttttaactctgacaaatcaattggttcagggagcgtctgttgaaataggtcgaggtttaggtcgtagacaagcaaacttgaagggaatgggccaagcaacattttatacacctggacttggtgcatgcggagaaacacacactgaatcagaaatggtggttgccatatcaaaagacctatttcaatcactaggaggagcaagctctaatccaaatcaaaatcctgtttgtggaaaaactgtaactgctagctaccagggaaaaagttgcagtgtcaagataattgacttgtgcatgagttgcgcacaaggtgacttggatctttccccggcagcctttaaacaactctccgacttatctgctggtcgtatatcaggtgttacctggggttaa

SEQ ID NO: 325 - DNA, 648 bp, Phakopsora pachyrhizi
  source 1..648: mol_type = genomic DNA
atgtttggtttaaaggctatcataatcgccactggcttgatctatcaggtcactgcggctgccaatacctctgccaccactgctcccggacaggatcagggccaaggtcaaggtcctgaatctggtggttgtgcagccaagtgtatggccgcaaagctggatgacgctgccaagtggtttggtccaggtaaccttgcatcctactgtcaacatgccgagttcatcactgcatacgacacatgtttgggcgataactgtgccaaccatgaagagctagatgccggtaagaaaagcggtcgtgaggcttgcgctgctgctggagtcacctcaccgggatcgattgccccacccacaggacatacccctcctaccagtgggaataactctctagcagatttgcctgccggtgctactaatcatagcatgagcactaattctactccaagtgcaactaccaccaactctacccttggtggtaccctcaataacacccttcatacccctaacaccacctccaccgctactaacagtagattttccagtgcggttaactctgcttctgctgctgccaacagtacagcttcaacactcactagctcttctatcttgcttggtatctcttctgtactggtgattgccaacctctaa

SEQ ID NO: 326 - DNA, 651 bp, Phakopsora pachyrhizi
  source 1..651: mol_type = genomic DNA
atgtttggtttaaaggctatcataatcgccactggcttgatctatcaggtcactgcggctgccaatacctctgccaccactgctcccggacaggatcagggccaaggtcaaggtcctgaatctggtggttgtgcagccaagtgtatggccgcaaagctggatgacgctgccaagtggtttggtccaggtaaccttgcatcctactgtcaacatgccgagttcatcactgcatacgacacatgtttgggcgataactgtgccaaccatgaagagctagatgccggtaagaaaagcggtcgtgaggcttgcgctgctgctggagtcacctcaccgggatcgattgccccacccacaggacatacccctcctaccagtgggaataactctctagcagatttgcctgccggtgctactaatcatagcatgagcactaattctactccaagtgcaactaccaccaactctacccttggtggtaccctcaataacacccttcatacccctaacaccacctccaccgctactaacagtagattttccagtgcggttaactctgctgcttctgctgctgccaacagtacagcttcaacactcactagctcttctatcttgcttggtatctcttctgtactggtgattgccaacctctaa

SEQ ID NO: 327 - DNA, 294 bp, Phakopsora pachyrhizi
  source 1..294: mol_type = genomic DNA
atgaggttctcagcttcaatcccactcttcctagcccttgttttcggtatgcttgcgaatgtgtccttaggtctgagtcagcaggacgaagtaaggatgcttttgtcacttccaacatgtatgaagaattgttatcttcagaacttgaatgctgccccatcgtgcaagggaagagcaaattgtttgtgtagctcccaaatttacacttataaagttaaggcctgtgcaacgggttgtgacttgagcgaacaattgcaaggtctgagttattcaagaactgtttgtaaccagtga

SEQ ID NO: 328 - DNA, 486 bp, Phakopsora pachyrhizi
  source 1..486: mol_type = genomic DNA
atgcttttcaactcaaacgtgatcctctttgcattaacttttgctctgggatcgttataccagggatcagaagctcaggatctggccggactacctaactgtggtcagaactgtctattgacagcctttacgacatcgagcgatggttgcacacagaccgactttgcatgtctttgcaagtcgcaaaagtttactggtacatcaaattcgtgctactcctcgtcctgtacctccactgaagcagcgactgccaagagctggggtgccaagacttgtgcttctgtaggagttaatgtgacggccacccttggaaacaccacttccgttggaactgtcatcggaaatggaactaatcctaacaccaactctacgcttcataatggcacaactgcctctacacatgcacagaactctggaggcctcatcaacgtgcaacccatctttttcatgtctttagtctctgtcctggctgcagtcttcatgtga

SEQ ID NO: 329 - DNA, 567 bp, Phakopsora pachyrhizi
  source 1..567: mol_type = genomic DNA
atgttcggtcttaagactatcatcgttgccatcgctgccatcctacaagttgcggaggctatcgacgactcaggttgtgcatccaagtgtatggctgtaaaattaaccgaagccgctgtctggttcggtcaaggcaatctctcaacctactgccaacatcctgaattcatcaccgcatacgattcgtgcttaggcgataactgcaccagtcgcgatgagctagaagtagccaagagaaatggccgcgctgcttgcgctgctgctagtatcaattccgttgcaaataatgtttcatccattggacgcaacaactctccagcagatatgcctgctggtgcagggaaccgaagcaccggttctccttcacctacagtcccaaccaacacaagcgttaacatcaattctaccttactaacacctaattctactgtaaacgccacaaacagccgattttccaacgcaccattccctgcgatttcggctgcggcaaacagcacggcctcaacaataacttgttcatcaatgataattggtttaacttctgctctggttttagcaagcctgtaa

SEQ ID NO: 330 - DNA, 567 bp, Phakopsora pachyrhizi
  source 1..567: mol_type = genomic DNA
atgttcggtcttaagactatcatcgttgccatcgctgccatcctacaagttgcggaggctatcgacgactcaggttgtgcttccaagtgtatggctgtaaaattaaccgaagccgctgtctggttcggtcaaggcaatctctcaacctactgccaacatcctgaattcatcaccgcatacgattcgtgcttaggcgataactgcaccagtcgcgatgagctagaagtagccaagagaaatggccgcgctgcttgcgctgctgctagtatcaattccgttgcaaataatgtttcatccattggacgcaacaactctccagcagatatgcctgctggtgcagggaaccgaagcaccggttctccttcacctacagtcccaaccaacacaagcgttaacatcaattctaccttactaacacctaattctactgtaaacgccacaaacagccgattttccaacgcaccattccctgcgatttcggctgcggcaaacagcacggcctcaacaaaaacttgttcatcaatgataattggtttaacttctgctctggttttagcaagcctgtaa

SEQ ID NO: 331 - DNA, 756 bp, Phakopsora pachyrhizi
  source 1..756: mol_type = genomic DNA
atgatagtgatagctcagctcctgattacgagttttctattcacgctgagcctcattcgcataccagcctcaacgaacgaatctttttcctgggtcgattcgttcccgcggctagcttcttcacctcaatgtcatttaagttccgcttccgctaaggtggcaaaaggcacccaacctttgcacaagagaaaatcctatcaaaactcgcttttgatatctaccacctcaggaaattttcttgggttcacaaatcgctccgtgactaataatgttgatttatggcgcggaataccttacgcagaaccaccaataggcacaaggagatttcgagctccaatactagctaaaccgccattggatgatccctcggccattcaagcggcgtatgagtttggaaatgcttgcccccagcaacctacccaatttgttcctgttcctatttcagaagattgtctatctttgaacatctatcgcccagaaaatacagaaaaggcttccgcacagttgcccgtcctgttatgggtacatggtggcggttttacaatgggcgtctccgcttcagctgatggcagtattcttgtgagctcaagcgtagatctggaaaaaccgatagtgtttgtgtcgataaattacaggttgaacacttttggatttcttaacactgacgaattaccgctagaagacctgcaagtggggctcaaagaccagattctcagtctagaatggctgaaaaaaaatatagctaactttggaggc

SEQ ID NO: 332 - DNA, 480 bp, Phakopsora pachyrhizi
  source 1..480: mol_type = genomic DNA
atgggatatacacgaattttaataattttgaccatatttcaggccttttatcttaccttgggagattcattaactaaaagtattcattctcgccaaaattcactctctacctgcgcgagagagtgctacgcaagtgcactagcaaactcaagtgcttcaggatcttgctcccagacagataacctttgtttatgtaggagggacgaatttagcgacaacgtaaaagactgctgggataaatgtaccgacattgaggaggccgctgcaaaaacatggtttaagacagagtgcgctgctcgcggggttgctgtgacttttaagaatgtcacaagtgccgtttcaggggcagcatcttctgtggcgaacacgatcactaacgcgagaaattcgcaacccaagacagtacctataccagtaacattagctttatgctctgttgtgttatacgctttattgggcctatctgaagtactttcctag

SEQ ID NO: 333 - DNA, 402 bp, Phakopsora pachyrhizi
  source 1..402: mol_type = genomic DNA
atgggatatacacgaattttaataattttgaccatatttcaggccttttatcttaccttgggagattcattaactaaaagtattcattctcgccaaaattcactctctacctgcgcgagagagtgctacgcaagtgcactagcaaactcaagtgcttcaggatcttgctcccagacagataacctttgtttatgtaggagggacgaatttagcgacaacgtaaaagactgctgggataaatgtaccgacattgaggaggccgctgcaaaaacatggtttaagacagttgagcaaccaactttttgtcctgattttctcccactattgttaaagctgatgttctggcttgctcactttataaataaaggagtgcgctgctcgcggggttgctgtgacttttaa

SEQ ID NO: 334 - DNA, 480 bp, Phakopsora pachyrhizi; identical to SEQ ID NO: 332
  source 1..480: mol_type = genomic DNA

SEQ ID NO: 335 - DNA, 750 bp, Phakopsora pachyrhizi
  source 1..750: mol_type = genomic DNA
atggcgtttagaagcttttcgacttcgatctttgctcttaagagcttatttttgattgttaaccctatccaaacatgggcagcagtgggtctagggaatattccaagttgctctcatctctgctttgaccaatcatcaagtcaatcttcatgctcagcaatgactgatctcaagtgtatctgccacgatcaaggcttcttaactgctctccattcttgttttcaattgagatgtgatcccgattcgttgccaggagctttgaggctaatgaacacctgcaaagagtcttttggaataacagttgagtctgcgaaacctcaattagggggccaatcgtctcctctagtgctttatctcggcgcccaaccacctctacccaacaattctagcttctcgacccttacatctaccgggttcgtttctagttccagtggtggagtgaatgttatatctcccccagctccggtcccagcccgagttcctgctcagtctacaggttatgttccaaccacaccaccagagttagctccatcaggtagcgctcttgctcattccatcaacgaaagcggggtctcagcggcttctccgtcgaccaatgccccctcaagagtctcaaatcaaactcctggcctcgctccccctccggcacaagcaacagctcaaaactccgccagtttgaggtacagctcgagcctgaataaaccagttatcatggtcgcagcaagcagctttgcaatgctattcgtgtaa

SEQ ID NO: 336 - DNA, 414 bp, Phakopsora pachyrhizi
  source 1..414: mol_type = genomic DNA
atgggatatacacgaattttaataattttgaccatatttcaggccttttatcttaccttgggagattcattaactaaaagtattcattctcgccaaaattcactctctacctgcgcgagagagtgctacgcaagtgcactagcaaactcaagtgcttcaggatcttgctcccagacagataacctttgtttatgtaggagggacgaatttagcgacaacgtaaaagactgctgggataaatgtaccgacattgaggaggccgctgcaaaaacatggtttaagacagagtgcgctgctcgcggggttgctgtgacttttaagaatgtcacaagtgccgtttcagggcatcttctgtggcgaacacgatcactaacgcgagaaattcgcaacccaagacagtacctataccagtaa

SEQ ID NO: 337 - DNA, 1284 bp, Phakopsora pachyrhizi
  source 1..1284: mol_type = genomic DNA
atgcaatcagcttcaaaactactgattaatatagtactgatttataacctaatcgtccacatcgcctgtcatggtttcattagctattgttcagcagatggaggaaagtttgaaaaggctcaaaagacacctctgagtgatacttcattcagaggtgcatcttcgaacaccggctggatcggtagcaaatttatcgactcagaatcttttacctgtggttcatctgatacacccaagggtactgttgcaccacctggtggaaccatcttcagtagacctgaacagagcgctaagaaaacattgaaagttaaggccggtggaaaagttgatataataatctctggtgaaaaaggtaaaggatttcctcatcctaaaggacacatagaaacttttctgggtaattgtggaccaaccgaatcagactgtcaaaactttgacgcgtcaaaggcaaagtactttaagatccaatccgagatggatgggatctctaatagtttaagaacaaagtataacagtgagttagatggaaatccttggggaattccaatcccatcaaatgttcaacctggtagctatattattcgagtacaaatgattgcttttggtcaatctggagaagttgaaggtaaccaggatcagtactacgtctactgtggacaaatatttatagatgatggtcagaaagatagtactctcaaatcaaccacacagttcatccaacaggaaccacaagaccttctgagtttcccaggcgcctacaagactggaaatattgacccaaagactttaccagatccgatcaatgttgcctcatccgctgcctcatcctcagctgatgatgttaactcaggagttccaccaaaagcagctgataaagataagcttcaaatcaaatcagttcaaccaccctcaatatcagatgatggttacggggggaatgaccagggagcagagacagtttctaaatctgagccttcaaagattgcttgcgcagataaatgctacaaggcaactctaccgaaaatcaaagacttcgccaaatcttgcactgccgaggattttaactgtttatgcaactctcctagattcataaaagcttatgagaactgttgcaaagataactgtgatgagggaaacgtcaaggctgcgatcaaggagattcatagtaggtgcgattcagctggtccatcaagaaaacgatcaaaaccgtccaatctacactcttcaacaccactctctactgaccgattgatgcataagagaagatctatcagaaaacgatag

SEQ ID NO: 338 - DNA, 1626 bp, Phakopsora pachyrhizi
  source 1..1626: mol_type = genomic DNA
atgcattcctcatctgtgggtttcctgattgctgcattggcgggtgctgctgctgcctccccctaccaggagcaggatgacctcaccttagctccgctggtgaccccacctccacctccgatgggtggtcctctctctcagtttgcaatccagtcaggtcgttcaccatctgactacctcatcaaggatcattacattgtcgtcttcaagaaggacattcacgagaagcatgcccgtgatcacaagcacaaggttcacaagctctggaaacaggatcgtaacgagaggattcaggtccagggctttggtattggtgatatctttgccggtatcaaacatcactttgacattggaacgagcgtctttaagggttattccattcatgttcccgagtcattgatttcgatcatcagaacccttcctgaggttgcctacgttgagagggactctaaggtctggatcaacgatccttactttgccactaacaccgtccctgcaaatgaacctaaagcctccagtgcctctgatgtcgacgttgagtatggtgcaccctggggtcttgctcgaatctctcacaggcacaagctgaagtttagcacttttaacgtctacccctttgaggacccagctggtaaaggagttgatgtctatgttattgacactggtatcaacattgagcacgaagagcttgagggcagagcagtctggggtaagacaattcctcaggatgctgacaaggacttgaacggacatggctctcatgttgccggtacgatagcctcccgatcgtatggtgtcgccaagaatgctactgttattgcggtcaaggtccttggtgcaggcggctctggtacaatgtctgatgttgttgctggtgttgtctgggctgctgagtctgcgcttaacaaggccaagactgaatctaccaagtctgactctaagcacaagggaagtgtggcaaatatgtcgcttggtggtgggaagtcgccctccctggatgatgccgttaacgctgccgtcgaaactggacttcactttgctgttgcagctggtaatgataaccgcgatgcttgcaccttttcgcctgctgctgctgccaaccccatcactgttggtgcctccactattcaggatgagcgtgcctatttcagcaatcacggtaaatgcgtagacatctttgctccagggctgaacattaagtctatttggaactctggacctcaaagcaccaatactatttcaggcacctcaatggccagcccgcacattgcgggccttgccgcatatttcttgtctatctaccctgagagccttgatctagctgcattgggacttcaggaagacgatgacgtggaagatgaggttgttgcctctaatggcgaagcttatgaccttcgtcaatacttcacctggaactcagctaactcacctggacagattgcctttggtaaattcaaggaagcggttggttttggtcaagctcaaaaaaccagggctagccgcaaaccattgtcaaccacccttttgaagaaagccatgcttaaactgtctactccaggcgctctagctgacttacctgctcagactgttaactatttggcctataacaatgttacagctacttag

SEQ ID NO: 339 - DNA, 648 bp, Phakopsora pachyrhizi
  source 1..648: mol_type = genomic DNA
atgagtctaagttttaaggctttactagtctttctaccagtgatcttcttctacctggtgttggctgtaaaacaaccatcagatcaaacaactcattctacccaaccacagaagcataacactcatcaaacatctatcaactcaaagcgagaactacaacagcaactcagtcagttcaatccaaatcatcaatctttagcttcatcaaccaggatgtcatcagtcaatgcttcaccaatcacagatattgactcaaacacaaacgttccaacagctgccaagggtgaggaggagtccacaaaacagaagcttgaagatcaaccagcaggcgatggtacaaaggcatcattatcagctaaccctggtgaatcgcaacagagtgacgagaagaatcttaaatctcagaatgtttccccgagtcaagaacacatgagtagatacattgtcactctctcgcgtgagactacttctgcccagttgaaggaatacgctgataaaatcattcagagaggtggaaagattaaccatacgtatgattcagcgattcttaaaggatttgctgtttctataagtgatagtctggttacaaccctggatgatgatccaaatgtaaagagtgttgaacctgatggtgaagtacacatctga

SEQ ID NO: 340 - DNA, 657 bp, Phakopsora pachyrhizi
  source 1..657: mol_type = genomic DNA
atgagtctaagttttaaggctttactagtctttctaccagtgatcttcttctacctggtgttggctgtaaaacaaccatcagatcaaacaactcattctacccaaccacagaagcataacactcatcaaacatctatcaactcaaagcgagaactacaacagcaactcagtcagttcaatccaaatcatcaatctttagcttcatcaaccaggatgtcatcagtcaatgatagaacagcttcaccaatcacagatattgactcaaacacaaacgttccaacagctgccaagggtgaggaggagtccacaaaacagaagcttgaagatcaaccagcaggcgatggtacaaaggcatcattatcagctaaccctggtgaatcgcaacagagtgacgagaagaatcttaaatctcagaatgtttccccgagtcaagaacacatgagtagatacattgtcactctctcgcgtgagactacttctgcccagttgaaggaatacgctgataaaatcattcagagaggtggaaagattaaccatacgtatgattcagcgattcttaaaggatttgctgtttctataagtgatagtctggttacaaccctggatgatgatccaaatgtaaagagtgttgaacctgatggtgaagtacacatctga

SEQ ID NO: 341 - DNA, 1590 bp, Phakopsora pachyrhizi
  source 1..1590: mol_type = genomic DNA
atgcattcctcatctgtgggtttcctgattgctgcattggcgggtgctgctgctgcctccccctaccaggagcaggatgacctcaccttagctccgctggtgaccccacctccacctccgatgggtggtcctctctctcagtttgcaatccagtcaggtcgttcaccatctgactacctcatcaaggatcattacattgtcgtcttcaagaaggacattcacgagaagcatgcccgtgatcacaagcacaaggttcacaagctctggaaacaggatcgtaacgagaggattcaggtccagggctttggtattggtgatatctttgccggtatcaaacatcactttgacattggaacgagcgtctttaagggttattccattcatgttcccgagtcattgatttcgatcatcagaacccttcctgaggttgcctacgttgagagggactctaaggtctggatcaacgatccttactttgccactaacaccgtccctgcaaatgaacctaaagcctccagtgcctctgatgtcgacgttgagtatggtgcaccctggggtcttgctcgaatctctcacaggcacaagctgaagtttagcacttttaacgtctacccctttgaggacccagctggtaaaggagttgatgtctatgttattgacactggtatcaacattgagcacgaagagcttgagggcagagcagtctggggtaagacaattcctcaggatgctgacaaggacttgaacggacatggctctcatgttgccggtacgatagcctcccgatcgtatggtgtcgccaagaatgctactgttattgcggtcaaggtccttggtgcaggcggctctggtacaatgtctgatgttgttgctggtgttgtctgggctgctgagtctgcgcttaacaaggccaagactgaatctaccaagtctgactctaagcacaagggaagtgtggcaaatatgtcgcttggtggtgggaagtcgccctccctggatgatgccgttaacgctgccgtcgaaactggacttcactttgctgttgcagctggtaatgataaccgcgatgcttgcaccttttcgcctgctgctgctgccaaccccatcactgttggtgcctccactattcaggatgagcgtgcctatttcagcaatcacggtaaatgcgtagacatctttgctccagggctgaacattaagtctatttggaactctggacctcaaagcaccaatactatttcaggcacctcaatggccagcccgcacattgcgggccttgccgcatatttcttgtctatctaccctgagagccttgatctagctgaattgggacttcaggaagacgatgacgtggaagatgaggctgttgcctctaatggcgaagcttatgaccttcgtcaatacttcacctggaactcagctaactcacctggacagattgcctttggtaaattcaaggaagcggttggttttggtcaagctcaaaaaaccagggctagccgcaaaccattgtcaaccacccttttgaagaaagccatgcttaaactgtctactccaggcgctctagctgtgagtcattgtttatcataa

SEQ ID NO: 342 - DNA, 1590 bp, Phakopsora pachyrhizi
  source 1..1590: mol_type = genomic DNA
atgcattcctcatctgtgggtttcctgattgctgcattggcgggtgctgctgctgcctccccctaccaggagcaggatgacctcaccttagctccgctggtgaccccacctccacctccgatgggtggtcctctctctcagtttgcaatccagtcaggtcgttcaccatctgactacctcatcaaggatcattacattgtcgtcttcaagaaggacattcacgagaagcatgcccgtgatcacaagcacaaggttcacaagctctggaaacaggatcgtaacgagaggattcaggtccagggctttggtattggtgatatctttgccggtatcaaacatcactttgacattggaacgagcgtctttaagggttattccattcatgttcccgagtcattgatttcgatcatcagaacccttcctgaggttgcctacgttgagagggactctaaggtctggatcaacgatccttactttgccactaacaccgtccctgcaaatgaacctaaagcctccagtgcctctgatgtcgacgttgagtatggtgcaccctggggtcttgctcgaatctctcacaggcacaagctgaagtttagcacttttaacgtctacccctttgaggacccagctggtaaaggagttgatgtctatgttattgacactggtatcaacattgagcacgaagagcttgagggcagagcagtctggggtaagacaattcctcaggatgctgacaaggacttgaacggacatggctctcatgttgccggtacgatagcctcccgatcgtatggtgtcgccaagaatgctactgttattgcggtcaaggtccttggtgcaggcggctctggtacaatgtctgatgttgttgctggtgttgtctgggctgctgagtctgcgcttaacaaggccaagactgaatctaccaaatctgactctaagcacaagggaagtgtggcaaatatgtcgcttggtggtgggaagtcgccctccctggatgatgccgttaacgctgccgtcgaaactggacttcactttgctgttgcagctggtaatgataaccgcgatgcttgcaccttttcgcctgctgctgctgccaaccccatcactgttggtgcctccactattcaggatgagcgtgcctatttcagcaatcacggtaaatgcgtagacatctttgctccagggctgaacattaagtctatttggaactctggacctcaaagcaccaatactatttcaggcacctcaatggccagcccgcacattgcgggccttgccgcatatttcttgtctatctaccctgagagccttgatctagctgcattgggacttcaggaagacgatgacgtggaagatgaggttgttgcctctaatggcgaagcttatgaccttcgtcaatacttcacctggaactcagctaactcacctggacagattgcctttggtaaattcaaggaagcggttggttttggtcaagctcaaaaaaccagggctagccgcaaaccattgtcaaccacccttttgaagaaagccatgcttaaactgtctactccaggcgctctagctgtgagtcattgtttatcataa

SEQ ID NO: 343 - DNA, 1626 bp, Phakopsora pachyrhizi
  source 1..1626: mol_type = genomic DNA
atgcattcctcatctgtgggtttcctgattgctgcattggcgggtgctgctgctgcctccccctaccaggagcaggatgacctcaccttagctccgctggtgaccccacctccacctccgatgggtggtcctctctctcagtttgcaatccagtcaggtcgttcaccatctgactacctcatcaaggatcattacattgtcgtcttcaagaaggacattcacgagaagcatgcccgtgatcacaagcacaaggttcacaagctctggaaacaggatcgtaacgagaggattcaggtccagggctttggtattggtgatatctttgccggtatcaaacatcactttgacattggaacgagcgtctttaagggttattccattcatgttcccgagtcattgatttcgatcatcagaacccttcctgaggttgcctacgttgagagggactctaaggtctggatcaacgatccttactttgccactaacaccgtccctgcaaatgaacctaaagcctccagtgcctctgatgtcgacgttgagtatggtgcaccctggggtcttgctcgaatctctcacaggcacaagctgaagtttagcacttttaacgtctacccctttgaggacccagctggtaaaggagttgatgtctatgttattgacactggtatcaacattgagcacgaagagcttgagggcagagcagtctggggtaagacaattcctcaggatgctgacaaggacttgaacggacatggctctcatgttgccggtacgatagcctcccgatcgtatggtgtcgccaagaatgctactgttattgcggtcaaggtccttggtgcaggcggctctggtacaatgtctgatgttgttgctggtgttgtctgggctgctgagtctgcgcttaacaaggccaagactgaatctaccaagtctgactctaagcacaagggaagtgtggcaaatatgtcgcttggtggtgggaagtcgccctccctggatgatgccgttaacgctgccgtcgaaactggacttcactttgctgttgcagctggtaatgataaccgcgatgcttgcaccttttcgcctgctgctgctgccaaccccatcactgttggtgcctccactattcaggatgagcgtgcctatttcagcaatcacggtaaatgcgtagacatctttgctccagggctgaacattaagtctatttggaactctggacctcaaagcaccaatactatttcaggcacctcaatggccagcccgcacattgcgggccttgccgcatatttcttgtctatctaccctgagagccttgatctagctgaattgggacttcaggaagacgatgacgtggaagatgaggctgttgcctctaatggcgaagcttatgaccttcgtcaatacttcacctggaactcagctaactcacctggacagattgcctttggtaaattcaaggaagcggttggttttggtcaagctcaaaaaaccagggctagccgcaaaccattgtcaaccacccttttgaagaaagccatgcttaaactgtctactccaggcgctctagctgacttacctgctcagactgttaactatttggcctataacaatgttacagctacttag

SEQ ID NO: 344 - DNA, 1626 bp, Phakopsora pachyrhizi
  source 1..1626: mol_type = genomic DNA
atgcattcctcatctgtgggtttcctgattgctgcattggcgggtgctgctgctgcctccccctaccaggagcaggatgacctcaccttagctccgctggtgaccccacctccacctccgatgggtggtcctctctctcagtttgcaatccagtcaggtcgttcaccatctgactacctcatcaaggatcattacattgtcgtcttcaagaaggacattcacgagaagcatgcccgtgatcacaagcacaaggttcacaagctctggaaacaggatcgtaacgagaggattcaggtccagggctttggtattggtgatatctttgccggtatcaaacatcactttgacattggaacgagcgtctttaagggttattccattcatgttcccgagtcattgatttcgatcatcagaacccttcctgaggttgcctacgttgagagggactctaaggtctggatcaacgatccttactttgccactaacaccgtccctgcaaatgaacctaaagcctccagtgcctctgatgtcgacgttgagtatggtgcaccctggggtcttgctcgaatctctcacaggcacaagctgaagtttagcacttttaacgtctacccctttgaggacccagctggtaaaggagttgatgtctatgttattgacactggtatcaacattgagcacgaagagcttgagggcagagcagtctggggtaagacaattcctcaggatgctgacaaggacttgaacggacatggctctcatgttgccggtacgatagcctcccgatcgtatggtgtcgccaagaatgctactgttattgcggtcaaggtccttggtgcaggcggctctggtacaatgtctgatgttgttgctggtgttgtctgggctgctgagtctgcgcttaacaaggccaagactgaatctaccaaatctgactctaagcacaagggaagtgtggcaaatatgtcgcttggtggtgggaagtcgccctccctggatgatgccgttaacgctgccgtcgaaactggacttcactttgctgttgcagctggtaatgataaccgcgatgcttgcaccttttcgcctgctgctgctgccaaccccatcactgttggtgcctccactattcaggatgagcgtgcctatttcagcaatcacggtaaatgcgtagacatctttgctccagggctgaacattaagtctatttggaactctggacctcaaagcaccaatactatttcaggcacctcaatggccagcccgcacattgcgggccttgccgcatatttcttgtctatctaccctgagagccttgatctagctgcattgggacttcaggaagacgatgacgtggaagatgaggttgttgcctctaatggcgaagcttatgaccttcgtcaatacttcacctggaactcagctaactcacctggacagattgcctttggtaaattcaaggaagcggttggttttggtcaagctcaaaaaaccagggctagccgcaaaccattgtcaaccacccttttgaagaaagccatgcttaaactgtctactccaggcgctctagctgacttacctgctcagactgttaactatttggcctataacaatgttacagctacttag

SEQ ID NO: 345 - DNA, 444 bp, Phakopsora pachyrhizi
  source 1..444: mol_type = genomic DNA
atggttttttcatccttgatgctaatagttaacattttaaatgtttttttaactctgacaaatcaattggttcagggagcgtctgttgaaataggtcgaggtttaggtcgtagacaagcaaacttgaagggaatgggccaagcaacattttatacacctggacttggtgcatgcggagaaacacacactgaatcagaaatggtggttgccatatcaaaagacctatttcaatcactaggaggagcaagctctaatccaaatcaaaatcctgtttgtggaaaaactgtaactgctagctaccagggaaaaagttgcagtgtcaagataattgacttgtgcatgagttgcgcacaaggtgacttggatctttccccggcagcctttaaacaactctccgacttatctgctggtcgtatatcaggtgttaccaaacagtggggttaa

SEQ ID NO: 346 - DNA, 657 bp, Phakopsora pachyrhizi; identical to SEQ ID NO: 340
  source 1..657: mol_type = genomic DNA

SEQ ID NO: 347 - DNA, 1020 bp, Phakopsora pachyrhizi
  source 1..1020: mol_type = genomic DNA
atggttgaagcaaaggtattggtagtcgttgcttctttactgttttttagctttttatctgccagtccggcaggctcgcatgtcaggcatcttacaagttcaaagagaccacctcccggggcactcgtcgtaagaaatcagcgaactagagttggagaattttcaaccataaatgatgccgttaaggaattgaagaataagaaaggcccccagttcatcttcattcacagtggcacttacaatgaagcagttcacatcaaatataaatcagcgctgacgattcaagggtatagtgaaaaccccttgtcttatgaagataaccaggtcacggttatcaaacctaccgttgcatctgaggctggaagtgattctaagagctctgttatctgggctcaagttgatgactttaaaatgtacaacataaacgctataaactcttatggagcaggaaaagataaacaggctgtggcggttaccgctgatggtgatcggcaaatgtacaaaagttgcagatttgatagctatcaggacacactgtatatcaggtcaactgcatctttctttgataagtgcttgatctcaggcggtgtcgatgttatctttggcgctggaagtgcttggttcgaaaaatgcactattggggttaaaccctctcctgacaatggtatttctacgataactgctcagaaaagagagcgtggtagtaactcgcggttcgtttttagcagatgtgtagtcgtaggccttagaaattcaaagaccggatcggtatatttaggaaggccttggtccgaatatgccagcgtcgcattccagttctgctttttacctgacctaataaatcctgaaggttggatgtcatggcaacctaatgatcccaagacccgacacgttaaatttctggagttcggaaattcaggcgatggatctcggggtcaaaggaaatatggaactcaggctcgtatgcctttcacagttgatgaagtcttgggttcttttcccgctacttgggatcagtga

SEQ ID NO: 348 - DNA, 1017 bp, Phakopsora pachyrhizi
  source 1..1017: mol_type = genomic DNA
atggttgaggcaaagatattggttgtcatttcttctttactattttttagctttttgtctgcaaatccagcaagcttgcatggtagagatcttgcaaactcaaggagcccacctcctggggcactcgtcgttagaaatcaaggaactagagttggagaatttccaaccataaatgacgctgttagagaattggagaataagaaaggtccccagtacatcttcatacacagtggcacttacaatgaagctgttcacattaaatacaaatcagcactgacgattcaagggtatagtgagaatccatcgtcgtatgaagataatcaggtcacagttatcaaacctaccgttgcgtctgaagctggaagtgattctaagagctctgttatctgggctaaagttgatgactttaagatgtacaacataaatgctataaactcttatggggcaggaaaagataaacaggcagtagcagtcaccgccgatggtgatcgacaaatatataaaggttgtaagtttgatagctatcaggacacactatatgtcaggtcaacagcatcttattttgataggtgttcgatctcaggcggtgttgacttcatctttggtgctggaagtgcttggttcgaaaaatgcacaattgggattaagccctctcctaacaatggtatttcgacgataactgctcagaagagagagcgtggtggtaactcatggttcgtgtttaacggatgtaaagtcgtaggcctaggaaatttgaagatcggatcagtatttctaggaaggccttggtcggaatatgccagcgtggcattccagttctgctttttacctgacttgataaatcctgaaggttggatggaatggcagcctagtgatcccaagacccaacacgttgaatttctggagtatagaaactcaggcgctggatctctgggtcaaaggaagtacggaactcaggctcgtagagctttcacaattgatgaagtcttgggttcttctcccgctacatggggttag

SEQ ID NO: 349 - DNA, 1020 bp, Phakopsora pachyrhizi; identical to SEQ ID NO: 347
  source 1..1020: mol_type = genomic DNA

SEQ ID NO: 350 - DNA, 330 bp, Phakopsora pachyrhizi
  source 1..330: mol_type = genomic DNA
atgttactgtctttaggattcaaacagttcttgattatttctttttttatcttcagaaccttgaatgctacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcgttagtacttgtttggcgaaattactgattttccatgcctatgtcaaagtaaatcgtttatga

SEQ ID NO: 351 - DNA, 333 bp, Phakopsora pachyrhizi
  source 1..333: mol_type = genomic DNA
atgttactgtctttaggattcaaacagttcttgattatttctttttttatcttcagaaccttgaatgctacttcagatattaaaactacagatgagaacaagaaacttttgaaggaagtaggaggggaagatgtcgagcctattagctatgaaaatcaaggggatgagaatcatttgaacaaaagacaactatctctagctgatctacctccctgtggtcagacctgttacgtgggatctttgaccgtttctggtggttgctcaatcgttagtacttgtttggcgaaattgtatgatagaaaccttcaaataaatttttttaaccggaattga

SEQ ID NO: 352 - DNA, 1284 bp, Phakopsora pachyrhizi; identical to SEQ ID NO: 337
  source 1..1284: mol_type = genomic DNA

SEQ ID NO: 353 - DNA, 468 bp, Phakopsora pachyrhizi
  source 1..468: mol_type = genomic DNA
atgagtttaatcaaccgttcaattctcattgtcgtagtggtcttgagcttgacctccctaggatctgttctttcagcggacggacaaaaacaacctaacggcaagatgattggacttctgagagaaattcagagtttacctccatgtgctcaacagtgcttaaggattgccccaccttctggttgtggccaaaccgatatcgcttgcttgtgcaaatctgaagcaaggaaaaatcaaatcaaggaatgtggagataaatgcaaacctgcagataatacggcattatcaaattttgggcaaaagacatgcgataccgtcgttaaaggcgcttctaccaacaaagacgcagttgctcctgatgaggaagatagcacaaaaaagggtggagcagagacctctaacaaaggccctgcaagtgcttctagtagcaccggcaggaagaaagtaactcttcccggccgcagatag

SEQ ID NO: 354 - DNA, 690 bp, Phakopsora pachyrhizi
  source 1..690: mol_type = genomic DNA
atgtcaaagctgtttttcatatggattggccttttcaccatctcaaagctcagcacaactctttctgttagctttggcaagggaataaatccattgtcactagcagacgagggcgagagtgtgctgaagcagtttcaaatctttggtaaaaataatgaggtcgtgaatgtcaagattgtttcaaagtttaactctgattctaactcattaagattcacaaagagagacctcctgggaattgaggatgagaagagtaatgagaagggaaacgataaggtgaagaatcaggcccagaatagaaattacactagagtcgatcttacacctgctcgtcctacttcagaaacctgttactctggttcattccaaggaccaaaccagagtgattgcgatgtgatattctatgcccagaagtataactcttatggtagtttaactgctttcccaggaacgtttgtttatgtttactactcttcgtgtgtggtggcttttcaaaaccccaatcatagtaactattcattagactacaattgggcaatgctaggtgctaaagcagagcagattaagaacagatgcctcagagatgaggagcagtcgattggtggatcttttctatttgaaaactatttgggctacacttttcaaaatgtgcttataagtcttcaaaggtatgctggcaatataacggcg

SEQ ID NO: 355 - DNA, 435 bp, Phakopsora pachyrhizi
  source 1..435: mol_type = genomic DNA
atggttttttcatccttgatgctaatagttaacattttaaatgtttttttaactctgacaaatcaattggttcagggagcgtctgttgaaagtcgaggtttaggtcgtagacaagcaaacttgaagggaatgggccaagcaacattttatacacctggacttggtgcatgcggagaaacacacactgaatcagaaatggtggttgccatatcaaaagacctatttcaatcactaggaggagcaagctctaatccaaatcaaaatcctgtttgtggaaaaactgtaactgctagctaccagggaaaaagttgcagtgtcaagataattgacttgtgcatgagttgcgcacaaggtgacttggatctttccccggcagcctttaaacaactctccgacttatctgctggtcgtatatcaggtgttacctggggttaa

SEQ ID NO: 356 - DNA, 384 bp, Phakopsora pachyrhizi
  source 1..384: mol_type = genomic DNA
atgccaaccttcagcttcacctggacagacgctgcactgttcgggctgactgcaggattgatcgccacgatcgccctgatcggtcgcagctcatcatcacaatctaaaaggagcgtcttagattcatccagaacaaaccagtcccagagctccaagaagagtaagaagaagaataaacctaagaagtctcctccccaacccagctctcagacaacaatcggctctaatcagaaatccgaaccaaggcataccgatgagaggaaggcttcaccccctaaatcttctaacccatcacccaaaccgatctctgagccgaagaagaaaaagattcaacagccccaaattcctaaactcgatgacaatgagtttcctacccttgcaaat

SEQ ID NO: 357 - DNA, 309 bp, Phakopsora pachyrhizi
  source 1..309: mol_type = genomic DNA
atgtctcttgcctcatccctgtcggttgcctttgtaattgcgcttggtatcggccaaggtactgcccaggttcctgagagtgcgccagcatgcgtaaggaattgtgccagagtaaaaattgccgagggctattgttctacgcccggcgtattaacagctgcatttaatcaatgcttaagcgagaactgtaatagcgctgacgcggcttcgggcgctgcttttgccaacgtcgtctgtgaaggagctaaccctaatagtaattttcttgacctttcccaatattctgttgtggacactgatataatttag

SEQ ID NO: 358 - DNA, 489 bp, Phakopsora pachyrhizi
  source 1..489: mol_type = genomic DNA
atgcttttcaactcaaacgtgatcctctttgcattaacttttgctctgggatcgttataccagggatcagaagctcaggatctggccggacaacctaactgtggtcagaactgtctattgacagcctttacgacattgagcaatggttgcacacagaccgactttgcatgtctttgcaagtcgcaaaagtttactggtacatcaaatttgcgctactccttgtcctgtacctccactgaagcagcgactaccaagagctggggtgccaagacttgtgcttctgtaggagttaatgtgacagccacccttggaaacaccacctccgtcagttctgacaaacagcaacagcagcagcaatcatcattgtcatcattgctggcaccgctatcattgttgccggcagaggtaggagcaggaggcttagtgcctcctctgttggaacaggctgactcagatgtagtcaatacgagcaagttgagcacgagctag

SEQ ID NO: 359 - DNA, 513 bp, Phakopsora pachyrhizi
  source 1..513: mol_type = genomic DNA
atgcttttcaactcaaacgtgatcctctttgcattaacttttgctctgggatcgttataccagggatcagaagctcaggatctggccggacaacctaactgtggtcagaactgtctattgacagcctttacgacattgagcaatggttgcacacagaccgactttgcatgtctttgcaagtcgcaaaagtttactggtacatcaaatttgcgctactccttgtcctgtacctccactgaagcagcgactaccaagagctggggtgccaagacttgtgcttctgtaggagttaatgtgacagccacccttggaaacaccacctccgtcagttctgacaaacagcaacagcagcagcaatcatcattgtcatcattgctggcaccgctatcattgttgccggcagaggtaggagcaggaggcttagtgcctcctctgttggaacaggctgactcagatgtagtcaatgtgggtagtggcactgaggaacagacagatatcaatctttcccaatag

SEQ ID NO: 360 - DNA, 672 bp, Phakopsora pachyrhizi
  source 1..672: mol_type = genomic DNA
atggttgaagcaaaggtattggtagtcgttgcttctttactgttttttagctttttatctgccagtccggcaggctcgcatgtcaggcatcttacaagttcaaagagaccacctcccggggcactcgtcgtaagaaatcagcgaactagagttggagaattttcaaccataaatgatgccgttaaggaattgaagaataagaaaggcccccagttcatcttcattcacagtggcacttacaatgaagcagttcacatcaaatataaatcagcgctgacgattcaagggtatagtgaaaaccccttgtcttatgaagataaccaggtcacggttatcaaacctaccgttgcatctgaggctggaagtgattctaagagctctgttatctgggctcaagttgatgactttaaaatgtacaacataaacgctataaactcttatggagcaggaaaagataaacaggctgtggcggttaccgctgatggtgatcggcaaatgtacaaaagttgcagatttgatagctatcaggacacactgtatatcaggtcaactgcatctttctttgataagtgcttgatctcaggcggtgtcgatgttatctttggcgctggaagtgcttggttcgaaaaatgcactattggggttaaaccctctcctgacaatgatgtgtag

SEQ ID NO: 361 - DNA, 147 bp, Phakopsora pachyrhizi
  source 1..147: mol_type = genomic DNA
atgagatctcagagtcccctcctattgattttaacacttatctcatggagaaaaatacactggttggtattgaacttttgcagttattttagaaactttcaaaaagaaaaaaaaaaaggcttctctgcgcaaagactcttgcgttga

SEQ ID NO: 362 - DNA, 273 bp, Phakopsora pachyrhizi
  source 1..273: mol_type = genomic DNA
atgtttttgttaaatgagagattcttaggtttaatcttactactcatatcaactctcgtcactacgattgtctcgatagcttggttcaggtttaagactctgaacccaggcaggatagctcaactgatcgaactggagatggatcaactcagtaataacagacagagatctctgatagatgataaagagctatggaatgacttggatccaatccctcaggttgactcagaacgtccgatggttcagatagtctattctactaaatgtaactag

SEQ ID NO: 363 - DNA, 438 bp, Phakopsora pachyrhizi
  source 1..438: mol_type = genomic DNA
atgagtctaagttttaaggctttactagtctttctaccactgatcttcttctacctggtgttggctgtaaaacaaccatcagatcaaacaactcattctacccaaccacagaagcataacactcatcaaacatctatcaactcaaagcgagaactacaacagcaactcagtcagttcaatccacatcatcaatctttagcttcatcaaccaggatgtcatcagtcaatgaacagcttcaccaatcacagatattgactcaaacacaaacgttccaacagctgccaagggtgaggaggagtccacaaaacagaagcttgaagatcaaccagcaggcgatggtacaaaggcatcattatcagctaaccctggtgaatcgcaacagagtgacgagaagaatcttaaatctcagaatgtttccccgagtcaagaacacatga

SEQ ID NO: 364 - DNA, 570 bp, Phakopsora pachyrhizi
  source 1..570: mol_type = genomic DNA
atgttcatattgagttggttttgggacgttttagctcagttcggactggtaaataaaaatgcaaagatactgtttctggggttagacaatgccggtaaaactacgttgttacacatgctgaagaacgatcgactagcaactctccaaccaacacttcatccaacttcggaagagctggcgatcggtaacatcaaatttaccacttacgatctcggtggtcatcaacaggcccgaaggttatggaaggagtatttcccagaggtcgatgggatcgtcttcttagttgattctcaagacttggagagatttccagagtcaaaggctgagttggatgccttactcagtatagaggaactttttaaagtacccttcttgatcctaggtaacaagatcgatgccccaggtgccgtaagcgaggaagagctcaggcacgccctaggcctataccaaactaccggaaagggtaaagtgcccttgaccgatatcagaccgatcgagatcttcatgtgttcgatcgttatgagacaaggctatggtgacggttttcgttggttggcccagtacatttaa

SEQ ID NO: 365 - DNA, 606 bp, Phakopsora pachyrhizi
  source 1..606: mol_type = genomic DNA
atgaacatgaaacttagagtattcatttccttaactgctgcgctttctttgattgctcaatccccaagcctagctgtaactttaaatagagatcggccttcaaatgcccatgcgctccaggctagacaggaacaagctgcagagctagctgctgtcacggtaccggaagaacaagctgccgctagtgctcccgagcagaaggctgctgctgctgcctccgctccagagcacgctgctgcttcctccgctcccgagcacgctgcttctgctgccgctcctcaaagtgacctagacaaagctgccaatagattgtacgacttaatggtaaaccttggaaatggattggagatcgtcacaaatcttgaggccagtccacaggatatcaaggcccagactcaaaaagtcaaacaatattttggtgaaatgaatgagctaagaaacaagttgctccagatgagccccatacattctggaaacctccccaaatctgttcaagatgccaatgaagctcaggaaacattacagaaagcgttgattgctattagtgattcggcagaagacgcaaatgttctaaaaaaagaactacccagtcctttcgaagacttt

SEQ ID NO: 366 - DNA, 1446 bp, Phakopsora pachyrhizi
  source 1..1446: mol_type = genomic DNA
atgaaaaatatttttagaggttctttatatatatttttgacactccttaatttgattgtaagcctcgaagcagccttagtagcatttgacgaggaagttagcttgttgaggaatacggatcgcataagccaaactcaacccaagactttagagctccaaaaatattcaaaggcttataatggtcctttgatatatgccccatccagctctgtagaaatcttcaaaagtgcagctcagttgcacactagtccaaaatctgataaatcttttactcgagattggagaagtatccacatgaatgatttattaaatccaaggattactcaacctataaaatctaaaaaagggtctttacttttatacttatttagaaaaaaacctaaaactcaatatgaggttaaaaagacattagttctttccgacaaaggttctaccgatacttcatctttattcaataaagaatctttagcgctaagttaccccagaaaagaaaaaaattccggaaactacttagagggcttagagattgaagttccaacgctcagtgatgaagcattagtagatctaagagactggaagcggagcacatcagtgttatattttatagaacagatcgagctaagaacaaataaggttgtagatttactaaaaaggccagatttaaatatagaagatgtagagggtctctcaaactttttgaaagacttaaacgttgatgagaatggagatttgaaaaacctcagggagggtgaaacgatctatctgatgattcaagacgtttggaaggcttcaaagtctaacttccctgccgaggtaaaaactcaaatcttttatagatacgagtatagactcaaaaagatcttcgataagctctcagagcaaaggtttttcattaaaaagggtaaaaaggaaactctaatcaaggaatcaatggagatagttctatacaggcttaaaaaatttaaagaaaaattggaagatgagatcatcatgttggacaattggcgcgacgaatccatgaaaattatcgaaaagttcaactcactctggtggatgatatcggatgattcaaaccttagggttaagcaggagaaaaacttagataagattttaaccgttagtgctgtgagatctggaaatgagaagttattcaaagaattttataagatctctgtgaaggttagaggagattctgaagaaaatgaaggtttactacagagtctgatctcggagatctcacccttgaaaaagaaagatccgaagaagagggcaattttctattatcaaactcatagtatctctgaggaactgtaccatgcaactatagctcaggctaaaaacgtcgggaccaagattctacttaatgaaaatgattttgataggttatatgatcaaaaacttttatctaagaaggcaaatcttttaaagccaaaggatgtaatattt

SEQ ID NO: 367 - DNA, 138 bp, Phakopsora pachyrhizi
  source 1..138: mol_type = genomic DNA
atgaactgtcaaaaaaaaaaaaagcattataaacttatacttctatataaatttgattggagagaggattatttggtactttttttttgttttatattatttataatgagacgtttaaggattgtagaaatgaataca

SEQ ID NO: 368 - DNA, 429 bp, Phakopsora pachyrhizi
  source 1..429: mol_type = genomic DNA
atgcttttcaactcaaacgtgatcctctttgcattaacttttgctctgggatcgttataccagggatcagaagctcaggatctggccggactacctaactgtggtcagaactgtctattgacagcctttacgacatcgagcgatggttgcacacagaccgactttgcatgtctttgcaagtcgcaaaagtttactggtacatcaaattcgtgctactcctcgtcctgtacctccactgaagcagcgactgccaagagctggggtgccaagacttgtgcttctgtaggagttaatgtgacggccacccttggaaacaccacttccgttggaactgtcatcggaaatggaactaatcctaacaccaactctacgcttcataatggcacaactgtaagcagtatactcattaagtatacgatagcggtctga

SEQ ID NO: 369 - DNA, 438 bp, Phakopsora pachyrhizi
  source 1..438: mol_type = genomic DNA
atgagtctaagttttaaggctttactagtctttctaccagtgatcttcttctacctggtgttggctgtaaaacaaccatcagatcaaacaactcattctacccaaccacagaagcataacactcatcaaacatctatcaactcaaagcgagaactacaacagcaactcagtcagttcaatccacatcatcaatctttagcttcatcaaccaggatgtcatcagtcaatgaacagcttcaccaatcacagatattgactcaaacacaaacgttccaacagctgccaagggtgaggaggagtccacaaaacagaagcttgaagatcaaccagcaggcgatggtacaaaggcatcattatcagctaaccctggtgaatcgcaacagagtgacgagaagaatcttaaatctcagaatgtttccccgagtcaagaacacatga

SEQ ID NO: 370 - DNA, 786 bp, Phakopsora pachyrhizi
  source 1..786: mol_type = genomic DNA
atgggtgtttcaaagctgtttttcatatggattggccttttcaccatctcaaagctcagcacaactctttctgttagctttggcaagggaataaatccattgtcactagcagacgagggcgagagtgtgctgaagcagtttcaaatctttggtaaaaataatgaggtcgtgaatgtcaagattgtttcaaagtttaactctgattctaactcattaagattcacaaagagagacctcctgggaattgaggatgagaagagtaatgagaagggaaacgataaggtgaagaatcaggcccagaatagaaattacactagagtcgatcttacacctgctcgtcctacttcagaaacctgttactctggttcattccaaggaccaaaccagagtgattgcgatgtgatattctatgcccagaagtataactcttatggtagtttaactgctttcccaggtaagtcaacacgtttatttttgtttatttttattttttggagggttggtgaggttcaatacttatataattgtgagccttatacaggaacgtttgtttatgtttactactcttcgtgtgtggtggcttttcaaaaccccaatcatagtaactattcattagactacaattgggcaatgctaggtgctaaagcagagcagattaagaacagatgcctcagagatgaggagcagtcgattggtggatcttttctatttgaaaactatttgggctacacttttcaaaatgtgcttataagtcttcaaaggtatgctggcaatataacggcgtaa

SEQ ID NO: 371 - DNA, 699 bp, Phakopsora pachyrhizi
  source 1..699: mol_type = genomic DNA
atgggtgtttcaaagctgtttttcatatggattggccttttcaccatctcaaagctcagcacaactctttctgttagctttggcaagggaataaatccattgtcactagcagacgagggcgagagtgtgctgaagcagtttcaaatctttggtaaaaataatgaggtcgtgaatgtcaagattgtttcaaagtttaactctgattctaactcattaagattcacaaagagagacctcctgggaattgaggatgagaagagtaatgagaagggaaacgataaggtgaagaatcaggcccagaatagaaattacactagagtcgatcttacacctgctcgtcctacttcagaaacctgttactctggttcattccaaggaccaaaccagagtgattgcgatgtgatattctatgcccagaagtataactcttatggtagtttaactgctttcccaggaacgtttgtttatgtttactactcttcgtgtgtggtggcttttcaaaaccccaatcatagtaactattcattagactacaattgggcaatgctaggtgctaaagcagagcagattaagaacagatgcctcagagatgaggagcagtcgattggtggatcttttctatttgaaaactatttgggctacacttttcaaaatgtgcttataagtcttcaaaggtatgctggcaatataacggcgtaa

SEQ ID NO: 372 - DNA, 417 bp, Phakopsora pachyrhizi
  source 1..417: mol_type = genomic DNA
atgattggaaagtttctcttgatggttcttttttcatcgattttcttggcttttggaaccattggccagagaatacctgaaggtttatacgtgtcttgtgccaatgccgttgtgagacagagctgttctgctgctttggatgttgcttttactgaagggaggatcagaagattcaccactgctcgtcaatacattcaaacccaagggtgcagactctcttggtgggtagacactggtatacttcgtattgctcaaaaagacgtacttcaagcagctcttcagcaaattgacgattcctgtactgcaaacggtgtttccgtcataaatcctagcaatcttccaggtcgaggggtttacgttggccagatggtcggcaatcaatacgctgttcttagagctgaaataggagctttcgcc

SEQ ID NO: 373 - DNA, 378 bp, Phakopsora pachyrhizi
  source 1..378: mol_type = genomic DNA
atgacaattggtcatctcaataaaggcttcattttctattgggttgcgttgatatttttgccaaagattgttattggtagtttcacgcctaaatccaaaggtttcgaaacgatcattggtgatgaaaacaaaattaaaaatgttcccttgaaagatctgagcaaagaatcctctagccaattgaaagaatcccgagacgatgtttttgggcctggaagttatgccatgagctataatgctaagtctcaagctacaaaacatgccccaggagatgatgtcagtaaatcagaggctggagtgtcatacaatattgcaagagatgtacaagattccaagcgaagcaagtcagaaggaaggtatgacgaattgcatatcaag

SEQ ID NO: 374 - DNA, 1497 bp, Phakopsora pachyrhizi
  source 1..1497: mol_type = genomic DNA
atgttttcgctcacagcttcaatactgattctttcattaattgcacggacctctgcgcaccttagtctgcttgcgatttatggctccaacaatgttattggacgaggttttggagtaaatactgatggtaaatatcctcgaaaattaggtagaagtggtgatgctggaggtgactctaccgtctttgaaactggaactgataatccatcacctgcgtgcggtcgcactcctgagcttggggaactagacattccagcatggctaagccaagccgagggagaaggccttcctgcagcctattcgaatggttcaattgtggcagaagcctttcaggttaatcgtgacggcggtggccccatgagctgcgaatataatcaggatgcaacggctacttcctggaaaagtatgtttttaactttgaatcaggcaggaaactcggggatatacaacgttaatagattcaattcgacggtggttattaactttcctcagggggctaaatgtaccggaggatggactggaactgcttgcattgtgagatgcagaacaggtgtaaataagcgatttggtggctgctttgcggcaaaactttctagttcgtcagcacctacactagtgcacagcgctaatgctgtttcaggatctcaatcattgagcgacgcccaattggatgttattgcacaaaaaattgtctctcaaatgaaacttgaaggccttttagtcaaagacgactcgcaatctacgaattctggcggacttacatctagatcgagctctaattctcagaacgtcatcgcctatgtctctcattcaaaaaaaaagagtgcgacatcgagtttaaagtccaatgccaagcccaaaacgaaaaagcatagcggaatgcttcctaacctcggggaggctgctccaactcctgtaaatgaagagcagcttttcagatttcccaaagaggatgaagatataagagagggagaggacatcaatcaaccacttacaaattctgatatcattattaatgatatcccagtacctcctcttgcaaccccaatcaacagctcaacatccaggaagacatcaaattcatgtaatctgaatcccaatagtcatcagaagacatccaaatctaccctaccaccgacaacatatgctgagctgtatgaggcaaaaacagatcgcaggtcagactatgaatcagggcgtatctcctgggagcgggagaaatggagtaaggaaagtgaattaaggatgaagcaggaggaagttaaaaaagaattagaatataaaaggcttgagtatgcaaaagaggaaaaagataaagatctgatttatgcaagagaagaaaaggaaaaagatcatgaattgaaattgtttctagcagaaaaagaaagagaagcattaaaaagaaaagaagaccgtgatatgttacttgctgttgtgggttcatcacgctcccttgcagagatttctgaaattgcaaagattttaaatattgcttag

SEQ ID NO: 375 - DNA, 654 bp, Phakopsora pachyrhizi
  source 1..654: mol_type = genomic DNA
atgctttttgctacactaatagccgtttgcttacttgctctgggaggcaaagctgagagtgatgtccaaagtgataccgcatctaaacttcagcgacgaggacatgatgactctctacctcctgtgacatttattatgagagatagcaacgagcatgtaggcggaaagctgttgatctacaattctgatggaacactagctttcacctttcgcagagcagttttgaactctgatggcctttccaacgttgaagtcagagatgtgcgaaataatttttcgataaacttagaatctgacgatgacacctgctttaaaaagtctcactatgttgaaagagaaaaaaatctgggtcaattcaaaattgatccacgaggtgcaaaagcagacagatggcatttcactaggaaaactccacacggagatttcaagtatgactttcatcgcaagtatttttcgaaggacggtaatatttacataaacgacacccatgtgcgagtggcttccctgacatccgaaattcgacatgaagcttggctccaaccggggaaacatggtgttccaacattctcattgcatcttcaatataacactgagcctatcttttttgtagctttgatgggcttagatctcaccagagtagacacatgtggactt

SEQ ID NO: 376 - DNA, 855 bp, Phakopsora pachyrhizi
  source 1..855: mol_type = genomic DNA
atgttatcatcaactcaactctccatcatcttggccctgtccatcagctatgcattcgtctcggcccagtcagtcgccaactctactgccactgctcctactcccaccactggtgccggtggtcttaccgctggactctcacccggatgtcaagccgccgctggtggacttctgagtaccgagttcagctcgtgtgctaacatcatcggattggtttctctgatctcagctcagggtggactcatcgatcctttgagtaactggatctctggagtttgctctgccagtccctgctcgagtgctactctctcaaccacctccacatccctcaacgctggatgtggggccgatattcagaaaggcgttccttctgccattgctttaaacactgtgatcactaactacaaccctatccgtgagatgatctgtacccagtactccacgaactctacattttgtgccacttcaatcttgagcaacattcaaactgccaccggcaagagcattaccctgaatgaggtccaagctgtggtatccggaggccttaacgctttaccactgtcctcagttcctgctagcgcgtactgtaacgattgtggacacgccctttttacaaaatctaaggccatcaatgtaaacactggttcatcgaacctcacatcagcctcttctggtgcttctgatgctgctgcccagacctgtggtgcttcattcaacgatggaaaggtcccaagtactgtccgcgttgcaggatcgactaaggctggagcacctaatgccggaggaaagaacaaagcttctgtggtcaatgctgttgctatgggcctaatggcattagtcgctatttgcattgtt

SEQ ID NO: 377 - DNA, 375 bp, Phakopsora pachyrhizi
  source 1..375: mol_type = genomic DNA
atggcgtttgtgtccttatcatttgcacagctactaccaagcgcctacagctgtagcagggttgatctttacaaacctccactaagacgtgaagattgcagagactccctaaatttgtttccagttccaaacggcgataatgttatttaccttggaagtggcaattggcgaggttgtggaagctgcaaggtgactatttacaataggggatcaagagaatctcgtgtgactgcgccaaaaggctgggcagctacggcagtacaccaagctttcgaccattgtgaaggaaagccgggatctgctactattggagatgatgggaaaattattgcaaaaatcgattacggaaattctggacaaggagattgtcctccc

SEQ ID NO: 378 - DNA, 699 bp, Phakopsora pachyrhizi; identical to SEQ ID NO: 371
  source 1..699: mol_type = genomic DNA

SEQ ID NO: 379 - DNA, 1143 bp, Phakopsora pachyrhizi
  source 1..1143: mol_type = genomic DNA
atgtcagtaaaaggttttgacaagagagctttactgctaataatatttggctacaactcttttatttcatgtcacggattcataaaatcttggtcaccggacgatggaaaaacttggattttaggacagaaacaacatcttgaaaagtcttctattagaagcttctcagaaaacacaggttggatcggaagcaactttctaactagtccagcaattgtctgcggctcgagtaaaactccaagtcagagggtagcaccgcctagtggggagatgttcagtcaagctgatcagagtgctggaaagactcttccagtagaatctggggctacagtacgtctactgatcaatgacgaagagggaaaagtatatccgcacaatgagggccacattcaggcttacttgggtcgatgcggtgacgctgaaaattcttgtcaaaactttgatgcagccgctattggatacttcaagattctgtccgaaaaagatggattagcttcaacgaaaattttcccatacaacaaagctaaagatggcaatgtgctaaaggttcatatgcctagaaatcttcctaagggtggttatattcttagagtagagctaatagcaacaggagaatcgtctgagaaggaaggaaagcaagaccaatactatgtgacttgtggccaaatctcattatcagaaccaaaaagcacacctgctatcctattacaagctataccagttgtaaaatttccaggggcttacaagaacggaaaccttgaattagaatctaaactcccagggccacgtatttttgaagggttttcaaccgataaaaagggcgctgctaatcagagaaactctaatgggtctgatagttctcaacagctcacacctagatgtgcagtacggtgtcttcagaaaatttcaaccgaccaagaacaaggtctttttgatgtttgtcatcaaaaagagggtcaagcaccttgccaatgtcaaaataagcaatttgttaaggctttttttgattgttcccaagaaaattgcaagggagaatgcgagcttgaaaacggtattgcagcgtttgacattatgtgcaaagtaagaacaggagataatttttgtcatccaaaatcaaaaaaagatgacaaagctcttgcataa

SEQ ID NO: 380 - DNA, 351 bp, Phakopsora pachyrhizi
  source 1..351: mol_type = genomic DNA
atggcaactttcttggcaaagaacgtagcccttgtacctctcttcgttgccgtggggttaggtctaggaggtggaataggatttggaatccattatttaaagaataaccaagacgtcgttttaagaaaaaagtcaaacccagacccatggaacaaagttccacaggacaacaacacgaagtttttctcttttaatccggacttctggcgagctagagcccaattgactgatccaagattaagttttatggaaagtaaaccagagaatgagagaactttacatgaacaagccatggtcgaaagggctaagcagatcagactgaatgacaaggaacgtaccattcacagttga

SEQ ID NO: 381 - DNA, 288 bp, Phakopsora pachyrhizi
  source 1..288: mol_type = genomic DNA
atggcaactttcttggcaaagaacgtagcccttgtacctctcttcgttgccgtggggttaggtctaggaggtggaataggatttggaatccattatttaaagaataaccaagacgtcgttttaagaaaaaagtcaaacccagacccatggaacaaagttccacaggacaacaacacgaagtttttctcttttaatccggacttctggcgagctagagcccaattgactgatccaagattaagttttatggaaagtaaaccagagaatgagagaactttacatgaacaa

SEQ ID NO: 382 - DNA, 429 bp, Phakopsora pachyrhizi
  source 1..429: mol_type = genomic DNA
atgcatgctgctaatatgaaagtttcaacccttatgatagtagtgttaagttttatccagctatattcttttactttatctcaaatcaccaatgggggcattacgcaggatccaaatgataaatacagggttagatgtttagccgactctaaatcaggggatataatttctgatgactgctatgattcattggatgacttttcccaagatgatggaattattaaattcaatgcaaaaggagaagataatgatgaagactcttgtaggttggagttagttggttatcctaatcaaatcaccaatagtgatgaaatcaaagtgccaaaaacatctgtgatcagcgcaatcaattttggcttagatggctgtaaaaattcttcagccatagttgaagcaacaggctttattctcaacatttacaaaaattaa

SEQ ID NO: 383 - DNA, 429 bp, Phakopsora pachyrhizi
  source 1..429: mol_type = genomic DNA
atgcatgctgctaatatgaaagtttcaacccttatgatagtagtgttaagttttatccagctatattcttttactttatctcaaatcaccaatgggggcattacgcaggatccaaatgataaatacagggttcgatgtttagccgactctaaatcaggggatataatttctgatgactgctatgattcattggatgacttttcccaagatgatggaattattaaattcaatgcaaaaggagaagataatgatgaagactcttgtaggttggagttagttggttatcctaatcaaatcaccaataatgatgaaatcaaagtgccaaaaacatctgtgatcagcgcaatcaattttggcttagatggctgtaaaaattcttcagccatagttgaagcaacaggctttattctcaacatttacaaaaattaa

SEQ ID NO: 384 - DNA, 825 bp, Phakopsora pachyrhizi
  source 1..825: mol_type = genomic DNA
atgaattctcacattggctcaaagaccattccccacattcttttacttccctatttcctaatccttcttacttcgatttaccattcggttattggtacatcaatagaatcacttgtcacagcagttgatcaaaaagatgtacatgaaggtataaattttatgcttgatggctcaacacatggtgcacggcatgaattgaaagttggtgaagaagataaaaatgttacattgaaggatcatggtcaaagtcatctacttgaaattgttagaaaattagaaaagaggtattcttctcacaccacgacgattgaaaaaacggaaaattttccaaaaagttctgctcggtcccatcattttaaaaggagtactccatcatctggagaaatcagaagatggcttagaagtcacaataaatttagagcccaatactctgcctctccgttggtgtgggatcagaacttggctgacaaagccaattcagagactaatacctgcgtttggagacactcttacaacgatatatatggcgaaaacattgcagccggccaggaatcgattgaagaggttgtggatgagtgggtcacaggttcagaagagagaagagtttattcaccaaataaccctacatattcacattttacccaagttgtttggggagacacaaggcgtttaggatgtgcaatgacctcttgtagaaatattcgaggctctggtttaccacaatctcctgtaaagttttgggcttgtgaatattatccgcctggaaatgtagatggccagtatcgtcaaaacgtcaaagctagatatggtggctctcctctataa

SEQ ID NO: 385 - DNA, 771 bp, Phakopsora pachyrhizi
  source 1..771: mol_type = genomic DNA
atgaaggtattaaactttagcctattgacaattctcttgttggccctactggtcagtatttctcaagggaaagaagaccaagaagagatcagtcccttggaggaatactttggtagacaggatttagagtccagaggccttgaagatgactggatagatatcagtgttggagtaatgatgaaaagggaagaagataaccaatttaacactagctctagtcttactaaacgatcaaccggtgattgggaaaccagcgaaatcttggtggctagagatccaaagtctaatagtttaatatttccgagtagctggagtgaagcttctcaaatgatcaaaagagatatacctcaatttgttggaagacattacaagcgtgcaaagagcgctatgagcgtgaaaatcacatggtacactggtcatgatttgcttaatccggcctgtgtacagggaggctcaggctgggcaccagttgacaaatctatggctgcagcggttactattgagtggccaggtaaaccaaagtgtggaaaatttgtaaggattcaaaagaaggaggaccctagtgtgagcgttgttgtaagagttgtcgactcgtgtggtggatgccgaccttattcacaacacattgatttaaccattggagccttcaaaaagctatacgcccaagaagtcgggcaagttgaaggactgagagctaggattgtaaaagctcctgtaggacacaagtggacgaaaaaagatacaaagatctatggtcctaagaacctttga

SEQ ID NO: 386 - DNA, 681 bp, Phakopsora pachyrhizi
  source 1..681: mol_type = genomic DNA
atgaaggtattaaactttagcctattgacaattctcttgttggccctactggtcagtatttctcaagggaaagaagaccaagaagagatcagtcccttggaggaatactttggtagacaggatttagagtccagaggccttgaagatgactggatagatatcagtgttggagtaatgatgaaaagggaagaagataaccaatttaacactagctctagtcttactaaacgatcaaccggtgattgggaaaccagcgaaatcttggtggctagagatccaaagtctaatagtttaatatttccgagtagctggagtgaagcttctcaaatgatcaaaagagatatacctcaatttgttggaagacattacaagcgtgcaaagagcgctatgagcgtgaaaatcacatggtacactggtcatgatttgcttaatccggcctgtgtacagggaggctcaggctgggcaccagttgacaaatctatggctgcagcggttactattgagtggccaggtaaaccaaagtgtggaaaatttgtaaggattcaaaagaaggaggaccctagtctatacgcccaagaagtcgggcaagttgaaggactgagagctaggattgtaaaagctcctgtaggacacaagtggacgaaaaaagatacaaagatctatggtcctaagaacctttga

SEQ ID NO: 387 - DNA, 1587 bp, Phakopsora pachyrhizi
  source 1..1587: mol_type = genomic DNA
atgctatcgatcttgcgatgggccctactcatgggctgcttaacttactctgctatggctgaagccgagcaggatgacgttgtaaagctcgacggttcaaactttgcaactattgtaaacccagcacctttgatattggtaaaattcatggccccttggtgtggtcattgtaaagcgctgaagccagagtaccatcatgcggctactgaattgaaggaacactcgatacctttgggtgttgttgattgtacggccgatgccgatctctgtagtactcacgaaattcaaggctatcctactttgaaggttttttctcatggaaaggcatcagagtatggcggcactcgaaaagctgatggaatcgtgacttttatgaaaaaacgatcccttcccgtcatatccattgtcacctctgggaaccacaccgatttcacacaatccgacaatgttgttattgttgcctatcttgacgaatccgacaaaaagtcttacgaaacattccaaagatatgcagagaccaagagggacgaatttatttttggagctgttcacgagcattcaaagatcgattcagtctcctcgctgcccaagcccagtattgtcctgtggaagaagtttgatgagggaaggaacgattttcacgggcaggaattcacagatgaaaacgtctccaagtttgtgtctgagaactctattcctctgttgaatgaggtatcacccaagaactttgccatgtacgaagcggcaggaattcctttagcttacttgttcattgagcctaacaatcctgagagagaggctcttatcaaggcgcttgaacctgtggccaaggaattcaagggaaagatcagttttgtttggatcgatgccaaccagttctcagaccacggaaagactctaggattgagtgcaaccaattggcccgcctttactattcaggatctgggcaagcagaccaagtatccttttgacgaaaaaaaggctatcgaccaaaatagcatcatgaattttgtcaagtcttttgttgatggaaagctttctcccagcttgaagagtcaaccaattccccagaaacaaggtcctggatcgcatgtccttgtctctgattcgtttgatgatgttgttttcggtgacgacagcaggagggatgtttttatcaaattttacgctccttggtgcggtcactgcaagcgtttgaagccaatttgggataacttagcagcaagcttcaaccaatcttccactgatgttgtcattacgaaattcgatgccactgagaacgatattccaccaactgcagagatcagcgtgagcggatacccaactcttaaatttaaagcggccggtcagaagggctttatagattacgagggagatcgaagcttggattctttgattgcctttgtcgaggagcattctaataacaaggttaaagctactaaggttgagttatcagaggaagagtttgagtatggaactggtggcgaacaggttgtctttgataaagaggaggctgatgccagtggtgatcttgatgatgagggtgttggaggtgatgaagataaagatgccgaacatgatgaattg

SEQ ID NO: 388 - DNA, 1368 bp, Phakopsora pachyrhizi
  source 1..1368: mol_type = genomic DNA
atgaagtactttgttttacttgccgtcgcttttcataattttgcattaaacaatgctcagggagttggaacaacgactccggagactcaaccaaagtttgtatatcatagttgcactggaaaggactcttgtaagcctgtagatggcaccgtcactgtcgattccaattggaggtgggctcatgacgcaagcgggaagaattgctacaccggtaacacttgggacaccactatctgcccggatggagcaacatgtgcaaagaattgtgttttggacggtgctgattacaagtcaacctatggtgtcacctctgacaaagatggtgcttcgctcacccttaagtttgttactaatgggccttattcaaagaactatggctctcggttgtatgccttagcttccgatgggaactatcaaatgtttatgctcaaaaacaaaaggttttccttcaccgttgatgtctctcaattaccatgcggtctcaacggggccctttatttctcacaaatgaatgctgacggtgataagtctgctactaacttggctggcgcgaagtacggaactggttattgtgatgctcaatgcccaactgacatcaaatggatcaagggaaaggctaactctgagggatggaaacccgcagaaaatgatccaggaaaaaacactggtaatggaaacttaggaaattgttgtcccgaagtagacatttgggaggctaactcaatctcacaagctttcacacctcacccttgcaaaagtgccaaaccaaccgtttgtgaaaaggaagcatgtggccatttcgaaggcactagatacaaatcaatttgtgacaaggatggttgtgattttgcttcgtacagatggggagcgaaggatttctacgggaagggaaaaaaggtggacacctcattacccattacggtccacaccgattttgtaaccaaggatggaacagataatggcaaactagtcgaaattagaaggtcctacgtgcagaatgggaaaactattgcaaatgagccagtaaaatttcaagggctttttaaaccagcagattcaatcactcaagaattttgcgatgctactaaaaaatttacaggtgatgtcaatgactttaaagccaaaggtggtatggaaggtgtcgatgcagcaatgacagcagggatggtcctggtgtttagtatttgggatgaccaggaagctcaaatggcttggcttgatggtacttaccctcctaacaaatcaggcgcaggtgtggagcgaggtacctgtaaagctggagaaggcaaaccagagactgtcaggcaagaacacccaaatgcttctgttacattttccgacgtcagaatcggggccatcacagataag

SEQ ID NO: 389 - DNA, 765 bp, Phakopsora pachyrhizi
  source 1..765: mol_type = genomic DNA
atgatctctttaagatttttgtcaattcttttagtcctcatttcaacctcacaacttactggctctagccctataactggagcaagcgaggaaaatgtaggtaaatcatccaagcaaaacaatcttgtgccaagaaatattccaggacttgaccaggtgccgggactagaccaggttccgggtggtgttccaatcattggaaaaaatgtggctgctgaccaacttgctggaattgttcgccgaaacattcctggacttgaccaggtgccaggactagaccaggttccgggtggtgttccaatcattggaaaaaatgtggctacagaccaacttgctggaattgttcgccgaaacattcccggacttgaccaggtaccaggactagatcaagttccaggtggcgttccaatcattggaaaaaatgtggctactgaccaacttgctggaattgtccgccgaaacattcctggacttgaccaggtaccaggactagaccaagttccaggtggtgttccaatcattggaaaaaatgtggctacagaccaacttgctggaattgttcgccgaaacattcctggacttgaccaggtgccaggactagaccaggttccgggtggtgttccaatcattggaaaaaatgtggctacagaccaacttgctggatttgttcgccgaaacattcccggacttgaccaggtaccaggactagaccaggttccaggtggtgttcccatcattggaaaaaatgccgttaccgatcagtaa

SEQ ID NO: 390 - DNA, 489 bp, Phakopsora pachyrhizi
  source 1..489: mol_type = genomic DNA
atggctaagcggttattatcattctactactttttaacctgttttgcgaatattttgtccactccaaagctgcagcctttggtatgcccgaatgattggtcacttgatccaatatcaagttcccaaaatactaggcgtaatgtttactgctatgatcaccaattcttgtgcgaagaaagcgattgttatgagtatccagtttgcagctcttgcactagtgataaagggaaattagccaagacagttgcatgcaaaaatgaataccatgcttccgatccaacaactgaccaacaagcaatctgtgtcagcagcgatgagaccttttggcaatgctctggcccttgcgaaggatacctaaaatgcataaatttcagtcgtaaaggccctttcatcaaaagccaactccgtaaaaggggcaatccgtttgcggttgcgatgaatgggagtccaggcggaacctcgagtaattttgattctgtcttgtccttt

SEQ ID NO: 391 - DNA, 525 bp, Phakopsora pachyrhizi
  source 1..525: mol_type = genomic DNA
atggcccaaagattccttccatcgatccttctctatctagtcgttttgtctttagtctcagcacaggatggtactcgaaacttggtctttcctaatcagaacagcaacaatcaatcaaacacgaacacgaacaccaacaccaacaccaacaataacgctaacactaacaccaacactaatgcagtaacaaacacccatcagtctaacacaacagcaaccaataataattccacctcttccggcaatatcagtagcaattccaccaacagggcaaatacttccaccacaaccaccaccaacctcacgtccatcaaagattccaccggtgccaaccaggtaccagccccaggcgcaacgggtggaaaggccaacgggaggtacggcccagacgatcagtacatctcatctgcccctaggaggaccctcgctgcacgatccacagacttgataataattttttcgtctttggaatctcaagcctggctcttaccggcccattgttcttttaagatccatcgttcatac

SEQ ID NO: 392 - DNA, 1446 bp, Phakopsora pachyrhizi
  source 1..1446: mol_type = genomic DNA
atggagaagctcttcactttacaaaatcttcttttcttgctaattttttggtcacagctgaccgataagacttcatactggctcattgggatcccttttggatgtgaagctgcagatgttgttgacgatgcgagcagagcggaggccatgataatgcgctaccaaaatacgctcttcgatactgaagaagaggaaatagaacagatgcacctcatattgaatcctcagatatcagggcccctgaatccgacccagataggcaaagaaactgctgaaggaggctcacagaaaaccaatacgtataccatggtaaactctatcaatgaccaaaaggaagaaaaaacaagagcggaatcatcttctctaaagaaccagtttccgggaaagtttgaatcggtccaaaaacaaaccttagatgataagaagaaacgcgcggaacaagacccgctaaatcagcttaaggtgctgaacttcataatacaggttgaaaatgatgagattgccgtagggattgtaaagagcagcctcgtcaatggctggccttctttaaagtatcttcaaagcttaataataagttactttgaacatgatgcagagatcttgtggggatacccgactctaaaggactttttactgagcgttaacaaaggcaggggctttgaaagagttttaagagtcctcgatgacgaaaactggaaaaatatttttcctcagctatgtagtacactttttgcacatctgaaaaaaacatcgaatcttgaagatgacgaaggaactatcactggtttagagattaaagaatttaacaaaatttatagtgcggacaactttatggcaatgatgagtccagaagataaaaacaaacaaactataaagctgttcaatagccttgtctctctgatgaacataaaaatatcaaaagtagcaagcatgaagcatcctggagcatctaacctgtggttcctcacttactacgtttttgagtttttggtgaaccggaaggaactaaaattctcaaaacaaattcggaacgggataagaatgaattcgatcgccttaaaggttgaggtttaccttaaattatttaccgtattttcgaaagcaagagttctcgtagagttgccacgcaggcttgaaaacaaaaaacagataactgtggatattccgaatggtcttcaaaacactgatttgatcgaatttttccgtgagcgatggctggtcatggaagcgctagagaacgacatcacccaacttatccctctgatttccaatctgtataaaatcaataaatcctccacagcgatagaaattttcgatgatctctacgattgggcctacaaaactcaagttaacttgaaaattctgaggatgaatgaagccggaactctttcaacagtcgacgctaagtctcctaagatttcacctaatgcaattaaggaagagtaa

SEQ ID NO: 393 - DNA, 768 bp, Phakopsora pachyrhizi
  source 1..768: mol_type = genomic DNA
atgttttctaaatcatttcaaggattgagcctactcgtagttctgatggcctctattatctttaactgtgatgaatcaaacgcccagaataactttaactcttttcaacaaccaggcacgaactgttacgaacagtcaaaatcctatggaatcaatttcgaacattgtgaaaaagctctaaggaaaatttcatacgattctaatggtaacttggataattcatccaagacggtttttgtctggcataaatcttgtgtggtcaaagttcaaaaggcaacatacgctcaaccatcgagacaacaagtagaatatggtgttagaaacttactacagacttgtcctacgaggggagggatttatatcccttcaaatgactttaggacctatgtttactcttcaaatcgtgaaaacgtttacaatttgaacagcccagtgtgcattaagcaccaatgtcatatcaaccctaatgactgtttgatggcctttaacaacattcctcttagtactaaaggttttttcttaccatctggttcaacctcttctaatgttataaagacttctagtggtaactgcactgttaaacttgtcactactgatggtgcaggattcagaattaaccatcctgaaattaactctggtatcaaaactctactctcaaaatgtggtagcagacctggttacaactatttcggtggtggcagtctaggaatgaatggtgatattaaaatcatcactcaaaacagttacaacaacggtcaatgtaattaa

SEQ ID NO: 394 - DNA, 1236 bp, Phakopsora pachyrhizi
  source 1..1236: mol_type = genomic DNA
atgatcaaaagtaatagtgtctacgccctagttgggttaatacctattgtctgggttttactgcccatgacttgtaaatcagcaatggttttccacaccttaagagaaccacctattggaaagatgcctcaggaactatcgggctgtaaaatggaaattttgtcaaataaaaatttcaaggagtctgagtacaaagcgggcgaaagtttagcatcgcgaaaaagcacagatgttcaaaatgttctagatgaacatttgaatagcgctatggagtctactaagaaaaaaaccccagtcaataggaattcaatcatttttgatgatggaaacgctagagccaacgattttatttcggcgtctcgagcagagcatattacatcaaaagaattgggagatttatttgaaaattacatgagagaaggcaaggataaaatcatcaagccagaaattatgctaagggagattgcttttggtttttcaagattttcaaaaagggtaattcaacttccctcgggtcccctaagcaaggttcttctctcagaagaaaatttgaaacttatcccagattctatcgcattttacgttaaaaatcttaaagaagaaaaatttgaatcatttataggttacgtgatcggtaatgaacttaataggttgaatgatttatacttaaacgatttttttggtcaattatcaattgaattcagcaagcccggattttattcctctttaaaagaattaaaaacagatgattccaaatctttggcgcaaggtttattcagctacttcagttcggcccctgtaggcaagggagaaaacttagaggtagctaaagagaagtacttgcttgtatatcgtataattgactccttgagtcatgatggaccaatatttgcgactagttttcaagagcaagcaagagatcgtgccgcttttgagaaatttaacataatcagtaaaattcaaaaactaacagataaaattaataatagtggtaagactgctgaagacaaactgcttcaggaagctcttgaattgagtgacaagttatttgacatcgaaagtaatgtatcagaacaaacccacgctgaattgctatctacttggactgatagcacagttgttaatagcataataagagacttcgacgaaaagttatacaagcaaattgattttgatgacgatgcagacttttacgctacccatagtcaaaatattaaacatgccataaaagctgta

SEQ ID NO: 395 - DNA, 177 bp, Phakopsora pachyrhizi
  source 1..177: mol_type = genomic DNA
atgaaattctttttaggattgacaagtctatttttgatcgcctctatagttggggtagttttctcatccccggttaatacaaatggagaagacaccagattacaaaagcgaaaaacggtagcgccagcaacctgcacacggggaggtagagttgttaaaaataaatgcaaagtatag

SEQ ID NO: 396 - DNA, 204 bp, Phakopsora pachyrhizi
  source 1..204: mol_type = genomic DNA
atgccatccaagttgtctggcctaaccgctgacgtggttctaaccacactcgtaacggcataccagttggaactagacaatctcagctttggatccatatctgctaccggtcaagtcgctcgaaagaccctcgtggaatgggtcctcagtggtcagatcagccctggtggtgacaggcaggacctggtcattaataaaaagtaa

SEQ ID NO: 397 - DNA, 117 bp, Phakopsora pachyrhizi
  source 1..117: mol_type = genomic DNA
atgagaggggcagttgtagagttgaacgaggcgcacgagtaccagaagaaggcgggcaagaagatgttgtgtctgctaatactttttatcgtgatatttgctataattctgattgca

SEQ ID NO: 398 - DNA, 147 bp, Phakopsora pachyrhizi
  source 1..147: mol_type = genomic DNA
atgtattcagtatacacctgttatttatctattttttttttcttacgtacacgagcgaaacgtaattctttttcctttaggggtaaatcactcagcactcatcccctagatggcattgcactagacagacaatttaataaaaaatga

SEQ ID NO: 399 - DNA, 144 bp, Phakopsora pachyrhizi
  source 1..144: mol_type = genomic DNA
atgggagctatatttgccaaaatgatgatggaactttcggctgtgaatcttacaagcgtagccctattacttgtgaaggctgtgttgatgcatgaaagttttcttaaacaggttactatttcactctttttctccaaaaaataa

SEQ ID NO: 400 - DNA, 348 bp, Phakopsora pachyrhizi
  source 1..348: mol_type = genomic DNA
atgtggatcagaaggttgatgatatctacaagaatcctaccagtgatagttataacgatctacatcctgaagaatagaagagttttgattaaagttgaacagaacaatagcaccagctttagaacccaaagatctgttaggttcagaggaatgacagacctgaagaatatcagctctaaagaagtctctactcacaccaccgttgattctgcctggactataatcgatggaaaagtttatgatgtgactgaattccttgaggagcatccaggtggaaaaaagattttgttaaagaattgtggaaaagattcaactgaactatttaatcagtatcataatgaaaaagtt

SEQ ID NO: 401 - DNA, 213 bp, Phakopsora pachyrhizi
  source 1..213: mol_type = genomic DNA
atgataagggtctcgatcagtaacaaagttttgaaatacatagctctttacttcttgataagctgcataggccattcaactcaagagagtcaacatccctatgaccttgaaggtcatgaccaagcacctaaccagaagctaaagattttgaatcgtaagttggaaggtgaaacagaagctacgatcccaatcagagatcactcactgtatgct

SEQ ID NO: 402 - DNA, 129 bp, Phakopsora pachyrhizi
  source 1..129: mol_type = genomic DNA
atgctaagtactgttatcgttaaactgttattttgggtgcatcggagtcacgaattgcaattcttttatgttcattatcagccctttgggttaaggctcgtatgtttccataaatgtctttgtctttaa

SEQ ID NO: 403 - DNA, 129 bp, Phakopsora pachyrhizi
  source 1..129: mol_type = genomic DNA
atgaaagatatcttcgttagagctgtatttttgttttggttctgccttgactttatcgagcctatagaaattcacactgacaaaaccccaaaaatttcgaaaaaagttcacatcatcattgatacttga

SEQ ID NO: 404 - DNA, 213 bp, Phakopsora pachyrhizi
  source 1..213: mol_type = genomic DNA
atgactaatctcagggtagctccctttttctttctcctcttctgcttcttactcttatccttttggtatgacctcacactcccttcgacttgtgataatcttaagtctgttctgaaagatgatctacttctcaactgcttctgcttgccattgatttcccagaatcactttcgcttgatgactttaaatatttttttttacaaaaacaattga

SEQ ID NO: 405 - DNA, 573 bp, Phakopsora pachyrhizi
  source 1..573: mol_type = genomic DNA
atgaagagcttcaacttaatcttatttattacagtcgctttggcattaatcactcaaattgattgtttacctaaaccaaggtcattagtatcatttaatcatttaccagcaaaagaaatagttaaaagacaggaagatcgattcaatgatgctgacgcgctaagtagagagacagaaggcgatggcgaaggtgaaggtgaaaatgatattggaggcgaaggcgaaaatggaagtgacaatgatgacgaaggcggaggtgaaagtgaagatgggagtgtgggtgacggagaggaagaagctgaaggagaagaagtaaacgaagatgtagtagaggtagatggagatggagataaggataaagaagttgaaggagaggaaggaggaggagtagagacagtagaaaaggtagaaggggtggacataggtagtaaggaagtaaatggaaatgtagaagaaggagataacaatgacaatgagaacgctgaagacttttctagaggtgttaatgacttgcaagagaaccaatttgtggacgatgattcagaagacgaagcattggctgatgaaagataa

SEQ ID NO: 406 - DNA, 159 bp, Phakopsora pachyrhizi
  source 1..159: mol_type = genomic DNA
atgagacgatggatgcagacgatgatgatgaggaggacagcaacgaggaagaggacgaagaggaagagaatggtgatgctgaggttgaagaagcatgaagttttttttttttttttatctatgtcactttcattttatatatatataaaaataagataa

SEQ ID NO: 407 - DNA, 423 bp, Phakopsora pachyrhizi
  source 1..423: mol_type = genomic DNA
atgaagagctacattcctttcattgtaacaatattgctcctggctgagctaagcgcgagaaatctaaaactttcctcaagaagtgatcaaggtgtcttaagctgtaccacctacagtgacacgaataccgatgcagctagctgtaacaatcttaccaatatctattgcgctcagtgtgaacccggaaaaactgtggggaataactgtgtcaaaaagcttgagagtgttgctaatcaaactgttgtgactaacctcactgtcattaacaacaacaacaacagtagtaggagtacacagggttgtaccatctcctttaagatggacgaagatgggagctatatttgccaaaatgatgatggaactttcggctgtgaatcttacaggcgtagccctattacttgtgaaggctgtgttgatgcatga

SEQ ID NO: 408 - DNA, 213 bp, Phakopsora pachyrhizi
  source 1..213: mol_type = genomic DNA
atggcatcagagtatcttaagatcaaggagatctatgaagcttccaaagcctttgagccaaacctatcaccacagcttcacagcccgatcgggtttgccctgttggctagtgcctttgggattggtttcctattcacaacattgccgaaaagaggtatacctctagcagaactgtttccagctatcctatcttctgtgctcactggtttcgga

SEQ ID NO: 409 - DNA, 213 bp, Phakopsora pachyrhizi
  source 1..213: mol_type = genomic DNA
atgtcctgtaggttactcctatttttttttttttttttttttttttttagattaactgtcctatatttcatattacctaaaacattatcagacagcttaaaaaactctcaacaattaagatcttggaacctagtctcaaaggtccatccaataaagaataaaaaacactgtagtaatagtaaatgcattacttacagcaatatatatatgtaa

SEQ ID NO: 410 - DNA, 423 bp, Phakopsora pachyrhizi
  source 1..423: mol_type = genomic DNA
atgaagagctacattcctttcattgtaacaatattgctcctggctgagctaagcgcgagaaatataaaactttcctcaagaattgatcaaggtgtcttaagctgtaccacctacagtgacacgaataccgatgcagctagctgtaacaatcttaccaatatctattgcgctcagtgtgaacccggaaaaactgtggggaataactgtgtcaaaaagcttgagagtgtagctaatcaaactgttgtgactaacctcactgtcattaacaacaacaacaacagtagtaggagcacacagggttgtaccatctcctttaagaaggacgaagatgggagctatatttgccaaaatgatgatggaactttcggctgtgaatcttacaagcgtagccctattacttgtgaaggctgtgttgatgcatga

SEQ ID NO: 411 - DNA, 87 bp, Phakopsora pachyrhizi
  source 1..87: mol_type = genomic DNA
atgcttacagttggtttatctttcttcttcttttttgatcaaatcacttgtacaaagaacaaaataaaatgtgtaaaaaaaagataa

SEQ ID NO: 412 - DNA, 555 bp, Phakopsora pachyrhizi
  source 1..555: mol_type = genomic DNA
atgagtggacaactactaaacttagcaattccattgatcggaatgcagatcgcaaggaagattccattcgatgatcctcaggtcttgacaggaatgaggatggcttacatcctgagccagttgtcatgcctctcgatctactactacaccgcctcaaagatcaagtctaagaatgataccaccgttctaaagtacgtggaaccaaaggctccaatgagtccagatcaggctaaactggtgacgacaacggtcagggattacgatctgaatcagatctcaactggtctgaggtcggtgatgatgggcgtcctgatgatgctcttcctacacctttacatgaagtatgacgcccctctgttcttacaatccctgatgcctctcaaaacgacgtatgagaacaaggtgatacagatccatttgctcggtcgatcggccactggtgatctgaaaaggccattcaaaactggtggcttcatgggagctgcaacggatgcaaagactgatgctaagagcataaaggaggcagagaaatctagtgttaagaaggaccaatga

SEQ ID NO: 413 - DNA, 123 bp, Phakopsora pachyrhizi
  source 1..123: mol_type = genomic DNA
atgaataaacttttctttttaagttccgctacatcttttagaggtttttactattgctattattgtttttttttatttccccccctcttttttttttttttttttttttttttttttttttaa

SEQ ID NO: 414 - DNA, 1023 bp, Phakopsora pachyrhizi
  source 1..1023: mol_type = genomic DNA
atgccaagtatcaattttttgatttttgcggcgatatttctgctgggaacaaccaagcatgtggcgtgggcatcgaagaacgaacttgaccttattacgggtcttgaaacttcaactgaccttataaatgttaacgtggggataaataatgaccctacaattatcaacaataaaaacattccttacaggcctggaagcgatgttaatttcaacccaaacgattttgacaacaacgagtcggaaaatggcaatactattgaaaatcatactgtcttgaaaaaaccaagagtatttcttccaatgaaaacttatttttcaaaggaaaacataacttcggaagattccattgttaaaaccgattctgtcacctccgcggccaacgaacctactaattcaaaatttcctggagtgcatcagaccgctcctgttaagtgcggaaatgggaagccaccaccttgtaatgaggatacaaagccaaataatactcgattttcagggactcatcaaactgctccagttacctgcggcaacggaaagcctccaccttgctcagaatcgagccagtacacttctccaataactgtacatggtacccaacggactgctccggtaacttgcggtgatggaaagactccaccttgctcaggatcaaaaccgcatactactcgaattccaggaactcatcagaccgctccggtaacttgcggcagcggaaagcctccaccttgctcagattcgagccatcacacttcaccaaggaatatacctggtgcccaccagactgctccggttacgtgtggaaatggcaaggctcctccttgttcaggatcaaactcaaataatcaatcaggacattttcctgggacacaccaaaccgctccagttaagtgtggcaatgggaagccaccaccatgcacgggatcagtcaattcaagcgtagagcctaaaaaccaccctgcaactcaaagatcagcctcggctagctccaaaaacccaaaacgctctcgacgtactgttttttaa

SEQ ID NO: 415 - DNA, 1068 bp, Phakopsora pachyrhizi
  source 1..1068: mol_type = genomic DNA
atgccaagtatcaattttttgatttttgcggcgatatttctgctgggaacaaccaagcatgtggcgtgggcatcgaagaatcaaatttatgattgttcaaattcaattttatgtatatatctaagcgaacttgaccttattacgggtcttgaaacttcaactgaccttataaatgttaacgtggggataaataatgaccctacaattatcaacaataaaaacattccttacaggcctggaagcgatgttaatttcaacccaaacgattttgacaacaacgagtcggaaaatggcaatactattgaaaatcatactgtcttgaaaaaaccaagagtatttcttccaatgaaaacttatttttcaaaggaaaacataacttcggaagattccattgttaaaaccgattctgtcacctccgcggccaacgaacctactaattcaaaatttcctggagtgcatcagaccgctcctgttaagtgcggaaatgggaagccaccaccttgtaatgaggatacaaagccaaataatactcgattttcagggactcatcaaactgctccagttacctgcggcaacggaaagcctccaccttgctcagaatcgagccagtacacttctccaataactgtacatggtacccaacggactgctccggtaacttgcggtgatggaaagactccaccttgctcaggatcaaaaccgcatactactcgaattccaggaactcatcagaccgctccggtaacttgcggcagcggaaagcctccaccttgctcagattcgagccatcacacttcaccaaggaatatacctggtgcccaccagactgctccggttacgtgtggaaatggcaaggctcctccttgttcaggatcaaactcaaataatcaatcaggacattttcctgggacacaccaaaccgctccagttaagtgtggcaatgggaagccaccaccatgcacgggatcagtcaattcaagcgtagagcctaaaaaccaccctgcaactcaaagatcagcctcggctagctccaaaaacccaaaacgctctcgacgtactgttttttaa

SEQ ID NO: 416 - DNA, 1014 bp, Phakopsora pachyrhizi
  source 1..1014: mol_type = genomic DNA
atgccaagtatcaattttttgatttttgcggcgatatttctgctgggaacaaccaagcatgtggcgtgggcatcgaagaaccttattacgggtcttgaaacttcaactgaccttataaatgttaacgtggggataaataatgaccctacaattatcaacaataaaaacattccttacaggcctggaagcgatgttaatttcaacccaaacgattttgacaacaacgagtcggaaaatggcaatactattgaaaatcatactgtcttgaaaaaaccaagagtatttcttccaatgaaaacttatttttcaaaggaaaacataacttcggaagattccattgttaaaaccgattctgtcacctccgcggccaacgaacctactaattcaaaatttcctggagtgcatcagaccgctcctgttaagtgcggaaatgggaagccaccaccttgtaatgaggatacaaagccaaataatactcgattttcagggactcatcaaactgctccagttacctgcggcaacggaaagcctccaccttgctcagaatcgagccagtacacttctccaataactgtacatggtacccaacggactgctccggtaacttgcggtgatggaaagactccaccttgctcaggatcaaaaccgcatactactcgaattccaggaactcatcagaccgctccggtaacttgcggcagcggaaagcctccaccttgctcagattcgagccatcacacttcaccaaggaatatacctggtgcccaccagactgctccggttacgtgtggaaatggcaaggctcctccttgttcaggatcaaactcaaataatcaatcaggacattttcctgggacacaccaaaccgctccagttaagtgtggcaatgggaagccaccaccatgcacgggatcagtcaattcaagcgtagagcctaaaaaccaccctgcaactcaaagatcagcctcggctagctccaaaaacccaaaacgctctcgacgtactgttttttaa

SEQ ID NO: 417 - DNA, 444 bp, Phakopsora pachyrhizi
  source 1..444: mol_type = genomic DNA
atgcactcgaaagtgttgatatcgtgggtcatgagtgcctttgtggcaatcgtcctacatgcagctaattcgatccaggacaatgaggagccaccaatcgttgcaaaggtgtattttgatatgaaacaaggctcaaagaatcttggtaggattgagatcggtttattcaaaaatacgcccaagactagtgaaaacttcagagcacttgcagttggtgataaaaaatcaaaagatgggattcctctggcctacaagggctctaagtttcacagagtcattaaccaatttatgattcaaggtggagattttactaaaggcgatggaactggcggactatctatctacggaccaaagtttcaagacgaaaactttataaacaaacatactggacctggaactgtttctatggcaaactctggtcctgatacgaacggctctcagtaa

SEQ ID NO: 418 - DNA, 303 bp, Phakopsora pachyrhizi
  source 1..303: mol_type = genomic DNA
atgtggatcagaaggttgatgatatctacaagaatcctaccagtgatagttataacgatctacatcctgaagaatagaagagttttgattaaagttgaacagaacaatagcaccagctttagaacccaaagatctgttaggttcagaggaatgacagacctgaagaatatcagctctaaagaagtctctactcacaccaccgttgattctgcctggactataatcgatggaaaagtttatgatgtgactgaattccttgaggagcatccaggtggaaaaaaggtccagtcaaaattttgttaa

SEQ ID NO: 419 - DNA, 885 bp, Phakopsora pachyrhizi
  source 1..885: mol_type = genomic DNA
atgcagaccaaaattcttctagctctttgcctagtagctatctctcaggttaatgcgcatggtgcaattacagctgtccaaggctctaacggcatgactggagaggcttttggagtagatcagagcactcctcgtgatggtacaaaacgaaacccatttcaaaccgactcttcaattattcgtgatcgtgagattgctagcggcaagtcatcagcctgcggcagaaccttggctggtggaaacaacgagattggagcagcaatgtctaaggccgagtctgctggtatcccaagtgttagctccgatggaaaggttcagatgactttacatcaagttaacggtgacggtggtggcccttacacctgtgatgttaatgcttccggtgatggaaagactttcacacctatgacaatttctactaacattccgggaaagaacagcagatctaatgccaaggctgaggatattcctttaattgcggagatgcccaaaggaatgacttgcaccggtggatctgacggacagacctgtatcgtaagatgcagaaacgctgccaacgctggtccctttggaggatgtgtagctgttactcaggctacggacgctacaaagcagaagcgggaactatcagcagaacagagaattgatgagatgatcaaacaggctgaggctgccgaggcatttggcgatgagactcttgcaaagcgtgaaatgtctgctgacgagagattggacgaaatgatcaggcaggccgaagccgcagaggcgttcggcgatgagacccttgcaaagcgcgagctttctgctgaccagcgattagacgaaatgattaaacaggctgaagatgctgagctggctgaggaaaacctttccaagcgggattaa

SEQ ID NO: 420 - DNA, 627 bp, Phakopsora pachyrhizi
  source 1..627: mol_type = genomic DNA
atgcgctctcttatcattcccgcccttttatgtgtactatttattcataaatcttgcgcaaccgttcacactcagtgctacaactactttttacaaaaggatggatgcgtattctctgcagctgatgacagaaatcgatgttctgcagatcccaagcccagtacagccgtcggagtggttcaagcaagcaacacaaatgttaaacgtcacactctggcacgacgctatgatactactctaccatctccttctgtgcgtggtgaagggatttgcggaaactataacacagcagaagctgagggggctagtctttgggttggccctaacccggatagcacaaaccctgaagaagcaggttggctcaacaagggaaagacatccaattgcaacaagcaattatatataataaaccctcgcacgcgtaaaactgtatacgtgaatgtcatagacggtcacgattttcagacaacgcagcctgatgttggatgcttccagattgcactcacccaaaataccttccttcagcttgatccaacagatgaagaaaaagaaaagggttccataggatctctcacatgggacttcaacaaccttaatggcgctagtcctcaggatggccctgtctaa

SEQ ID NO: 421 - DNA, 1122 bp, Phakopsora pachyrhizi
  source 1..1122: mol_type = genomic DNA
atgcagaccaaaattcttctagctctttgcctagtagctatctctcaggttaatgcgcatggtgcaattacagctgtccaaggctctaacggcatgactggagaggcttttggagtagatcagagcactcctcgtgatggtacaaaacgaaacccatttcaaaccgattcttcaattattcgtgatcgtgagattgctagcggcaagtcatcagcctgcggcagaaccttggctggtggaaacaacgagattggagcagcaatgtctaaggccgagtctgctggtatcccaagtgttagctccgatggaaaggttcagatgactttacatcaagttaacggtgacggtggtggcccttacacctgtgatgttaatgcttccggtgatggaaagactttcacacctatgacaatttctactaacattccgggaaagaacagcagatctaatgccaaggctgaggatattcctttaattgcggagatgcccaaaggaatgacttgcaccggtggatctgacggacagacctgtatcgtaagatgcagaaacgctgccaacgctggtccctttggaggatgtgtagctgttactcaggctacggatgctacaaagcagaagcgggaactatcagcagaacagagaattgatgagatgatcaaacaggctgaggctgccgaggcatttggcgatgagactcttgcaaagcgtgaaatgtctgctgacgagagattggacgaaatgatcaggcaggctgaagatgctgagctggctgaggaaaacctttccaagcgggagttgactgctgaccagcgattggatgaaatgatcaaacaggccgaagccgctgaaattgcagaggagaacttggttaaacgggaattgactgccgaccaaagactagacgagatgatcaagcaagccgaagccgctgagcttgcagaggagaacttggccaagcgtgagctgactgctgaccaaaggctggaccagatgatcaaacaagccgaagccgctgaaattgcagaggagaacttggctaaacgggagttgactgccggccaaagactggacgagatgattaagcaagctgaagcagctgagcttgccgaagagttgatctaa

SEQ ID NO: 422 - DNA, 483 bp, Phakopsora pachyrhizi
  source 1..483: mol_type = genomic DNA
atgcgctctcttatcattcccgcccttttatgtgtactatttattcataaatcttgcgcaaccgttcacactcagtgctacaactactttttacaaaaggatggatgcgtattctctgcagctgatgacagaaatcgatgttctgcagatcccaagcccagtacagccgtcggagtggttcaagcaagcaacacaaatgttaaacgtcacactctggcacgacgctatgatactactctaccatctccttctgtgcgtggtgaagggatttgcggaaactataacacagcagaagctgagggagctagtctttggacaacgcagcctgatgttggatgcttccagattgcactcacccaaaataccttccttcagcttgatccaacagatgaagaaaaagaaaagggttccataggatctctcacatgggacttgttagtgctttgcttacaattaaaagaagaagaagaaggaatgatctaa

SEQ ID NO: 423 - DNA, 249 bp, Phakopsora pachyrhizi
  source 1..249: mol_type = genomic DNA
atggctaaacctggtgtaggtagattaccacctggtaaagcacttggacttttaacgtttggtactggattaggttggatagtgatgaagatcgtaacaccttcagaagaaaagctatacagcagactctcaccagacttaaaactcaaggtagatcaacgtagggctgaatccaaagagtgggataagacctacggcaacacaactgtgattgaacagtacgaaaaatcggtcaagagggataaatag

SEQ ID NO: 424 - DNA, 1209 bp, Phakopsora pachyrhizi
  source 1..1209: mol_type = genomic DNA
atgcagaccaaaattcttctagctctttgcctagtagctatctctcaggttaatgcgcatggtgcaattacagctgtccaaggctctaacggcatgactggagaggcttttggagtagatcagagcactcctcgtgatggtacaaaacgaaacccatttcaaaccgactcttcaattattcgtgatcgtgagattgctagcggcaagtcatcagcctgcggcagaaccttggctggtggaaacaacgagattggagcagcaatgtctaaggccgagtctgctggtatcccaagtgttagctccgatggaaaggttcagatgactttacatcaagttaacggtgacggtggtggcccttacacctgtgatgttaatgcttccggtgatggaaagactttcacacctatgacaatttctactaacattccgggaaagaacagcagatctaatgccaaggctgaggatattcctttaattgcggagatgcccaaaggaatgacttgcaccggtggatctgacggacagacctgtatcgtaagatgcagaaacgctgccaacgctggtccctttggaggatgtgtagctgttactcaggctacggacgctacaaagcagaagcgggaactatcagcagaacagagaattgatgagatgatcaaacaggctgaggctgccgaggcatttggcgatgagactcttgcaaagcgtgaaatgtctgctgacgagagattggacgaaatgatcaggcaggccgaagccgcagaggcgttcggcgatgagacccttgcaaagcgcgagctttctgctgaccagcgattagacgaaatgattaaacaggctgaagatgctgagctggctgaggaaaacctttccaagcgggagttgactgctgaccagcgattggatgaaatgatcaaacaggccgaagccgctgaaattgcagaggagaacttggttaaacgggaattgactgccgaccaaagactagacgagatgatcaaacaagccgaagccgctgagcttgcagaggaaagcctttccaagcgcgagttaactgccgatcagcgattggatgaaatgatcaaacaagccgaagccgctgaaattgcagaggagaacttggctaagcgggaattgactgccggccaaagactggacgagatgattaagcaagctgaagctgctgagcttgccgaagagttgatctaa

SEQ ID NO: 425 - DNA, 1482 bp, Phakopsora pachyrhizi
  source 1..1482: mol_type = genomic DNA
atgaattatcaaagaataaattatggcttgatttcttacgtagcttttggcatgctcctactgtgcacggtagctcaatgctccttcgcaagaacggagaatagattgcggagcctgagagatctacctgtgctatcagatttaaaattagaagaaagggtagatacagaaaaatatgatactataactcagaaatctggtcgtttcttagtagtcaattcctttccagcaccaaaggatcgttacccagaagaaaacttttttagtaagaatttacttaagactcttagatcggatgtggaggatggcagagagttcaatgaggatcagaagcttcagatctatcaaggtcttaaaatttattttgaacatcaatcattccaattcttacctggaatcggtataactaaagttccaggactgagtgaaattcttgcgagactcagtcagctggcaggtacttcgaagtctaatggacttgatatcaatcaaatcttctcagatatcgatgctctaaagggagttgtctggtacatcggttcaaaaatcactgagaatcttggtttttctgggatttttaagaccatcgacataagacaattcatccttgagcatcccgacctgattagtataaagaccttgctcaagaactttgaagataaacagtggaaagctatcgaattaggctttctccgttgtcacttggagattggttataatcctcacctactatcagacgtaggctacagatatctgatcaaaaagtttgaaaaagtgatccctagagtagaaaatcatgagtccttcaatccagaagttctcaactcagccaaaagcgttattaattacgaatacgaggagagatacgatcaattcttgataaaaaatgtctttgtctatcacatcataaattactttgagagatacttcaacactgatctagatgattcgatcaagtatcagacctatatgaacagatacatgatccagatgatcaagacctccttggagtttagagaatcggtcatagatcccttgtacttcaaaaaagtcatcttcagacataaccatcctaccttctcgataattgaggagctccaacccaatcgaaacttaccattggttaacgccagaggaacaattagaattgacatcaggaacagatcattcttcttgaggaaggatgacatcaagaccctcgaaccctggttgaagaaggtaaagctccaagctcaggctgtcaaagaacaggaatcgatattcacctcagtactaaatgatctttacaagatatctgagaccgatctggggtttaaacaatggttaatgagtgataaaatgcaaattctgttggatgacatcagaaaaagtctggaggatgctacacattacagctcagtagacataggtctagccccaaagctcaaagaacagttcaagttgagtaatgttgaaggcaaaaccattgttaaccaatcatga

SEQ ID NO: 426 - DNA, 267 bp, Phakopsora pachyrhizi
  source 1..267: mol_type = genomic DNA
atggtacccacaaatcaaaagacgttaatactaccgagaatggtcaagttggtcccggctttgctagtcagtctactgcggctcctccgccacagggttatatttgttacaggtgtggccaaaagggtcattggatccagacgtgtccgacaaatgataaccctgaatttgaaggaaggcctcgaatcaagcgaacgactggaattcctaaaagttttctacagaaggttgaaggcaagccaggcgatgggcagaatgtcatggtga

SEQ ID NO: 427 - DNA, 108 bp, Phakopsora pachyrhizi
  source 1..108: mol_type = genomic DNA
atggcttcaaaggttatagttgttggtggtggtctgagtggtctgtctgctgctcatacgctttacgacagaggtgcaaacctactggttctcgacaagaatccattg

SEQ ID NO: 428 - DNA, 198 bp, Phakopsora pachyrhizi
  source 1..198: mol_type = genomic DNA
atgaagccagtcggcccggttaattgtaggagaggaaaaggctttctgcttttttttctttttcttttttttattttgaatctaagcttcgatgtaccaggaacacagttggtggtgtgtcctgatggtgaatgttatctgttcctctctctctatctctctcttcgaagggataacctagtcgttggctgtggctga

SEQ ID NO: 429 - DNA, 156 bp, Phakopsora pachyrhizi
  source 1..156: mol_type = genomic DNA
atgatgatgatgatgatgataaaaagtcagaagatctactatatctatatattcatatgtctcgaattaatgataactgatagaaactatgaactcaaaaaccgtcaatcctctaaagcctgcccagtatctattcaaagccgaggcacccactaa

SEQ ID NO: 430 - DNA, 771 bp, Phakopsora pachyrhizi
  source 1..771: mol_type = genomic DNA
atgacatggaaaaccagattggtctcttccttgctgagtcagctgagaatcagttataaaagtgttggaagcatctcaaacctgagtgtaaaaaagccagaaagcagcctaaaattcagagatttcttttctacaaaatcaatagcatcagcaacaagttctacatcattaccaattagaccaactttaaagtcctcatcaggaacatttaaggaatccttgttgaatgacaacttatcaagcatctcaacctcaatggccctaacgaagttaaggtctcaaccaaaccattacgttatcagttttttggttggcaagcggtacagattgacaaccaatgaccttgtaacagttccacatatcaaagacttgagagttggagatttgatcaagctcacaagattaacagaggtaggttccagaaactttacaatcagagcaatcggaaatggcacagttggacatgcagcacttgaaaccaaaccaccaccttctaatcaaaaatttaccaatactttcaaaaactctgatgatgctgaagagtctcctgggttaaaaagagttatttgctttaaaccagagatacctcattacttggatgatggattggtagatgccactgcaattgttgttgaacacaccaggggaaagatgtcaactgttatcaagaagaaaaggaggaaaggttacagaaagaccatcaaaagcaaaccttactacactcagattagactctgtgaaataaaatttccaactacagctgaaacataa

SEQ ID NO: 431 - DNA, 213 bp, Phakopsora pachyrhizi
  source 1..213: mol_type = genomic DNA
atgatgatgatgatgatgatgaagtttatcagaaactgtcatcaacagcagagcatcgttccaacagcagcaacaacactttctcacctcaaatcctcatcaatcctatcaatcaacaatcacaaccacaactctcatcatctcaacagaaggtacagatctaacctcaccctcgatgatatcctaccccccaacaatcaacaacagatccaa

SEQ ID NO: 432 - DNA, 717 bp, Phakopsora pachyrhizi
  source 1..717: mol_type = genomic DNA
atggctaataatctcacagtttctaccttcttactgctagccctttcaccatttgtttcttacgctgccgttcaccctcagctcgtcgccttcaaaaccttttctttccccaagggtacttcgccaagctcagaaaatggtaaaaatcttcaattttttaaagcagctgactctccagcagccactgaggtacccagtcagggcaacaatctggttaatagtggcctaacttcaaccgtctgcaaatcttccgagggcaaggaatttactttcactaatcaagactgtctgaatgctgctagagtcatcgccaatgaatccacttcatctgcacagtgcggtaactgcgttatcggcttgttcgatgtcaataagaagaaagcatttacaccacctggcccggtgtcaccctcagttctagagaatcaagttaataaggttttatcggaatgtagcaacagagctagcagtaccaacccaccagtgaataataagcgcagtcttttaccttcacaatctggagttgaaagtgtcagcggaagtgatagtatagctaatcaccctgcagttgcaaacgctggtgggcataataacttggccactcaacctggaactacaagccccggtgggtcgcaacctgggagtggaagtggaaactcagattcagctacagttcaaatggtcatgggatacaaccccacttcaaaagcttgctga

SEQ ID NO: 433 - DNA, 273 bp, Phakopsora pachyrhizi
  source 1..273: mol_type = genomic DNA
atgaaggtcttcaccatcttttcagctgttatgctcttatttgccgcaggtgctatggctgcccccgaggaaatcagcgccaattctgctgataatgtcgacgaaaagcaattcaggccataccctacctggagaaatggcttcaattactaccgctacggaaactactggctcccacccaacgcctacacccgacttccaggtggccgcattagggttacccaagggtacttcgaccaaggacgccgatgggttcccttcgtcggcatcttg

SEQ ID NO: 434 - DNA, 1326 bp, Phakopsora pachyrhizi
  source 1..1326: mol_type = genomic DNA
atgagaatgtttgaagatttcatcaaaagctccaaatcaacccatctaactttggcagtgcttggaactttaattttccatgaagtaaagggaagtttcgcccaatccggagagatgataaaaacttccatgttaacacatccagatgggtttttaaacaaatatccagattctaaaataatttcgtctcctttattcgaaaatgatattcgaagttcgcaaggtgtgagagctttagcaggatcgctaaaaaacgagcagctatcttatgtaggcaaagattctgagtcctttgcgaaatccataaaaggcttacctatctcgcaggaagatgaactactagaacaaacaaccaaatatctcacttcaaaatcacatccagatcttattctccaaaatattgacaatctctgtcaaaaaattttagggtcaacctcaaaaaatctagatggtgcaatgtcattgaattacaatgtcaagcgaacatggctcacaaaattaataaacgtattttctaaaatatctgatgatgaaactcgctcaaaattgtttggaaacaataatgttatttcacttcttgcgagtgaatatcattttataataaacaaaccggatcacaaattggccttacagacatctgtagcggggatgattttcattgagaaagagattgatcacctacagccgattttatctttctttaatgaagagaaacaaaaactgttcagacataagtattttgtttatgaggttgagtctcttagcgcttttggatcacagaaactaaacaagaaattttatgaattgtttagtcttctaccatcgctcaaaaaagaaaaaggacttgaagtcaagaaagatcttattgagaagcttctgatgaagaaagatctacctaagcttgataagtttttgacatacaatgaagcacgagaacagtatgaaaccctcgcccaaatattagattacttaatccattataatgatgaaatcaaagcggaagactttgagaatattccagaaaataaactttatgagctcttaaaattgatttctgatgagcacgagaattttcaaagtcaggtctacaatgataagtatctgacgacatacaaaaataagatcgatgaggtaacgaagacattaaatgagcaacagaaagattcttcgatctttaaaataagcaaggaattgcttgtacatttggaatatttaaaatattcaaataatgaaatgaaacagctggaaaatcttttggatgcacgggtctcagggttggccagaggtcaatccaaaaattctaggatgagcagaaagttgctataa

SEQ ID NO: 435 - DNA, 351 bp, Phakopsora pachyrhizi
  source 1..351: mol_type = genomic DNA
atggcaactttcttggcaaagaacgtagcccttgtacctctcttcgttgccgtggggttaggtctaggaggtggaataggatttggaatccattatttaaagaataaccaagacgtcgttttaagaaaaaagtcaaacccagacccatggaacaaagttccacaggacaacaacacgaagttgttctcttttaatccggacttctggcgagctagagcccaattgactgatccaagattaagttttatggaaagtaaaccagagaatgagagaactttacatgaacaagccatggtcgaaagggctaagcagatcagactgaatgacaaggaacgtaccattcacagttga

SEQ ID NO: 436 - DNA, 294 bp, Phakopsora pachyrhizi
  source 1..294: mol_type = genomic DNA
atggcaaagcagggtgtaaaaggaaggttggtcctgacctcatcgatcctaggcttcttcggtctgccaggatactcggcctatacgacatccaaacatgcactcagaggcttagcagagtctttgaggttagagatgattctttatgatatcaaggttcactgttacttcccagccaccatactgtctcctggatttgacgaggaacagaagtgtaaaccggaactgacaaaggagatcgaaggggtggatgagggacagagcccagagaaatgcgccaagaaattgatcaga

SEQ ID NO: 437 - DNA, 213 bp, Phakopsora pachyrhizi
  source 1..213: mol_type = genomic DNA
atgcgctgcttcatctttgctcttgctctcctttcttctgctcaatttgtcttgggggcaggagattcgtcccagaaggtgaatcgaagaagtcttgtccaagttaacgcccttaccagtggaagacgaccattcaattctggcaattcgctggttgaggttaatgccttaaaaaactaccagaaagacaactctaagatttccgggtattaa

SEQ ID NO: 438 - DNA, 225 bp, Phakopsora pachyrhizi
  source 1..225: mol_type = genomic DNA
atgcgctgcttcatctttgctcttgctctcctttcttctgctcaatttgtcttgggggcaggagattcgtcccagaaggtgaatcgaagaagtcttgtccaagttaacgcccttaccagtggaagacgaccattcaattctggcaattcgctggttgaggttaatgccttaaaaaactaccagaaagacaactctaagatttccgggaacgctcaacagtattaa

SEQ ID NO: 439 - DNA, 1368 bp, Phakopsora pachyrhizi
  source 1..1368: mol_type = genomic DNA
atggctttaaaagctcattttttacggcttctgtcttcattatcattaaatttattgatagctgagctcgcagcttgcgaatacaatggagccacagctattacgagttctagttcaacttacatgatgcaatcttctagccaagctagtgttgatctcacagtaattcctcctatcagtaatcttacaggagatcatggaaaaaagagaaataaaaaaaaatactcggcatattatcccagctataatgcagatcagcgtcctgtatctgatattaaatatgagcactatgatgagctggtattttttgtagccaccaccaatcaaaacttcacaataggtctaggagacatcgaacaagaagtctgggattccagagctcatgaatttgttcatagatgcaagaaagctggggtaactcctttttattgtcttggcggttggacgggttctatctatttttcttctttagtctctactgcttcaaacagaacagaatacgcaacttcagctgttaatttcgctaagaaatacggttttgagggcatagatattgattgggagtacccgagtattcaaggaatcggatgtaatatcgttagcgacgcggacgtcgaaaactaccagttatttttaaaggaaatcaaacgaatttggcccgagggaaagcttagtacagctgtcagcattggaggaataagggcttcggattacagcccattacctgcctccaacttgacgactcttattgatgtagtggataaattgaatataatggcatatgatgtctacgggagctggagcactaccaccggcccgctctcgccgctgcaaagcacttgtgctgatgacgaaaatcaaatatccgtcgagagcgcggttgaagtttatctccagcaaggatttaagcccagtcagatggccttaggaatcccaacctacggaagaagttggacactagcaagtcctattttgacaccaagaactgttcagaattacaccacttactattatcaaaacttcactgatatacctcggggtgggcagtttgatgatgagcctggagttgatgtttgtggtcaaaattctactagctggggaggtactatccttttcacggagctcctttcaagaggatacttggatgaggatgagatggcctctgcgggttttgtaagatactacgacgaatgtagtaggcagccattcatcgtaaacggcacccttatgatttcatacgatgacactcgaagtactttgcgcaaggtacaatacgcaaagtccaagaatctagccggagtttatttctttgacaccttgggcaccactgaacgctccctcagagctgttcggcaagttttaaatagc

SEQ ID NO: 440 - DNA, 921 bp, Phakopsora pachyrhizi
  source 1..921: mol_type = genomic DNA
atgatctctttaagatttttgtcaattcttttagtcctcatttcaacctcacaacttactggctctagccctataactggagcaagcgaggaaaatgtaggtaaatcatccaagcaaaacaatcttgtgccaagaaatattccaggacttgaccaggtgccgggactagaccaggttccgggtggtgttccaatcattggaaaaaatgtggctgctgaccaacttgctggaattgttcgccgaaacattcccggacttgaccaggtaccaggactagatcaagttccaggtggtgttccgatcattggaaaaaatgtggttactgaccaacttgctggaattgttcgccgaaacattcccggacttgaccaggtaccaggactagatcaagttccaggtggcgttccaatcattggaaaaaatgtggctactgaccaacttgctggaattgtccgccgaaacattcctggacttgaccaggtaccaggactagaccaagttccaggtggtgttccaatcattggaaaaaatgtggctacagaccaacttgctggaattgttcgccgaaacattcctggacttgaccaggtgccaggactagaccaggttccgggtggtgttccaatcattggaaaaaatgtggctacagaccaacttgctggaattgttcgccgaaacattcccggacttgaccaggtaccaggactagatcaagttccaggtggtgttccaatcattggaaaaaatgtggctacagaccaacttgctggatttgttcgccgaaacattcccggacttgaccaggtaccaggactagaccaggttccaggtggtgttcccatcattggaaaaaatgccgttaccgatcagctcgccggactcgttcgccgaaatagtgttgcttccagagttggattatag

SEQ ID NO: 441 - DNA, 537 bp, Phakopsora pachyrhizi
  source 1..537: mol_type = genomic DNA
atgaagtacaaccttgtcacgattttgctatccatctatgtcttcaatttatattcttgtagccctatacctaaccatcatcagagcggtgctcaaggaatttctggtcaggaatccaataacaattccttaaaccagaggagagctgaatctcaacagaccaaatccgattccaacaatcagattcagaacaccaataacgaaatcaaccagaaccaactcaacagacgagaaattatcaatgattctaagagtcaagatcattcctcaaccacgaacgttgctcagaatgaccaacacaccaacaaccagagtcgaggagactctatcgaaaacaagagcatttgggaaataggaaataaagctgatcattccaataacattcagcaaaatgaaaatataatatcgaagaatgttcaagattttttttttttttttttttatagcaatctaataacaacggtagcaacaaggcagattcgattgataagattattcaaaatgactcccaacatcaaaacgagcaaccaaatatag

SEQ ID NO: 442 - DNA, 780 bp, Phakopsora pachyrhizi
  source 1..780: mol_type = genomic DNA
atgaagtacaaccttgtcacgattttgctatccatctatgtcttcaatttatattcttgtagccctatacctaaccatcatcagagcggtgctcaaggaatttctggtcaggaatccaataacaattccttaaaccagaggagagctgaatctcaacagaccaaatccgattccaacaatcagattcagaacaccaataacgaaatcaaccagaaccaactcaacagacgagaaattatcaatgattctaagagtcaagatcattcctcaaccacgaacgttgctcagaatgaccaacacaccaacaaccagagtcgaggagactctatcgaaaacaagagcatttgggaaataggcaataaagctgaccaatccaacaacattcagcagaatgagaataattctcaacagactaaatcttctaacaaccagaacactaacaaccaatccaatcagaatcagctcaacagacgggaaagtatcaatgattccaagagtcaagatcattcctcaaccacgaacattgatcagaatgaacaacacaccaacaaccagaggcgaggagactctatcgaaaacaagagcatttgggaaataggaaataaagctgatcattccaataacattcagcaaaatgaaaatataatatcgaagaatgttcaagattttttttttttttttttttatagcaatctaataacaacggtagcaacaaggcagattcgattgataagattattcaaaatgactcccaacatcaaaacgagcaaccaaatatag

SEQ ID NO: 443 - DNA, 744 bp, Phakopsora pachyrhizi
  source 1..744: mol_type = genomic DNA
atgaagtacaaccttgtcacgattttgctatccatctatgtcttcaatttatattcttgtagccctatacctaaccatcatcagagcggtgctcaaggaatttctggtcaggaatccaataacaattccttaaaccagaggagagctgaatctcaacagaccaaatccgattccaacaatcagattcagaacaccaataacgaaatcaaccagaaccaactcaacagacgagaaattatcaatgattctaagagtcaagatcattcctcaaccacgaacattgatcagaatgaacaacacaccaacaaccagaggcgaggagactctatcgaaaacaagagcatttgggaaataggaaataaagctgatcattccaataacattcagcaaaatgaaaatataatatcgaagaatgttcaagaaaacaacgacgcttccaacaagcagcaatctaataacaacggtagcaacaaggcagattcgattgataagattattcaaaatgactcccaacatcaaaacgagcaaccaaatatagtcagaagatcaaatgttggaactgagaatgttcaaggccagtctcaagagaaagcgttaaaccaaggatctgagcaaaactgggctccaattcagggtaacaactggaatgtggccgtcagacgtactgtcaatgagaacgttcaatctcagaacaatcctacctcacaattacaaaaccctcagaacaattggtcaaatcaatga

SEQ ID NO: 444 - DNA, 264 bp, Phakopsora pachyrhizi
  source 1..264: mol_type = genomic DNA
atggttttacctctccagatcattgtcatcagcatatttatttctctgaacaacctaagcgctgctaggctgtttggaagtgacggtataaggagaataatcagtggccggtctactcatccttctgttggagaccgacatggtctttcagctaggaatttgttaaatcaagaaagctacgacgattataaatccgatgaaaatttatatggatcgacaaccaagacaaccggtttaacaactgaacctgttaatctagtgcct

SEQ ID NO: 445 - DNA, 549 bp, Phakopsora pachyrhizi
  source 1..549: mol_type = genomic DNA
atgtcttctattctaaaatcgcttctatcatggatcagatcaatttttttctcaaaagaactagaaattactatcattggtcttcaaaattctggaaagactagcttggtgaacgtattaatgtctggacaattctctgagactatggttcctactgttggtttcaatatgaggaaatacacaaaaggatcagtgacaatcaaactttgggatattggaggtcagcctagatttagatctatgtgggagagatactgccgaggagtctcggcgatactatttgtggttgattcggccgacaagcagtcgatcgaccaatccaaggttgaattacaaagcctgatctctcaacctcagctaagcggaatcccactcctagttcttggaaacaaaaacgacctggctgagtctttgaaagtaggcgaattgatcgaaaagcttgaactttccaaggtgactgggagagaggttagtatatattcaatctcatgcaaagagtctataaacgtcgacgtcacccttaattggctattaaaaagagcaacatag

SEQ ID NO: 446 - DNA, 414 bp, Phakopsora pachyrhizi
  source 1..414: mol_type = genomic DNA
atgtcaaacaaggccaaggctagcacaaaatttcgcatgacccttgccctccctgtgggtgctgtggtcaactgtgccgataactctggtgccaaaaatctctatatcatttccgttgttgggtttggtgctcggttgaacaggctgccggctgcatcatgtggcgatatggtgatggcaactgtcaagaagggtaaaccagagctgagaaagaaggtccatcctgcaattattgttcgtcagcgaaaaccttggcgtcgacgtgatggcatcttcctttattttgaagataatgcaggtgtgatagtcaatcccaagggtgaaatgaaaggttcagcgatagctggaccagttgcgaaggagtgtgcagatctttggccccgtattgcgtcaaatgcaggaacagttgtttaa

SEQ ID NO: 447 - DNA, 381 bp, Phakopsora pachyrhizi
  source 1..381: mol_type = genomic DNA
atgtactccatcagatttaatccggtcactcttttaagctttctctttgtattctcgatcgtacattccactccaatggactcattagtgccaagtaattatggccttaggcctactgggccttatacggcataccccaaaggaggcgatcacccagattggaaatatctcaatcaacgagaatggtacaaaattgatgccactcaacaggatcatgctcgatttcattcatatctctcaatgtctatttatggcgactataataaaacttgtcctcaaacttttactcagggatataaaatcattcaatctttttccaacttctacgttgcttatatccctgaaatggacaagattgttgttgtatttaaaggaccgggg

SEQ ID NO: 448 - DNA, 555 bp, Phakopsora pachyrhizi
  source 1..555: mol_type = genomic DNA
atgggtgtttcaaagctgtttttcatatggattggccttttcaccatctcaaagctcagcacaactctttctgttagctttggcaagggaataaatccattgtcactagcagacgagggcgagagtgtgctgaagcagtttcaaatctttggtaaaaataatgaggtcgtgaatgtcaagattgtttcaaagtttaactctgattctaactcattaagattcacaaagagagacctcctgggaattgaggatgagaagagtaatgagaagggaaacgataaggtgaagaatcaggcccagaatagaaattacactagagtcgatcttacacctgctcgtcctacttcagaaacctgttactctggttcattccaaggaccaaaccagagtgattgcgatgtgatattctatgcccagaagtataactcttatggtagtttaactgctttcccaggaacgtttgtttatgtttactactcttcgtgtgtggtggcttttcaaaaccccaatcatagtaactattcattagacgttgagtttcagatatcgatttaa

SEQ ID NO: 449 - DNA, 303 bp, Phakopsora pachyrhizi
  source 1..303: mol_type = genomic DNA
atgaatttcggatcaagctcaaagaagaaccttgtagctagaccttcagttggtataaggagactagtgctggtatgcaccgcgatggtctggagtatcactctgatactcagtcaaccgagtggtgtaaactcatctccgtttttaagagtcgccaagagcatcggaccagaagttgctaaaggtgccgccggaggtgttggattagttgcagtagaacaaattgctaacaaagtgattgggagaagtaccgatggtaaaaatgtagaagatgaaaattctggttcgaaaaactttccataa

SEQ ID NO: 450 - DNA, 414 bp, Phakopsora pachyrhizi; identical to SEQ ID NO: 446
  source 1..414: mol_type = genomic DNA

SEQ ID NO: 451 - DNA, 303 bp, Phakopsora pachyrhizi
  source 1..303: mol_type = genomic DNA
atgaatttcggatcaagctcaaagaagaaccttgtagctagaccttcagttggtataaggagactagtgctggtatgcaccgcgatggtctggagtatcactctgatactcagtcaaccgagtggtgtaaactcatctccgtttttaagagtcgccaagagcatcggaccagaagttgctaaaggtgccgccggaggtgttggattagttgcagtagaacaaattgctaacaaagtgattgggagaagtaccgatggtaaaaatgtagaagaagaaaattctggttcgaaaaacttttcataa

SEQ ID NO: 452 - DNA, 858 bp, Phakopsora pachyrhizi
  source 1..858: mol_type = genomic DNA
atgtttaagggtttgtcattagccgttctttccattgccctgagggagaccatcggtcaggctacgaccgtctctacagattcattgaacgccatctcgaaccagtttgactcatcttacttgtcatctcagtatcctgatgggttcgggatcccactaaaggcccaagcgattctgaatgtgaattacccctttggaaacgtccagctgggtaaagcttacaatgcatcagacgttgccaatcaacctacaatcagcatcagtcctctggaatccagccaggccagcttcaaggctccaagtgcttttacattgatgttggccgatgccaatgcccttggtaatcctgatccaaagggaaactatcgccacttcttagagaacggggtgacgtttggagatccaacctcgaatggtacgatgtcaatgaaccccggctcaggcacggttgtgacctcgtacgccgggccgggtcctctacccaacgagggtcctcatcgttacgcctggctactgtttaaccagcctggtgacttccaggccccttcgaacctgtccagtgctggtgtgtctcccggccactggtacgtcaatggctatatcagcgggtctaagcttggtgacttggtagctgctagcttcttcactgtcgaaaatggacatgcaaacttcacgcccaagcctctagtctctccagtccataacggcaacaagaccaacaacaccagcaatctcagtggtaactctaccaactcgtctggcaatgctaagatcagtggtgcagctgtcagtgccagagtacttgatggtactctgatgggtctcgtcgcagtgggtcttggattggcctttgtatag

SEQ ID NO: 453 - DNA, 702 bp, Phakopsora pachyrhizi
  source 1..702: mol_type = genomic DNA
atgaagtacaaccttgtcacgattttgctatccatctatgtcttcaatttatattcttgtagccctatacctaaccatcatcagagcggtgctcaaggaatttctggtcaggaatccaataacaattccttaaaccagaggagagctgaatctcaacagaccaaatccgattccaacaatcagattcagaacaccaataacgaaatcaaccagaaccaactcaacagacgagaaattatcaatgattctaagagtcaagatcattcctcaaccacgaacgttgctcagaatgaccaacacaccaacaaccagagtcgaggagactctatcgaaaacaagagcatttgggaaataggcaataaagctgaccaatccaacaacattcagcagaatgagaataattctcaacagactaaatcttctaacaaccagaacactaacaaccaatccaatcagaatcagctcaacagacgggaaagtatcaatgattccaagagtcaagatcattcctcaaccacgaacattgatcagaatgaacaacacaccaacaaccagaggcgaggagactctatcgaaaacaagagcatttgggaaataggaaataaagctgatcattccaataacattcagcaaaatgaaaatataatatcgaagaatgttcaagaaaacaacgacgcttccaacaagcaggtaattttttaa

SEQ ID NO: 454 - DNA, 987 bp, Phakopsora pachyrhizi
  source 1..987: mol_type = genomic DNA
atgaagtacaaccttgtcacgattttgctatccatctatgtcttcaatttatattcttgtagccctatacctaaccatcatcagagcggtgctcaaggaatttctggtcaggaatccaataacaattccttaaaccagaggagagctgaatctcaacagaccaaatccgattccaacaatcagattcagaacaccaataacgaaatcaaccagaaccaactcaacagacgagaaattatcaatgattctaagagtcaagatcattcctcaaccacgaacgttgctcagaatgaccaacacaccaacaaccagagtcgaggagactctatcgaaaacaagagcatttgggaaataggcaataaagctgaccaatccaacaacattcagcagaatgagaataattctcaacagactaaatcttctaacaaccagaacactaacaaccaatccaatcagaatcagctcaacagacgggaaagtatcaatgattccaagagtcaagatcattcctcaaccacgaacattgatcagaatgaacaacacaccaacaaccagaggcgaggagactctatcgaaaacaagagcatttgggaaataggaaataaagctgatcattccaataacattcagcaaaatgaaaatataatatcgaagaatgttcaagaaaacaacgacgcttccaacaagcagcaatctaataacaacggtagcaacaaggcagattcgattgataagattattcaaaatgactcccaacatcaaaacgagcaaccaaatatagtcagaagatcaaatgttggaactgagaatgttcaaggccagtctcaagagaaagcgttaaaccaaggatctgagcaaaactgggctccaattcagggtaacaactggaatgtggccgtcagacgtactgtcaatgagaacgttcaatctcagaacaatcctacctcacaattacaaaaccctcagaacaattggtcaaatcaatga

SEQ ID NO: 455 - DNA, 489 bp, Phakopsora pachyrhizi
  source 1..489: mol_type = genomic DNA
atggcccctcaaattcttctcgtcctaccttttcccggctctccaacaatggtcttaactcacccttgccactcctctgaccaggattcgatactcgaagcagtctgtcgtcagaatcagcttcctctttcttttgcttcttcccttcgtctatcgcgatgtggacgaccttggaacggtatacttgctgaagatgaattctcagatgttaacttcgtagtggctgatgttgcaatgcggctaaggggtggcggtcctaagaagaggtgtcagcatgccaagaatagtgtaaatgaatctcagtgtggccaacctgctttaaggctcgttggggactgccctcattgcacccttcagttctgtgctcgtcacaggcttccggaggatcatgcttgccttaatatgacttcttgtcgcgaagaagcatttgcgaagaacaaggcaaaacttgaaagcgagcgaactgttggaagtaaaatggttggagca

SEQ ID NO: 456 - DNA, 768 bp, Phakopsora pachyrhizi
  source 1..768: mol_type = genomic DNA
atggttgaagcaaaggtattggtagtcgttgcttctttactgttttttagctttttatctgccagtccggcaggctcgcatgtcaggcatcttacaagttcaaagagaccacctcccggggcactcgtcgtaagaaatcagcgaactagagttggagaattttcaaccataaatgatgccgttaaggaattgaagaataagaaaggcccccagttcatcttcattcacagtggcacttacaatgaagcagttcacatcaaatataaatcagcgctgacgattcaagggtatagtgaaaaccccttgtcttatgaagataaccaggtcacggttatcaaacctaccgttgcatctgaggctggaagtgattctaagagctctgttatctgggctcaagttgatgactttaaaatgtacaacataaacgctataaactcttatggagcaggaaaagataaacaggctgtggcggttaccgctgatggtgatcggcaaatgtacaaaagttgcagatttgatagctatcaggacacactgtatatcaggtcaactgcatctttctttgataagtgcttgatctcaggcggtgtcgatgttatctttggcgctggaagtgcttggttcgaaaaatgcactattggggttaaaccctctcctgacaatggtatttctacgataactgctcagaaaagagagcgtggtagtaactcgcggaaggccttggtccgaatatgccagcgtcgcattccagttctgctttttacctga

SEQ ID NO: 457 - DNA, 126 bp, Phakopsora pachyrhizi
  source 1..126: mol_type = genomic DNA
atgcaaaagctctacattctgcttcttttttccattattaaactctgctcttccgggcttataggttttgaagctgaaataattaataatccaaacacatggaaaacagttggcgaaatccactaa

SEQ ID NO: 458 - DNA, 285 bp, Phakopsora pachyrhizi
  source 1..285: mol_type = genomic DNA
atgtttggactaggaaagatcttttacgtttctctcctgcttatcaactcagtggcaatcttaaacgaggatagatttttggcaagaattggttggtcgtcaaatagtggtaaccctaactttggtacgacgaatcaatttcatcatgatccatcaatttctccgaatggccaatcagtcaaagaaaaaattatcaatttgatcggtgcggtaaggacattgatgaggattccactaataggactgaatattgttgttattatttatgaattgattctgggttga

SEQ ID NO: 459 - DNA, 186 bp, Phakopsora pachyrhizi
  source 1..186: mol_type = genomic DNA
atgatagctttactgggcttcctgagctccccaaggctccttacagtgaacaactcactaataaagctgtgccaaattacaccaaacctaagcctgacgtgccttctggcttcaactccatctcagagaaaaaagatcaaaaatggcacagtcaaaagcagaattttgatccgccccggatcttag

SEQ ID NO: 460 - DNA, 186 bp, Phakopsora pachyrhizi
  source 1..186: mol_type = genomic DNA
atgacatgctgcactccaacactccccctaatttttatcctaacaggatataaattaccaaaccatcatatatatttttattttattttcatctcccccatctctcacaatctgttactccccggcagacttgttaactgccaactttcacaattcttctatattacagtttacaggtcatgttga

SEQ ID NO: 461 - DNA, 261 bp, Phakopsora pachyrhizi
  source 1..261: mol_type = genomic DNA
atgaaatttttctgcagcagcatttgtttttcaacattctatttcttgatgattttaggattttcaacatttaacttcacgttagcatttaggtgcggcgtcacatcagttggattctgtgctaacactaatggcggcggcaaattacaagcacaatttttaaggcaaaggctcaattccaatgaatatgtgaattgcccagctaatagacccctgaatttatgttgcagtgctggaggtcgtggttgttcacctgcataa

SEQ ID NO: 462 - DNA, 192 bp, Phakopsora pachyrhizi
  source 1..192: mol_type = genomic DNA
atggttctagatattaaaaaacttgatggtttctctgttgagagcctttacaaattccagactcgaacagtcattctgatatttttgatagctgttacactatttgaatcagctcgtgctgcagtgatgacagcgaaagataccgaatgctttcgcttcaagtattgcgcctgtcgttgctctattgtttga

SEQ ID NO: 463 - DNA, 276 bp, Phakopsora pachyrhizi
  source 1..276: mol_type = genomic DNA
atgaggtttaacaatttccacagattctcttcgttcgaagcttcgaaggttaaattacttttcatcgtatgcgctttatccattagcctgaccagctctttacaattaaacattttgaagggtaccgaacctggttgcggttcccttcctgagaataatatgattaatatgcaattatcacaatattcggatgcgcgagatcctggtaaaggtagttctcctaagaccaataaggaatggccatactctgatgataatcaaaccgatttaaagtga

SEQ ID NO: 464 - DNA, 432 bp, Phakopsora pachyrhizi
  source 1..432: mol_type = genomic DNA
atgaattttcttttgatgaattttatgagtatggtagtcttgatcattctatctacttcaagcctatcaagatcttctccctcagcagacccttcgactcacaatcatcactccatgttaatcagaacgaacgatgaactcttgagtaaaccgtcaatcaagaagcgaaagattgtggattttgatgttgacaagaaggtgatgaagaagaagaggaacgatgttgtttatcaggagggggatgagggcagtgaagaaaggatgaaggagctggatggagatgtagagatctctgaggaggactttgatcaacttgctgtcattccagacggtagtggtagcgagcgacaatacatctggtcgagatggagcgttgctagaaacatctttggtgcaggtgacccaaaaaaagaagtcaaaagtaattcctga

SEQ ID NO: 465 - DNA, 711 bp, Phakopsora pachyrhizi
  source 1..711: mol_type = genomic DNA
atgaagttctcttactcccaattgattgttctgctgtctgtgatttctctgagctttgttgcatctgaagaaactaaaagtcagagggcagaatcttccgatgagaaatggttcggactaggttacaacagcttgaggtggaactactggaacggccttgccggatggggtggtataggatcattatacccttggctgggtgcttacagcggtgcctatgggattggtctatgctcaggaaactacctcaacagctggttcaagtctgcccaaggcgaaaagcagcgccgctctttcgagttcaattcaggagctcaactccaaacccgtggtgagccagacttattggagactgtgacgtgcaagaacaccaagggtgaatctcagcagttcctcacaagcagttgtttgaaggcagcagagcagttggtcgagaaacaaacttcatctgccacttgtggtagctgcactctccaactccatggtccatcaggagatctatcagcaaagagcattcctgcctcagagctaaccaccgcagcctctaacatcctgaaggcctgctccaaggctgagaacaagattcttagcacctcagagctccagagacgtggaaacgaagctgagaacagctcttcggacaacaatgctaaggattcgaagaactcctttgccgttgtgctcctcaaaggcaatggtccagagtgctag

SEQ ID NO: 466 - DNA, 843 bp, Phakopsora pachyrhizi
  source 1..843: mol_type = genomic DNA
atgtttaagggtttgtcattagccgttctttccattgccctgagggagaccatcggtcaggctacgaccgtctctacagattcattgaacgccatctcgaaccagtttgactcatcttatcctgatgggttcgggatcccactaaaggcccaagcgattctgaatgtgaattacccctttggaaacgtccagctgggtaaagcttacaatgcatcagacgttgccaatcaacctacgatcagcatcagtcctctggaatccagccaggccagcttcaaggctccaagtgcttttacattgatgttggccgatgccaatgcccttggtaatcctgatccaaagggaaactatcgccacttcttagagaacggggtgacgtttggagatccaacctcgaatggtacgatgtcaatgaacccaggctcaggcacggttgtgacctcgtacgccgggccgggtcctctacccaacgagggtcctcatcgttacgcctggctactgtttaaccagcctggtgacttccaggccccttcgaacctgtccagtgctggtgtgtctcccggccactggtacgtcaatggctatgtcagcgggtctaagcttggtgacttggtagctgctagcttcttcactgtcgaaaatggacatgcaaacttcacgcccaagcctctagtctctccagtccataacggcaacaagaccaacaacaccagcaatctcagtggtaactctaccaactcgtctggcaatgctaagatcagtggtgcagctgtcagcgccagagtacttgatggtactctgatgggtctcgtcgcagtgggtcttggattggcctttgtatag

SEQ ID NO: 467 - DNA, 858 bp, Phakopsora pachyrhizi
  source 1..858: mol_type = genomic DNA
atgtttaagggtttgtcattagccgttctttccattgccctgagggagaccatcggtcaggctacgaccgtctctacagattcattgaacgccatctcgaaccagtttgactcatcttacttgtcatctcagtatcctgatgggttcgggatcccactaaaggcccaagcgattctgaatgtgaattacccctttggaaacgtccagctgggtaaagcttacaatgcatcagacgttgccaatcaacctacgatcagcatcagtcctctggaatccagccaggccagcttcaaggctccaagtgcttttacattgatgttggccgatgccaatgcccttggtaatcctgatccaaagggaaactatcgccacttcttagagaacggggtgacgtttggagatccaacctcgaatggtacgatgtcaatgaacccaggctcaggcacggttgtgacctcgtacgccgggccgggtcctctacccaacgagggtcctcatcgttacgcctggctactgtttaaccagcctggtgacttccaggccccttcgaacctgtccagtgctggtgtgtctcccggccactggtacgtcaatggctatgtcagcgggtctaagcttggtgacttggtagctgctagcttcttcactgtcgaaaatggacatgcaaacttcacgcccaagcctctagtctctccagtccataacggcaacaagaccaacaacaccagcaatctcagtggtaactctaccaactcgtctggcaatgctaagatcagtggtgcagctgtcagcgccagagtacttgatggtactctgatgggtctcgtcgcagtgggtcttggattggcctttgtatag

SEQ ID NO: 468 - DNA, 711 bp, Phakopsora pachyrhizi
  source 1..711: mol_type = genomic DNA
atgttcaacatgttaatagcattagctattactcttgtgcacgctggcagtgtattctgccagcaagcagcctcagctcaacaagattcatccactacaattgtggctaatacaaaccttaacggagcttatggagtttcgggtcagatctcgttcacccttattcccacgacagaagaggttgctgtcaagatttcagttaaagggttgatggccattaactctaccgcccaatttggctatcacattcataccaatccaatttcatccgatggaaactgctcttctgcccttggtcatctcgaccccctgcaggtcaccgattccgtggtatgcaacggaacggcgcggttatgtcagatcggggatctatcaggtcgtaacggcaagctaccagggaatcaatctgttgcaaatgtctcctacacggataaattcctcagattctggcctcaaccatttacccttcttggacgaagtatagtcattcatgctcccaataccacaagaatcgcctgtggaaacattacctctgttgttgatggaactgcggacaatcaagggaagcctaccaaccaaagctcaaactatgtgacagtgtatgcttttttatcttgcatgcttttcactaggcctttattgacttcttttaaaaaaacagctactctacacgagcacctcctccttctaataacactgccaagtgagtaa

SEQ ID NO: 469 - DNA, 258 bp, Phakopsora pachyrhizi
  source 1..258: mol_type = genomic DNA
atgcttaatctatcattctcaggcctcttgatctttgcctgtgttctttttcagtttacgtctatatcagcaaagaatttaactgctactgctgtaaaaaatgctctgtattcttcttgtgaaaagtctgccttagccaagccctgtggtggggccctggataagctatatgacactggacgatttaaaagtttcacgatcgagaaacaagtacactttgataaaacttcaaattgcaaattcacctggtggacgtaa

SEQ ID NO: 470 - DNA, 258 bp, Phakopsora pachyrhizi
  source 1..258: mol_type = genomic DNA
atgatgggcgtcctgatgatgctcttcctacacctttacatgaagtatgacgcccctctgttcttacaatccctgatgcctctcaaaacgacgtatgagaacaaggtgatacagatccatttgctcggtcgatcggccactggtgatctgaaaaggccattcaaaactggtggcttcatgggagctgcaacggatgcaaagactgatgctaagagcataaaggaggcagagaaatctagtgttaagaaggaccaatga

SEQ ID NO: 471 - DNA, 144 bp, Phakopsora pachyrhizi
  source 1..144: mol_type = genomic DNA
atgaaaggaaacttattttctctgagttatttttttgcttttaaaatattgaatttgacaagatttttacagatttgttttgattgtattatacagaaaaggactataattaaagttttgattgatataggatattttgggtag

SEQ ID NO: 472 - DNA, 381 bp, Phakopsora pachyrhizi
  source 1..381: mol_type = genomic DNA
atgtcgttcgcaaaacaggttcttctctcaatttgcttcttcgctgttttcagcaagataaacgctttgacggtagcacaaccctcagttcggtgcttgggggataccaatcctgctgccattaaagcggaagattgtgataatgccatgtctcagtttagtgtggattctaacggctacattcagtacgatccccaaggagaaaaaagagattttgggagttgtagaattcagattcaaagtacttcagctgcatcaaacttagcaactattaatcctgaagtcttgaagatatatatatcaactggtttaacatcatgcaacggaggaacatcagccattgagccacaaggcctagttgtgaatgttattaagctctaa

SEQ ID NO: 473 - DNA, 381 bp, Phakopsora pachyrhizi; identical to SEQ ID NO: 472
  source 1..381: mol_type = genomic DNA

SEQ ID NO: 474 - DNA, 1065 bp, Phakopsora pachyrhizi
  source 1..1065: mol_type = genomic DNA
atgcgcttttctttgatagttgccgtttaccttgcattctccgcttattcattggcgttgccagtagccttgaacagcttgactccccttcaagcaacacctaactatttgggccgtgaaacttcttctaatttgacaagttcagtttatcctcaaaacgattatgaggatatgcaagttggaagctcgaatttaggtaatcaacttggctcatatggaggtgaagagagctatgaagaggaaggcaatgaaaatcaagaaggatacggagaggatggaaatgggtatgaagatgagtcgtacggaaaccaaaactatgagtaccaggattacgataaccaatacgaagctggtaaagagcagggtggttatcaaacacagggaaacaaccaaggctatcgctcgggatcaaataaaaactacagcccgggatttaatgctggctcctacggcttcgggtccgtaaacaaccgcgattatggcattgttggaggatacggctctattcaaggcagtaatcaaaactataactttagatcaaacattggctatagctctggaattggcgctggaattttcggcggctacggatcagcaaacagtcgcgactatggaattccgtattacgcccctagggattactcagaagctggatatggggctggaacgggcgatgaagggcaagtatatcgctacacagtaggagaaggaatgccgaatggcgctggattaggtgctggagtaggcgccggcgtaggcgccggtgttgaatatggagaaggtagctcactggggaaaggcattgaatatggtgttggcgccggatatggcgctggatacggtgccggtgttggtgcaggggaaggcgcaggctatggtgccggtgtcggtgcaggagtaggcgcatggctatggtgccggtgttggtgcaggagtaggcgcaggttatggtgccggcgccagctgctggatatggtgtcgacactgactctagctctgatgatttcgcttcgggtggagaatataattcagaacattattagcaggagtattacacttgtttaa

SEQ ID NO: 475 - DNA, 1059 bp, Phakopsora pachyrhizi
  source 1..1059: mol_type = genomic DNA
atgcgcttttctttgatagttgccgtttaccttgcattctccgcttattcattggcgttgccagtagccttgaacagcttgactccccttcaagcaacacctaactatttgggccgtgaaacttcttctaatttgacaagttcagtttatcctcaaaacgattatgaggatatgcaagttggaagctcgaatttaggtaatcaacttggctcatatggaggtgaagagagctatgaagaggaaggcaatgaaaatcaagaaggatacggagaggatggaaatgggtatgaagatgagtcgtacggaaaccaaaactatgagtaccaggattacgataaccaatacgaagctggtaaagagcagggtggttatcaaacacagggaaacaaccaaggctatcgctcgggatcaaataaaaactacagcccgggatttaatgctggctcctacggcttcggatcaaacattggctatagctctggaattggcgctggaattttcggcggctacggatcagcaaacagtcgcgactatggaattccgtattacgcccctagggattactcagaagctggatatggggctggaacgggcgatgaagggcaagtatatcgctacacagtaggagaaggaatgccgaatggcgctggattaggtgctggagtaggcgccggcgtaggcgccggtgttgaatatggagaaggtagctcactggggaaaggcattgaatatggtgttggcgccggatatggcgctggatacggtgccggtgttggtgcaggggaaggcgcaggctatggtgccggtgtcggtgcaggagtaggcgcatggctatggtgccggtgttggtgcaggagtaggcgcaggatatggtgccggtgctggtgctgggctcggtgctggtgttggcgccggtgtcggggctggtgttggcgcaggttatggtgccggcgccagctgctggatatggtgtcgacactgactctagctctgatgatttcgcttcgggtggagaatataattcagaacattattagcaggagtattacacttgtttaa

SEQ ID NO: 476 - DNA, 1095 bp, Phakopsora pachyrhizi
  source 1..1095: mol_type = genomic DNA
atgcgcttttctttgatagttgccgtttaccttgcattctccgcttattcattggcgttgccagtagccttgaacagcttgactccccttcaagcaacacctaactatttgggccgtgaaacttcttctaatttgacaagttcagtttatcctcaaaacgattatgaggatatgcaagttggaagctcgaatttaggtaatcaacttggctcatatggaggtgaagagagctatgaagaggaaggcaatgaaaatcaagaaggatacggagaggatggaaatgggtatgaagatgagtcgtacggaaaccaaaactatgagtaccaggattacgataaccaatacgaagctggtaaagagcagggtggttatcaaacacagggaaacaaccaaggctatcgctcgggatcaaataaaaactacagcccgggatttaatgctggctcctacggcttcggatcaaacattggctatagctctggaattggcgctggaattttcggcggctacggatcagcaaacagtcgcgactatggaattccgtattacgcccctagggattactcagaagctggatatggggctggaacgggcgatgaagggcaagtatatcgctacacagtaggagaaggaatgccgaatggcgctggattaggtgctggagtaggcgccggcgtaggcgccggtgttgaatatggagaaggtagctcactggggaaaggcattgaatatggtgttggcgccggatatggcgctggatacggtgccggtgttggtgcaggggaaggcgcaggctatggtgccggtgtcggtgcaggagtaggcgcatggctatggtgccggtgttggtgcaggagtaggcgcaggttatggtgccggtgttggtgcgggagtaggtataggatatggtgccggtgctggtgctgggctcggtgctggtgttggcgccggtgtcggggctggtgttggcgcaggttatggtgccggcgccagctgctggatatggtgtcgacactgactctagctctgatgatttcgcttcgggtggagaatataattcagaacattattagcaggagtattacacttgtttaa

SEQ ID NO: 477 - DNA, 351 bp, Phakopsora pachyrhizi
  source 1..351: mol_type = genomic DNA
atgtcgttcgcaaaacaggttcttctctcaatttgcttcttcgctgttttcagcaagataaacgctttgacggtagcacaaccctcagttcggtgcttgggggataccaatcctgctgccattaaagcggaagattgtgataatgccatgtctcagtttagtgtggattctaacggctacattcagtacgatccccaaggagaaaaaagagattttgggagttgtagaattcagattcaaagtacttcagctgcatcaaacttagcaactattaatcctgaagtcttgaagatatatatatcaactggtttaacatcatgcaacggaggaccattgagccacaaggcctag

SEQ ID NO: 478 - DNA, 303 bp, Phakopsora pachyrhizi
  source 1..303: mol_type = genomic DNA
atgaatttcggatcaagctcaaagaagaaccttgtagccagaccttcagttggcataaggagactagtgctggtatgcaccgcgatggtctggagtatcactctgatactcagtcaaccgagtggtgtaaactcatctcctattttcaaaatcgccaagaccatagcgccgacagttgttaaagcagcagccgcaggtggtggtttaatagcagtggagcaaattgctagcaaagtgattggaagaagcaccgacggtcaaaatatcgaagaagaaacttctggcttgataaacttttcttaa

SEQ ID NO: 479 - DNA, 744 bp, Phakopsora pachyrhizi
  source 1..744: mol_type = genomic DNA
atgttttctaaatcatttcaaggattgagcctactcgtagttctgatggcctctattatctttaactgtgatgaatcaaacgcccagagtaactttaactcttttcaacaaccaggcacgaactgttacgaacagtcaaaatcctatggaatcaatttcgaacattgtgaaaaagctctaaggaaaatttcatacgattctaatggtaacttggataattcatccaagacggtttttgtctggcataaatcttgtgtggtcaaagttcaaaaggcaacatacgctcaaccatcgagacaacaagtagaatatggtgttagaaacttactacagacttgtcctacgaggggagggatttatatcccttcaaatgactttaggacctatgtttactcttcaaatcgtgaaaacgtttacaatttgaacagcccagtgtgcattaagcaccaatgtcatatcaaccctaatgactgtttgatggcctttaacaacattcctcttagtactaaaggttttttcttaccatctggttcaacctcttctaatgttataaagacttctagtggtaactgcactgttaaacttgtcactactgatggtgcaggattcagaattaaccatcctgaaattaactctggtatcaaaactctactctcaaaatgtggtagcagacctggttacaactatttcggtggtggcagtctaggaatgaatggtgatattaaaatcatcactcaaaacagt

SEQ ID NO: 480 - DNA, 1560 bp, Phakopsora pachyrhizi
  source 1..1560: mol_type = genomic DNA
atggctttattaaatcgaattgtttttgttttgctcgccttgacctttgcaagagttattgaatctagaactggcgttaaattagactctaggctgaacactcgtaatacgcaagagcaattaaaccatcctcgtgtaaaatgtggtcgatctactgctactcaagctactggatccacttatagatgtaggaacagctcaaatgatggaaagaagcacaaaaaggtacccaaaggttatggtaaaaaagagaaaggcaaatacacgccaaaaggtcctaaacatgaatcctatgctccgaaagctccatcaaaacttcccaccggcggttatggtggacatgagcaggaacaatatgcgccaaagggacattcctcagagccttacgttccgaaaggagttgtctcgtcgcctgtcacacttgctgcacttgtcacggtgataacttatgtgagcaaagtaaactggcttctgtgcattatcaaacagcgttttcaacatgatcttgtgagacaatcggccactgcgaaagtaattcaatcgtctggcccgtcaggccgacactctaactcaggtctcgttgcgactgtttttggagctactggtttcctcggacgttacttggttcacaagctagcgaagaagggcacccaggtgattgtcccataccgagaagaagatagtaaacgacatcttcgtgtcatgggagacctcggtcagattgtgcctctggaattcgaccttcagaacgaaacttcgataaacgaatgtgttcgacactctcatctggtctacaatctgattgggagagactacgagaccaagaactatagctttgattcggtttttgtcgatggtgcagcgaagataacgcagataactaaagataatggcgtaaatcgactgatacatatctctcatctcaacgcctcacacgattcaccatcaaaattttatcgagctaaggcttctggtgaagataaggttaaatcgatctttccagacgtcacgatagttaggcctagtacattctttggacaggaagatcgatttctgaacaagatggttaccaaaccattcacttttaacgtcaaccgtttggagacaattactagaccagttcatgtcctcgatgttgccgaagcgataagtttgatgggaacagcccagtctaccgttggaaagacttttgaactggctggtccaagaacatactcctacgaagatctttttgaaacgctagaaaatcttactttgaaatctctaacaccttcgctcaatctacctctaccaatcttcaagctgttcaacaaaattgtcagtagggctgtctggtggccaacgttgagtgaggatgagattgtcagaaggtgtattgatgataaagaggagctaagtgagggggcattaggttttgattactttggtattgagccagatttggttgaggatgctgcgataacatacttaaggttttacagatcggctgccaattttgataagcctattcaaaggggtggattcaaagttaaacccaagacttataatgttgtaaactga

SEQ ID NO: 481 - DNA, 123 bp, Phakopsora pachyrhizi
  source 1..123: mol_type = genomic DNA
atgcaaacactccagttaatggtgctggccaaggtagaggtggctcacctggtgacgtcggtggagcaccaattggtggaaggccagagaacgcaagggaagatagggccgaagctcctatga

SEQ ID NO: 482 - DNA, 99 bp, Phakopsora pachyrhizi
  source 1..99: mol_type = genomic DNA
atgaactgtaacgatgcatgttcttgggttatcttagctgtttttcttggaatttcggccttaaatattgcttgtgtagaaggagccagcgtagttggg

SEQ ID NO: 483 - DNA, 129 bp, Phakopsora pachyrhizi
  source 1..129: mol_type = genomic DNA
atgaattatatcttccgttgtactatcatttgtatctggcttttgaggaagaaggtagttaatcttgctgaagtttatctgatcaaaccggttgtttccccaaccaatcatctgattcctatagattag

SEQ ID NO: 484 - DNA, 243 bp, Phakopsora pachyrhizi
  source 1..243: mol_type = genomic DNA
atggttttgcctagaaacacctacagagatccatgggcgaaaaatgaggcttggagaaaccatcctattttttcgagacgtcaacaattaagaaacttctttccgggtttcggatgggcatcagttgcctttacaatctacgttttgtacgacaacaactatcgagcgaagaataaaaacattgaggacgtcaagggtgacttcaataagattgttgatgatgttaaaaatcttaagttttga

SEQ ID NO: 485 - DNA, 528 bp, Phakopsora pachyrhizi
  source 1..528: mol_type = genomic DNA
atgcttcgcttaatttcgctaggacttttggtatgctctgccgcaagattgaccgtgagcagtccaagcgatctctcactaagacctcgtcagcttggcttaggtgccggcggagcagccgcaggttccttcagcagtagtaccagcgtatcgacatttagctccttcgttcaaggttggagcgtcattccaactgctttcggaacttgcgcggccaccttccaacaaagagttactgttcaagttgctgttcagtcggtacagcagttatacagtactgtgaatggagttctaggacattacggtggttgtggtggttgcggtggtgcttctgctgctggtgcttacgcttctaaatatcaatccatcatcacccagagtttcacttcctggcaaactatacttcaagttgggcaatcctcatacgccaatgcttgggactctcaatttattcctctcttccgacaattcaaccctttccttacagcggtacaacagaattccggactttttggcatcaatctcgat

SEQ ID NO: 486 - DNA, 1107 bp, Phakopsora pachyrhizi
  source 1..1107: mol_type = genomic DNA
atggttttactaaaccttcgagctcaaatattaatttcgcttaacgcgagattatttaaagtctttaatcttagagataataaagttttcacaatttcattacttaaaaatattctcggaagttcattccatgttccagagcaccttattcaatttacccttgtttcaggccgctacacgaaagatagtgatgcgctcgttgatttgcgtgccagcgatggaaactttgcttcattagacgttggggtcctaccacctcattactgctcttctttatctgaagctattgttagctttgaattcagagctaacccaaccccaacgtatcttgacggagtatctgtcgccggaatccaggaaattccccttatgtatcaaactctacgcccggtctcagaccgtctgaataacgagctaaataatttgaatatttcaaataaaaaattgaccaaggatcaaagaatagctcttcgagcaagatatgaggatccatttagcattagctcaaaatcttgctttgaggaggacagcgatgacgactcgctctatgaagatgaagattctgaatcggaacttggagactctgatgatgatttgtatagtgatgatgaagtagaatcaactggttttaccgagtcaaggtctgctttctcaaactatgaagcttgccacaaggctgattcaggacagtactcttctttttggtcctctgactcatccaagctaaatccatataaactccgtcaattgctttccgtagttccaaactctgactctcacaaagaaatccctcttcgggaaagtgttttgcgactacaatttcaaactcgcgtatataatcatcttataaagacttacaatcaccaatcaaggtcatccacctcctcagattcctcatcatcctctatctcttcgcaaacatcatccttatcatcttcatcaatggtctccaatttatctgatcacgagagtgatgaatcgcaattatatagtgaaaactatcaaaacaaattcacaaacgctacaagttctatagctcaaaaaccgactcaaagtaaaaggaagcaagagtttttgaagattttgattccaaataaattcttttag

SEQ ID NO: 487 - DNA, 399 bp, Phakopsora pachyrhizi
  source 1..399: mol_type = genomic DNA
atgttccacagagcaacagtcttcatcactctcttttctgctctgtcaattctgaatgacaaccgtgtctcagctctaatggcatctgtacctgatactcagacctgcagcttctacaccggcgcatacaccaactcagccacttgcaacgaacaaccaaacgtggtctgcactcagggatgctacggtccctatgttgtggctactggctgtgtcttggagaacgactcaagggctggagcttcaagctcttctcaagtgtgcactatgggctttggacgcaacacggcagctgcaaaggcttgtattaacgaactgggaaccttccgctgcactggccaaactagtggctcagctacctgctacggctgtcgcaattcagctcaaaactctaagtag

SEQ ID NO: 488 - DNA, 567 bp, Phakopsora pachyrhizi
  source 1..567: mol_type = genomic DNA
atgtttatgacgtctctctgcacttcagctgttagatcaacagctattggtggaggtatcaaagttctaagctcattaacatcattagtatcagttccatcatcaaccagcttgcttcctcaacctggtaaccaatcaaggcgtggtaaagtgattttaagaggaatgtctagtcaaaccaaaagtacgatcgtatctaccgaggacgctccaaaagctattggaccatactctcaagctatcaaaactgctaatggattagtgttttctagtggctgtatcccactcgatccaaaaactatggagattgttggagaaaatgacgtccaagctcaaaccgaacaatgtttaaagaatctagaatcggttttaaaggcttctggtagctgtttaaattcggttgtgaagaccacagtctacataaaggacatgaaccaattttcaaaaataaatgaggtttactcaaaattcttcaaggacttcaaacctgctaggagttgtgtggaggtggctagattaccgaaagacgttctggttgagattgaggctatcgctactgtaatttaa

SEQ ID NO: 489 - DNA, 177 bp, Phakopsora pachyrhizi
  source 1..177: mol_type = genomic DNA
atgaaattctttctgggattgacaaatctgtttttgatcgcctctatagttggggtagttttctcatccccggttaatacaaatggagaagacaccagattacaaaagcgaaaaacggtagcgccagcaacctgcacacagggaggtagagttattaaaaataaatgcaaaatatag

SEQ ID NO: 490 - DNA, 552 bp, Phakopsora pachyrhizi
  source 1..552: mol_type = genomic DNA
atgctgttctgcaaatttttattaattttttcgttgatgtgctatggcgctctagctgctccgataatgagagttttttgggcggcttttccacttttaaatcttccagtcttcaaattcctcattcacccccaagagtctttgaagttacttcttggtttagagagaggatttcaggaaattaaagatctagaattgagtaagagtgcttttagacttcaaaagttgattttaggtgatcagaaagtgtttctgaaggaaattgacggcaaaattgaaaacgaagcaacaaccttcgcaaatgtcatgagacttgataataaaaagcagtttgaaaacatacccttcagactacctctaaatacaaaggatttgcatgaaagtatttataattctttagtagaagtagttaatgatcctaaaattattggcctccaaaggattcccaacccaaaattgacgcaaaaacgtttaagtgaattgaaacccatatcggagcccattgcagatattaatatacttcgtactgcagtggcgcattcaaaattataa

SEQ ID NO: 491 - DNA, 228 bp, Phakopsora pachyrhizi
  source 1..228: mol_type = genomic DNA
atgaactgtaacgatgcatgttcttgggttatcttagctgtttttcttggaatttcggccttaaatattgcttgtgtagaaggagccagcgtagttgggcccgaaaaatcaattaaagcccgtcaaaacgaggtgtcgtcgtactgtcgagttccgggagggtgtcgtcatactgtcgagttccgggagggtgtggtcgacgagatggccttctcgcttccctgctag

SEQ ID NO: 492 - DNA, 231 bp, Phakopsora pachyrhizi
  source 1..231: mol_type = genomic DNA
atgaactgtaacgatgcatgttcttgggttatcttagctgtttttcttggaatttcggccttaaatattgcttgtgtagaaggagccagcgtagttgggcccgaaaaatcaattaaagcccgtcaaaacgaggtgtcgtcgtactgtcgagttccgggagggtgtggtcgacgagatggccttctcgcttccctaaggagccagcgtagttgggtccgaaaaatcaattaa

SEQ ID NO: 493 - DNA, 204 bp, Phakopsora pachyrhizi
  source 1..204: mol_type = genomic DNA
atgaactgtaacgatgcatgttcttgggttatcttagctgtttttcttggaatttcggccttaaatattgcttgtgtagaaggagccagcgtagttgggcccgaaaaatcaattaaagcccgtcaaaacgaggtgtcgtcgtactgtcgagttccgggagggtgtggtcgacgagatggccttctcgcttccctgctaggttaa

SEQ ID NO: 494 - DNA, 156 bp, Phakopsora pachyrhizi
  source 1..156: mol_type = genomic DNA
attccattgatattagcttgggcaatgtcgattcataagagtcaaggacaaactttgaatagggttaaagtagatcttggtaaagttttgaaaaagctcttttccagagcaacctcactagaaggcttacaggttctgaggtttgatcatcgcaaa

SEQ ID NO: 495 - DNA, 891 bp, Phakopsora pachyrhizi
  source 1..891: mol_type = genomic DNA
atgaccctcagactgtttaaatgggttggctttcttcaactacttctgaggctcaccagcgcccaagacaccagtggaaccactcctctttaccagaaaaagtttccctggcccaacgtaccatacagagcggatacaggtgacggcccaagggggactcagcagggctacaataagtgtaattctactactcaaaatcaagattcattatgccaaaccacgtttttaaactcaatccaagagttctgtctctggggagccatcaagccaaactctattgttgggaatattgagggagaaatggtggcgtactgcacaacaaagaagtttggcactaggcttattccatcaggggcaataaccggcgtccaactaactcggaccccgggatatatccaagttgttggttttattgatcaaactgcgctcaacctacaagctaatgatacaggaggcgaagaagatcctcatggtgctgatcagcgcgggaaccccctaggagctctgatgtactctagcgcttttaactctaattctggggcctcttccggccaatcttcaaactctacaagttcatcctcttacactcaagtgagtgagtggagcttcttcattggtagcggagtgttctgctataaggcgtgtgacccggctggtcctaatgcacctcaactttgccaacatatatatgaccggattggctgtgcatacaacgctccagcaaactatccttccattaacggcacctaccaatcttgtcaaggagagaaccaattacctgcaggaacgtatattgaaaatggagtaactaagacttatcagcagcctccagagtcgcttggtccaattacgttggttgatttaattacctcgagtgattatttaggttga

SEQ ID NO: 496 - DNA, 447 bp, Phakopsora pachyrhizi
  source 1..447: mol_type = genomic DNA
atgtttgtttccctcaaggccataatttcgaccacaatcgctacccttgcggtgatcggagtcgtttctgggcagaatattactatagtctctccacagagcgggcaagtcatcagggcaggtgatgtgttaacagttgaactagcccaacaatctaccacatcatcggtccaaaacatcgcattcagcaccggtttcagttcaaggcccacagatactatttccttgggcagacctttaggcggaacccagtttttgactaccagaggaaacataacctttcaaattaatgtgcccaaagcaagtgaatttattgacgggatttccaatgtcccttacgaattcaaagtagcccattactatttccttggtccagcgttccagttagcgtacaacaataacttaaaaatcaaatcaaaacgagttctttcgattttaaatttttaa

SEQ ID NO: 497 - DNA, 258 bp, Phakopsora pachyrhizi
  source 1..258: mol_type = genomic DNA
atggttggatgtgagagcggtgcaacgaaaaagtctatgataatcaagatgattggattcttgtgcatttggtttcaactattagaaatagttttgactcaatctactgctgcaactactagtgccactgaaactaaaaattataatagttattttaaaaggcttgaaaaaagaagcccatcgaccagctctggtcgaattgagaaaagacatcgcagatgtggaccgccatatgggggatactatggacctggttaa

SEQ ID NO: 498 - DNA, 312 bp, Phakopsora pachyrhizi
  source 1..312: mol_type = genomic DNA
atggttggatgtgagagcggtgcaacgaaaaagtctatgataatcaagatgattggattcttgtgcatttggtttcaactattagaaatagttttgactcaatctactgctgcaactactagtgccactgaaactaaaaattataatagttattttaaaaggcttgaaaaaagaagcccatcgaccagctctggtcgaattgagaaaagacatcgcagatgtggaccgccatatgggggatactatggacctggatatagtagtggttattataactatggctattactctggatataagaaaaagcaataa

SEQ ID NO: 499 - DNA, 915 bp, Phakopsora pachyrhizi
  source 1..915: mol_type = genomic DNA
atgtttttctccaaatcgcttctcattacaatcttcatgggttgtctcattgatcggctccagtccctccccatcccctcattctatggcactagctctgtcgatcagaatccagattcaacccaggttgggttaaataactgtaatagtcaactctctgggacagagctcggatcttctggcggtagatctacgatcacctccccgtcgatcgcttcaggtgtctctggcaacggtgaaatcagttctgccgatcgaaacaaataccctgttacacagaatgcaggaaaaataaaccaaatggcaggctcagagaaagaagttggatcaggcctggcaaaggagcagtccgagagagatgctaagattcaaaaggatacccagacgcggcaggcgcaagagcaggcgattaccaacgctcaccagcaaaaggatgatgattctcgtaaagcccaggatcaggaagaaaagagaagagtgaaagaggaagagaatagaaaggaagcagatgaagagaagagaaaggaagcagatgaagagaagaagaaagaagtgggagaggaagaggaaaacaaaaagagggaagaggctcggcaagaagaggaaaataaaaagaaggaagaggctcggcaagaagaggaaaacaaaaagaaggaagaggctcggcaaaaagaagaaaataaaaagaaggaagagattcgacaagaagaagaacggaaagccaaagaagctgaggatcgtcaggcagccgaacaggccaatgaacaagaatctcgaaagcaccaagacaaaatacaagctccaccaccagggacagccggtgacgattcaggaaacaagccttcgaaaccctcgcaatcaagctcccaactcaaatccggtgatgccacctgtaagatatcatctgactgctaa

SEQ ID NO: 500 - DNA, 366 bp, Phakopsora pachyrhizi
  source 1..366: mol_type = genomic DNA
atgttccacgacttgaactactttgcacctgcccttcttgccttcacctgtcttcttgagctcagtgctgctcaagaacagaggtcctgcagtttctacactggtgccaataccacttccgccacctgcaacgagcaaccaaatgttgtatgcaccaagggctgcactgggcccttcgtcactgcaactgagtgtacgccggttaatgagtcggaagaagctattgcgagcactcaggtgtgcagctttggttttggtcggaacacagctgctgcaaaggcctgcataaatgaattaggtacttttagatgcactggtcaaaccacaggctcagctacctgtgacggatgtcaagctaggtcctaa

SEQ ID NO: 501 - DNA, 609 bp, Phakopsora pachyrhizi
  source 1..609: mol_type = genomic DNA
atgatgagagctagcaagtcaatcgtttttatttcagcatgtttttacctgttttctggagaaacacttgcagccccagctttaaaaggagtcgaaactgaggccaacagtattcctgatgcggcaaagtttgccgagagtctttcaatcaagacagatagtctttccgagctgccgttaaaatcgtctcctacaattccaagtacaccgtctggtttagatggcaatcctcaaagttcactgctagataaaagtgcaatgaaagaaattgatgcttcagaatcagcagaaaaaataaagagccattcttttacatctggtatctctgccaaaaggcctacaaagtcgcttgacgcgtcagcaacaggctccgacggactagttcaacaggtgcacgtcggtgattcgcaaatagctgttgatgcgaaaacggaggttccacccgcagccgcttatgagcacaataaagtaactcgaaaatgtaaccatgatcatgatcatgatcaccaccaccaccatcaccatcatcatggccctggtgacaatcatgatcatgatcaccatcaccatcaccatcatggtcatgatcatgaccatgaccatgcataa

SEQ ID NO: 502 - DNA, 1203 bp, Phakopsora pachyrhizi
  source 1..1203: mol_type = genomic DNA
atgtttttctccaaatcgcttctcattacaatcttcatgggttgtctcattgatcggctccagtccctccccatcccctcattctatggcactagctctgtcgatcagaatccagattcaacccaggttgggttaaataactgtaatagtcaactctctgggacagagctcggatcttctggcggtagatctacgatcacctccccgtcgatcgcttcaggtgtctctggcaacggtgaaatcagttctgccgatcgaaacaaataccctgttacacagaatgcaggaaaaataaaccaaatggcaggctcagagaaagaagttggatcaggcctggcaaaggagcagtccgagagagatgctaagattcaaaaggatacccagacgcggcaggcgcaagagcaggcgattaccaacgctcaccagcaaaaggatgatgattctcgtaaagcccaggatcaggaagaaaagagaagagtaaaagaggaggaggaaaagagaagagtgaaagaggaagagaatagaaaggaagcaggtgaagagaagagaaagggagcagatgaagagaagaagaaagaagtgggagaggaagaggaaaacaaaaagagggaagaggctcggcaagaagaggaaaataaaaagaaggaagaggctcggcaagaagaggaaaacaaaaagaaggaagaggctcggcaaaaagaagaaaataaaaagaaggaagagattcgacaagaagaagaacggaaagccaaagaagctgaggatcgtcaggcagccgaacaggccaatgaacaagaatctcgaaagcaccaagacaaaatacaagctccaccaccagggacagccggtgacgattcaggaaacaagccttcgaaaccctcgcaatcaagctcccaactcaaatccggtgatgccaccttttatggaactggacttggttcttgtggaaaaaccgataccgatacagattacatttgcgctgtctccaaaattctttacgacagtttcaacgatatgggtaatccgaacaataatcccgtgtgcggaaaaaaaattagggctcactacaacggaaattcagtcgtggtgaccgtgcatgatcgatgtgaggcttgtgctgttcatgatctggatttctctccagccgcgtttcagcagcttggatctaaggacgagggtcggctacatggcatgcaggtttaa

SEQ ID NO: 503 - DNA, 771 bp, Phakopsora pachyrhizi
  source 1..771: mol_type = genomic DNA
atgttcaagtcaaacttccttcttctgagtattttgatagctctatccggagtttactctctcccgaatggaaataatcggccatcagactcgagtgaccacccccagtcagagccacaagagggtcgaggtgtagggcgcgaacgcgtacgagtgcgttattcttatcgcggagatttccgtgacggcgacggagggcatcgctcatcccatcacaggcatggtcgaaagcgccctcatcatcgtcaccgcggtgatcaggatgatgactcaaaaggtgataaatcctttggcgatgaaggaaaggaccagtatgccaacgatctgagcagccaaagctctaacggctatgtccatggctctgagggcggatacaagagggatcctagcggaccgcaggggtatacttccaatccctcggctgaagaggcaagcaaagagctttacagttcatcagctcctgccacaccaaccaatgatcaccctgattcttcgacttccaccagtggccacgataattctggtggggatgataaaccaagcaaggatccctctggctatagctcatcaggacaagacccatctaaagattatagcaaacctagcaaggatagttatacggcggatcaatcatccggaaataagcagggtggatccggttctccgacaaaggaagagtccggaagtggcgataaacctaactctgggggctcaggctcagaaccatacttttcggctggaaaaagttctaagaatcaatatagaggataa

SEQ ID NO: 504 - DNA, 1008 bp, Phakopsora pachyrhizi
  source 1..1008: mol_type = genomic DNA
atgcgtataaaaatacttttactgataactttatggccagcgcttttgagttttgtggcagccactccggctcttttccaggctccaaatagttatagtggagaccaacctcctctaggagctcttgttgtaagacagattcaagcggtacctggagagttctcaaacttaagtcaagcaattggatccttggcaacaaaagaggggcctcaagttatttttatatatccaggacactatcgagaatcagtcaaagtcaactacaaatttcccttaataatccagggatatacaaaaaactcttcagattaccgacataaccaagttaaagtagaaaatgccgtaagtgcagcatcggtaggaggtaatgcgcaaagttctgcaatttgggctcgaacagatttttttactatgcttaacatcaacgtaacaaactcatatgggaatggaactgatactcaagcggtagccgtaaccgcagacggcgattaccaggtttacaagggttgcgtaatcgacagttttcaggatactttatatatcaaatcaacttcatcttatttttttagatgcttaatagctggaagtgttgactttatatttggtgctggaacagcatggtttgagaaatccaagatagcagtgaagcaatctcactccatcaacataattacagctcaaagaaaaacagtcctaaaaacccaattcgttttcaatgattcgcgagtagtagcacttggaaacgtggcaaatcagtcaacatacctagggagaccttggtctgaaaatgctagcgtgacttaccaattttgcaaactttcaaatatcatcaaacctgaaggatggagattctggaatcctaatgaccagaggacccagaacattaaattcttcgaatttcaaaacacaggccccggttctttgggtgaaagaaaatatgggaggagagctgaagcggcattaaacatcacagaagtattgggttcagaccctgaatcatggatttag

SEQ ID NO: 505 - DNA, 933 bp, Phakopsora pachyrhizi
  source 1..933: mol_type = genomic DNA
atggttttacctctccagatcattgtcatcagcatatttatttctctgaacaacctaagcgctgctaggctgtttggaagtgacggtataaggagaataatcagtggccggtctactcatccttctgttggagaccgacatggtctttcacctaggaatttgttaaatcaagaaagctacgacgattataaatccgatgaaaatttatatggatcgacaaccaagacaaccggtttaacaactgaacctgttaatctagtgcctgatcaatcaaaatttaaagtagctggcacatcgactaagcaagactatgttcaagctgttcctgctagcggactttctaccgtttcaagcgagagtccgaaggtccgtcaaatgagtcaagctgctgttacagaccttaatacttgtgataaggggcatccaaaagctgagatcaacaggtacttgctcgattgcaatgcaggaatcgggtcctactcagctgttataattgccaactgcaagtctattaataaatcaaacgcccagcagaccggaacattgattctggatggcctgagaggcatgaacaatgttttgaaattgacatccagctcaattacgagctgtggaatggactctaaaccactggctgttgataagggcgtcgatttgtcgattcttgctaactctgcagttggcatagttggcacactttaccctgtttttgagtctatctcgaaattgtatgaaccttacccattgattgagaagagctgcagtgaaactttgacaaacgtatctacaagtctttcgaatgtgattgcttcatgtggagaacaagtgaatctgtttgatacgaagttttttaaggagatcaaaaactacatgtctcaattctcggacgtaacaaacaacttcaacgctttcattggttcatttgattcataa

SEQ ID NO: 506 - DNA, 933 bp, Phakopsora pachyrhizi
  source 1..933: mol_type = genomic DNA
atggttttacctctccagatcattgtcatcagcatatttatttctctgaacaacctaagcgctgctaggctgtttggaagtgacggtataaggagaataatcagtggccggtctactcatccttctgttggagaccgacatggtctttcagctaggaatttgttaaatcaagaaagctacgacgattataaatccgatgaaaatttatatggatcgataaccaagacaaccggtttaacaactgaacctgttaatctagtgcctgatcaatcaaaatttaaagtagctggcacatcgactaagcaagactatgttcaagctgttcctgctagcggactttctaccgtttcaagcgagagtccgaaggtccgtcaaatgagtcaagctgctgttacagaccttaatacttgtgataaggggcatccaaaagctgagatcaacaggtacttgctcgattgcaatgcaggaatcgggtcctactcagctgttataattgccaactgcaagtctattaataaatcaaacgcccagcagaccggaacattgattctggatggcctgagaggcatgaacaatgttttgaaattgacatccagctcaattacgagctgtggaatggactctaaaccactggctgttgataagggcgtcgatttgtcgattcttgctaactctgcagttggcatagttggcacactttaccctgtttttgagtctatctcgaaattgtatgaaccttacccattgattgagaagagctgcagtgaaactttgacaaacgtatctacaagtctttcgaatgtgattgcttcatgtggagaacaagtgaatctgtttgatacgaagttttttaaggagatcaaaaactacatgtctcaattctcggacgtaacaaacaacttcaacgctttcattggttcatttgattcataa

SEQ ID NO: 507 - DNA, 747 bp, Phakopsora pachyrhizi
  source 1..747: mol_type = genomic DNA
atgcactcgaaagtgttgatatcgtgggtcatgagtgcctttgtggcaatcgtcctacatgcagctaattcgatccaggacaatgaggagccaccaatcgttgcaaaggtgtattttgatatgaaacaaggctcaaagaatcttggtaggattgagatcggtttattcaaaaatacgcccaagactagtgaaaacttcagagcacttgcagttggtgataaaaaatcaaaagatgggattcctctggcctacaagggctctaagtttcacagagtcattaaccaatttatgattcaaggtggagattttactaaaggcgatggaactggcggactatctatctacggaccaaagtttcaagacgaaaactttataaacaaacatactggacctggaactgtttctatggcaaactctggtcctgatacgaacggctctcagttttttatctgcactgctcttacttcttggttagatggtagacacgtagtttttggaaaagttcttagaggaatggatgttgtttttcgaattgaaactttacctactgacaatggagacaggcccaaggaagatatcgttatagccgactctggtgaactcaagatcgatgatgaacttgatgcagacggaaacaagtttgagaaagtaaagctagttacaggttccaataagcaattagatcaatcgatcaattcgaagtctgactcagtccagttctctagagaagctgatgggttaaaatga

SEQ ID NO: 508 - DNA, 678 bp, Phakopsora pachyrhizi
  source 1..678: mol_type = genomic DNA
atgtttagatctttatttttaggaattttagcttgttctgcagttaaacttaccatttgtagtccaagtgatgaggtagttgtcaaacctcgacagataggttatggagctggtcttggtgctggaggagcttcctcaagctctttcagtagtagtaccagcgtttcttccttcagttcgttcgttcaaggttggagtgtaattccaactgctttcggtacctgtgcagctgtctttcagagacaggtaacagttgaggtggccattcaatctgttcaggagttacatagtacagtatctggtgttctgggtaattatggaagttgttctagctgtggtggtgcttcggcagctagctcttactcgtctcactaccaaagtataattgtaaagacctttacctcatggcaatctatcatgtcagtcggtcattcactatatgacaatgtctgggaacctcagtttgctcctctctttcgacaattcaacccattccttacagccgttcaacagaactcaggtttctttggtatcaaccttggaaacattcttggtggtcttcatctaaacttaaacctattctcatcttgtggtcttaacattggtggattacttggaggtgttttgagtacggttggtggattgttaggtggtcttaaagatcatgaagaagcttga

SEQ ID NO: 509 - DNA, 348 bp, Phakopsora pachyrhizi
  source 1..348: mol_type = genomic DNA
atgttcacagttcttaaggtagtaccatcgatgttcctgatcctacaaactcttctggccttactagcaacgagtctgaatatcggcatggcaaaggatataccaccggcctcagtctgttatagttattcgaattcggataacgatcgtgccacttgtcacggtgccacctgccccgatggttgttcctggccctttataactgcccaaaactgtgttccaactggaccaacttcatctaaatctaccacccagacctgtcatattgggtattggaaagatggaactgacggcatatgcactaataggttaagagaaatgtatcgctgcagtggtaaaactactgga

SEQ ID NO: 510 - DNA, 564 bp, Phakopsora pachyrhizi
  source 1..564: mol_type = genomic DNA
atgttcttctctctctctcttcttcttctntctgcttcttcttcttccctgtcaataatctgtcccttgcccaggagatctcagactcatcatccttaccaacgaatcagacccaggagcaatcttatcaggatcaagcactcctcaaacgattcctcatccgaatctgactcagattctgactcatctctgaaatctaaccagatcggtagtggtaacgatggtcaaacgtttggtcaggatgatgaggtcctatctgaattcttatcactcttcaatccatcatcgaaagggtcgtttttgatcctatctgaaaacttgtcaaacaatatgacccatggctttgagaatgcggagtgcacttctgttggtagcaacctcaatgactttaaccagtcaatccatcaagtcagcggtagcgaacaagattggattgcttcttcgccaatttcaaggtgtctcaatccgtttagtaggcaagaaggcaataaaaacttgagatcggatgataagattcaagacaaggagggaggattgattgagaactttaaagagttgctgaag

SEQ ID NO: 511 - DNA, 873 bp, Phakopsora pachyrhizi
  source 1..873: mol_type = genomic DNA
atgtatcacttaacctttgtcctcttttcactcttttttttgtccgctaacggagcttctacttcggagccaagcggacagaccctagattgctcccactacactggcgcgaacaccaaagaagcaacttgtaacgaattccctggcagaatttgtcacggaggttgcactggagcagttgtggccagtaattgtaccttgaaccccggcgaagaaccaaaagatcaaacctgcaccatagcttttggaaaatcttcagctacgatttcaatctgtcgcaatgagaagggatcgtattcttgcacaggacctatcaaaggaagcgcaaagtgctctgtttgtgttgaccctccaacaggttccaatcaaagcccaaccacgcctgctcctgctccaggaaacaacggatcgacttcaaaagaaggccaaactctacagtgcactcattttactggtgctaatactcaaagcgcaacttgtaacgaggttcctggcagagtttgcaacaagggttgtacctcatcagtcgttgccaccaagtgtacgttgaatcctggtgaccaagagagtcaacaaaactgtacccaagctttcgggaaatcctcagctgcgatatccatttgcatcaatgacaaagggtcattttcatgcacgggaagcgtcagcggaaatgccacttgctctggatgtaccgatcaatcatcatctggttcctctgaataccctggttcttctggaaccaccaagccccctactgaccctactggtggtgaaaacaaagaccaagacaagaaggccgaagcaaccagcttgcaattcgccatgagctctttctgcttagctttgtctctaatgattggtgtcgccgtgctc

SEQ ID NO: 512 - DNA, 933 bp, Phakopsora pachyrhizi
  source 1..933: mol_type = genomic DNA
atggttttacctctccagatcattgtcatcagcatatttatttctctgaacaacctaagcgctgctaggctgtttggaagtgacggtataaggagaataatcagtggccggtctactcatccttctgttggagaccgacatggtctttcacctaggaatttgttaaatcaagaaagctacgacgattataaatccgatgaaaatttatatggatcgataaccaagacaaccggtttaacaactgaacctgttaatctagtgcctgatcaatcaaaatttaaagtagctggcacatcgactaagcaagactatgttcaagctgttcctgctagcggactttctaccgtttcaagcgagagtccgaaggtccgtcaaatgagtcaagctgctgttacagaccttaatacttgtgataaggggcatccaaaagctgagatcaacaggtacttgctcgattgcaatgcaggaatcgggtcctactcagctgttataattgccaactgcaagtctattaataaatcaaacgcccagcagaccggaacattgattctggatggcctgagaggcatgaacaatgttttgaaattgacatccagctcaattacgagctgtggaatggactctaaaccactggctgttgataagggcgtcgatttgtcgattcttgctaactctgcagttggcatagttggcacactttaccctgtttttgagtctatctcgaaattgtatgaaccttacccattgattgagaagagctgcagtgaaactttgacaaacgtatctacaagtctttcgaatgtgattgcttcatgtggagaacaagtgaatctgtttgatacgaagttttttaaggagatcaaaaactacatgtctcaattctcggacgtaacaaacaacttcaacgctttcattggttcatttgattcataa

SEQ ID NO: 513 - DNA, 1008 bp, Phakopsora pachyrhizi; identical to SEQ ID NO: 504
  source 1..1008: mol_type = genomic DNA

SEQ ID NO: 514 - DNA, 204 bp, Phakopsora pachyrhizi
  source 1..204: mol_type = genomic DNA
atgaagaaaattttggatttatatatgatctgtgctttgagttttctctacagttcacttgcatcgatggctatgtgccagcttggcttttatagtttaagaacaattctgctattgctatcaacatgccccaaagcactgctaaaaaaggaattaacatttaattcgaaaacgagggctacatcaatcagtttaatggtggaa

SEQ ID NO: 515 - DNA, 162 bp, Phakopsora pachyrhizi
  source 1..162: mol_type = genomic DNA
atggtacttggtcgagtaactcattacgcattcgatctagccttagtatcgactgtccttgctggagtcaagcgaaacactggatacagtttgaaaactgattccttgcctgaaggtgtgcctcagagtttaatctctacttacctcgttgtcggtgaaaac

SEQ ID NO: 516 - DNA, 426 bp, Phakopsora pachyrhizi
  source 1..426: mol_type = genomic DNA
atggctttattaaatcgaattgtttttgttttgctcgccttgacctttgcaggagctattgaatctagaactggcgttaaattagactctaggctgaacactcgtaatacgcaagagcaattaacccatcctcgtgtaaaatgtggtcgatctactgctactcaagctactggatccacttatagatgtaggaacagctcaaatgatggaaagaagcacaaaaaggtacccaaaggttatggcaaaaaagagaaaggcaaatacacgccaaaaggtcctaaacatgaatcctatgctccgaaagctccatcaaaactccccaccggcggttatggcggacatgagcaggaacaatatgcgccaaagggacattcctcagagccttacgttccgaaaggagttgtctcgtcgcctggttactcttaa

SEQ ID NO: 517 - DNA, 141 bp, Phakopsora pachyrhizi
  source 1..141: mol_type = genomic DNA
atggttttccctcagcaaatttctcgcagtcgaaaaatcccactccgtttagtattgctgagtctggagatggtggtattattgacccgcgatagccctctggtgtcaacaactgatcagcgatattttgacttaaactag

SEQ ID NO: 518 - DNA, 225 bp, Phakopsora pachyrhizi
  source 1..225: mol_type = genomic DNA
atgaggggccgtcagattgatacaggctttcttgtcttttgggttaagttgtacttcctgttgaagacggttacgggtggcttggtgacaaagcctgatagcatcgaaaaactaagtggtagtggtgtcaaaattgaagactacacttcaaaaggttattctccgctgttttttcttcccttctatctcccgtcacaggatgtaataaaaaacttcataagctga

SEQ ID NO: 519 - DNA, 219 bp, Phakopsora pachyrhizi
  source 1..219: mol_type = genomic DNA
atggcgagatcagttctaaagttcaaagattatctccaactcgccatagtactccttacgatctatcaatccatcttgtgtgttggttccaatgtccgaaatcatattcacaggaggcatcagccatcagcttctgatccaaaagctagtccaaccagacctttggaatggggcgatttaaacatcattcatacaactgattctcatgtttgtttgtga

SEQ ID NO: 520 - DNA, 174 bp, Phakopsora pachyrhizi
  source 1..174: mol_type = genomic DNA
atgaacatgatcttaaagaagatcaacagcctgattttagcatttctgttaaagttaaagagcagtttacaaggtcagaaatatcatcatttgataatagatatagatctacaatttttgaaagcgcatttcgcattcaatggaatggtttcgagacactcaaaaaatacataa

SEQ ID NO: 521 - DNA, 117 bp, Phakopsora pachyrhizi
  source 1..117: mol_type = genomic DNA
atgacgataatgataatgaaagtgatgatgatgatgacgatgaagggcaggatcaagaagatgagtcggatacaaatgacgataacaataacgtgtagagatggctataatttttag

SEQ ID NO: 522 - DNA, 249 bp, Phakopsora pachyrhizi
  source 1..249: mol_type = genomic DNA
atgaaggtttggataggttcactggcagtcctttcggtgctgtcggtctacatgttggttcacggtagagagttcattaacctacccaagttgaacaccagggaagtcataatcaagtacgacggacctggataccagagctctaaaaacggaactggtctcaggggccatcagagctacacgagcctcaccagtagtaataagaggagtggtggtggcgataaagactttatcaagaagactttttga

SEQ ID NO: 523 - DNA, 261 bp, Phakopsora pachyrhizi
  source 1..261: mol_type = genomic DNA
atgtttattaaatcatattgttttcttcttggtctcacgcttttcctttcatgtctttcgactactataactggagagttaaacaatatggatgaccaaatactagcagctaaatttcgagcaaggcgttctattgaaacaatcaaagaaaatagtcctataaaacaagaacggaaaaaacttcgtaatgaaccagtaaacacgctagtcgcttatccagtgagggatcccaattgcgttgatgatgatgctccaagatag

SEQ ID NO: 524 - DNA, 255 bp, Phakopsora pachyrhizi
  source 1..255: mol_type = genomic DNA
atgcgctgcttcattttatcgatagtcattttggccgtagcccaggcagtcacggcgatcccgcttgttgaacgagctgaagttaactcatcaaaaggtgatactttgaaaaagcgttttggctgcattgacccgctagttagcgttgatgttctaaaccgaggcaatggagcgagaggaagtcttctagacgtaaacatcttgaacggattcttgaaagatgcccaaaagaagtctcaggctaaaaatgagtag

SEQ ID NO: 525 - DNA, 426 bp, Phakopsora pachyrhizi
  source 1..426: mol_type = genomic DNA
atggctttattaaatcgaattgtttttgttttgctcgccttgacctttgcaagagttattgaatctagaactggcgttaaattagactctaggctgaacactcgtaatacgcaagagcaattaacccatcctcgtgtaaaatgtggtcgatctactgctactcaagctactggatccacttatagatgtaggaacagctcaaatgatggaaagaagcacaaaaaggtacccaaaggttatggtaaaaaagagaaaggcaaatacacgccaaaaggtcctaaacatgaatcctatgctccgaaagctccatcaaaactccccaccggcggttatggcggacatgagcaggaacaatatgcgccaaagggacattcctcagagccttacgttccgaaaggagttgtctcgtcgcctggttactcttaa

SEQ ID NO: 526 - DNA, 777 bp, Phakopsora pachyrhizi
  source 1..777: mol_type = genomic DNA
atgtttaagaagagtactatgattcttacttttctctttttggctttacacgggactctgtcttcaccagtgccagattctttgacctctaatgataataacgatataaactttagctcagaaatatttgcgcgagctgtccaggatgaatatccacaagagcaggagaactcaaggagtggcataccagggaacgaatttgaaaatagcaacgtagtcaataatgcaagagacccagcagagttgaacgagcagaatgaacaaattcgcggtgataatcaacctactactaactctaatattcaactcaatcttaaatcaagcaccgaggacaagacggcaaaagattcaccaagaaaaaggtctccagaatcctttgatacaactagcagcgtacaaccttctcccagagaatcaaatggggaaatcacaaatcctcaagtcaatgatgtttctcacagcgatcagattaactcaggtaactctccgactgagggctttcatccaggagatttgaacacgagcggagatcaagggttgtctaaaatctggccaaacaaggatgaaccccaagtgaactatgatcctcacaaagatcaaacaacctcagaaagttttccgacacagggctctactcaaggtaattttcaacctcagccatctagggataccactcagattaacgaaaatccttcgattgatcaaacaaactcaggtacaactactccaaatccagtcaattctcaagatggtaataagccacatggtgataaatga

SEQ ID NO: 527 - DNA, 153 bp, Phakopsora pachyrhizi
  source 1..153: mol_type = genomic DNA
atggtcatgattctaactgtctactggtactcgatcctgcaaagttttgttgaagatgatgatgaaaaaaatcagatgttttcattctactctgcttattctcccctttactcatctgtctctgtctctaaagatctatctccaggcgcttga

SEQ ID NO: 528 - DNA, 963 bp, Phakopsora pachyrhizi
  source 1..963: mol_type = genomic DNA
atgatgatgatgatgatgatgatgatgaggcaagcgaccaagaagagtctaggtagcgttaacctgtcgtctaccagggccatccatcacatagctcccttctctaaaatagcctcagtagcaccattactctcagcgttaacatcctcttcccactctcaaaggttttctttaaataatcaactcactcacacctcatcctcggttcgaagtttctttagtctgccttcctttcctcctagtctgaatgtttcaaaggatattcatgggtacgaagacttaaacgtcagagacaggtattctcagagaggagacgttcttacctacaaggagactaaacgattatctttctctaaacatcagctatactacatcgcagctgatatagagtcatattcgagcttcttgccctactgtgctcaatcaaacctaagatcggtcagagcgttggacaagaccagcgatgacagtttagaaagcaatctgataactaatgacgagaggagacaatggttgaggggtggaaaggttggtgagacttttcttatcgagttggagcaggtcaacggtttcaagggcttggacgagctcaatagaacctctgtagaatgtagaaagttcgatctggtaaagttgacaatgtgtgattcaaaacttttcaagagcttgagcataacttggaagttcaaatccccaagagagatcttcaacatcgattcgatcttaaataaccaaaatggcttaacttcacagtcatcctcgttgagagaggaggataatgaggaaactggggatgctttcgtgtcaatcgagacgtcacttaccttactctctggtgatatcaaccagcagtcaggtcaagttaaaggaccaaagtttagtgttaacgatctgagcggagaactctattggaaagccatgagtgaaaaggtcttggccagctacgagagtagactcaatcag

SEQ ID NO: 529 - DNA, 900 bp, Phakopsora pachyrhizi
  source 1..900: mol_type = genomic DNA
atgatgtttcagagcaaactcttgggtcttttaattttgtccctttatctctgtaaaacttctttcgctatcccgttgggcgatgatcagaaggctgatccagtagatacctctggattcaccatcatatctgatctctataataaatcttacaacaatcataacgtcaaaactgatcatccggaaccacccaagtacacagatccaaaaaaaccagatccaccgaactggaaggagcccaaacatccggaaccacccaagtacacagatccaaaaaaaccagatccaccgaactggaaggagcccaaacatccggaaccacccaagtacacagatcccaaaaaaccagagccaccgaactggaaagagcctcaacatccggagccaccaaactggaaagagcctaatcacccagacccacctgaatggaaagggcccgaacatcctgagccgccaaagttgaaagaaccaaagcatcctgagccgccaaagtggaaggtaccaaagcatcctgagccgccaaagttgaaagaaccaaagcatcctgagccgccaaagtggaaagaaccaaagcatcctgagccgccgaagtggaaggtaccaaagcatcctgagccgccaaattggaaagaaccaaagcatcctgagccgccaaagtggaaagagccaaagcatcctgagccgcctaagtggaaagagccagagaaacctgagccgccaaagtggaaagagccaaagcatcctgagccgccaaagtggaaagagccagagaaacctgagccgccaaagtggaaagagccagagaaacctgagccaccaaagtggaaagagccagagaaacctgaaccaccgaaatcgaaggagccagggtatactgatcctcctaaggagaggaactga

SEQ ID NO: 530 - DNA, 171 bp, Phakopsora pachyrhizi
  source 1..171: mol_type = genomic DNA
atgacgaatggcagttgggtgatagagagtggtgtgatgaggttaaagatgtttatcagggaaaaacaacccaaaactgacaatatcctaagtattatttcctatcgtctatctatctctctctctctctctctctctctctctctctctcacgcacaaagacttcaaaaa

SEQ ID NO: 531 - DNA, 375 bp, Phakopsora pachyrhizi
  source 1..375: mol_type = genomic DNA
atgaggataattgcctttttcgcactacttgctgccatctgcctcccatttgtagagaacagatgccatagaagtggagccacattcggatcgtctcccaatagagcttatgctactcagttattgcctggcgtatgcaaagaattccaaggcacctatggaccaaatcaggacaagtccgtttgtagaaatggcagagatagagataccagcttcagatactatatcaaacacgtctcagatgggtataggagcattggcccaactgagtgtacagatggcctcaacaaagagatagtcaattgcgatagaggtggaaaaacggcttatggcaactgggaatacaatgttgaccctaacaaaggaaggtgctag

SEQ ID NO: 532 - DNA, 426 bp, Phakopsora pachyrhizi
  source 1..426: mol_type = genomic DNA
atgatcagcaaaaatctgaagcttgacttaaagagcattttgatcattggatgttttttaacttcctgcctgctgcacatggttgtgactattgatatcacttgtgatacaaaagttgaattaaacgatgatgataaagacaatccaaaagttatttgttcttcatttggattacctcctgcgccttttgagtgcccaaggaaaagttgtaacgattttccaattggtttgcaatgtgttcaatttatgaatcagaagaacttgccaaacttccgcccctctaaaatacacaaatctgaactcacatgtcaaacgtttgaatttgatctgggaaaaaatgtttgtattacagaagatggcgaaaagtaccgttgcgctgggaatcgagaaccaatgaaatgtactgattgtaaaagaatacgatga

SEQ ID NO: 533 - DNA, 99 bp, Phakopsora pachyrhizi
  source 1..99: mol_type = genomic DNA
atgagaaacaaatggcgtgtaatttatttttgttttattaagctaattatttcatttggttttcgaaagtttaattttttttataaagtaaaaacataa

SEQ ID NO: 534 - DNA, 246 bp, Phakopsora pachyrhizi
  source 1..246: mol_type = genomic DNA
atgcgctgcttcattttatcgatagtcattttggccgtagcccaggcagtcacggcgatcccgcttgttgaacgagctgaagttaactcatcaaaaggtgatactttgaaaaagcgttttggctgcattgacccgctagttagcgttgatgttctaaaccgaggcaatggagcgagaggaagtcttattgatttaaacatcttgaacggattcttgaaagatgcctcaaagaagtctcaggcttag

SEQ ID NO: 535 - DNA, 417 bp, Phakopsora pachyrhizi
  source 1..417: mol_type = genomic DNA
atgatgatgaaatcattatacctgtcaacatttctctttggtattttcctaagatctagcgctggattacctcatcacagtgacccaagtacaattttagctagaggccttgagaatagatataataattggggaaagggaacttactattatgtttcagacggaaatcaagtagcttgtggagggtattatcaagactctgacatggtttgcgcagctagtgcaacggacttcaacaacaaaaatagttgtggagaattaattactgtgaatcactcgggcaagagtgttcaatgcactattgttgatcagtgcgaaggttgttctgctggatcgcttgatctatcaccagctgcttttcaagccctagctgaactttcagtaggcagaattgacatatcgtggaattttaattaa

SEQ ID NO: 536 - DNA, 174 bp, Phakopsora pachyrhizi
  source 1..174: mol_type = genomic DNA
atggttcgatctacgattaaattattattctttttttttaacctgtcttctctgtctgtttttttgaacagtaccaaagatgcaagtttcctctcttctgagatccttttacaaatcaatcaaactttcagattcgtttcgatcaaaaaaaaaaaaaaaaaaaaaaaaagataa

SEQ ID NO: 537 - DNA, 186 bp, Phakopsora pachyrhizi
  source 1..186: mol_type = genomic DNA
atggttcgatctacgattaaattattattctttttttttaacctgtcttctctgtctgtttttttgaacagtaccaaagatgcaagttttttaacaaacttcctctcttctgagatccttttacaaatcaatcaaactttcagattcgtttcgatcaaaaaaaaaaaaaaaaaaaaaaaaagataa

SEQ ID NO: 538 - DNA, 156 bp, Phakopsora pachyrhizi
  source 1..156: mol_type = genomic DNA
tcagatgcagtaaaagcggatgactactggagtactcatagatcgtgatatgaaagaaaacttgttgccaaaaaaacccaagcaacactttatagtcctttggtagcaaagagtcctaaaacatagtttaagcggacttctggtaaaaaaaaacat

SEQ ID NO: 539 - DNA, 420 bp, Phakopsora pachyrhizi
  source 1..420: mol_type = genomic DNA
atgattggaaagtttcttttgatggttcttttttcatcgattttcttggcttttggaaccattggccagagaatacctgaaggtttatacgtgtcttgtgccaatgccgttgtgagacagagctgttctgctgctttggatgttgcttttactgaagggaggatcagaagattcaccactgctcgtcaatacattcaaacccaagggtgcagactctcttggtgggtagacactggtatacttcgtattgctcaaaaagacgtacttcaagcagctcttcagcaaattgacgattcctgtactgcaaacggtgtttccgtcataaatcctagcaatcttccaggtcgaggggtttacgttggccagatggtcggcaatcaatacgctgttcttagagctgaaataggagctttcgcctaa

SEQ ID NO: 540 - DNA, 201 bp, Phakopsora pachyrhizi
  source 1..201: mol_type = genomic DNA
atgaacaccatcagctcgatttgccttgctattgcagcttttttgattgcccagaacaatttcgccggcgccactgacctaaaccctcgggatgaggttgcgaattcggctcatacacaattggaaccaagagctgttgggcggtacaatcaccttgaggctcgtaagaagaagcctaaaacacgaaagggcggtagatga

SEQ ID NO: 541 - DNA, 663 bp, Phakopsora pachyrhizi
  source 1..663: mol_type = genomic DNA
atggtttactcaaaggcaaccttcaccctggtctcactcctacctatgatcctggcccagcagagcatcaacaacagtgataccacaaccacggctgctaccccggccagtcccaccataggctcaactggtactaccaacagcagcagctctaccactacttccactaccacctctaacactgccagtggagctgctcagtccttccctgcgccaacagtctcaggaacatcctgtggtacagcccagcagaactttaaccagtgtgttaccaaagtatcaaaggatatctcctcatgtccctctaccgataacacctgtctttgccaaacgtatgcaaacctagcctactgttacaacgcctgtccagatctggcatcatcaggggccggatacctgcagcagtccacagtgaactgtgatgcagcaggtatcaagccaaacgctacttccaacgtcaccaccacccctgtcacctccaccactaacaggaacaccaccaacacctcacccatctctaacacaaacaagaacaattctacctcgggcaacactgctgctacatttgctgctggaaactccaaggcaagtgggattgaggccccgttactgagcgttgctgttgttgggctgtgtggtatcattgctagcctatttgcatga

SEQ ID NO: 542 - DNA, 870 bp, Phakopsora pachyrhizi
  source 1..870: mol_type = genomic DNA
atgatgtctaaactttttcttattgctgctattactgcattcgttccgtcaagctcttatggaagctccaacacacctgcttcaccacaaggtagcttgggaaacatatctccattcaaagaaaacgtcttaagcttaagctgtggactgaatcttttagttggtggaagagttgccccattagcacaatctggctacgcaaatttatgccttcagtgtaaaattgacctctttggtgttcgcgtgagctttgcttacaccagcgctctttcagaatctatcaacacccatggtttatcggttagcgacacctcccgcctccagggcttcgttcagggacggctgttagactcggctgctcaatgttcagccaaaaatgagtgctcgcggaaatgctcagacgatcaattctgtcaatccagcgatttcagtaatgggcgctgcacttttacagctaaaaatctagccagcaacaacaaaaacggtgccctcctagcccaagcatttgcagacgtatttaaccaaggaggaagccagtattgtggattttgcagtggtgattcatcatgtgtcaaccctagcgctcgtgcgcgaagttataagcgccatgcggaaattgcaaggagaagtcaattgcagacaaaatgtcccactggccttactgcctgtccaattgttcaaatgaatcagacggcagccgaagtctggagtccatcaccggcagtgtcttatgagtgcttgaaggtagatgaagaattgagctcctgcggtggttgcgcaacgctgggcaaagggacagactgttcagctgttgagggtgttttgtctgctggatgctctgatgttcagttaggaagaatccgcagttga

SEQ ID NO: 543 - DNA, 984 bp, Phakopsora pachyrhizi
  source 1..984: mol_type = genomic DNA
atgatgtctaaactttttcttattgctgctattactgcattcgttccgtcaagctcttatggaagctccaacacacctgcttcaccacaaggtagcttgggaaacatatctccattcaaagaaaacgtcttaagcttaagctgtggactgaatcttttagttggtggaagagttgccccattagcacaatctggctacgcaaatttatgccttcagtgtaaaattgacctctttggtgttcgcgtgagctttgcttacaccagcgctctttcagaatctatcaacacccatggtttatcggttagcgacacctcccgcctccagggcttcgttcagggacggctgttagactcggctgctcaatgttcagccaaaaatgagtgctcgcggaaatgctcagacgatcaattctgtcaatccagcgatttcagtaatgggcgctgcacttttacagctaaaaatctagccagcaacaacaaaaacggtgccctcctagcccaagcatttgcagacgtatttaaccaaggaggaagccagtattgtggattttgcagtggtgattcatcatgtgtcaaccctagcgctcgtgcgcgaagttataagcgccatgcggaaattgcaaggagaagtcaattgcagacaaaatgtcccactggccttactgcctgtccaattgttcaaatgaatcagacggcagccgaagtctggagtccatcaccggcagtgtcttatgagtgcttgaaggtagatgaagaattgagctcctgcggtggttgcgcaacgctgggcaaagggacagactgttcagctgttgagggtgttttgtctgctggatgctctgatggtaaatgctatactttctcttgtcaagatggatacaaactttcaaatattacaaacaaatgcactatgataaaatctaaatccaacttataccaaaaataccctccatccgtagttcagttaggaagaatccgcagttga

SEQ ID NO: 544 - DNA, 597 bp, Phakopsora pachyrhizi
  source 1..597: mol_type = genomic DNA
atgcaatccaactttctcctcgctttttgcttgattgctgctgtctctgttccagttagcagggctcacggtgttattacttcagttgagggcgcaaatggtcaaactggatctgcctttggaatggtagagtccactcctcgtgatggcactcgaacgaacccattccaaactgacagttccatcattcgagaccgtgaggtttcaagcggaaaggcgtctgcttgtggacgcactcttgcgggcggaaacaacgatattgctagtgacatgtcatcggctgaatctgccggattggccagtattggacctgacggaaaagtcagaatgactcttcaccaggtcaacggtgacggtggaggcccatacagctgtgaagttgacaccaccgccactggtgacaacttcaagaaaatgaacatcgacacaaacgttcctggcaagaacagtcgatcaagggcaaaggctactgaatttccacttgttgcaaccatgcccgctggcgctacctgcactggtggtgctgacggccaaacatgcttggtacgatgcagaaatgtgaggtttatttttaaatatcagatgtttatttcttga

SEQ ID NO: 545 - DNA, 183 bp, Phakopsora pachyrhizi
  source 1..183: mol_type = genomic DNA
atgataatggacttgcttcttgtggtttcactagttggtctttcctatggatccgagcatcaggaatatagaagttattatccagattttgaggaaagaaacctttttcagcaaaccggtgaaaacttgctacagagtttgggtctggaatatggctgcttgaaattttggttgggagcataa

SEQ ID NO: 546 - DNA, 384 bp, Phakopsora pachyrhizi
  source 1..384: mol_type = genomic DNA
atgtttatctctcctttaattttagcatttacttacattatagctagtataggtcaatcgtgctgtctaggttccttagatcagaatcagccccaaaagtctcactcatcactccagaaaagagcagtttgtctagttcctaatgcccgaaaggggtgtaagatttggaacatagacgcatgttgccagtcttgctgctctcaaaaatatgccaccagaagatggctagaggattgtcactgtgtgaatcaaggctcttctgtggtttatcaggcaccaggcacggttagctatgtaccagtccaacagcctccgacgacatatgtcgtttctagtcctcctcctcctcctcctcctaccgttgtaacagtttaccatggctaa

SEQ ID NO: 547 - DNA, 438 bp, Phakopsora pachyrhizi
  source 1..438: mol_type = genomic DNA
atgaattcgaagatttttttgatgctggtggtttgcacctttttttataccccttctggtgctagcaaaagtcttaaagttcgtgctgaaaaaggctaccattcttctggtcacaaaggtcacgggcataaatttaagattagggctgagactggggacaacattgtcacacagccaaccattaaccgcagctatggccatcacaagtatcaccacaagggctatagcaagcactacagaaagcgtgcgctgaaagacgttggaaaagaagctttagaggctaatgcagcgagtggaaaagccgcacctactgctgatagcggagctaaagcttcagttgttagcagaaagcatgggcatggccaccacaaaatgcacaacgcttatcacaaaggttaccgcaagcattatcgaaagcgagaaaaagctgtctattag

SEQ ID NO: 548 - DNA, 702 bp, Phakopsora pachyrhizi
  source 1..702: mol_type = genomic DNA
atgaattcgaagatttttttgatgctggtggtttgcacctttttttataccccttctggtgctagcaaaagtcttaaagttcgtgctgaaaaaggctaccattcttctggtcacaaaggtcacgggcataaatttaagattagggctgagactggggacaacattgtcacacagccaaccattaaccgcagctatggccatcacaagtatcaccacaagggctatagcaagcactacagaaagcgtgcgctgaaagacgttggaaaagaagctttagaggctaatgcagcgagtggaaaagccgcacctactgctgatagcggagctaaagcttcagttgttagcagaaagcatgggcatggccaccacaaaatgcacaacgcttatcacaaaggttaccgcaagcattatcgaaagcgagcttttggcagtgaaaaaaataaagcgaacaaaacccaagccagcacaaagaagaatttcggcgagaaaaagctgtctattagagcaacccctgagctatctggcaaaacggcttctattgatcaaacacccgttgcgaaagctggcttaactgaaaaggctgagaaaagcattgctaatggaactgtaccctcacaaggtacggcagtgcttcgaaaaactgagtcgtactacaggccgaagcactatggctacaatggatatagaaactcgtactactag

SEQ ID NO: 549 - DNA, 619 bp, Phakopsora pachyrhizi
  source 1..619: mol_type = genomic DNA
atgaattcgaagatttttttgatgctggtggtttgcacctttttttataccccttctggtgctagcaaaagtcttaaagttcgtgctgaaaaaggctaccattcttctggtcacaaaggtcacgggcataaatttaagattagggctgagactggggacaacattgtcacacagccaaccattaaccgcagctatggccatcacaagtatcaccacaagggctatagcaagcactacagaaagcgtgcgctgaaagacgttggaaaagaagctttagaggctaatgcagcgagtggaaaagccgcacctactgctgatagcggagctaaagcttcagttgttagcagaaagcatgggcatggccaccacaaaatgcacaacgcttatcacaaaggttaccgcaagcattatcgaaagcgagcttttggcagtgaaaaaaataaagcgaacaaaacccaagccagcacaaagaagaatttcggcgagaaaaagctgtctattagagcaacccctgagctatctggcaaaacggcttctattgatcaaacacccgttgcgaaagctggcttaactgaaaaggctgagaaaagcattgctaatggaactgtaccctcacaag

SEQ ID NO: 550 - DNA, 1206 bp, Phakopsora pachyrhizi
  source 1..1206: mol_type = genomic DNA
atgcagaccaaaattcttctagctctttgcctagtagctatctctcaggttaatgcgcatggtgcaattacagctgtccaaggctctaacggcatgactggagaggcttttggagtagatcagagcactcctcgtgatggtacaaaacgaaacccatttcaaaccgattcttcaattattcgtgatcgtgagattgctagcggcaagtcatcagcctgcggcagaaccttggctggtggaaacaacgagattggagcagcaatgtctaaggccgagtctgctggtatcccaagtgttagctccgatggaaaggttcagatgactttacatcaagttaacggtgacggtggtggcccttacacctgtgatgttaatgcttccggtgatggaaagactttcacacctatgacaatttctactaacattccgggaaagaacagcagatctaatgccaaggctgaggatattcctttaattgcggagatgcccaaaggaatgacttgcaccggtggatctgacggacagacctgtatcgtaagatgcagaaacgctgccaacgctggtccctttggaggatgtgtagctgttactcaggctacggatgctacaaagcagaagcgggaactatcagcagaacagagaattgatgagatgatcaaacaggctgaggctgccgaggcatttggcgatgagactcttgcaaagcgtgaaatgtctgctgacgagagattggacgaaatgatcaggcaggctgaagatgctgagctggctgaggaaaacctttccaagcgggagttgactgctgaccagcgattggatgaaatgatcaaacaggccgaagccgctgaaattgcagaggagaacttggttaaacgggaattgactgccgaccaaagactagacgagatgatcaagcaagccgaagccgctgagcttgcagaggagaacttggccaagcgtgagctgactgctgaccaaaggctggaccagatgatcaaacaagctgaagatgctgagctggctgaggaaagcctttccaagcgcgagttgactgctgatcagcgattggatgaaatgatcaaacaggccgaagccgctgaaattgcagaggagaacttggctaaacgggagttgactgccggccaaagactggacgagatgattaagcaagctgaagcagctgagcttgccgaagagttgatctaa

SEQ ID NO: 551 - DNA, 1206 bp, Phakopsora pachyrhizi
  source 1..1206: mol_type = genomic DNA
atgcagaccaaaattcttctagctctttgcctagtagctatctctcaggttaatgcgcatggtgcaattacagctgtccaaggctctaacggcatgactggagaggcttttggagtagatcagagcactcctcgtgatggtacaaaacgaaacccatttcaaaccgattcttcaattattcgtgatcgtgagattgctagcggcaagtcatcagcctgcggcagaaccttggctggtggaaacaacgagattggagcagcaatgtctaaggccgagtctgctggtatcccaagtgttagctccgatggaaaggttcagatgactttacatcaagttaacggtgacggtggtggcccttacacctgtgatgttaatgcttccggtgatggaaagactttcacacctatgacaatttctactaacattccgggaaagaacagcagatctaatgccaaggctgaggatattcctttaattgcggagatgcccaaaggaatgacttgcaccggtggatctgacggacagacctgtatcgtaagatgcagaaacgctgccaacgctggtccctttggaggatgtgtagctgttactcaggctacggatgctacaaagcagaagcgggaactatcagcagaacagagaattgatgagatgatcaaacaggctgaggctgccgaggcgttcggcgatgagacccttgcaaagcgcgagctttctgctgaccagcgattagacgaaatgattaaacaggctgaagatgctgagctggctgaggaaaacctttccaagcgggagttgactgctgaccagcgattggatgaaatgatcaaacaggccgaagccgctgaaattgcagaggagaacttggttaaacgggaattgactgccgaccaaagactagacgagatgatcaagcaagccgaagccgctgagcttgcagaggagaacttggccaagcgtgagctgactgctgaccaaaggctggaccagatgatcaaacaagctgaagatgctgagctggctgaggaaagcctttccaagcgcgagttgactgctgatcagcgattggatgaaatgatcaaacaggccgaagccgctgaaattgcagaggagaacttggctaaacgggagttgactgccggccaaagactggacgagatgattaagcaagctgaagcagctgagcttgccgaagagttgatctaa

SEQ ID NO: 552 - DNA, 633 bp, Phakopsora pachyrhizi
  source 1..633: mol_type = genomic DNA
atgaatcacgctttgatcatctgtctacttggacttgcctcctttggcttgatctcggggcattttaccatggactatcctccaacccgaggttttatggaggatgacgaggataaattctgtggaggattccctaccaatgctaccggtagacatccgttcccgatatccggtccagctcatgttgcgctcatggctcaccacgggtcccccaaaatcagtatttccataagctttaagcacaattttactagtgaagccgagtttaacggcactgagttgatatctaacgctcctctgcctcatcctggaaatgcttgttttgaaattgatctgtcgaacgttactagtaagatgacgcctaagccttccaacggaacattagcaactctagaggttaaatatgatgccggaggtgaggctttgtaccagtgttcagatttagttctggtccaggatgctgcggtgacaaatcaaagccttagcagctgctctgccggccgtggcagtaatggcggaagcggctctgatgccaagcaaaatgctcaaaatagctctgctaactctttgtggttccaacctactccgactatgatctgccttagtctaattcttttatcgctctccattctt

SEQ ID NO: 553 - DNA, 198 bp, Phakopsora pachyrhizi
  source 1..198: mol_type = genomic DNA
atgtttttattgttgataattgtagggtcattatttatccccacgttaacatttataaggtcagttgaagtttcaagacccgtaataaggttcttcgatgcccacgccacatgcttggttgttcccagcagaaatatcgccgcaaaaatcaaaaaattgatacttggcattgtaaaagtggttaaagcttggtattag

SEQ ID NO: 554 - DNA, 93 bp, Phakopsora pachyrhizi
  source 1..93: mol_type = genomic DNA
ttacacctctgtaaagtctggtgcgtccttgaggatccttgaaaaaccgagggattgaaaaagtcttgtgcccagccgtaccaatatccgcat

SEQ ID NO: 555 - DNA, 147 bp, Phakopsora pachyrhizi
  source 1..147: mol_type = genomic DNA
atgagaagcacagagattaaaaaaaaaaaaagagagagagagaaggacaaggaaagggagaagcaaagaagatattcaaagtcacaagtattcactcgtctttttttttcacttgctggttttcaaatcctcggtgttgaaagataa

SEQ ID NO: 556 - DNA, 1293 bp, Phakopsora pachyrhizi
  source 1..1293: mol_type = genomic DNA
atgcagaccaaaattcttctagctctttgcctagtagctatctctcaggttaatgcgcatggtgcaattacagctgtccaaggctctaacggcatgactggagaggcttttggagtagatcagagcactcctcgtgatggtacaaaacgaaacccatttcaaaccgattcttcaattattcgtgatcgtgagattgctagcggcaagtcatcagcctgcggcagaaccttggctggtggaaacaacgagattggagcagcaatgtctaaggccgagtctgctggtatcccaagtgttagctccgatggaaaggttcagatgactttacatcaagttaacggtgacggtggtggcccttacacctgtgatgttaatgcttccggtgatggaaagactttcacacctatgacaatttctactaacattccgggaaagaacagcagatctaatgccaaggctgaggatattcctttaattgcggagatgcccaaaggaatgacttgcaccggtggatctgacggacagacctgtatcgtaagatgcagaaacgctgccaacgctggtccctttggaggatgtgtagctgttactcaggctacggatgctacaaagcagaagcgggaactatcagcagaacagagaattgatgagatgatcaaacaggctgaggctgccgaggcatttggcgatgagactcttgcaaagcgtgaaatgtctgctgacgagagattggacgaaatgatcaggcaggccgaagccgcagaggcgttcggcgatgagacccttgcaaagcgcgagctttctgctgaccagcgattagacgaaatgattaaacaggctgaagatgctgagctggctgaggaaaacctttccaagcgggagttgactgctgaccagcgattggatgaaatgatcaaacaggccgaagccgctgaaattgcagaggagaacttggttaaacgggaattgactgccgaccaaagactagacgagatgatcaagcaagccgaagccgctgagcttgcagaggagaacttggccaagcgtgagctgactgctgaccaaaggctggaccagatgatcaaacaagctgaagatgctgagctggctgaggaaagcctttccaagcgcgagttgactgctgatcagcgattggatgaaatgatcaaacaggccgaagccgctgaaattgcagaggagaacttggctaaacgggagttgactgccggccaaagactggacgagatgattaagcaagctgaagcagctgagcttgccgaagagttgatctaa

SEQ ID NO: 557 - DNA, 231 bp, Phakopsora pachyrhizi
  source 1..231: mol_type = genomic DNA
atgaacatcatcagctcgatctacctcgctgttgcagctattttgatttctcaaaacaatttcaccagcgccgcaaatcttaatacccgggacgaggttccgaaacctgttcatttcagatatgaagccagatctgctgagcggtacaatcaccttgaggctcgtagaaagaggcggaaaccacaaaacttcaatggaaactcagttaatacggtaattagctctaagtaa

SEQ ID NO: 558 - DNA, 108 bp, Phakopsora pachyrhizi
  source 1..108: mol_type = genomic DNA
atgaatacacacctaggacgagattctcgcctactacaattgagcctattactagcaaacactctgcctactgtaaaatatgaaatgatacaacgtgtcgggtgctag

SEQ ID NO: 559 - DNA, 222 bp, Phakopsora pachyrhizi
  source 1..222: mol_type = genomic DNA
atgtccaaaccatcacttttttccttacttttttttttaaaaaaaaatagaacaaatggaataaagaaaccaccctgcccctttttgaagtttaagcatccagggagaaattataaaaactttatattaaaaaaatgtaatggagtgaatgttgggggtatcttggagatttgcagggatagctcttttttatttgaaaaggttcttcagggtgctgtatga

SEQ ID NO: 560 - DNA, 120 bp, Phakopsora pachyrhizi
  source 1..120: mol_type = genomic DNA
atggtgttcctggttagctctgtggttgaattcagaatcctattttgctccatagccaaaaagtgggtgtattcagtattagcctacattgttcagttttgctatgtcttgatttattga

SEQ ID NO: 561 - DNA, 210 bp, Phakopsora pachyrhizi
  source 1..210: mol_type = genomic DNA
atgacgcttattattggtccctatggtaagataaaactaattgccttcgtgcttcatggatttatggccttccaatcggtcagctgcatgtatgaggatgctataaaatcaacaggatccaaaccttttggagaactccagaaagaattgaaggatctaagtgatggtgagaatgatagacacacacatagtacctttttctctacctaa

SEQ ID NO: 562 - DNA, 180 bp, Phakopsora pachyrhizi
  source 1..180: mol_type = genomic DNA
atgatctctgcaggtcatagggaatcacttttattatcggcagggctagcagaggacaattgttatagaattagaagaaaaaggactttattgatctaccatattccgatcttgaaatcttttgtagttttatgtttctttgttgctgcagcagactgctattttttcaaagcgttataa

SEQ ID NO: 563 - DNA, 126 bp, Phakopsora pachyrhizi
  source 1..126: mol_type = genomic DNA
atgattgattttgatcctgtccttggctcctgttcttgttcttgttcctgttctttctttctttctttcttttttttaattttcaatacagctttcctttttatcaatacagctttcctttattga

SEQ ID NO: 564 - DNA, 408 bp, Phakopsora pachyrhizi
  source 1..408: mol_type = genomic DNA
atgatggcgtttaaccgatttgcatatctgcttttggtgagcattatggttgcgtcctttacaactggacagctccttccaagcgcctacaaatgtaacagtgtcgattcatctcgatttccaatcaatcgagaagactgtcgagattctttaaacctgtttttgtctcccaggaacgtacttacttttgcaaatggaaatactcagagctgtggaacctgcagggtaacaataattaagagcggaacttctgcaccacctagaactgctcaaaaaagttgggcagttacagcattacatgaagggtatgacaactgcaacggaaaccctgtgactgcaaccattggagacagtgccatgatcgacgtgtctctaagctatggaaacggaactccctgtccaccgtaa

SEQ ID NO: 565 - DNA, 147 bp, Phakopsora pachyrhizi
  source 1..147: mol_type = genomic DNA
atgtatagatttatctttttatttttttgtatcttctttttgttcacttcaaatgtttttaattttatcttaaactttcataatagtgataaaaagagaggagagagatacctaccaatctatttctcatgccatcctattgtttag

SEQ ID NO: 566 - DNA, 657 bp, Phakopsora pachyrhizi
  source 1..657: mol_type = genomic DNA
atgctttttgctacactaatagccgtttgcttacttgctctgggaggcaaagctgagagtgatgtccaaagtgataccgcatctaaacttcagcgacgaggacatgatgactctctacctcctgtgacatttattatgagagatagcaacgagcatgtaggcggaaagctgttgatctacaattctgatggaacactagctttcacctttcgcagagcagttttgaactctgatggcctttccaacgttgaagtcagagatgtgcgaaataatttttcgataaacttagaatctaacgatgacacctgctttaaaaagtctcactatgttgaaagagaaaaaaatctgggtcaattcaaaattgatccacgaggtgcaaaagcagacagatggcatttcactaggaaaactccacacggagatttcaagtatgactttcatcgcaagtatttttcgaaggacggtaatatttacataaacgacacccatgtgcgagtggcttccctgacatccgaaattcgacatgaagcttggctccaaccggggaaacatggtgttccaacattctcattgcatcttcaatataacactgagcctatcttttttgtagctttgatgggcttagatctcaccagagtagacacatgtggactttga

SEQ ID NO: 567 - DNA, 138 bp, Phakopsora pachyrhizi
  source 1..138: mol_type = genomic DNA
atgaatacccctagttctatggcactgggatggggtgttttactgttagctgccggtggtggcctgtactttgctaaaaaggatattaatcaaaaacggagagacttacacaaatctggtgcacaaattttaacttaa

SEQ ID NO: 568 - DNA, 435 bp, Phakopsora pachyrhizi
  source 1..435: mol_type = genomic DNA
atgctgagtactagggttatcatgaagctgatcctgctcggtgttttttcaaaccttctgattgttgcctgtgatgagggctttaactgtaaggatccgtctaagggtcatccgcaccgtctggtgaatgctacccaatgtgtaaatgccatcaaatcgtttccagtcataaatggtgatttctctgtttcgttgaacccagccaaaagttacgtacgacactgtgggacttgtaaacttgaactgagtggatctgaaaaggccaaacaagcaggactgcctcttgcaaaagatgaaacgttgatcaagataaaggatgctgttaaccactgtaataacgattcgtatggagatttttcaatgtacaacgagaggagtaaagtcactgttcgaatcgactgggaatactctacgaaaggtgcctactgcccctag

SEQ ID NO: 569 - DNA, 984 bp, Phakopsora pachyrhizi
  source 1..984: mol_type = genomic DNA
atgatgatgatgatgatgatgaggcaagcgaccaagaagattctaggtagcgttaacctgtcgtctaccagggccatccatcacatagctcccttctctaaaatagcctcagtagcaccattactctcagggttaacatcctcttcccactctcaaaggttttctttaaataatcaactcactcacacctcatcctcggttcgaagtttctttagtctgccttcctttcctcctagtctgaatgtttcaaaggatattcatgggtacgaagacttaaacgtcagagacaggtattctcagagaggagacgttcttacctacaaggagactaaacgattacctttctctaaacatcagctatactacatcgcagctgatatagagtcatattcgagcttcttgccctactgtgctcaatcaaacctaagatcggtcagagcgttggacaagaccagcgatgacagtttagaaagcaatctgataactaatgacgagaggagacaatggttgaggggtggaaaggttggtgagacttttcttatcgagttggagcaggtcaacggtttcaagggcttggacgagctcaatagaacctctgtagaatgtagaaagttcgatctggtaaagttgacaatgtgtgattcaaaacttttcaagagcttgagcataacttggaaattcaaatccccaagagagatcttcaacatcgattcgatcttaaataaccaaaatggcttcacttcacagtcatcctcgttgagagaggaggataatgagggaactggggatgctttcgtgtcaatcgagacgtcacttaccttactctctggttatatcaaccagcagttaggtcaagttaaaggatcaaagtttagtgctaacgatctgagcggagaactctattggaaagccatgagtgaaaaggtcttggccagctacgagagtagactcaatcaggtctacctcaacaatcgccagaattaa

SEQ ID NO: 570 - DNA, 966 bp, Phakopsora pachyrhizi
  source 1..966: mol_type = genomic DNA
atgatgtttcagagcaaactcttgggtcttttaattttgtccctttatctctgtaaaacttctttcgctatcccgttgggcgatgatcagaaggctgatccagtagatacctctggattcaccatcatatctgatctctataataaatcttacaacaatcataacgtcaaaactgatcatccggaaccacccaagtacacagatccaaaaaaaccagatccaccgaactggaaggagcccaaacatccggaaccacccaagtacacagatcccaaaaaaccagagccaccgaactggaaagagcctcaacatccggagccaccaaactggaaagagcctaatcacccagacccacctgaatggaaagggcccgaacatcctgagccgccaaagttgaaagaaccaaagcatcctgagccgccaaagtggaaggtaccaaagcatcctgagccgccaaagttgaaagaaccaaagcatcctgagccgccaaagtggaaggtaccaaagcatcctgagccgccaaagttgaaagaaccaaagcatcctgagccgccaaagtggaaagaaccaaagcatcctgagccgccgaagtggaaggtaccaaagcatcctgagccgccaaattggaaagaaccaaagcatcctgagccgccgaagtggaaggtaccaaagcatcctgagccgccaaattggaaagaaccaaagcatcctgagccgccaaagtggaaagagccaaagcatcctgagccgcctaagtggaaagagccagagaaacctgagccgccaaagtggaaagagccaaagcatcctgagccgccaaagtggaaagagccagagaaacctgagccgccaaagtggaaagagccagagaaacctgagccaccaaagtggaaagagccagagaaacctgaaccaccgaaatcgaaggagccagggtatactgatcctcctaaggagaggaactga

SEQ ID NO: 571 - DNA, 867 bp, Phakopsora pachyrhizi
  source 1..867: mol_type = genomic DNA
atgatgtttcagagcaaactcttgggtcttttaattttgtccctttatctctgtaaaacttctttcgctatcccgttgggcgatgatcagaaggctgatccagtagatacctctggattcaccatcatatctgatctctataataaatcttacaacaatcataacgtcaaaactgatcatccggaaccacccaagtacacagatccaaaaaaaccagatccaccgaactggaaggagcccaaacatccggaaccacccaagtacacagatcccaaaaaaccagagccaccgaactggaaagagcctcaacatccggagccaccaaactggaaagagcctaatcacccagacccacctgaatggaaagggcccgaacatcctgagccgccaaagttgaaagaaccaaagcatcctgagccgccaaagtggaaggtaccaaagcatcctgagccgccaaagttgaaagaaccaaagcatcctgagccgccaaagtggaaggtaccaaagcatcctgagccgccaaattggaaagaaccaaagcatcctgagccgccgaagtggaaggtaccaaagcatcctgagccgccaaattggaaagaaccaaagcatcctgagccgccaaagtggaaagagccaaagcatcctgagccgcctaagtggaaagagccagagaaacctgagccgccaaagtggaaagagccaaagcatcctgagccgccaaagtggaaagagccagagaaacctgagccgccaaagtggaaagagccagagaaacctgagccaccaaagtggaaagagccagagaaacctgaaccaccgaaatcgaaggagccagggtatactgatcctcctaaggagaggaactga

SEQ ID NO: 572 - DNA, 249 bp, Phakopsora pachyrhizi
  source 1..249: mol_type = genomic DNA
atggtacgttccttgtcattcagtctgatctgcttagccctttcgaccatggcttgttcatgtaaagttctctcattctctggtttactttccataaaacttaatcttggatcagtcaattgggctctagctcgccagaagtccggattaaaagagaacaacttcgtgttgttgtcctgtggaactttgttccatgggtctgggtttgacttttttcttaaaacgacgtcttggttattctttaaataa

SEQ ID NO: 573 - DNA, 93 bp, Phakopsora pachyrhizi
  source 1..93: mol_type = genomic DNA
atgaagcttcaaatttcagcgttagtcatctctattgttttaggcagcctgattggttctgtgatttccaacccaacatttacaaagagacaa

SEQ ID NO: 574 - DNA, 189 bp, Phakopsora pachyrhizi
  source 1..189: mol_type = genomic DNA
atggctatcgcaccaattactggaaagttgaaaaagcgattgttcctagatctatccattggactagggcttggcactgctggagcttattcttggtggtatggctatcatgtaccaaaaatgcgccatcaagaaagtatttatgctaaaatgaacgcggatcgagctaaacacgaagcagcaatataa

SEQ ID NO: 575 - DNA, 303 bp, Phakopsora pachyrhizi
  source 1..303: mol_type = genomic DNA
atgaatttcggatcaagctcaaagaagaaccttgtagcaagaccttcagttggtataaggagactagtgctggtatgcaccgcgatggtctggagtatcactctgataatcaaccaaccgagtggtgtaaactcatctccgtttttaagagtcgccaagagcatcggaccagaagttgctaaaggtgccgccggaggtgttggattagttgcagtagaacaaattgctaacaaagtgattgggagaagtaccgatggtcaaaatgtagaagaagaaaattctggttcgaataaattttcataa

SEQ ID NO: 576 - DNA, 78 bp, Phakopsora pachyrhizi
  source 1..78: mol_type = genomic DNA
atgaattatgttgaaatgcttctctcacccttgctattattaaaactaaagcaatctgattttatatggaataaataa

SEQ ID NO: 577 - DNA, 315 bp, Phakopsora pachyrhizi
  source 1..315: mol_type = genomic DNA
atgatgataagttttaagtatagaattaaatcatcagtcgtgattctctttacgatcctcctatcctgcttgctcatagagataatcagttctactcagtcgccctcgacaatacaagcaagaagcgctgtgaagaagattgaaaaggatcatcaacagaggcggttgaagataaggaagagacaagtgacagatgcacctaatacacccaacctggatcttaacatcactgatgccatagccattcaggctgatactattccttggtacatgctggatcagtatcaacaggaccatattagatttcattggtaa

SEQ ID NO: 578 - DNA, 492 bp, Phakopsora pachyrhizi
  source 1..492: mol_type = genomic DNA
atgcgcagctttgttacgtgtgccttgctcatcttattttttcagcttattgtaagctcccccattgagcaatcttcggagaaacaaattcgacgtccccctcctccgccaccaccgcctgtgcgccgcccgccgccaccaccgcactctgggcgcccccatccacgagacacaggaaataaggacaaccaccaaaacgaaaagagatatggtgttgaggcttcccaagagcaattgtctgaaaaactaattcgacgtatcgcttccccacagcgaccgcctgtgcgccgccctccaccacctccatcccccggacgcccccgtccacgagatacaggaaataaggacaaccaccaaaacgaaaagagatatggtgttgaggcttcccaagagcaattgtcggaaaaattaattcgacgtatcgcttccccacagcgaccgcctgtgcgccgccctacaccacctccgccctctggacgcccccgtcaatga

SEQ ID NO: 579 - DNA, 186 bp, Phakopsora pachyrhizi
  source 1..186: mol_type = genomic DNA
atgtggagaggcttttggtatggttgggtaacagcagtttgtttattttttatgatccgaaataaaacctgtaggatagataggcaatcagaactgccaacggtaccaggtaaccaaacccaccaccatcactactactgctatcacccatactgctactcttttcatcagtcgatctgtcgttag

SEQ ID NO: 580 - DNA, 231 bp, Phakopsora pachyrhizi
  source 1..231: mol_type = genomic DNA
atggctggaaaagagatttatgaaccaagaaagtcaatgatgattaaaatgattgggttcttatgcatttggtttcaactattaggagtagtttttacacaatctactgctgcaactacaagtgccactaaggccaaaacttatgatacttatttcaagaggcttgcaaaaagaagcccattgactaactctggccgaattgagagaaggcataaatgtggaccgccatag

SEQ ID NO: 581 - DNA, 606 bp, Phakopsora pachyrhizi
  source 1..606: mol_type = genomic DNA
atgcaatccaactttctcctcgctttttgcttgattgctgctgtctctgttccagttagcagggctcacggtgttattacttcagttgagggcgcaaacggtcaaactggatctgcctttggaatggtagagtccactcctcgtgatggtacccgaacgaacccattccaaactgacagttccatcattcgagaccgtgaggtttcaagcggaaaggcgtctgcttgtggacgcactcttgcgggcggaaacaacgatattgctagtgacatgtcatcggctgaatctgccggattggccagtattggccctgacggaaaagtcagaatgactcttcaccaggtcaacggtggaggcccatacagctgtgaagttgacaccaccgccactggtgacaacttcaagaaaatgaacatcgacacaaacgttcctggcaagaacagtcgatcaagggcaaaggctactgaatttccacttgttgcaaccatgcccgctggcgctacctgcactggtggtgctgacggccaaacatgcttggtacgatgcagaaatgctgcaaacgctggtccatttggtggatgtgttgctgttacccaagcaaactag

SEQ ID NO: 582 - DNA, 612 bp, Phakopsora pachyrhizi
  source 1..612: mol_type = genomic DNA
atgcaatccaactttctcctcgctttttgcttgattgctgctgtctctgttccagttagcagggctcacggtgttattacttcagttgagggcgcaaacggtcaaactggatctgcctttggaatggtagagtccactcctcgtgatggtacccgaacgaacccattccaaactgacagttccatcattcgagaccgtgaggtttcaagcggaaaggcgtctgcttgtggacgcactcttgcgggcggaaacaacgatattgctagtgacatgtcatcggctgaatctgccggattggccagtattggccctgacggaaaagtcagaatgactcttcaccaggtcaacggtgacggtggaggcccatacagctgtgaagttgacaccaccgccactggtgacaacttcaagaaaatgaacatcgacacaaacgttcctggcaagaacagtcgatcaagggcaaaggctactgaatttccacttgttgcaaccatgcccgctggcgctacctgcactggtggtgctgacggccaaacatgcttggtacgatgcagaaatgctgcaaacgctggtccatttggtggatgtgttgctgttacccaagcaaactag

SEQ ID NO: 583 - DNA, 159 bp, Phakopsora pachyrhizi
  source 1..159: mol_type = genomic DNA
atgtttcaccgcacttcggctttaaaagctgtcgcttttttctttattttttcttggtcagtcttcattctagaggtcttaagttttccaaccaaatattcaccgagcgaaaacaagatagtacttgagaaaggtcaaggtagtagccgtggcgattaa

SEQ ID NO: 584 - DNA, 207 bp, Phakopsora pachyrhizi
  source 1..207: mol_type = genomic DNA
atgcgcagctttgtttattgtgccttgctcgtcttaatttgtcagcttgctacaagctcgcctattaatgaaagatctgaggttgaggcttccaaagagcagtcttcggagaagttttttccacttttccctcccccaccaccagtgccgtttatacgcccacctttaccaccttttggatttaaagacaaacaccaaaatgagaag

SEQ ID NO: 585 - DNA, 573 bp, Phakopsora pachyrhizi
  source 1..573: mol_type = genomic DNA
atgaagagcttcaacttaatcttatttattacagtcgctttggcattaatcactcaaattgattgtttacctaaaccaaggtcattagtatcatttaatcatttaccagcaaaagaaatagttaaaagacaagaagatcgattcaatgatgctgacgcgctaagtagagagacagaaggcgatggcgaaggtgaaggtgaaaatgatgttggaggcgaaggcgaaaatggaagtgacaatgatgacgaaggcggaggtgaaagtgaagatgggagtgtgggtgacggagaggaagaagctgaaggagaagaagtaaacgaagatgtagtagaggtagatggagatggagataaggataaagaagttgaaggagaggaaggaggaggagtagagacagtagaaaaggtagaaggggtggacataggtagtaaggaagtaaatggaaatgtagaagaaggagataacaatgacaatgagaacgctgaagacttttctagaggtgttaatgacttgcaagagaaccaatttgtggacgatgattcagaagacgaagcattggctgatgaaagataa

SEQ ID NO: 586 - DNA, 246 bp, Phakopsora pachyrhizi
  source 1..246: mol_type = genomic DNA
atgcgcagctttgtttattgtgccttgctcgtcttaatttgtcagcttgctacaagctcgcctattaatgaaagatctgaggttgaggcttccaaagagcagtcttcggagaagttttttccacttttccctcccccaccaccagtgccgtttatacgcccacctttaccaccttttggatttaaagacaaacaccaaaatgagaaggtgagatttataaatcaaaatattaaaggcattagataa

SEQ ID NO: 587 - DNA, 645 bp, Phakopsora pachyrhizi
  source 1..645: mol_type = genomic DNA
atgcgcagctttgtttattgtgccttgctcgtcttaatttgtcagcttgctacaagctcgcctattaatgaaagatctgaggttgaggcttccaaagagcagtcttcggagaagttttttccacttttccctcccccaccaccagtgccgtttatacgcccacctttaccaccttttggatttaaagacaatcaccaaaatgcgaagagatccgaggttgagactgccaaagaggagtcttcggagaaattttttcccattctacctccccccccaccaccaccatttttccgcccccatctaccaccctttggattcaaagacagccatcaaaatgagaaaaggtctgaggttgctgctcaccaagagagccagtcgacagagaaattttttccagggtatggtttcccgatcctaccaccaccacatccatttttttaccaccgtcgtccttttggttatggcttttttccaccaccaccttttggatttaaagacaaacaccaaaatgagaagcgatctgaggtcgaatcctctaaggaaaagtcaactgagaagtttttcccaggtctcttaccgattttgccaccttttcccccatttggcttcctaaaagatgccaagagcaatgatgagtccgcttaa

SEQ ID NO: 588 - DNA, 621 bp, Phakopsora pachyrhizi
  source 1..621: mol_type = genomic DNA
atgcgcagctttgtttattgtgccttgctcgtcttaatttgtcagcttgctacaagctcgcctattaatgaaagatctgaggttgaggcttccaaagagcagtcttcggagaagttttttccacttttccctcccccaccaccagtgccgtttatacgcccacctttaccaccttttggatttaaagacaatcaccaaaatgcgaagagatccgaggttgagactgccaaagaggagtcttcggagaaattttttcccattctacctccccccccaccaccaccatttttccgcccccatctaccaccctttggattcaaagacagccatcaaaatgagaaaagccagtcgacagagaaattttttccagggtatggtttcccgatcctaccaccaccacatccatttttttaccaccgtcgtccttttggttatggcttttttccaccaccaccttttggatttaaagacaaacaccaaaatgagaagcgatctgaggtcgaatcctctaaggaaaagtcaactgagaagtttttcccaggtctcttaccgattttgccaccttttcccccatttggcttcctaaaagatgccaagagcaatgatgagaagtccgcttaa

SEQ ID NO: 589 - DNA, 621 bp, Phakopsora pachyrhizi
  source 1..621: mol_type = genomic DNA
atgatgaagtttttctccattgcttttttttctgcgatcatcgcgttggcagtcgcatctggagatcagaaggacgaagtttctgctaagacttttgtcggttcgaatggcggcattggatttggaaatggtggtagataccacggtggatatggtggtggtgccagctggggagttggaggctctttgccaaatttcgatagattttacaatgaatactactcactcatcccctccttccaaagagttcatgtgacccttgagtcctcacaaatcgatgcggtcgcggcaacaaggttgatcggatcattgcaaaatgacttctactccaaggttaactctcttggtgctcgttgcgctggagcccaacaattaagccttgaaagaaagtctagcttaagagtttgctacagaagatggccaaaccaaattggaaatttcaagtattcattggctagacttggagaagctttcaaaatatctttttcgttgatccaaaacttgggtttccaacctaatgaaatcttgggtagactcaacttagacatattttctcagttcgacttgaacttgaactcgttcgcccatagttcaggctttgtttctgggatcggatactaa

SEQ ID NO: 590 - DNA, 577 bp, Phakopsora pachyrhizi
  source 1..577: mol_type = genomic DNA
gcaaggcaagctactaatttcaaagaaaagatgcatacgatctcccaaagactcaatagcgtttcagctttgacgacaacctctataatcatcttaacgattgccattactatcatcagttttattcaaaggccttcgatccaaccagctaggattgagatctcagatattgaaacgttgtgggggcatgataaaagggaccactatgatagaaaagatcgagaatggacaaacattaggtttgacattgaagcagatcttcgaccactatttaattacaacaccaagcagatctttgtcttcttgagtgcaaaattcgggactgataatttccccaacaacgaggttatactctgggaccggatcatcaagagttctaaagatgcaaagatcaatctgactggcgctaagaacaagtatgcgttcaaacatattggcggctcgttcaaaaattctaatgcgacttatattttgcattataaccttatgcctaaggtgggagcgctgatctggggtgaagctggtaaaaccaagaatcaagtaccattcccagaagtcaactatcgaaaacctattc

SEQ ID NO: 591 - DNA, 199 bp, Phakopsora pachyrhizi
  source 1..199: mol_type = genomic DNA
tacagcgcaattgcagtcgtcgtagctctcttgattgcaatagcggtgacatccaggatactatacattagaaggagtcgtcgattagcagcagctccaagatcaatcgatcccgagcagtcaaacaaccgcgagaatgaggagagactggcaatggaacggagaggtgaagaggctggtttaccgacctatcgtcaga

SEQ ID NO: 592 - DNA, 507 bp, Phakopsora pachyrhizi
  source 1..507: mol_type = genomic DNA
atgtatcgatttccactcatttcccttgccatttatgcttcagtcgtatatggtcaaggaactaccgtcactcctactacgaacgtcaacccaaacacatcaggtaatccgccaaaccaacccactaatggtacccctaccaatccggcaactcctacggcatctcctacgacgactcctactcccaaaattcttgctgttaattgtacacagtcttactttgagatcactgatgattcaatgaacggaacttctatcggtgcatgtacgaagattggagacggagaaaaaggcggttccttctgccaaaccaaagagtgttttggctttgccacttgccaagattgtaacctagcaacccctggtcctaataatacgactgtaatctcaaacaccactatcaaagcagtaccctgcaacaagggctactatatccctggaccaggagataacgttactgtgacttgtaaatcagttttgatgcatatctaccatctaaagccttag

SEQ ID NO: 593 - DNA, 555 bp, Phakopsora pachyrhizi
  source 1..555: mol_type = genomic DNA
atgtatcgatttccactcatttcccttgccatttatgcttcagtcgtatatggtcaaggaactaccgtcactcctactacgaacgtcaacccaaacacatcaggtaatccgccaaaccaacccactaatggtacccctaccaatccggcaactcctacggcatctcctacgacgactcctactcccaaaattcttgctgttaattgtacacagtcttactttgagatcactgatgattcaatgaacggaacttctatcggtgcatgtacgaagattggagacggagaaaaaggcggttccttctgccaaaccaaagagtgttttggctttgccacttgccaagattgtaacctagcaacccctggtcctaataatacgactgtaatctcaaacaccactatcaaagcagtaccctgcaacaagggctactatatccctggaccaggagataacgttactgtgacttattgctccgcccaaaacaatgcgaatccggatgactacatttgcaagggcccatgcacaagttttacaggtaagctgcccaaattatga

SEQ ID NO: 594 - DNA, 597 bp, Phakopsora pachyrhizi
  source 1..597: mol_type = genomic DNA
atgtatcgatttccactcatttcccttgccatttatgcttcagtcgtatatggtcaaggaactaccgtcactcctactacgaacgtcaacccaaacacatcaggtaatccgccaaaccaacccactaatggtacccctaccaatccggcaactcctacggcatctcctacgacgactcctactcccaaaattcttgctgttaattgtacacagtcttactttgagatcactgatgattcaatgaacggaacttctatcggtgcatgtacgaagattggagacggagaaaaaggcggttccttctgccaaaccaaagagtgttttggctttgccacttgccaagattgtaacctagcaacccctggtcctaataatacgactgtaatctcaaacaccactatcaaagcagtaccctgcaacaagggctactatatccctggaccaggagataacgttactgtgacttattgctccgcccaaaacaatgcgaatccggatgactacatttgcaagggcccatgcacaagttttacagcgtgtggagattgtatcaatataaacgatccagcgattgcgggcctgaaagatactccttaa

SEQ ID NO: 595 - DNA, 315 bp, Phakopsora pachyrhizi
  source 1..315: mol_type = genomic DNA
atgttccacagagcaacagtcttcatcactctcttttctgctctgtcaattctgaatgacaaccgtgtctcagctctaatggcatctgtacctgatactcagacctgcagcttctacaccggcgcttacaccaactcagccacttgcaacgagcaaccaaacgtggtctgcactcagggatgctacggtccctattggctactggctgtgtcttggagaacggctcaacgggtggtgcttcaagctcctctcaagtgtgcactatgggatttggacgcaacacggcagctgcaaaagaagaagtacggaccttga

SEQ ID NO: 596 - DNA, 471 bp, Phakopsora pachyrhizi
  source 1..471: mol_type = genomic DNA
atgctttttgctacactaatagccgtttgcttacttgctctgggaggcaaagctgagagtgatgtccaaagtgataccgcatctaaacttcagcgacgaggacatgatgactctctacctcctgtgacatttattatgagagatagcaacgagcatgtaggcggaaagctgttgatctacaattctgatggaacactagctttcacctttcgcagagcagttttgaactctgatggcctttccaacgttgaagtcagagatgtgcgaaataatttttcgataaacttagaatctaacgatgacacctgctttaaaaagtctcactatgttgaaagagaaaaaaatctgggtcaattcaaaattgatccacgaggtgcaaaagcagacagatggcatttcactaggaaaactccacacggagatttcaagtatgactttcatcgcaagtatttttcgaaggacggtgagtaa

SEQ ID NO: 597 - DNA, 657 bp, Phakopsora pachyrhizi
  source 1..657: mol_type = genomic DNA
atgctttttgctacactaatagccgtttgcttacttgctctgggaggcaaagctgagagtgatgtccaaagtgataccgcatctaaacttcagcgacgaggacatgatgactctctacctcctgtgacatttattatgagagatagcaacgagcatgtaggcggaaagctgttgatctacaattctgatggaacactagctttcacctttcgcagagcagttttgaactctgatggcctttccaacgttgaagtcagagatgtgcgaaataatttttcgataaacttagaatctaacgatgacacctgctttaaaaagtctcactatgttgaaagagaaaaaaatctgggtcaattcaaaattgatccacgaggtgcaaaagcagacagatggcatttcactaggaaaactccacacggagatttcaagtatgactttcatcgcaagtatttttcgaaggacggtaatatttacataaaagacacccatgtgcgagtggcttccctgacatccgaaattcgacatgaagcttggctccaaccggggaaacatggtgttccaacattctcattgcatcttcaatataacactgagcctatcttttttgtagctttgatgggcttagatctcaccagagtagacacatgtggactttga

SEQ ID NO: 598 - DNA, 281 bp, Phakopsora pachyrhizi
  source 1..281: mol_type = genomic DNA
atgatgataagttttaagtatagaattaaatcatcagtcgtgattctctttacgatcctcctatcctgcttgctcatagagataatcagttctactcagtcgccctcgacaatacaagcaagaagcgctgtgaagaagattgaaaaggatcatcaacagaggcagttgaagataaggaagagacaagtgacagatgcacctaatacacccaacctggatcttaacatcactgatgccatagccattcaggctgatactattccttggtacatgctggatca

SEQ ID NO: 599 - DNA, 954 bp, Phakopsora pachyrhizi
  source 1..954: mol_type = genomic DNA
atgttcaacatgttaatagcattagctattactcttgtgcacgctggcagtgtattctgccagcaagcagcctcagctcaacaagattcatccactacaattgtggctaatacaaaccttaacggagcttatggagtttcgggtcagatctcgttcacccttattcccacgacagaagaggttgctgtcaagatttcagttaaagggttgatggccattaactctaccgcccaatttggctatcacattcataccaatccaatttcatccgatggaaactgctcttctgcccttggtcatctcgaccccctgcaggtcaccgattccgtggtatgcaacggaacggcgcggttatgtcagatcggggatctatcaggtcgtaacggcaagctaccagggaatcaatctgttgcaaatgtctcctacacggataaattcctcagattctggcctcaaccatttacccttcttggacgaagtatagtcattcatgctcccaataccacaagaatcgcctgtggaaacattacctctgttgttgatggaactgcggacaatcaagggaagcctaccaaccaaagctcaaactatgtgacagtctactctacacgagcacctcctccttctaataacactgccaacgttgcggaacctcttcctgatgtaacagagacccccaatgtgattttggttaaaaaacaagttactcaaatgatgaataattcttctgaaacggttgaggtgaatgctgcagctccagctgcgactaatttcacctacactcccggagctcaacttccaagtcaagaagacttcaacattcttaacggcaccaaccttaatagcactacttcggctaagccacaaactgggtcaagtagcggtattgagaagatcgaataccctttattttttgggttttgtgctataatgatgatttcagtcgtgatttaa

SEQ ID NO: 600 - DNA, 612 bp, Phakopsora pachyrhizi
  source 1..612: mol_type = genomic DNA
atgcaatccaactttctcctcgctttttgcttgattgctgctgtctctgttccagttagcagggctcacggtgttatcacttcagttgagggcgcaaacggtcaaactggatctgcctttggaatggtagagtccactcctcgtgatggtactcgaacgaacccatttcaaactgacagctccatcattcgagaccgtgaggtttcaagcggaaaggcgtctgcttgtggacgcactcttgcgggcggaaacaacgatattgctagtgacatgtcatcggctgaatctgccggattggccagcattggccctgacggaaaagtcagaatgactcttcaccaggtcaacggtgacggtggaggcccatacagctgtgaagttgacaccaccgccactggtgacaacttcaagaaaatgaatatcgacacaaacgttcctggcaagaacagtcgatcaagggcaaaggctactgaatttccacttgttgcaaccatgcccgctggcgctacctgcactggtggtgctgacggccaaacatgcttggtacgatgcagaaatgctgcaaacgctggtccatttggtggatgtgttgctgttacccaagcaaactag

SEQ ID NO: 601 - DNA, 597 bp, Phakopsora pachyrhizi
  source 1..597: mol_type = genomic DNA
atgcaatccaactttctcctcgctttttgcttgattgctgctgtctctgttccagttagcagggctcacggtgttattacttcagttgagggcgcaaacggtcaaactggatctgcctttggaatggtagagtccactcctcgtgatggtacccgaacgaacccattccaaactgacagttccatcattcgagaccgtgaggtttcaagcggaaaggcgtctgcttgtggacgcactcttgcgggcggaaacaacgatattgctagtgacatgtcatcggctgaatctgccggattggccagtattggccctgacggaaaagtcagaatgactcttcaccaggtcaacggtgacggtggaggcccatacagctgtgaagttgacaccaccgccactggtgacaacttcaagaaaatgaacatcgacacaaacgttcctggcaagaacagtcgatcaagggcaaaggctactgaatttccacttgttgcaaccatgcccgctggcgctacctgcactggtggtgctgacggccaaacatgcttggtacgatgcagaaatgtgaggtttaattttaaatatcagatctttatttcttga

SEQ ID NO: 602 - DNA, 606 bp, Phakopsora pachyrhizi
  source 1..606: mol_type = genomic DNA
atgcaatccaactttctcctcgctttttgcttgattgctgctgtctctgttccagttagcagggctcacggtgttattacttcagttgagggcgcaaacggtcaaactggatctgcctttggaatggtagagtccactcctcgtgatggtacccgaacgaacccattccaaactgacagttccatcattcgagaccgtgaggtttcaagcggaaaggcgtctgcttgtggacgcactcttgcgggcggaaacaacgatattgctagtgacatgtcatcggctgaatctgccggattggccagtattggccctgacggaaaagtcagaatgactcttcaccaggtcaacggtgacggcccatacagctgtgaagttgacaccaccgccactggtgacaacttcaagaaaatgaacatcgacacaaacgttcctggcaagaacagtcgatcaagggcaaaggctactgaatttccacttgttgcaaccatgcccgctggcgctacctgcactggtggtgctgacggccaaacatgcttggtacgatgcagaaatgctgcaaacgctggtccatttggtggatgtgttgctgttacccaagcaaactag

SEQ ID NO: 603 - DNA, 606 bp, Phakopsora pachyrhizi; identical to SEQ ID NO: 581
  source 1..606: mol_type = genomic DNA

SEQ ID NO: 604 - DNA, 318 bp, Phakopsora pachyrhizi
  source 1..318: mol_type = genomic DNA
atgttccacagagcaacagtcttcatcactctcttttctgctctgtcaattctgaatgacaaccgtgtctcagctctaatggcatctgtacctgatactcagacctgcagcttctacaccggcgcttacaccaactcagccacttgcaacgagcaaccaaacgtggtctgcactcagggatgctacggtccctatgttgtggctactggctgtgtcttggagaacggctcaacgggtggtgcttcaagctcctctcaagtgtgcactatgggatttggacgcaacacggcagctgcaaaagttttgaaaatgctttaa

SEQ ID NO: 605 - DNA, 954 bp, Phakopsora pachyrhizi
  source 1..954: mol_type = genomic DNA
atgttcaacatgttaatagcattagctattactcttgtgcacgcgggcagtgtattctgccagcaagcagcctcagctcaacaagattcatccactacaattgtggctaatacaaaccttaacggagcttatggagtttcgggtcagatctcgttcacccttattcccacgacagaagaggtcgctgtcaagatttcagttaaagggttgatggccattaactctaccgcccaatttggctatcacattcataccaatccaatttcatccgatggaaactgctcttctgcccttggtcatctcgaccccctgcaggtcaccgatgccgtggtatgcaacggaacggcgcggttatgtcagatcggggatctatcaggtcgtaacggcaagctaccagggaatcaatctgttgcaaatgtctcctacacggataaattcctcagattctggcctcaaccatttacccttcttggacgaagtatagtcattcatgctcccaataccacaagaatcgcctgtggaaacattacctctgttgttgatggaactgcggacaatcaagggaagtctaccaatcaaagctcaaactacgtgacagtctactctacacgagcacctcctccttctaataacactgccaacgttgcggaacctcttcctgatgtaacagagacccccaatgtgattttggttaaaaaacaagttactcaaataatgaataattcttctgaaacggttgaggtgaatgctgcagctccagctgcgactaatttcacctacactaccggagctcaacttccaagtcaagaagacttcaacattcttaacggcaccaaccttaatagcactacttctgctaagccacaaactgggtcaagtagcggtattgagaagatcgaataccctttattttttgggatttgtgctataatgatgatttcagtcgtgatttaa

SEQ ID NO: 606 - DNA, 531 bp, Phakopsora pachyrhizi
  source 1..531: mol_type = genomic DNA
atgcaatccaactttctcctcgctttttgcttgattgctgctgtctctgttccagttagcagggctcacggtgttattacttcagttgagggcgcaaatggtcaaactggatctgcctttggaatggtagagtccactcctcgtgatggcactcgaacgaacccattccaaactgacagttccatcattcgagaccgtgaggtttcaagcggaaaggcgtctgcttgtggacgcactcttgcgggcggaaacaacgatattgctagtgacatgtcatcggctgaatctgccggattggccagtattggacctgacggaaaagtcagaatgactcttcaccaggtcaacggtgacggtggaggcccatacagctgtgaagttgacaccaccgccactggtgacaacttcaagaaaatgaacatcgacacaaacgttcctggcaagaacagtcgatcaagggcaaaggctactgaatttccacttgttgcaaccatgcccgctggcggtaactacctgcactggtggtgctga

SEQ ID NO: 607 - DNA, 1374 bp, Phakopsora pachyrhizi
  source 1..1374: mol_type = genomic DNA
atgaaatatctaaggtttattcaactgtttttaatcttaaaagcagttgatctctacactttggtagctaaaaaggaagagggaataaattctcacaaagcttacgatgcatctgcagtccttgtcatctctgaaaaaaacattgcagcagactgcaccatcagaccgtcaactgtgatcaatggtacacttcctggccctttgttgagattcaaagaaggtcaaaaaattcgaataactgttatcaatctcttacaacacgaaaatacaacaattcactatcatggtttatctcaacgtggatctccattctctgacggaactcagaaaacttcacagtgggctattccgcctcgaggtggaagatttgactatgaattttttttagaaaaaggttcagcaggaacttacatgtaccatgctcacaatctatttcaagtctcaacagcctatggtcctttaatagtcgatgaagcaacaaagccgccatttaattacttcgaagatcggatgtttatgataggggattattaccattcaactgaccaagaacttatttcaaaacttctgggaactcctttcgcctggattggtgatcctcaaagcgttttagtcaatggtcaggctttgaatgtttgtaataccaccagccctcatggttgtgaaaacagttgtgcgcatcatataacttacgtcaaacctgataaggtgtatagaattcgagttgcaggaattacttcgttagcttatttgtatatggccattgaatcccatacattttttgaaatcattgaagtcgatagcgcatttgtgaaacctttaaaagtgaatcacatgcaattgaactcaggccaaaggtattcttttttattaaagacaaagagtgaagcggagattaaaaaacttggaaaaacaagtttttggataagatttgagagcaaatatagaactacaaaagttcatggacaatttattcttcaatatcaatcagtggacaaggacgatcatttaagtaaatcaaaaaccgctgttgttggagatttaccaaaaaaaacctcaaattcactattggaaccttcaaataaagagatcaacaataagtcgccaccaccaccacttttgtactttcaaaagcttgtacctataccttctgaaagctccaaagatgaatggtcaacttcagatttctcacctctattagaagagagaccagctagaagatctgaggtaaatagacgtattatcgtaagaggtcaacagattaaatctgcagatggatcgattaagtggtatgctaacggccagactgtcagctattttgataatttaattttgggattttctttgttatctacttacaaaataaaacgttga

SEQ ID NO: 608 - DNA, 765 bp, Phakopsora pachyrhizi
  source 1..765: mol_type = genomic DNA
atgaacatgaaacttagagtattcatttccttaactgttgcgctttctttgattgctcaatccccaagcctagctgtaactttaaatagagatcggccttcaaatgcccatgcgctccaggctagacaggaacaagctgcagagctagctgctgtcacggtaccggaagaacaagctgccgctagtgctcccgagcagaaggctgctgctgctgcctccgctccagagcacgctgctgcttcctccgctcccgagcacgctgcttctgctgccgctcctcaaagtgacctagacaaagctgccaatagattgtacgacttaatggtaaaccttggaaatggattggagatcgtcacaaatcttgaggccagtccacaggatatcaaggcccagactcaaaaagtcaaacaatattttggtgaaatgaatgagctaagaaacaagttgctccagatgagccccatacattctggaaacctccccaaatctgttcaagatgccaatgaagctcaggaaacattacagaaagcgttgattgctattagtgattcggcagaagacgcaaatgttctaaaaaagaactacccagtcctttcgaagacttttaaatccgtaactactgctggagaagatctgttctcaactttgtaccctggctatggtgagaaaactcaggaaggacaagatgctcaacaacacagccagccccaagcaacgcaagagggtgctgctcctgtagctgctgctgaagcccctcaagcttaa

SEQ ID NO: 609 - DNA, 510 bp, Phakopsora pachyrhizi
  source 1..510: mol_type = genomic DNA
atgagttaccagaggatgtttctgatgtttcaactggtgattctggcagtgatgatgaatgatcagtcgaactctgcacgagcaacgttacttattcaggatgagcccttacatctgatcaagactcagtttgagatcaaacctcagaagctcatcacagtgatcaggcagccaacccagcaggaagctttggatctcagcaggattggtcaatcaactggtactactactactaccggtgttgatggatctatcataagggtcagaccgtcatccagagggatgatgttcaggttcgaggatggtgttggtggtggtgttgagtcagactctgggattggtggaatgctgagtaagtttctcagtaggatcagtagaccgagtgtatcatctggtagtagtgttgaatctaaccttggagggtttgatcttggttctatgttgaagagtgtaaagagctctataagtgatgggatgactactgtcagaagattagtcgtagtctattaa

SEQ ID NO: 610 - DNA, 696 bp, Phakopsora pachyrhizi
  source 1..696: mol_type = genomic DNA
atgaatatctctttaaaaaactttgtgcaagtttcaatcttgctaattctcgtgtctggagtcatcagtcttgatgcttctactccaaacagccatcacctatcaagaagacagaataaggatcaggtcaaggatcagacaaagaagactcaacaacaggtacaagaaatcaaagatggaaaaggtggagcagcccagggcggtactcctacagagaaagctgcaaaccagatgatgcagatgcttatgcagattggtcaaagtttagatgcagtcaccagcctggggtccacccctgagaccgtaaagtcgacagcagaggcaatcataaaaatggttcctgatgtcggcaaggcaacaaagtctttgatagacactatgccgaacaaagcacaacttgagggcgtcgctgataaagccactcaatcagcggaacagctaggaaaagtccttaaaaccattgagcaaagtccagaagatgccaactttatcaaaaaacattataaagccctgggtgacgcctttacaggaatctttggtgctgtggatcccgtcttcagctctgcatttcctgatgataaaaatggtggacaacaacaagccggtaaaaccgacgggagacaactaaacactcagactgaaggggcccaaaaggatcaaaaagatacaaaggaaaaaaacaaaaagaaaacctag

SEQ ID NO: 611 - DNA, 219 bp, Phakopsora pachyrhizi
  source 1..219: mol_type = genomic DNA
atggatttttttttgaaattcacgaaatggctattggttgcctcaattataggcgctgttatgtcttctccacctactattactcggaaggatgcctccagagtggagcggcgaaaaactaaacctttaagtacccaagatcctcagaaaaaggttacttgcccaccaaatgaagttgtgaagggaggaaaatgtgtaccttttaaaccgcatttttaa

SEQ ID NO: 612 - DNA, 1122 bp, Phakopsora pachyrhizi
  source 1..1122: mol_type = genomic DNA
atgaccctcagactgtttaaatgggttggctttcttcaactacttctgaggctcaccagcgcccaagacaccagtggaaccactcctctttaccagaaaaagtttccctggcccaacgtaccatacagagcggatacaggtgacggcccaagggggactcagcagggctacaataagtgtaattctactactcaaaatcaagattcattatgccaaaccacgtttttaaactcaatccaagagttctgtctctggggagccatcaagccaaactctattgttgggaatattgagggagaaatggtggcgtactgcacaacaaagaagtttggcactaggcttattccatcaggggcaataaccggcgtccaactaactcggaccccgggatatatccaagttgttggttttattgatcaaactgcgctcaacctacaagctaatgatacaggaggcgaagaagatcctcatggtgctgatcagcgcgggaaccccctaggagctctgatgtactctagcgcttttaactctaattctggggcctcttccggccaatcttcaaactctacaagttcatcctcttacactcaagtgagtgagtggagcttcttcattggtagcggagtgttctgctataaggcgtgtgacccggctggtcctaatgcacctcaactttgccaacatatatatgaccggattggctgtgcatacaacgctccagcaaactatccttccattaacggcacctaccaatcttgtcaaggagagaaccaattacctgcaggaacgtatattgaaaatggagtaactaagacttatcagcagcctccagagtcgcttggtccaattacctctataccatacacagcccctatacctgccgtatctaactgcgtcacctacaaagacccagtggctttatggcccgatctccctaagactcaaactgtagccaacacaagcagtactgtgaataggaaaggagtcagtagcagcacatccgatggatcaaaacagagcgcgcagggtggagcccagtcagcagcttcgtctattgagctacctgtcatttctcttttgcatccaatagtgcttggtatggtttttgtcatgctggcctga

SEQ ID NO: 613 - DNA, 723 bp, Phakopsora pachyrhizi
  source 1..723: mol_type = genomic DNA
atgaagatgaatatgcttcaccattctttgttgctcttcactttcttctgtataaaactgttgatttgcttacccatgaatggtaagactccagcagttgttgccgatgccgtttcagcaattcaggaaaccggaaaagttgcaagcaccagcatagaagtaccaagcgcaaatttgggtgcggaagtcaaggaactttcagcgattgatgcgtctgtgttcacaaatgttcctgctatcaatccgaaggattcggcccaagtagaaaatctggggcaatcaactttgcaggagaaagaaagcccagttatattgaagggtgatgtaaatcatgagaaaaaaaatctaataaaaaccgaatctaaatctgcagatagtgaaataaaaaaagaagttgatgaaaaacccttgaaagaaaaatcacagggtgaagacggtcctgatggccctgaaaaaaaaaacattgagggcgaaggaggagatctgaaaaatgacaaaaatttgaatcaatcagaacaaaaaactaggttgagcaggattaagaactggtttataaggcaataccttatgttgaaaaacaatgtataccctaaaataaaagaaaaaagtcaacaattttgggcttggacaaagaaaaaattttcaagaggcaataacaaggtagctacaaatgaaattgaattaaagccatgttgccgaggaaaaaagacacatttcagctaa

SEQ ID NO: 614 - DNA, 354 bp, Phakopsora pachyrhizi
  source 1..354: mol_type = genomic DNA
atgagcgccatcactcagacggcaaaaggtgcagcagttgcaggaggtttatatttcttgttacattcttacattgcaaaccgaagccaattgatctccaaatccctgtctgatttgactaatcaattcagttacttaacaatccaagaagcaaattatggaaacaatagcaaaagtgatttgttcttctatcatcaaagattaaaggagaccatggctgaaagaattaaatttaattggaatcaaaacattcgaaagatttattattcaatctacgactttgatccaataaatcggttaaatagattttatattgagaccaaaaatacttcaaatattgcgtcaaaaaactag

SEQ ID NO: 615 - DNA, 477 bp, Phakopsora pachyrhizi
  source 1..477: mol_type = genomic DNA
atgatgaaatttttgaacgttgtactattttcccctttatttttgatgctttcttctaattctagtccattactaagctcccggcaaagtattccgatttataatcaatgtcgagtcgagaatagcttttccttgacaattgatgatggattttcagcacttagcaaaagcttgacacaggtctttgatgataaccaagttcgagctacattctttattaatgggaacaatgctgcttgcatatactacaatgctgatgatttgaaagcaagacatagaagtggtcatcttcttgccaaccacggttggagtcatgcacacatgacacagctaagtcgtgcgaatatggttagagaagtggaaagagtagaacaggcatttattaagatccttgggcttaaacctctttacttccgtcccccttacggtaagttaatttctttcggaaatttgaggccaacgcaaaaggaattatga

SEQ ID NO: 616 - DNA, 159 bp, Phakopsora pachyrhizi
  source 1..159: mol_type = genomic DNA
atgattagtattactgttgcagcactcgttggtccctcaaccaggttgccaatcaatcaatcactcagttcagtttctggtatcatcatcatccttacaacagtagtcatctcaatctgtcacttcatcgtccagtgtcccaggctgttccagcgatga

SEQ ID NO: 617 - DNA, 174 bp, Phakopsora pachyrhizi
  source 1..174: mol_type = genomic DNA
atgcgttcacttatcgttcccacccttttatgcgtgctatttattcacaaatcttgcgcaaccgttcacactcagtgctacaactattttttacaaaaggatggatgcgttttctctgcagctgatgacagaaatcgatgttctgcagatcccaagcccagtacagccgttgga

SEQ ID NO: 618 - DNA, 273 bp, Phakopsora pachyrhizi
  source 1..273: mol_type = genomic DNA
atgtatttccttacttttattaaaaaaaatgcaatttatatttattcagtgcttatacttttctttgtaaataagaaaagtatcaacattctatgtagcgagtcaattaataataaaattaagtgttgttttcttgtttatttcgaaaattttgttctaaccaattataagttttccctcttcatcattaaaatcgtaagccggaacttccaaattcaaaggagctggacctcttctaattctaccactgatatcatagtgcgatccatgacc

(19) United States
(12) Patent Application Publication
(10) Pub. No.: US 2026/0327637 A1
(43) Pub. Date: Oct. 22, 2026

(54) CCRPP1 COGNATE EFFECTORS AND METHODS OF USE

(71) Applicant: PIONEER HI-BRED INTERNATIONAL, INC., JOHNSTON, IA (US)

(72) Inventors: SHIJIAN DING, ORLANDO, FL (US); BAILIN LI, JOHNSTON, IA (US); YURONG LI, JOHNSTON, IA (US); SHAWN THATCHER, URBANDALE, IA (US)

(73) Assignee: PIONEER HI-BRED INTERNATIONAL, INC., JOHNSTON, IA (US)

(21) Appl. No.: 19/111,431

(22) PCT Filed: Sep. 14, 2023
(86) PCT No.: PCT/US2023/074142

§ 371 Date: Mar. 13, 2025

Related U.S. Application Data
(60) Provisional application No. 63/375,770, filed on Sep. 15, 2022.

(51) Int. Cl. C12N 15/82; C07K 14/415

(52) U.S. Cl. CPC C12N 15/8202; C07K 14/415

(57) ABSTRACT

Disclosed herein are compositions and method for identifying genes that improve or enhance pathogen resistance. Compositions comprising effector polypeptides encoded by the polynucleotides disclosed herein are useful in identifying genes for improving resistance to Asian Soybean Rust (ASR). Methods for selecting an ASR resistant plant to grow in an area of cultivation is also disclosed.

### REFERENCE TO SEQUENCE LISTING SUBMITTED ELECTRONICALLY

The official copy of the sequence listing is submitted electronically via Patent Center as an XML formatted sequence listing with a file named 108283_SequenceListing created on Aug. 7, 2023 and having a size of 763,782 bytes and is filed concurrently with the specification. The sequence listing comprised in this XML formatted document is part of the specification and is herein incorporated by reference in its entirety.

### FIELD

This disclosure relates to the field of molecular biology, and particularly, relates to compositions and methods useful in identifying genes that enhance resistance to the causal agent of Asian soybean rust (ASR) in legume plants. The disclosure further relates to effector polypeptides capable of interacting with the polypeptides encoded by the genes enhancing ASR resistance, methods of using these polypeptide sequences to isolate the polypeptides encoded by the genes enhancing ASR resistance, and methods for selecting plants to grow in an area of cultivation.

### BACKGROUND

Soybean diseases are a major threat for soybean production, resulting in yield losses and decrease in grain quality. Asian soybean rust (ASR) caused by the biotrophic fungus Phakopsora pachyrhizi and, to a lesser extent, the closely related fungus Phakopsora meibomiae, can cause yield losses ranging from 10-90%.

Accordingly, there is a need to develop compositions and methods for identifying ASR resistance genes. This disclosure provides such compositions and methods.

### SUMMARY

Provided are nucleic acid constructs and expression cassettes comprising a polynucleotide encoding an effector polypeptide having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8 or 10-309, or a functional fragment thereof, operably linked to a regulatory element. In certain embodiments, the at least one regulatory sequence is a promoter (e.g., heterologous promoter).

Also provided are chimeric polypeptides comprising an effector polypeptide comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8 or 10-309, or a functional fragment thereof, operably linked to a heterologous peptide (e.g., protein tag). Further provided are polynucleotides encoding the chimeric polypeptides and nucleic acid constructs and expression cassettes comprising the polynucleotides encoding the chimeric polypeptides.

Also provided are host cells (e.g., bacterial cell, fungal cell, plant cell) comprising any of the polynucleotides (e.g., isolated polynucleotides, recombinant polynucleotides, nucleic acid constructs, expression cassettes) encoding effector polypeptides, functional fragments thereof, or chimeric polypeptides described herein.

Provided are methods for identifying CcRpp1-like proteins comprising identifying one or more polypeptides that bind to a chimeric polypeptide comprising an effector polypeptide comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8, or a functional fragment thereof, operably linked to a heterologous peptide (e.g., protein tag). In certain embodiments, the method further comprises expressing in a host cell the chimeric polypeptide and the one or more identified interacting polypeptides and assaying the host cell for the presence of HR cell death, H₂O₂ accumulation, marker gene or marker metabolite expression, or any combination thereof.

Also provided are methods for identifying CcRpp1-like proteins comprising identifying one or more polypeptides that bind to an effector polypeptide having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8, or a functional fragment thereof. In certain embodiments, the method further comprises expressing in a host cell the effector polypeptide or functional fragment thereof and the one or more identified interacting polypeptides and assaying the host cell for the presence of HR cell death, H₂O₂ accumulation, marker gene or marker metabolite expression, or any combination thereof.

Further provided are methods for selecting a plant for growing in an area of cultivation comprising providing a sample comprising a plurality of Phakopsora pachyrhizi isolates from an area of cultivation, assaying for the presence of a polynucleotide comprising a nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8, and selecting a plant expressing a CcRpp1 polypeptide, a CcRpp1-like polypeptide or a combination thereof for growing in the area of cultivation when the polynucleotide is present.

Also provide are methods for identifying ASR26 alleles comprising providing a population comprising a plurality of Phakopsora pachyrhizi isolates and detecting in the population ASR26 alleles. In certain embodiments, the method further comprises expressing the identified ASR26 allele and a CcRPP1 polypeptide, a CcRPP1-like polypeptide or a combination thereof in a host cell and assaying the host cell for the presence of HR cell death, H₂O₂ accumulation, marker gene or marker metabolite expression, or any combination thereof.

Provided are methods for identifying ASR resistance polypeptides that do not compete with CcRpp1 comprising identifying one or more polypeptides that bind to an effector polypeptide having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 10-309, or a functional fragment thereof, assaying the one or more identified polypeptides for interaction with a polypeptide having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8, and selecting the one or more polypeptides that do not interact with the polypeptide having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8.

Further provided are methods for detecting a soluble CcRpp1 protein in a sample comprising incubating a mixture, the mixture comprising an ASR26 polypeptide having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 1-8 and a sample comprising a CcRpp1 protein, isolating the ASR26 polypeptide and polypeptides bound thereto from the mixture, and detecting in the isolated ASR26 polypeptide and polypeptides bound thereto the presence of a CcRpp1 protein, thereby detecting a soluble CcRpp1 protein.

### BRIEF DESCRIPTION OF THE SEQUENCE LISTING

The disclosure can be more fully understood from the following detailed description and the accompanying Sequence Listing, which form a part of this application. The sequence descriptions and sequence listing attached hereto comply with the rules governing nucleotide and amino acid sequence disclosures in patent applications as set forth in 37 C.F.R. §§ 1.831-1.835.

### DETAILED DESCRIPTION

The present disclosure provides compositions and methods for using effector proteins for identifying and selecting Asian soybean rust (ASR) resistance genes. The methods involve expressing in host cells nucleic acid sequences encoding effector proteins from Phakopsora pachyrhizi and Phakopsora meibomiae and identifying interacting polypeptides or incubating isolated and/or chimeric effector proteins and identifying interacting polypeptides. The methods also involve selecting plants for growing in an area of cultivation by determining the presence of specific ASR effectors in the area of cultivation.

Pathogens, such as Phakopsora pachyrhizi and Phakopsora meibomiae, which cause ASR, can overcome first-tier immunity of a plant by secreting molecules known as “effector proteins” or “effectors.” Plants have evolved, in certain cases, a second tier of immunity in which R gene products (e.g., NLR proteins) recognize specific effectors resulting in an effector triggered immunity. One such NLR protein, CcRpp1 (U.S. Pat. No. 10,842,097), has been shown to provide robust resistance to ASR when expressed in a plant. The identification of the effector protein recognized by CcRpp1 allows for the identification of CcRpp1-like proteins that may share a similar or the same mode of action, as well as the identity of non-competing ASR resistance genes that have a different mode of action (e.g., recognizes a different effector) than CcRpp1.

Accordingly, one aspect of the disclosure provides effector-encoding polynucleotides (e.g., isolated polynucleotides and recombinant polynucleotides) encoding effector polypeptides (e.g., isolated polypeptides and recombinant polypeptides) comprising, consisting essentially of, or consisting of an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8 and 10-309, or functional fragments thereof. In certain embodiments, the effector-encoding polynucleotide comprises a nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 310-317 and 319-618.

The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

In the present disclosure, “nucleic acid” refers to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

An “isolated” polynucleotide (or DNA) is used herein to refer to a nucleic acid sequence (or DNA) that is no longer in its natural environment, for example in an in vitro or in a heterologous recombinant bacterial or plant host cell. An isolated polynucleotide, or biologically active portion thereof, is substantially free of other cellular material or culture medium when produced by recombinant techniques, or substantially free of chemical precursors or other chemicals when chemically synthesized. An isolated polynucleotide is free of sequences (optimally protein encoding sequences) that naturally flank the nucleic acid (i.e., sequences located at the 5′ and 3′ ends of the nucleic acid) in the genomic DNA of the organism from which the nucleic acid is derived. A “recombinant” polynucleotide (or DNA) is used herein to refer to a nucleic acid sequence (or DNA) that is in a recombinant bacterial or plant host cell. In some embodiments, an “isolated” or “recombinant” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the disclosure, “isolated” or “recombinant” when used to refer to nucleic acid molecules excludes isolated chromosomes. For example, in various embodiments, the recombinant nucleic acid molecules encoding the effector polypeptides of the disclosure can contain less than about 5 kb, 4 kb, 3 kb, 2 kb, 1 kb, 0.5 kb or 0.1 kb of nucleic acid sequences that naturally flank the nucleic acid molecule in genomic DNA of the cell from which the nucleic acid is derived.

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

As used herein, “functional fragment,” “fragment that is functionally equivalent,” “functionally equivalent fragment” and the like refer to a portion or subsequence of a polypeptide sequence of the present disclosure in which its native ability is retained. For example, a functional fragment of an effector polypeptide, such as those described herein, maintains the ability to be recognized by and bind to an NLR protein. Accordingly, in certain embodiments, a functional fragment of an ASR26 effector polypeptide (e.g., SEQ ID NOs: 1-8) binds to the CcRpp1 polypeptide sequence of SEQ ID NO: 9. In certain embodiments, the functional fragments of the polypeptides described herein comprises an N-terminal and/or a C-terminal truncation of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more amino acids.

Functional fragments of a particular polypeptide of the embodiments (i.e., the reference polypeptide) can also be evaluated by comparison of the percent sequence identity between the polypeptide and the reference polypeptide. Percent sequence identity between any two polypeptides can be calculated using sequence alignment programs known in the art.

Functional fragments of a native protein of the embodiments can have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence for the native protein as determined by sequence alignment programs known in the art. A functional fragment of a protein of the present disclosure can differ from that protein by as few as about 1-15 amino acid residues, as few as about 1-10, such as about 6-10, as few as about 5, as few as 4, 3, 2, or even 1 amino acid residue.

The polynucleotides described herewith can be used to isolate corresponding sequences from other organisms, particularly other Phakopsora pachyrhizi and Phakopsora meibomiae. In this manner, methods such as PCR or hybridization can be used to identify such sequences based on their sequence identity to the sequences set forth herein. Sequences isolated based on their sequence identity to the entire sequences set forth herein or to variants and fragments thereof are encompassed by the present disclosure. Such sequences include sequences that are orthologs or alleles of the disclosed sequences. The term “orthologs” refers to genes derived from a common ancestral gene and which are found in different species as a result of speciation. The term “allele” refers to a variation of a nucleotide sequence (e.g., SNP) that encodes the synthesis of a gene product at the same place on a DNA molecule. Genes found in different species are considered orthologs when their nucleotide sequences and/or their encoded protein sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode for an effector protein that hybridize to the sequences disclosed herein, or functional fragments thereof, are encompassed by the present disclosure.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference).

In certain embodiments the effector-encoding polynucleotide encodes a CcRpp1 effector polypeptide, also referred to herein as ASR26, comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8, or a functional fragment thereof. In certain embodiments, the CcRpp1 effector-encoding polynucleotide comprises a nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 310-317.

In certain embodiments, the polynucleotide encoding the polypeptide is operably linked to at least one (e.g., at least 1, 2, 3, 4, 5, 6, 7 or more) regulatory element. In certain embodiments, the regulatory element is a promoter. In certain embodiments, the regulatory element is a heterologous regulatory element (e.g., heterologous promoter). In certain embodiments the heterologous regulatory element is heterologous to the polynucleotide sequence encoding one or more of the effector polypeptides disclosed herein. In certain embodiments, the heterologous regulatory element operably linked to the polynucleotide is heterologous to the host cell.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

As used herein “operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame.

As used herein “regulatory element” generally refers to a transcriptional regulatory element involved in regulating the transcription of a nucleic acid molecule such as a gene or a target gene. The regulatory element is a nucleic acid and may include a promoter, an enhancer, an intron, expression modulating elements (EMEs), a 5′-untranslated region (5′-UTR, also known as a leader sequence), or a 3′-UTR or a combination thereof. A regulatory element may act in “cis” or “trans”, and generally it acts in “cis”, i.e., it activates expression of genes located on the same nucleic acid molecule, e.g., a chromosome, where the regulatory element is located.

An “enhancer” element is any nucleic acid molecule that increases transcription of a nucleic acid molecule when functionally linked to a promoter regardless of its relative position. Various enhancers are known in the art including for example, introns with gene expression enhancing properties in plants, the ubiquitin intron (i.e., the maize ubiquitin intron 1 (see, for example, NCBI sequence S94464)), the omega enhancer or the omega prime enhancer (Gallie, et al., (1989) Molecular Biology of RNA ed. Cech (Liss, New York) 237-256 and Gallie, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used. The above list of transcriptional enhancers is not meant to be limiting. Any appropriate transcriptional enhancer can be used in the embodiments described herein.

A “repressor” (also sometimes called herein silencer) is defined as any nucleic acid molecule which inhibits the transcription when functionally linked to a promoter regardless of relative position. The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature mRNA derived from the gene but is not necessarily a part of the sequence that encodes the final gene product. The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes. The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

As used herein “promoter” refers to a region of DNA upstream from the start of transcription and involved in recognition and binding of RNA polymerase and other proteins to initiate transcription. A “plant promoter” is a promoter capable of initiating transcription in plant cells. In certain embodiments, the polynucleotides described herein are operably linked to a promoter that drives expression in a plant cell. Any promoter known in the art can be used in the methods of the present disclosure including, but not limited to, constitutive promoters, pathogen-inducible promoters, wound-inducible promoters, tissue-preferred promoters, and chemical-regulated promoters. The choice of promoter may depend on the desired timing and location of expression in the transformed plant as well as other factors, which are known to those of skill in the art. Such constitutive promoters include, for example, the core promoter of the Rsyn7 promoter and other constitutive promoters disclosed in WO 99/43838 and U.S. Pat. No. 6,072,050; the core CaMV 35S promoter; rice actin; ubiquitin; pEMU; MAS; ALS; and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142; and 6,177,611, which are known in the art, and can be contemplated for use in the present disclosure.

Generally, it can be beneficial to express the gene from an inducible promoter, particularly from a pathogen-inducible promoter. Such promoters include those from pathogenesis-related proteins (PR proteins), which are induced following infection by a pathogen, e.g., PR proteins, SAR proteins, beta-1,3-glucanase, chitinase, etc.

Of interest are promoters that are expressed locally at or near the site of pathogen infection. Additionally, as pathogens find entry into plants through wounds or insect damage, a wound-inducible promoter can be used in the constructions of the disclosure. Such wound-inducible promoters include potato proteinase inhibitor (pin II) gene, wun1 and wun2, win1 and win2, systemin, WIP1, MPI gene, and the like.

Chemical-regulated promoters can be used to modulate the expression of a gene in a plant through the application of an exogenous chemical regulator. Depending upon the objective, the promoter can be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (e.g., the glucocorticoid-inducible promoter, and tetracycline-inducible and tetracycline-repressible promoters).

Tissue-preferred promoters can be utilized to target enhanced expression of the target genes or proteins within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2): 255-265; Kawamata et al. (1997) Plant Cell Physiol. 38(7): 792-803; Hansen et al. (1997) Mol. Gen Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascini et al. (1996) Plant Physiol. 112(2): 513-524; Yamamoto et al. (1994) Plant Cell Physiol. 35(5): 773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20): 9586-9590; and Guevara-Garcia et al. (1993) Plant J. 4(3): 495-505. Such promoters can be modified.

Leaf-specific promoters are known in the art. See, for example, Yamamoto et al. (1997) Plant J. 12(2) 255-265; Kwon et al. (1994) Plant Physiol. 105:357-67; Yamamoto et al. (1994) Plant Cell Physiol. 35(5): 773-778; Gotor et al. (1993) Plant J. 3:509-18; Orozco et al. (1993) Plant Mol. Biol. 23(6): 1129-1138; and Matsuoka et al. (1993) Proc. Natl. Acad. Sci. USA 90(20): 9586-9590.

“Seed-preferred” promoters include both “seed-specific” promoters (those promoters active during seed development such as promoters of seed storage proteins) as well as “seed-germinating” promoters (those promoters active during seed germination). Such seed-preferred promoters include, but are not limited to, Cim1 (cytokinin-induced message), cZ19Bl (maize 19 kDa zein), milps (myo-inositol-1-phosphate synthase), and celA (cellulose synthase) (see WO 00/11177, herein incorporated by reference). Gama-zein is a preferred endosperm-specific promoter. Glob-1 is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also WO 00/12733, where seed-preferred promoters from end1 and end2 genes are disclosed; herein incorporated by reference.

Also provided is a nucleic acid (e.g., DNA) construct comprising any of the effector-encoding polynucleotides described herein. The use of the term “nucleic acid construct”, “nucleotide constructs” or the like herein is not intended to limit the embodiments to nucleotide constructs comprising DNA. Those of ordinary skill in the art will recognize that nucleotide constructs, particularly polynucleotides and oligonucleotides composed of ribonucleotides and combinations of ribonucleotides and deoxyribonucleotides, may also be employed in the methods disclosed herein. The nucleotide constructs, nucleic acids, and nucleotide sequences of the embodiments additionally encompass all complementary forms of such constructs, molecules, and sequences. Further, the nucleotide constructs, nucleotide molecules, and nucleotide sequences of the embodiments encompass all nucleotide constructs, molecules, and sequences which can be employed in the methods of the embodiments for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the embodiments also encompass all forms of nucleotide constructs including, but not limited to, single-stranded forms, double-stranded forms, hairpins, stem-and-loop structures and the like.

In certain embodiments, the effector-encoding polynucleotides described herein are provided in expression cassettes (e.g., a plasmid, cosmid, virus, autonomously replicating sequence, phage, or linear or circular single-stranded or double-stranded DNA or RNA nucleotide sequence) for expression in a host of interest or any plant of interest. The cassette can include 5′ and 3′ regulatory sequences operably linked to an effector-encoding polynucleotide. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the effector-encoding polynucleotide to be under the transcriptional regulation of the regulatory regions. The expression cassette may additionally contain selectable marker genes.

The expression cassette can include in the 5′-3′ direction of transcription, a transcriptional and translational initiation region (e.g., a promoter), an effector-encoding polynucleotide, and a transcriptional and translational termination region (e.g., termination region) functional in plants. The regulatory regions (e.g., promoters, transcriptional regulatory regions, and translational termination regions) and/or the effector-encoding polynucleotide may be native/analogous to the host cell or to each other. Alternatively, the regulatory regions and/or the effector-encoding polynucleotide may be heterologous to the host cell or to each other.

The termination region may be native with the transcriptional initiation region, with the plant host, or may be derived from another source (i.e., foreign or heterologous) than the promoter, the effector-encoding polynucleotide, the plant host, or any combination thereof.

The expression cassette may additionally contain a 5′ leader sequences. Such leader sequences can act to enhance translation. Translation leaders are known in the art and include viral translational leader sequences.

Generally, the expression cassette can comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, imidazolinones, and 2,4-dichlorophenoxyacetate (2,4-D). The above list of selectable marker genes is not meant to be limiting. Any selectable marker gene can be used in the present disclosure.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided are host cells comprising at least one of the effector-encoding polynucleotide sequences, nucleic acid constructs, or expression cassettes described herein, so that the host cell expresses any of the effector polypeptides described herein. In certain embodiments, the host cell is a bacterial cell, a fungal cell, or a plant cell. In certain embodiments, the plant cell is a plant protoplast. In certain embodiments, the plant cell or plant protoplast is a tobacco cell, tobacco protoplast, Arabidopsis cell, Arabidopsis protoplast, soybean cell or soybean protoplast. In certain embodiments, the nucleic acid construct or expression cassette, described herein, is expressed in the host cell. In certain embodiments, the host cell further comprises one or more (e.g., 2, 3, 4, 5, 6, 7, 8, 9, 10, 20, or 50, or more) candidate ASR resistance genes. In certain embodiments, the candidate ASR resistance gene is introduced into the host cell using a nucleic acid construct comprising the candidate ASR resistance gene operably linked to a regulatory element, such as, for example a heterologous promoter.

As used herein, “introduced”, “introducing” or the like is intended to mean presenting to the host cell the inventive polynucleotide or resulting polypeptide in such a manner that the sequence gains access to the interior of the cell. The methods of the disclosure do not depend on a particular method for introducing a sequence into a host cell, only that the polynucleotide or polypeptide gains access to the interior of the cell.

In certain embodiments, the polynucleotides described herein are transiently expressed in the host cell using a transient transformation technique. In certain embodiments, the polynucleotides are stably expressed in the host cell using a stable transformation technique. In certain embodiments, the polynucleotides are introduced into the plant, plant cell, plant part, seed or grain using a nucleic acid construct or an expression cassette described herein. In certain embodiments, the polynucleotides are introduced into the plant, plant cell, plant part, seed or grain using a genome editing technique.

“Stable transformation” is intended to mean that the polynucleotide introduced into a plant integrates into the genome of the plant of interest and is capable of being inherited by the progeny thereof. “Transient transformation” is intended to mean that a polynucleotide is introduced into the plant of interest and does not integrate into the genome of the plant or organism, or a polypeptide is introduced into a plant or organism.

Transformation protocols as well as protocols for introducing polypeptides or polynucleotide sequences into host cells may include but are not limited to microinjection, electroporation, Agrobacterium-mediated transformation, Ochrobacterium-mediated transformation, direct gene transfer, viral mediated, ballistic particle acceleration, heat shock, and lipid-mediated transfection. The type of transformation or transfection method for use with the compositions and method described herein may vary depending on the type of host cell, e.g., bacterial cell, fungal cell, or plant cell, targeted for transformation.

Also provided herein are chimeric polypeptides and polynucleotides encoding chimeric polypeptides comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8 and 10-309, or functional fragments thereof, operably linked to a heterologous peptide. In certain embodiments, the polynucleotide encoding the chimeric polypeptide is in a nucleic acid construct comprising the polynucleotide operably linked to a regulatory element, such as, for example, a heterologous promoter. In certain embodiments, the chimeric polypeptide binds to a CcRpp1 polypeptide sequence comprising an amino acid sequence having at least 90%, 91%, 92%, 93%, 94%, 95%, 96%, 97%, 98%, or 99% or more sequence identity to SEQ ID NO: 9. In certain embodiments, the chimeric polypeptide comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8, or functional fragments thereof.

In certain embodiments, the heterologous peptide comprises a protein tag. As used herein, a “protein tag” includes, but is not limited to, affinity tags, solubilization tags, chromatography tags, epitope tags, and fluorescent tags. The location of the protein tag in the chimeric polypeptide is not particularly limited so long as the chimeric polypeptide maintains binding to the CcRpp1 polypeptide or corresponding resistance polypeptide, such that in certain embodiments the protein tag may be operably linked to the N-terminus, C-terminus, or both the N- and C-terminus of the amino acid sequence. In certain embodiments, the protein tag is a FLAG tag (e.g., 3×-FLAG), a myc tag, a His tag, an MBP tag, a glutathione-s-transferase tag, an HA tag, or an Fc tag.

Also provided herein are methods for identifying CcRpp1-like proteins by identifying one or more polypeptides that bind to a chimeric effector polypeptide or an effector polypeptide or functional fragment thereof described herein such as those comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8.

As used herein a “CcRpp1-like” protein refers to a protein that has the same or similar biological activity to CcRpp1 but does not share 100% sequence identity with CcRpp1 (i.e., SEQ ID NO: 9) over the full length of the protein. In certain embodiments, the CcRpp1-like protein binds to at least one of the ASR26 effector protein alleles described herein (e.g., SEQ ID NOs: 1-8).

In certain embodiments, the method comprises providing a population of polypeptides from a plant cell, optionally an ASR resistant plant cell, incubating the population of polypeptides with at least one (e.g., at least 1, 2, 3, 4, 5, 6, 7, 8, 9, 10, 20 or more) effector polypeptide, or functional fragment thereof, described herein (e.g., SEQ ID NOs: 1-8) or at least one (e.g., at least 1, 2, 3, 4, 5, 6, 7, 8, 9, 10, 20 or more) chimeric polypeptide described herein or a combination of at least one effector polypeptide and at least one chimeric polypeptide, isolating the at least one effector polypeptide and/or the at least one chimeric polypeptide along with polypeptides interacting therewith from the population, and identifying polypeptides interacting with the effector polypeptide or chimeric polypeptide.

In certain embodiments, the method comprises providing a population comprising a plurality of R-gene polypeptides, incubating the population of R-gene polypeptides with at least one (e.g., at least 1, 2, 3, 4, 5, 6, 7, 8, 9, 10, 20 or more) effector polypeptide described herein (e.g., SEQ ID NOs: 1-8), or functional fragment thereof, or at least one (e.g., at least 1, 2, 3, 4, 5, 6, 7, 8, 9, 10, 20 or more) chimeric polypeptide described herein or a combination of at least one effector polypeptide and at least one chimeric polypeptide, isolating the at least one effector polypeptide and/or the at least one chimeric polypeptide along with R-gene polypeptides interacting therewith from the population, and identifying the R-gene polypeptides interacting with the effector polypeptide or chimeric polypeptide.

In certain embodiments of the methods described herein, the incubation is performed under conditions that allow for binding of the at least one chimeric polypeptide or the at least one effector polypeptide, or functional fragment thereof, to one or more polypeptides of the population. Those of ordinary skill in the art will recognize the conditions that can be manipulated to allow for, or improve, the capture of protein-protein interactions. For example, conditions that can be altered include, but are not limited to, buffer composition, pH, incubation time, and temperature. In certain embodiments, the incubation is performed in the presence of a crosslinker. The type of crosslinker used is not particularly limited so long as the crosslinker enhances the binding of the effector and bound polypeptide. Types of crosslinkers for use in the methods described herein include, but are not limited to, homobifunctional crosslinkers and photoreactive crosslinkers.

“Isolating” as used in the methods described herein encompasses removing a polypeptide and bound polypeptides from a biological environment (e.g., incubation culture). The method for isolating the effector or chimeric polypeptides and bound polypeptides (e.g., R-gene candidates) of the methods described herein is not particularly limited and may be any method known in the art that can isolate the effector or chimeric polypeptide and allow for subsequent identification of the bound polypeptides. In certain embodiments, the method comprises immunoprecipitation using an antibody specific for the effector protein and/or chimeric protein. In certain embodiments, the antibody is specific for the protein tag of the chimeric polypeptide (e.g., anti-HA antibody). In certain embodiments, the method comprises a pulldown assay, such as, for example pulldown using a peptide that recognizes the protein tag of the chimeric polypeptide. In certain embodiments, the method comprises column chromatography, e.g., affinity chromatography. In certain embodiments, the column chromatography uses an antibody that binds to the effector polypeptide or functional fragment. In certain embodiments, the column chromatography uses a ligand (e.g., antibody, Protein A and/or G, peptide, glutathione) that binds to the protein tag of the chimeric polypeptide. In certain embodiments, the method comprises an enzyme linked immunosorbent assay (ELISA).

The method for detecting a polypeptide (e.g., R-gene proteins or polypeptides from the ASR resistant plant cell) interacting with or bound to the effector polypeptide or chimeric polypeptide is not particularly limited such that the polypeptide may be identified using any method known in the art. For example, in certain embodiments, the method comprises mass spectrometry.

In certain embodiments the plant cell from which the population is provided is from an ASR resistant plant or a legume crop species from a genus selected from the group consisting of Glycine, Vigna, and Lablab.

In certain embodiments, the legume crop species or legume-derived gene is derived from the genus Glycine. Examples of Glycine species include, but are not limited to, Glycine arenaria, Glycine argyrea, Glycine cyrtoloba, Glycine canescens, Glycine clandestine, Glycine curvata, Glycine falcata, Glycine latifolia, Glycine microphylla, Glycine pescadrensis, Glycine stenophita, Glycine syndetica, Glycine soja, Glycine tabacina and Glycine tomentella.

In another embodiment, the legume crop species or legume-derived gene is derived from the genus Vigna. Vigna is a pantropic genus that comprises approximately 100 species. It is a taxonomic group subdivided into the subgenera Vigna, Haydonia, Plectotropis (African), Ceratotropis (Asian), Sigmoidotropis, and Lasiopron. The genus includes economically relevant species such as Vigna unguiculata (L.) Walp (cowpea), Vigna radiata (L.) Wilczek (mung bean), Vigna angularis (Willd.) Ohwi and Ohashi (azuki bean), Vigna mungo (L.) Hepper (black gram), and Vigna umbellata (Thunb.) Ohwi and Ohashi (rice bean). Four subspecies are recognized within Vigna unguiculata: dendtiana, a wild relative of cultivated subspecies; cylindrica, cultivated catjang; sesquipedalis, cultivated yardlong bean; and unguiculata, cultivated black-eyed pea. Vigna unguiculata ssp. unguiculata is further divided into cultivar groups Unguiculata, grown as a pulse; Biflora or Cilindrica (catjang), mainly used as a forage; Sesquipedalis (yardlong or asparagus bean), grown as a vegetable; Textilis, cultivated for the fibres of its long floral peduncles; and Melanophthalmus (black-eyed pea). Susceptibility of several Vigna species, including Vigna radiata, Vigna mungo and Vigna unguiculata to Phakopsora pachyrhizi has been reported under field and greenhouse conditions.

In another embodiment, the legume crop species or legume-derived gene is derived from the genus Lablab. Lablab purpureus (L.) Sweet is a leguminous species (Verdcourt (1971) Flora of Tropical East Africa, pp. 696-699, Crown Agents, London, UK; and Duke et al. (1981) Handbook of Legumes of World Economic Importance, pp. 102-106, Plenum Press, New York, USA and London, UK) native to Asia and Africa (Pengelly and Maass, (2001) Gen. resour. crop ev. 48:261-272). It is commonly known as lablab bean, hyacinth bean, bonavist bean, field bean, Egyptian bean, poor man's bean, Tonga bean (English) and by at least 20 additional vernacular names. It is grown in Africa, Asia, and the Caribbean as either a pulse crop or as a green vegetable (Duke et al. (1981) Handbook of Legumes of World Economic Importance, pp. 102-106, Plenum Press, New York, USA and London, UK); and Pengelly and Maass, (2001) Gen. resour. crop ev. 48:261-272).

Lablab purpureas has been reported as an alternative host for Phakopsora pachyrhizi (Perez-Hernandez, (2007) Alternative hosts of Phakopsora pachyrhizi in the Americas: An analysis of their role in the epidemiology of Asian soybean rust in the continental U.S. M.Sc. thesis. Iowa State University. Ames, Iowa. U.S.A.; Vakili (1981) Plant Dis. 65:817-819; and Poonpolgul and Surin, (1980) Soybean Rust Newsletter, 3:30-31).

In an aspect, the legume crop species or legume-derived gene is derived from the genus Cicer, Cajanus, Medicago, Phaseolus, Pisum, Pueraria, or Trifolium. Examples of Cicer species include, but are not limited to, Cicer arietinum, Cicer echinospermum, Cicer reticulatum and Cicer pinnatifldum. An example of the Cajanus species include, but is not limited to, Cajanus cajan. Examples of the Medicago species include, but are not limited to, Medicago truncatula and Medicago sativa. Examples of the Phaseolus species include, but are not limited to, Phaseolus vulgaris, Phaseolus lunatus, Phaseolus acutifolius and Phaseolus coccineus. Examples of the Pisum species include, but are not limited to, Pisum abyssinicum, Pisum sativum, Pisum elatius, Pisum fulvum, Pisum transcaucasium and Pisum humile. An example of the Pueraria species includes, but is not limited to, Pueraria lobata. Examples of the Trifolium species include, but are not limited to, Trifolium aureum and Trifolium occidentale.

R gene polypeptides are proteins that recognize the activity of specific effectors resulting in an effector-triggered immunity. R genes typically encode proteins that feature C-terminal leucine-rich repeats (LRRs) and nucleotide-binding site (NBS) domains. Such nucleic acid binding LRRs are designated nucleotide-binding LRR (NLR) proteins. The NBS domain functions as a molecular switch depending on the bound nucleotide: ADP-bound in the resting state and ATP-bound in the active state. The LRR domain is generally thought to be involved in effector recognition and autoinhibition (Ting et al., Immunity, 28 (2008), pp. 285-287). Typical plant NLRs almost universally feature the additional coiled-coil (CC) or Toll/interleukin-1 receptor (TIR)N-terminal domain. These N-terminal domains are used to sort plant NLRs into two main groups termed CNLs (CC-NLRs) and TNLs (TIR-NLRs). Both CC and TIR domains have been demonstrated to play key roles in the formation of dimers and oligomers.

The population comprising a plurality of R-gene polypeptides may be produced using any method known in the art. In certain embodiments, the population is generated by isolating R-gene polypeptides from ASR resistant plant species, such as legume crop species disclosed herein. In certain embodiments, the population is generated by isolating R-gene polypeptides from one or more legume crop species. In certain embodiments, the population is generated by producing variants of known ASR R-gene polypeptides. As used herein, a variant refers to a protein derived from the native protein by deletion or addition of one or more amino acids at one or more sites in the native protein and/or substitution of one or more amino acids at one or more sites in the native protein. Methods for such manipulations are known in the art. For example, amino acid sequence variants and fragments of the resistance proteins can be prepared by mutations in the DNA. Methods for mutagenesis and polynucleotide alterations are known in the art, including, but not limited to, procedures such as DNA shuffling. Strategies for such DNA shuffling are known in the art. As would be understood by a person of ordinary skill in the art, the population of R gene polypeptides may be obtained from multiple sources or methods, such that, for example, the population may contain R-genes from a resistant plant species along with R gene variants.

In certain embodiments, the method for identifying CcRpp1-like polypeptides comprises expressing in a host cell at least one of the polynucleotides described herein (e.g., polynucleotide encoding effector polypeptide, or functional fragment thereof, or chimeric polypeptide), isolating the expressed effector polypeptide and/or chimeric polypeptide and proteins bound thereto, and identifying one or more polypeptides interacting with the expressed effector polypeptide and/or chimeric polypeptide. The methods for isolating the effector polypeptide may be any method described herein or known in the art. Similarly, the method for identifying the bound (e.g., interacting) polypeptides may be any method described herein or known in the art. In certain embodiments, the host cell comprises one or more candidate ASR resistance genes (e.g., R genes). The one or more candidate ASR resistance genes may be introduced into the host cells by a nucleic acid construct, may be native ASR resistance genes or may be a combination of introduced and native genes. The host cell may be any host cell provided herein. In certain embodiments, the host cell is a plant cell such as, for example, Nicotiana benthamiana or maize (optionally using the methods for detecting interaction as described in International Patent Application Publication Number WO 2021/211227).

In certain embodiments, the method for identifying CcRpp1-like polypeptides further comprises validating the one or more identified polypeptides as a polypeptide increasing resistance to ASR. In certain embodiments, the method comprises expressing in a host cell at least one of the effector proteins or chimeric proteins described herein and one or more of the identified polypeptides and assaying the host cells for hypersensitive response (HR) cell death, H₂O₂ accumulation, marker gene or marker metabolite expression, or any combination thereof. In certain embodiments, the effector protein or chimeric protein comprises an ASR26 allele (e.g., SEQ ID NOs: 1-8) or a functional fragment thereof. Host cells showing either HR cell death, H₂O₂ accumulation, marker gene or marker metabolite expression, or any combination thereof would indicate that the identified polypeptide is expected to increase resistance to ASR, such that embodiments in which the effector protein or chimeric protein comprises an ASR26 allele or a functional fragment thereof encoding a polypeptide producing HR cell death, H₂O₂ accumulation, marker gene or marker metabolite expression, or any combination thereof would indicate that the identified polypeptide is a CcRpp1-like polypeptide. The host cell may be any host cell provided herein. In certain embodiments, the host cell is a plant cell such as, for example, Nicotiana benthamiana. Methods for assaying HR cell death and H₂O₂ accumulation are known in the art and include, but not limited to, visual identification of HR cell death and DAB staining for H₂O₂ accumulation. Examples of changes in metabolite expression or gene expression in response to pathogen infection can be found in, for example, Silva et al. (Metabolites, 11(3): 179 (2021)), Castro-Moretti et al. (Metabolites, 10(2): 52 (2020)), and Qi et al (PLOS Pathog, 12(9): e1005827 (2016)).

In certain embodiments, the method for validating the identified polypeptide comprises expressing the polypeptide in a legume plant, plant cell, tissue or organ (e.g., soybean) and assaying the plant for resistance, immunity, or susceptibility to ASR. General methods for determination of resistance, immunity, or susceptibility of a plant to a particular pathogen are known to one skilled in the art. For example, a method for screening or assaying legume plants for resistance, immunity or susceptibility to a plant disease may comprise exposing a plant cell, tissue or organ (e.g., leaf) to a pathogen (e.g., Phakopsora pachyrhizi) and then determining and/or measuring in the exposed plant, the degree of resistance, immunity and/or susceptibility to a plant disease (e.g., ASR) caused by the pathogen. The method can further comprise measuring any observable plant disease symptoms on the plant exposed to the plant pathogen and then comparing the plant disease symptoms to a reference standard to determine the degree or extent of disease resistance. Methods of exposing a plant cell, tissue or organ to a pathogen are known in the art. Methods of measuring, comparing, and determining the level of resistance, immunity and/or susceptibility (e.g., plant disease symptoms) to a disease, such as, for example, ASR, caused by the pathogen are also known in the art.

In certain embodiments, the method for identifying CcRpp1-like polypeptides comprises expressing one or more effector polypeptides, or functional fragments thereof, or chimeric polypeptides or any combination thereof in a host cell comprising one or more candidate ASR resistance genes and assaying the host cell for HR cell death, H₂O₂ accumulation, marker gene expression, marker metabolite expression, or any combination thereof. In certain embodiments, the effector polypeptide, chimeric polypeptide, or functional fragment comprises an ASR26 allele. In certain embodiments, the host cell is a cell isolated from an ASR resistant plant such that the cell comprises candidate ASR resistance genes. In certain embodiments, the candidate ASR resistance genes are introduced into the host cell.

Also provided herein is a method for selecting a plant for growing in an area of cultivation comprising providing a sample comprising a plurality of Phakopsora pachyrhizi isolates, Phakopsora meibomiae isolates or a combination thereof from an area of cultivation, assaying the isolates for the presence of an ASR26 allele or ortholog thereof, and selecting a plant expressing a CcRpp1 polypeptide, a CcRpp1-like polypeptide, or a combination thereof for growing in the area of cultivation when the polynucleotide is present. In certain embodiments, the ASR26 allele comprises a nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 310-317. In certain embodiments, the ASR26 allele comprises a polypeptide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 1-8. In certain embodiments, the CcRpp1 polypeptide comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to SEQ ID NO: 9. In certain embodiments, the plant expressing a CcRpp1 polypeptide, a CcRpp1-like polypeptide, or a combination thereof is selected when the polynucleotide is present in at least 5%, 10%, 15%, 20%, 25%, 30%, 35%, 40%, 45%, 50%, 55%, 60%, 65%, 70%, 75%, 80%, or 90% of the isolates tested. Methods for detecting the presence of a nucleotide sequence in a sample (e.g., Phakopsora pachyrhizi, Phakopsora meibomiae isolates) are well known in the art and include, for example, PCR.

As used herein, an “area of cultivation” comprises any region in which one desires to grow a plant. Such areas of cultivations include, but are not limited to, a field in which a plant is cultivated (such as a crop field, a sod field, a tree field, a managed forest, a field for culturing fruits and vegetables, etc), a greenhouse, or a growth chamber.

In certain embodiments, the plant expressing the CcRpp1 polypeptide, the CcRpp1-like polypeptide, or a combination thereof described herein are elite plant lines (e.g., elite soybean line). In certain embodiments, plant cells, plant parts, seeds, and grain are isolated from or produced by an elite plant line. As used herein, “elite line” refers to any line that has resulted from breeding and selection for superior agronomic performance that allows a producer to harvest a product of commercial significance. Numerous elite lines are available and known to those of skill in the art of plant breeding (e.g., soybean, canola, and sunflower breeding). An “elite population” is an assortment of elite individuals or lines that can be used to represent the state of the art in terms of agronomically superior genotypes of a given crop species, such as soybean.

The plant species of the compositions and methods of the present disclosure (e.g., plants from which host cells are isolated and plants expressing the CcRpp1 polypeptide, the CcRpp1-like polypeptide, or a combination thereof) can be any plant species, including, but not limited to, monocots and dicots. Examples of plants of interest include, but are not limited to, corn (Zea mays), Brassica spp. (e.g., Brassica napus, Brassica rapa, Brassica juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatas), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Per sea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

In certain embodiments, the plant of the compositions and methods described herein is a legume crop species, including, but not limited to, alfalfa (Medicago sativa); clover or trefoil (Trifolium spp.); pea, including (Pisum satinum), pigeon pea (Cajanus cajan), cowpea (Vigna unguiculata) and Lathyrus spp.; bean (Fabaceae or Leguminosae); lentil (Lens culinaris); lupin (Lupinus spp.); mesquite (Prosopis spp.); carob (Ceratonia siliqua), soybean (Glycine max), peanut (Arachis hypogaea) or tamarind (Tamarindus indica). The terms “legume species” and “legume crop species” are used herein to refer to plants, and can be for example, a plant of interest. In certain embodiments, the legume species or legume crop species is a plant, plant part or plant cell.

Also provided herein are methods for identifying ASR26 alleles comprising providing a population comprising a plurality of Phakopsora pachyrhizi isolates, Phakopsora meibomiae isolates or a combination thereof and detecting in the population alleles of ASR26. In certain embodiments, the plurality of isolates is taken from an area of cultivation. The method for identifying the allele is not particularly limited and may be any method known in the art. In certain embodiments, the detecting comprises genomic sequencing or RNA sequencing.

In certain embodiments, the method further comprises assaying the identified ASR26 alleles to determine if the identified allele is recognized by the CcRpp1 polypeptide, a chimeric CcRpp1 polypeptide, or the CcRpp1-like polypeptide, or any combination thereof. The assay can be any of the assays described herein, or known in the art, that determines an interaction between the ASR26 allele and CcRpp1, CcRpp1-like, or chimeric CcRpp1 polypeptide. In certain embodiments, the assay is a pulldown assay or immunoprecipitation assay to determine binding between the allele and CcRpp1, CcRpp1-like, or chimeric CcRpp1 polypeptide. In certain embodiments, the ASR26 allele and CcRpp1, CcRpp1-like, or chimeric CcRpp1 polypeptide are expressed in a host cell and the presence of HR cell death, H₂O₂ accumulation, marker gene expression and/or marker metabolite expression is measured.

As would be understood by a person of ordinary skill in the art, the methods described herein to identify CcRpp1-like polypeptides using an ASR26 allele or functional fragment thereof, could be adapted to identify candidate ASR resistance polypeptides using the effector polypeptides (e.g., non-ASR26 polypeptides) described herein. Accordingly, also provided herein are methods for identifying ASR resistance proteins by identifying one or more polypeptides that bind to a effector polypeptide comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to any one of SEQ ID NOs: 10-309, a functional fragment thereof or a chimeric polypeptide produced therefrom. The methods for identifying the one or more polypeptides may be any method described herein, or known in the art, that allows for the identification of interacting proteins. The methods may further include validating the identified interacting protein using any of the validation methods described herein including, but not limited to, expression in host cells to assay HR cell death, H₂O₂ accumulation, marker gene or marker metabolite expression, or any combination thereof and expressing in plants to determine resistance to ASR.

In certain embodiments, the candidate resistance genes are assayed for binding to an ASR26 allele. In certain embodiments, candidate resistance genes are selected that do not bind an ASR26 allele. In certain embodiments, the population of R-gene polypeptides for use in identifying candidates binding to or interacting with the effector polypeptides (e.g., SEQ ID NOs: 10-309) is pre-screened to remove R-gene polypeptides that bind to an ASR26 allele. Methods to remove polypeptides from a sample are known in the art and include, but are not limited to, pulldown assays, immunoprecipitation, and column chromatography.

Also provided herein are methods for identifying ASR resistance polypeptides that do not compete with CcRpp1 (e.g., have a different mode of action). In certain embodiments, the method comprises identifying one or more polypeptides that bind to an effector polypeptide having at least 90% sequence identity to any one of SEQ ID NOs: 10-309, or a functional fragment thereof, assaying the one or more identified polypeptides for interaction with a polypeptide having at least 90% sequence identity to any one of SEQ ID NOs: 1-8, and selecting the one or more polypeptides that do not interact with the polypeptide having at least 90% sequence identity to any one of SEQ ID NOs: 1-8. The one or more identified ASR resistance polypeptides that do not compete with CcRpp1 can be expressed in a plant either alone or in combination with another ASR resistance polynucleotide, such as, for example, CcRpp1 using the methods described herein or known in the art, to generate a plant having ASR resistance. The method for identifying the one or more polypeptides that bind to an effector polypeptide having at least 90% sequence identity to any one of SEQ ID NOs: 10-309 may be any method described herein (e.g., binding assays and/or measuring HR cell death, H₂O₂ accumulation, marker gene or marker metabolite expression, or any combination thereof in a host cell). In certain embodiments, the method comprises providing a population of polypeptides from an ARS resistant plant cell, incubating the population of polypeptides with the effector polypeptide, a functional fragment thereof or a chimeric polypeptide comprising the effector polypeptide or the functional fragment thereof under conditions that allow for binding of the at least one effector polypeptide, functional fragment thereof or chimeric polypeptide to one or more polypeptides of the population, isolating the effector polypeptide or the chimeric polypeptide and polypeptides interacting therewith from the population, and identifying the one or more polypeptides. In certain embodiments, the one or more polypeptides are identified by providing a population comprising a plurality of R-gene candidates, optionally pre-screened to remove candidates that bind to an ASR26 allele, incubating the population with the effector polypeptide or a chimeric polypeptide comprising the effector polypeptide or the functional fragment thereof under conditions that allow for binding of the effector polypeptide or the chimeric polypeptide to one or more R-gene candidates in the population, isolating the effector polypeptide or the chimeric polypeptide and interacting R-gene candidates from the population, and identifying the R-gene candidates. In certain embodiments, the one or more polypeptides are identified by introducing into a host cell comprising one or more candidate ASR resistance genes a nucleic acid construct comprising a polynucleotide encoding the effector polypeptide, or a functional fragment thereof, or a chimeric polypeptide comprising the effector polypeptide or the functional fragment thereof, isolating the expressed effector polypeptide or chimeric polypeptide, and identifying one or more polypeptides interacting with the expressed effector polypeptide or chimeric polypeptide. The method for assaying the one or more identified polypeptides for interaction with a polypeptide having at least 90% sequence identity to any one of SEQ ID NOs: 1-8 may be any method described herein (e.g., binding assays and/or measuring HR cell death, H₂O₂ accumulation, marker gene or marker metabolite expression, or any combination thereof in a host cell).

In certain embodiments, the method for identifying ASR resistance polypeptides that do not compete with CcRpp1 comprises expressing in a host cell comprising one or more R-gene candidate polypeptides a polynucleotide encoding an effector polypeptide having at least 90% sequence identity to any one of SEQ ID NOs: 10-309, or a functional fragment thereof or a chimeric polypeptide comprising an effector polypeptide having at least 90% sequence identity to any one of SEQ ID NOs: 10-309, or a functional fragment thereof, assaying the host cell for the presence of HR cell death, H₂O₂ accumulation, marker gene or marker metabolite expression, or any combination thereof, identifying R-gene candidate polypeptides that result in HR cell death, H₂O₂ accumulation, marker gene or marker metabolite expression in the presence of the effector polypeptide, expressing in a second host cell the one or more identified R-gene candidate polypeptides and an ASR26 polypeptide having at least 90% sequence identity to any one of SEQ ID NOs: 1-8, assaying the host cell for the presence of HR cell death, H₂O₂ accumulation, marker gene or marker metabolite expression, or any combination thereof, and selecting the one or more identified R-gene candidate polypeptides that do not result in HR cell death, H₂O₂ accumulation, marker gene or marker metabolite expression, or any combination thereof in the presence of the ASR26 effector polypeptide.

Further provided are methods for detecting and/or isolating the soluble form (e.g., active form) of CcRpp1 protein in a sample. In certain embodiments, the method comprises incubating a mixture, the mixture comprising an ASR26 polypeptide having at least 90% sequence identity to any one of SEQ ID NOs: 1-8 and a sample comprising a CcRpp1 protein comprising an amino acid sequence having at least 90%, 91%, 92%, 93%, 94%, 95%, 96%, 97%, 98%, or 99% or more sequence identity to SEQ ID NO: 9, isolating the ASR26 polypeptide and polypeptides bound thereto from the mixture, and detecting the presence of a CcRpp1 protein in the isolated ASR26 polypeptide and polypeptides bound thereto. In certain embodiments, the ASR26 polypeptide is operably linked to a protein tag, such as those described herein. In certain embodiments, the ASR26 polypeptide is operably linked to an MBP tag. In certain embodiments, the CcRpp1 polypeptide is operably linked to a protein tag, such as those described herein. In certain embodiments, the protein tag operably linked to the CcRpp1 polypeptide is a different protein tag from the protein tag operably linked to ASR26. The method for isolating the ASR26 polypeptide is not particularly limited and may be any method described herein or known in the art that can isolate a protein from a mixture while maintaining the protein-protein interactions, such as, for example, immunoprecipitation and pull-down assays. The method for detecting the CcRpp1 polypeptide may be any method described herein or known in the art for detecting the presence of a protein such as, for example, Western blotting, mass spectrometry and ELISA.

The following are examples of specific embodiments of some aspects of the invention. The examples are offered for illustrative purposes only and are not intended to limit the scope of the invention in any way.

### Example 1

Identification of Predicted Phakopsora pachyrhizi Effectors.

The Phakopsora pachyrhizi (causal agent of ASR) transcriptome was generated by aligning paired-end RNA-seq reads from infected soybean plants to the Phakopsora pachyrhizi genome. Transcripts which were significantly expressed in the dataset were then utilized to generate a predicted Phakopsora pachyrhizi proteome.

The resulting proteins were then assessed for effector characteristics, including presence of a signal peptide (SignalP 3.0 and Phobius), sequence-based machine learning (EffectorP 3.0), conserved domains (HMMer) and lack of a transmembrane domain (TMHMM).

### Example 2

### Identification of Candidate Avirulence (Avr) Genes for CcRpp1.

CcRpp1 is an NLR gene from pigeon pea and confers resistance to Phakopsora pachyrhizi. A biochemical approach was used to identify a CcRpp1 cognate effector. FLAG-tagged CcRpp1 protein was expressed in E. coli cells and then incubated with extracts of ASR-infected soybean leaf tissues. CcRpp1-interacting proteins were pulled down with anti-FLAG agarose beads, followed by mass spectrometry analysis. Eight putative ASR effectors were identified when searched against the predicted Phakopsora pachyrhizi effector list.

### Example 3

### Identification of ASR26 as the Cognate Effector Recognized by CcRpp1.

Recognition of pathogen effectors by NLR proteins leads to effector-triggered immunity, often culminating in a HR cell death accompanied with reactive oxygen species production. An Agrobacterium-mediated transient expression system in Nicotiana benthamiana was used to confirm the recognition between the CcRpp1 and the putative effectors. All eight ASR candidate effectors identified in Example 2 and CcRpp1 were individually cloned into a binary vector and introduced into Agrobacterium tumefaciens AGL1. Only one of the effectors, ASR26, caused HR cell death (visual) and accumulated H₂O₂ (DAB staining) when co-expressed with CcRpp1 in N. benthamiana, indicating that CcRpp1 can recognize ASR26 and trigger an immune response.

Co-immunoprecipitation was performed to examine the physical interaction between CcRpp1 and ASR26. The result from these studies confirmed that CcRpp1 interacts with ASR26 in planta.

### Example 4

### CcRpp1 Recognizes Four Known ASR26 Alleles.

Surveying ASR26 alleles with genomic sequences and/or RNA-seq data sets from thirteen P. pachyrhizi isolates resulted in the identification of a total of four ASR26 alleles. When co-expressed with CcRpp1 in N. benthamiana, all four ASR26 alleles caused HR cell death and accumulated H₂O₂, indicating that CcRpp1 can recognize all four ASR26 alleles and is expected to provide resistance against all the thirteen isolates.

### Example 5

Soluble CcRpp1 Protein, but not Insoluble CcRpp1 Protein, Physically Interacts with ASR26.

CcRpp1 protein with a His tag (CcRpp1-His) was produced using either the Bl21Gold E. coli expression system or the Sf9 insect cell expression system. CcRpp1-His protein produced from E. coli cells was insoluble, whereas the CcRpp1-His protein from insect cells was soluble. ASR26 protein fused with an MBP tag (MBP-ASR26) was produced in E. coli cells and determined to be soluble. To determine if ASR26 can interact with the soluble CcRpp1 protein, which is predicted to be the active form, in vitro pulldown assays were performed using both the soluble and insoluble versions of the expressed CcRpp1 protein as prey and the tagged ASR26 protein as bait.

For the in vitro pull-down assays, purified CcRpp1-His (prey) and MBP-ASR26 (bait) proteins were obtained. The MBP-ASR26 (bait) protein was immobilized onto anti-MBP magnetic beads, and any unattached proteins were removed through washes using MBP Column Binding/Washing Buffer. Following this, the CcRpp1-His (prey) protein was introduced to the beads bound with MBP-ASR26 and allowed to incubate for a minimum of 30 minutes. The protein complex formed was released from the beads after washing away unbound proteins. The eluted proteins were subjected to analysis using SDS-PAGE and Western blotting with anti-MBP antibodies and/or anti-His antibodies to visualize and detect the protein interactions.

These results indicate that ASR26 could be used to determine if a purified CcRpp1 protein is in a soluble form.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A nucleic acid construct comprising a polynucleotide encoding an effector polypeptide having at least 90% sequence identity to any one of SEQ ID NOs: 1-8, or a functional fragment thereof, operably linked to a heterologous regulatory element.

2. (canceled)

3. The nucleic acid construct of claim 1, wherein the heterologous regulatory element is a promoter functional in a plant cell.

4-8. (canceled)

9. A host cell comprising the nucleic acid construct of claim 1.

10. The host cell of claim 9, wherein the host cell is a bacterial cell, a fungal cell, or a plant cell.

11. (canceled)

12. The host cell of claim 10, wherein the plant cell is a plant protoplast.

13. The host cell of claim 10, wherein the plant cell further comprises a candidate resistance gene.

14-20. (canceled)

21. A method for identifying a CcRpp1-like protein, the method comprising identifying one or more polypeptides that bind to an effector polypeptide having at least 90% sequence identity to any one of SEQ ID NOs: 1-8, or a functional fragment thereof.

22. The method of claim 21, wherein the method comprises:
a. providing a population of polypeptides from an ARS resistant plant cell;
b. incubating the population of polypeptides with at least one effector polypeptide under conditions that allow for binding of the at least one effector polypeptide to one or more polypeptides of the population;
c. isolating the at least one effector polypeptide and polypeptides interacting therewith from the population; and
d. identifying the polypeptides interacting with the at least one effector polypeptide.

23. The method of claim 21, wherein the method comprises:
a. providing a population comprising a plurality of R-gene candidates;
b. incubating the population with at least one effector polypeptide under conditions that allow for binding of the at least one effector polypeptide to one or more R-gene candidates in the population;
c. isolating the at least one effector polypeptide and interacting R-gene candidates from the population; and
d. identifying the R-gene candidates interacting with the at least one effector polypeptide.

24. The method of claim 22, wherein the incubation is performed in the presence of a crosslinker.

25. The method of claim 24, wherein the crosslinker is a homobifunctional crosslinker or a photoreactive crosslinker.

26. The method of claim 21 wherein the method comprises:
a. introducing into a host cell comprising one or more candidate ASR resistance genes a nucleic acid construct comprising a polynucleotide encoding an effector polypeptide having at least 90% sequence identity to any one of SEQ ID NOs: 1-8, or a functional fragment thereof;
b. isolating the expressed effector polypeptide; and
c. identifying one or more polypeptides interacting with the expressed effector polypeptide.

27. The method of claim 26, wherein the method further comprises expressing in a host cell the at least one effector polypeptide and the one or more identified interacting polypeptides and assaying the host cell for the presence of HR cell death, H₂O₂ accumulation, marker gene or marker metabolite expression, or any combination thereof.

28. The method of claim 27, wherein the one or more identified interacting polypeptides are expressed individually in the host cell.

29. The method of claim 26, wherein the host cell is a plant cell.

30-38. (canceled)

39. A method for identifying ASR resistance polypeptides that do not compete with CcRpp1, the method comprising:
a. identifying one or more polypeptides that bind to an effector polypeptide having at least 90% sequence identity to any one of SEQ ID NOs: 10-309, or a functional fragment thereof;
b. assaying the one or more identified polypeptides for interaction with a polypeptide having at least 90% sequence identity to any one of SEQ ID NOs: 1-8; and
c. selecting the one or more polypeptides that do not interact with the polypeptide having at least 90% sequence identity to any one of SEQ ID NOS: 1-8.

40. The method of claim 39, wherein the one or more polypeptides are identified by
a. providing a population of polypeptides from an ARS resistant plant cell;
b. incubating the population of polypeptides with the effector polypeptide, or the functional fragment thereof or a chimeric polypeptide comprising the effector polypeptide or the functional fragment thereof under conditions that allow for binding of the at least one chimeric polypeptide to one or more polypeptides of the population;
c. isolating the effector polypeptide or the chimeric polypeptide and polypeptides interacting therewith from the population; and
d. identifying the one or more polypeptides.

41. The method of claim 39, wherein the one or more polypeptides are identified by
a. providing a population comprising a plurality of R-gene candidates;
b. incubating the population with the effector polypeptide, the functional fragment thereof or a chimeric polypeptide comprising the effector polypeptide or the functional fragment thereof under conditions that allow for binding of the effector polypeptide or the chimeric polypeptide to one or more R-gene candidates in the population;
c. isolating the effector polypeptide or the chimeric polypeptide and interacting R-gene candidates from the population; and
d. identifying the R-gene candidates.

42. The method of claim 41, wherein the population comprising a plurality of R-gene candidates is pre-screened to remove candidates that bind to an ASR26 allele.

43. The method of claim 39, wherein the one or more polypeptides are identified by:
a. introducing into a host cell comprising one or more candidate ASR resistance genes a nucleic acid construct comprising a polynucleotide encoding the effector polypeptide, the functional fragment thereof or a chimeric polypeptide comprising the effector polypeptide or the functional fragment thereof;
b. isolating the expressed effector polypeptide or chimeric polypeptide; and
c. identifying one or more polypeptides interacting with the expressed effector polypeptide or chimeric polypeptide.

44-46. (canceled)
